(19) United States
(12) Patent Application Publication
(10) Pub. No.: US 2025/0186589 A1
(43) Pub. Date: Jun. 12, 2025

(54) MODIFIED ALLOGENEIC CELLS AND METHODS AND COMPOSITIONS FOR THE PREPARATION THEREOF

(71) Applicant: Beam Therapeutics Inc., Cambridge, MA (US)

(72) Inventor: Colby Maldini, Cambridge, MA (US)

(73) Assignee: Beam Therapeutics Inc., Cambridge, MA (US)

(21) Appl. No.: 19/062,951

(22) Filed: Feb. 25, 2025

Related U.S. Application Data
(63) Continuation of application No. PCT/US2023/072911, filed on Aug. 25, 2023.
(60) Provisional application No. 63/373,644, filed on Aug. 26, 2022.

(51) Int. Cl. A61K 40/50; A61K 40/11; A61K 40/22; A61K 40/41; C07K 14/74

(52) U.S. Cl. CPC A61K 40/50; A61K 40/11; A61K 40/22; A61K 40/418; C07K 14/70539; C07K 2319/00

(57) ABSTRACT

The present disclosure features allogeneic modified cells (e.g., T- or NK-cells) having increased persistence, increased resistance to immune rejection, or decreased risk of eliciting a host-versus-graft reaction, or a combination thereof. Methods for producing and using the same are also provided.

### CROSS REFERENCE TO RELATED APPLICATIONS

This application is a continuation under 35 U.S.C. § 111 (a) of PCT International Patent Application No. PCT/US2023/072911, filed Aug. 25, 2023, designating the United States and published in English, which claims priority to and the benefit of U.S. Provisional Application No. 63/373,644, filed Aug. 26, 2022, the entire contents of each of which are incorporated by reference herein.

### SEQUENCE LISTING

This application contains a Sequence Listing which has been submitted electronically in XML format and is hereby incorporated by reference in its entirety. The Sequence Listing XML file, created on Aug. 7, 2023, is named 180802-046502PCT_SL.xml and is 1,154,284 bytes in size.

### BACKGROUND

Autologous and allogeneic immunotherapies are treatment approaches in which cells are administered to a subject. Autologous and allogeneic immunotherapies can be used to treat any of a variety of diseases including autoimmune diseases, graft-versus host disease (GVHD), and the like. For example, some diseases can be treated by administering to a subject in need thereof autologous or allogeneic immune effector cells capable of targeting and killing or inhibiting activity of disease-associated cells (e.g., neoplasia cells or autoantibody-producing cells) in a subject. Allogeneic cells (e.g., cells that are not recognized by a host's immune system as other) may also be useful for organ and tissue replacement and repair.

In some instances, autologous or allogeneic immunotherapies can be used to treat a disease (e.g., a cancer or autoimmune disease) by administering to a subject an immune effector cell modified to express a chimeric antigen receptor. To generate an immune cell that expresses a chimeric antigen receptor (CAR), the immune cell is first collected from the subject (autologous) or a donor separate from the subject receiving treatment (allogeneic) and genetically modified to express the chimeric antigen receptor. The resulting cell expresses the chimeric antigen receptor on its cell surface (e.g., CAR-T cell), and upon administration to the subject, the chimeric antigen receptor binds to a marker expressed by the neoplastic cell. This interaction with the marker activates the CAR-T cell, which then kills the neoplastic cell.

Autologous cell therapies face numerous challenges, including obtaining suitable biological materials from the patient, the expense of carrying out patient-specific therapies, and long manufacturing times. Allogeneic cell therapy is also subject to certain challenges, such as graft-versus-host disease (GVHD) and host rejection of CAR-T cells provide additional challenges. Currently, allogeneic T cells are susceptible to lysis by immune effector cells (e.g., alloreactive T cells or natural killer cells) in patients.

Thus, there is a significant need for techniques to reduce susceptibility of allogeneic immune effector cells and other modified immune cells to lysis by immune effector cells of an allogeneic subject (e.g., alloreactive T cells or natural killer cells).

### SUMMARY

The present disclosure features allogeneic modified cells (e.g., T- or NK-cells) having increased persistence, increased resistance to immune rejection, or decreased risk of eliciting a host-versus-graft reaction, or a combination thereof. Methods and compositions for producing and using the same are also provided. In embodiments, the methods for preparing the modified cells leverage base editing. Among other things, the present disclosure shows that allogeneic cells can be modified to be resistant to immune rejection. The present disclosure also features allogeneic modified immune cells (e.g., T- or NK-cells) having increased persistence, increased resistance to immune rejection, and/or decreased risk of eliciting a host-versus-graft reaction, and methods of producing and using such cells, for example, in the treatment of neoplasias without the disadvantages of autologous cell therapies such as long manufacturing times, or the need for an adequate supply of sufficiently healthy autologous donor cells.

In one aspect, the disclosure features a fusion polypeptide containing an HLA heavy chain polypeptide, or a functional fragment thereof, and a beta-2-microglobulin (B2M) polypeptide, or a functional fragment thereof.

In another aspect, the disclosure features a fusion polypeptide containing an amino acid sequence with at least 85% sequence identity to a polypeptide sequence listed in Table 8A.

In another aspect, the disclosure features a polynucleotide encoding the fusion polypeptide of any of any aspect of the disclosure delineated herein, or embodiments thereof.

In another aspect, the disclosure features a vector containing a polynucleotide encoding the fusion polypeptide of any aspect of the disclosure delineated herein, or embodiments thereof.

In another aspect, the disclosure features a cell containing the fusion polypeptide of any aspect of the disclosure delineated herein, or embodiments thereof.

In another aspect, the disclosure features a pharmaceutical composition containing the polynucleotide of any aspect of the disclosure delineated herein, or embodiments thereof, and a pharmaceutically acceptable excipient.

In another aspect, the disclosure features a method for preparing a modified cell. The method involves a) modifying a cell to knock-out expression of an endogenous beta-2-microglobulin (B2M) polypeptide in the cell. The method further involves b) contacting the cell with a polynucleotide encoding a fusion polypeptide containing an HLA heavy chain polypeptide, or a functional fragment thereof, and a beta-2-microglobulin (B2M) polypeptide, or a functional fragment thereof, and expressing the polypeptide in the cell.

In another aspect, the disclosure features a method for preparing a modified cell. The method involves a) modifying a cell to knock-out expression of an endogenous beta-2-microglobulin (B2M) polypeptide in the cell. The method further involves and b) contacting the cell with a polynucleotide encoding a B2M polypeptide and expressing the B2M polypeptide in the cell. Expression of the B2M polypeptide is reduced relative to an unmodified cell, and/or the B2M polypeptide contains an alteration at one or more amino acids positions that effects a reduction in binding of the B2M polypeptide to an HLA heavy chain polypeptide

In another aspect, the disclosure features a modified cell prepared according to the method of any aspect of the disclosure delineated herein, or embodiments thereof.

In another aspect, the disclosure features a pharmaceutical composition containing the modified cell of any aspect of the disclosure delineated herein, or embodiments thereof, and a pharmaceutically acceptable excipient.

In another aspect, the disclosure features a method for killing a neoplastic cell. The method involves contacting the neoplastic cell with the cell or the pharmaceutical composition of any aspect of the disclosure delineated herein, or embodiments thereof.

In another aspect, the disclosure features a method for treating a subject having a neoplasia. The method involves administering to the subject the cell or the pharmaceutical composition of any aspect of the disclosure delineated herein, or embodiments thereof.

In another aspect, the disclosure features a kit for use in the method of any aspect of the disclosure delineated herein, or embodiments thereof, where the kit contains the fusion polypeptide, polynucleotide, vector, pharmaceutical composition, and/or cell of any aspect of the disclosure delineated herein, or embodiments thereof.

In any aspect of the disclosure delineated herein, or embodiments thereof, the HLA heavy chain polypeptide and/or the B2M polypeptide contains an amino acid alteration that reduces affinity of binding to a CD8 polypeptide relative to a wild-type HLA heavy chain polypeptide and/or B2M polypeptide.

In any aspect of the disclosure delineated herein, or embodiments thereof, the HLA heavy chain polypeptide is an HLA-A, HLA-B, HLA-C, or HLA-E polypeptide. In any aspect of the disclosure delineated herein, or embodiments thereof, the HLA heavy chain polypeptide contains one or more amino acid alterations to one or more of amino acid positions 183-274. In any aspect of the disclosure delineated herein, or embodiments thereof, the HLA heavy chain polypeptide contains one or more amino acid alterations at an amino acid position selected from one or more of A73, D227, T228, and A245. In any aspect of the disclosure delineated herein, or embodiments thereof, the HLA heavy chain polypeptide contains one or more amino acid alterations selected from one or more of A73T, D227K, T228A, and A245V. In any aspect of the disclosure delineated herein, or embodiments thereof, the HLA heavy chain polypeptide contains the alterations D227K and T228A, or the alterations D227K, T228A, and A245V.

In any aspect of the disclosure delineated herein, or embodiments thereof, the B2M polypeptide contains an amino acid alteration. In any aspect of the disclosure delineated herein, or embodiments thereof, the B2M polypeptide contains an amino acid alteration at position K58. In any aspect of the disclosure delineated herein, or embodiments thereof, the B2M polypeptide contains the amino acid alteration K58E.

In any aspect of the disclosure delineated herein, or embodiments thereof, the fusion polypeptide further contains a cognate peptide (cPep). In embodiments, the cPep contains from about 8 to about 10 amino acids. In embodiments, the cPep contains 9 amino acids. In embodiments, the cPep contains an amino acid sequence selected from one or more of QYDDAVYKL (SEQ ID NO: 520), RYRPGTVAL (SEQ ID NO: 521), LSSPVTKSF (SEQ ID NO: 522), EEVHDLERKY (SEQ ID NO: 523), RLRAEAQVK (SEQ ID NO: 524), IIDKSGAAV (SEQ ID NO: 529; IV9 (AA)), IIDKSGEEV (SEQ ID NO: 530; IV9 (EE)), TIDKSGLAV (SEQ ID NO: 531; IV9 (LA)), IIDKSGSTV (SEQ ID NO: 532; IV9 (WT)), and the cPep sequences listed in Table A. In some embodiments, the inclusion of the cPep in the fusion protein causes trimerization of the fusion protein when expressed on the surface of a cell (e.g., modified immune cell).

In any aspect of the disclosure delineated herein, or embodiments thereof, the fusion polypeptide contains one or more linkers. In embodiments, the one or more linkers contain the amino acid sequence (GGGGS)ₙ (SEQ ID NO: 247). In embodiments, n is 3 or 4.

In any aspect of the disclosure delineated herein, or embodiments thereof, the fusion polypeptide further contains a signal peptide. In embodiments, the signal peptide is a B2M signal peptide. In embodiments, the signal peptide contains the amino acid sequence MSRSVALAVLALLSLSGLEA (SEQ ID NO: 525).

In any aspect of the disclosure delineated herein, or embodiments thereof, the fusion polypeptide further contains a transmembrane domain. In embodiments, the transmembrane domain is a cluster of differentiation 4 (CD4) transmembrane (CD4TM) domain. In embodiments, the CD4TM domain contains an amino acid sequence with at least about 85% sequence identity to the amino acid sequence MALIVLGGVAGLLLFIGLGIFFCVRC (SEQ ID NO: 437).

In any aspect of the disclosure delineated herein, or embodiments thereof, the HLA heavy chain polypeptide contains a sequence with at least 85% sequence identity to an amino acid sequence selected from one or more of:

HLA-A*02 (HLA-A2)
(SEQ ID NO: 441)
GSHSMRYFFTSVSRPGRGEPRFIAVGYVDDTQFVRFDSDAASQRM
EPRAPWIEQEGPEYWDGETRKVKAHSQTHRVDLGTLRGYYNQSEA
GSHTVQRMYGCDVGSDWRFLRGYHQYAYDGKDYIALKEDLRSWTA
ADMAAQTTKHKWEAAHVAEQLRAYLEGTCVEWLRRYLENGKETLQ
RTDAPKTHMTHHAVSDHEATLRCWALSFYPAEITLTWQRDGEDQT
QDTELVETRPAGDGTFQKWAAVVVPSGQEQRYTCHVQHEGLPKPL
TLRWEPSSQPTIPIVGIIAGLVLFGAVITGAVVAAVMWRRKSSDR
KGGSYSQAASSDSAQGSDVSLTACKV;
HLA-A*03
(SEQ ID NO: 442)
GSHSMRYFFTSVSRPGRGEPRFIAVGYVDDTQFVRFDSDAASQRM
EPRAPWIEQEGPEYWDQETRNVKAQSQTDRVDLGTLRGYYNQSEA
GSHTIQIMYGCDVGSDGRFLRGYRQDAYDGKDYIALNEDLRSWTA
ADMAAQITKRKWEAAHEAEQLRAYLDGTCVEWLRRYLENGKETLQ
RTDPPKTHMTHHPISDHEATLRCWALGFYPAEITLTWQRDGEDQT
QDTELVETRPAGDGTFQKWAAVVVPSGEEQRYTCHVQHEGLPKPL
TLRWELSSQPTIPIVGIIAGLVLLGAVITGAVVAAVMWRRKSSDR
KGGSYTQAASSDSAQGSDVSLTACKV;
HLA-B*57
(SEQ ID NO: 445)
GSHSMRYFYTAMSRPGRGEPRFIAVGYVDDTQFVRFDSDAASPRM
APRAPWIEQEGPEYWDGETRNMKASAQTYRENLRIALRYYNQSEA
GSHIIQVMYGCDVGPDGRLLRGHDQSAYDGKDYIALNEDLSSWTA
ADTAAQITQRKWEAARVAEQLRAYLEGLCVEWLRRYLENGKETLQ
RADPPKTHVTHHPISDHEATLRCWALGFYPAEITLTWQRDGEDQT
QDTELVETRPAGDRTFQKWAAVVVPSGEEQRYTCHVQHEGLPKPL
TLRWEPSSQSTVPIVGIVAGLAVLAVVVIGAVVAAVMCRRKSSGG
KGGSYSQAACSDSAQGSDVSLTA;
HLA-B*44
(SEQ ID NO: 446)
GSHSMRYFYTAMSRPGRGEPRFITVGYVDDTLFVRFDSDATSPRK
EPRAPWIEQEGPEYWDRETQISKTNTQTYRENLRTALRYYNQSEA
GSHIIQRMYGCDVGPDGRLLRGYDQDAYDGKDYIALNEDLSSWTA
ADTAAQITQRKWEAARVAEQDRAYLEGLCVESLRRYLENGKETLQ
RADPPKTHVTHHPISDHEVTLRCWALGFYPAEITLTWQRDGEDQT
QDTELVETRPAGDRTFQKWAAVVVPSGEEQRYTCHVQHEGLPKPL
TLRWEPSSQSTVPIVGIVAGLAVLAVVVIGAVVAAVMCRRKSSGG
KGGSYSQAACSDSAQGSDVSLTA;
HLA-C*04
(SEQ ID NO: 449)
GSHSMRYFSTSVSWPGRGEPRFIAVGYVDDTQFVRFDSDAASPRG
EPREPWVEQEGPEYWDRETQKYKRQAQADRVNLRKLRGYYNQSED
GSHTLQRMFGCDLGPDGRLLRGYNQFAYDGKDYIALNEDLRSWTA
ADTAAQITQRKWEAAREAEQRRAYLEGTCVEWLRRYLENGKETLQ
RAEHPKTHVTHHPVSDHEATLRCWALGFYPAEITLTWQWDGEDQT
QDTELVETRPAGDGTFQKWAAVVVPSGEEQRYTCHVQHEGLPEPL
TLRWKPSSQPTIPIVGIVAGLAVLAVLAVLGAMVAVVMCRRKSSG
GKGGSCSQAASSNSAQGSDESLIACKA;
HLA-C*05
(SEQ ID NO: 450)
CSHSMRYFYTAVSRPGRGEPRFIAVGYVDDTQFVQFDSDAASPRG
EPRAPWVEQEGPEYWDRETQKYKRQAQTDRVNLRKLRGYYNQSEA
GSHTLQRMYGCDLGPDGRLLRGYNQFAYDGKDYIALNEDLRSWTA
ADKAAQITQRKWEAAREAEQRRAYLEGTCVEWLRRYLENGKKTLQ
RAEHPKTHVTHHPVSDHEATLRCWALGFYPAEITLTWQRDGEDQT
QDTELVETRPAGDGTFQKWAAVVVPSGEEQRYTCHVQHEGLPEPL
TLRWGPSSQPTIPIVGIVAGLAVLAVLAVLGAVMAVVMCRRKSSG
GKGGSCSQAASSNSAQGSDESLIACKA;
and
HLA-C*07
(SEQ ID NO: 451)
CSHSMRYFDTAVSRPGRGEPRFISVGYVDDTQFVRFDSDAASPRG
EPRAPWVEQEGPEYWDRETQKYKRQAQADRVSLRNLRGYYNQSED
GSHTLQRMSGCDLGPDGRLLRGYDQSAYDGKDYIALNEDLRSWTA
ADTAAQITQRKLEAARAAEQLRAYLEGTCVEWLRRYLENGKETLQ
RAEPPKTHVTHHPLSDHEATLRCWALGFYPAEITLTWQRDGEDQT
QDTELVETRPAGDGTFQKWAAVVVPSGQEQRYTCHMQHEGLQEPL
TLSWEPSSQPTIPIMGIVAGLAVLVVLAVLGAVVTAMMCRRKSSG
GKGGSCSQAACSNSAQGSDESLITCKA.

In any aspect of the disclosure delineated herein, or embodiments thereof, the B2M polypeptide contains a sequence with at least 85% sequence identity to the amino acid sequence

(SEQ ID NO: 426)
IQRTPKIQVYSRHPAENGKSNFLNCYVSGFHPSDIEVDLLKNGERIEKV
EHSDLSFSKDWSFYLLYYTEFTPTEKDEYACRVNHVTLSQPKIVKWDRD
M.

In any aspect of the disclosure delineated herein, or embodiments thereof, the fusion polypeptide contains from N-terminus to C-terminus: A) the B2M polypeptide and the HLA heavy chain polypeptide; B) the B2M polypeptide, a linker, and the HLA heavy chain polypeptide; C) a cognate peptide (cPep), the B2M polypeptide, and the HLA heavy chain polypeptide; D) a cognate peptide (cPep), the B2M polypeptide, a linker, and the HLA heavy chain polypeptide; E) a cognate peptide (cPep), a linker, the B2M polypeptide, a linker, and the HLA heavy chain polypeptide; F) a signal peptide, the B2M polypeptide, a linker, and the HLA heavy chain polypeptide; G) a signal peptide, the B2M polypeptide and the HLA heavy chain polypeptide; H) a signal peptide, the B2M polypeptide, a linker, and the HLA heavy chain polypeptide; I) a signal peptide, a cognate peptide (cPep), the B2M polypeptide, and the HLA heavy chain polypeptide; J) a signal peptide, a cognate peptide (cPep), the B2M polypeptide, a linker, and the HLA heavy chain polypeptide; or K) a signal peptide, a cognate peptide (cPep), a linker, the B2M polypeptide, a linker, and the HLA heavy chain polypeptide.

In any aspect of the disclosure delineated herein, or embodiments thereof, the fusion polypeptide further contains a transmembrane domain C-terminal to the HLA heavy chain polypeptide.

In any aspect of the disclosure delineated herein, or embodiments thereof, the polynucleotide contains a sequence with at least 85% sequence identity to a polynucleotide sequence listed in Table 8B.

In any aspect of the disclosure delineated herein, or embodiments thereof, the vector is a viral vector or a transposon. In any aspect of the disclosure delineated herein, or embodiments thereof, the vector is a lentiviral vector.

In any aspect of the disclosure delineated herein, or embodiments thereof, the cell is an immune cell, a hepatocyte, a stem cell, an induced pluripotent stem cell, an islet cell, or a progenitor thereof.

In any aspect of the disclosure delineated herein, or embodiments thereof, the method further involves contacting the cell with a vector containing the polynucleotide.

In any aspect of the disclosure delineated herein, or embodiments thereof, modifying the cell to knock-out expression of B2M is carried out using base editing. In embodiments, the base editing involves contacting the cell with a base editor and a guide polynucleotide targeting the base editor to effect an alteration of a nucleobase of an endogenous B2M gene in the cell, thereby knocking out expression of the endogenous B2M polypeptide in the cell. In embodiments, the base editor is a cytidine deaminase base editor, an adenosine deaminase base editor, or a cytidine adenosine deaminase base editor. In embodiments, the adenosine deaminase is TadA or a TadA variant. In embodiments, the TadA variant is a TadA*8 or TadA*9. In embodiments, the cytidine deaminase is APOBEC or an APOBEC variant. In embodiments, the deaminase is TadA*8.20 In embodiments, the guide polynucleotide contains a spacer containing the nucleotide sequence CUUACCCCACUUAACUAUCU (SEQ ID NO: 537). In embodiments, the guide polynucleotide contains a scaffold containing the nucleotide sequence

(SEQ ID NO: 626)
GUUUUAGAGCUAGAAAUAGCAAGUUAAAAUAAGGCUAGUCCGUUAUCAA
CUUGAAAAAGUGGCACCGAGUCGGUGCUUUU.

In any aspect of the disclosure delineated herein, or embodiments thereof, the method further involves expressing a chimeric antigen receptor in the cell.

In any aspect of the disclosure delineated herein, or embodiments thereof, the chimeric antigen receptor targets an antigen expressed on the surface of a neoplastic cell.

In any aspect of the disclosure delineated herein, or embodiments thereof, the cell is a T cell, a natural killer cell, a hepatocyte, a stem cell, an induced pluripotent stem cell, an islet cell, or a progenitor thereof. In any aspect of the disclosure delineated herein, or embodiments thereof, the cell is a CD4+ or CD8+ cell. In any aspect of the disclosure delineated herein, or embodiments thereof, the cell is an allogeneic cell. In any aspect of the disclosure delineated herein, or embodiments thereof, the cell is suitable for use in tissue regeneration.

In any aspect of the disclosure delineated herein, or embodiments thereof, the modified cell shows increased resistance to lysis by an immune effector cell relative to a reference cell. In embodiments, lysis is reduced by at least about 60%. In embodiments, lysis is reduced by at least about 90%.

In any aspect of the disclosure delineated herein, or embodiments thereof, an alloreactive T cell contacted with the modified cell shows reduced levels of cytokine production relative to levels produced when the alloreactive T cell is contacted with a reference cell. In embodiments, granzyme B (GZMB) secreted by the alloreactive T cell is reduced by at least about 10%. In embodiments, granzyme B (GZMB) secreted by the alloreactive T cell is reduced by at least about 50%. In embodiments, INF-gamma secreted by the alloreactive T cell is reduced by at least about 10%. In embodiments, INF-gamma secreted by the alloreactive T cell is reduced by at least about 40%. In embodiments, TNF-alpha secreted by the alloreactive T cell is reduced by at least about 10%. In embodiments, TNF-alpha secreted by the alloreactive T cell is reduced by at least about 40%. In any aspect of the disclosure delineated herein, or embodiments thereof, a natural killer cell contacted with the modified cell shows reduced levels of granulation relative to levels measured when the natural killer cell is contacted with a reference cell.

In any aspect of the disclosure delineated herein, or embodiments thereof, the modified cell further contains virtually undetectable levels of HLA-A and HLA-B.

In any aspect of the disclosure delineated herein, or embodiments thereof, the B2M polypeptide is expressed in the cell under the control of a promoter. In embodiments, the promoter is an EF1a promoter.

In any aspect of the disclosure delineated herein, or embodiments thereof, the B2M polypeptide contains an alteration at amino acid position K58, W60, and/or position W95. In any aspect of the disclosure delineated herein, or embodiments thereof, the B2M polypeptide contains a K58E, W60G, and/or W95G amino acid alteration.

In any aspect of the disclosure delineated herein, or embodiments thereof, the method effects a reduction in levels of surface expression of an HLA-A, HLA-B, and/or HLA-C polypeptide on the cell relative to a reference cell. In embodiments, surface expression is reduced by at least about 40%. In embodiments, surface expression is reduced by at least about 80%.

In any aspect of the disclosure delineated herein, or embodiments thereof, expression of the B2M polypeptide is reduced by at least about 75% relative to an unmodified cell. In any aspect of the disclosure delineated herein, or embodiments thereof, expression of the B2M polypeptide is reduced by at least about 90% relative to an unmodified cell.

In any aspect of the disclosure delineated herein, or embodiments thereof, the cell expresses a chimeric antigen receptor that binds an antigen expressed on the surface of a neoplastic cell.

In any aspect of the disclosure delineated herein, or embodiments thereof, the HLA heavy chain polypeptide comprises the alterations D227K, T228A, and A245V. In any aspect of the disclosure delineated herein, or embodiments thereof, the B2M polypeptide comprises the alteration K58E. In any aspect of the disclosure delineated herein, or embodiments thereof, the B2M polypeptide comprises the alteration W60G. In any aspect of the disclosure delineated herein, or embodiments thereof, the B2M polypeptide comprises the alteration W95G. In any aspect of the disclosure delineated herein, or embodiments thereof, the B2M polypeptide comprises the alteration K58E and W60G.

In any aspect of the disclosure delineated herein, or embodiments thereof, the cell is a T cell or a natural killer (NK) cell or a progenitor thereof.

In any aspect of the disclosure delineated herein, or embodiments thereof, the HLA heavy chain polypeptide belongs to an HLA-A superfamily selected from the group consisting of A1, A2, A3, A24, and A6X. In any aspect of the disclosure delineated herein, or embodiments thereof, the HLA heavy chain polypeptide belongs to an HLA-B superfamily selected from the group consisting of B7, B8, B27, B44, B58, and B62.

In any aspect of the disclosure delineated herein, or embodiments thereof, the HLA heavy chain polypeptide is an HLA-E polypeptide and the B2M polypeptide contains an amino acid alteration that reduces affinity for CD8 binding. In embodiments, the HLA-E polypeptide is a wild-type polypeptide.

In any aspect of the disclosure delineated herein, or embodiments thereof, the fusion polypeptide further comprises an effector domain. In embodiments, the effector domain is an immunosuppressive domain. In embodiments, the effector domain is selected from one or more of a CTLA4 domain, a PDL1 domain, an additional B2M polypeptide domain, an HLA heavy chain polypeptide domain, and a CD47 domain.

In any aspect of the disclosure delineated herein, or embodiments thereof, the fusion polypeptide further comprises a tag or marker (e.g., a fluorescent protein, such as green fluorescent protein, a His tag, or a FLAG tag). In some embodiments, the fusion protein comprises a kill switch (e.g., RQR8 or ADC target).

In any aspect of the disclosure delineated herein, or embodiments thereof, the cells express two or more fusion polypeptides, where the cells expressing the two or more fusion polypeptides have improved resistance to lysis by natural killer cell (NK) cells having two or more different KIR types relative to cells expressing only one of the fusion polypeptides. In any aspect of the disclosure delineated herein, or embodiments thereof, the cells express two or more fusion polypeptides, where natural killer (NK) cells having two or more different KIR types contacted with the cells expressing the two or more fusion polypeptides show reduced granulation levels relative to the natural killer (NK) contacted with cells expressing only one of the fusion polypeptides. In embodiments, each of the two or more fusion polypeptides contain a different HLA polypeptide. In embodiments, each of the different HLA polypeptides inhibits natural killer cells having a different KIR type. In some embodiments, the modified cells express at least two fusion proteins, wherein each fusion protein targets KIR2DL1, KIR2DL2, KIR2DL3, or KIR3DL1. In some embodiments, one or more of the fusion proteins targets at least two KIRS (e.g., two or more KIRS selected from KIR2DL1, KIR2DL2, KIR2DL3, and KIR3DL1).

In any aspect of the disclosure delineated herein, or embodiments thereof, the methods further involve base editing the cells to knock-out expression of HLA-A and/or HLA-B.

In any aspect of the disclosure delineated herein, or embodiments thereof, the polynucleotide is inserted into the genome of a cell using homology-directed repair (HDR). In embodiments, the homology-directed repair involves the use of a nucleic acid programmable DNA binding protein. In some embodiments, the nucleic acid programmable DNA binding protein is a Cas12b polypeptide.

In any aspect provided herein, or embodiments thereof, the method is not a process for modifying the germline genetic identity of human beings.

### Definitions

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “adenine” or “9H-Purin-6-amine” is meant a purine nucleobase with the molecular formula C₅H₅N₅, having the structure

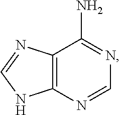

and corresponding to CAS No. 73-24-5.

By “adenosine” or “4-Amino-1-[(2R,3R,4S,5R)-3,4-dihydroxy-5-(hydroxymethyl)oxolan-2-yl]pyrimidin-2 (1H)-one” is meant an adenine molecule attached to a ribose sugar via a glycosidic bond, having the structure

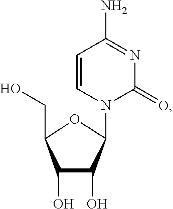

and corresponding to CAS No. 65-46-3. Its molecular formula is C₁₀H₁₃N₅O₄.

By “adenosine deaminase” or “adenine deaminase” is meant a polypeptide or fragment thereof capable of catalyzing the hydrolytic deamination of adenine or adenosine. In some embodiments, the deaminase or deaminase domain is an adenosine deaminase catalyzing the hydrolytic deamination of adenosine to inosine or deoxy adenosine to deoxyinosine. In some embodiments, the adenosine deaminase catalyzes the hydrolytic deamination of adenine or adenosine in deoxyribonucleic acid (DNA). The adenosine deaminases (e.g. engineered adenosine deaminases, evolved adenosine deaminases) provided herein may be from any organism (e.g., eukaryotic, prokaryotic), including but not limited to algae, bacteria, fungi, plants, invertebrates (e.g., insects), and vertebrates (e.g., amphibians, mammals). In some embodiments, the adenosine deaminase is an adenosine deaminase variant with one or more alterations and is capable of deaminating both adenine and cytosine in a target polynucleotide (e.g., DNA, RNA) and may be referred to as a “dual deaminase”. Non-limiting examples of dual deaminases include those described in PCT/US22/22050. In some embodiments, the target polynucleotide is single or double stranded. In some embodiments, the adenosine deaminase variant is capable of deaminating both adenine and cytosine in DNA. In some embodiments, the adenosine deaminase variant is capable of deaminating both adenine and cytosine in single-stranded DNA. In some embodiments, the adenosine deaminase variant is capable of deaminating both adenine and cytosine in RNA. In embodiments, the adenosine deaminase variant is selected from those described in PCT/US2020/018192, PCT/US2020/049975, PCT/US2017/045381, and PCT/US2020/028568, the full contents of which are each incorporated herein by reference in their entireties for all purposes.

By “adenosine deaminase activity” is meant catalyzing the deamination of adenine or adenosine to guanine in a polynucleotide. In some embodiments, an adenosine deaminase variant as provided herein maintains adenosine deaminase activity (e.g., at least about 30%, 40%, 50%, 60%, 70%, 80%, 90% or more of the activity of a reference adenosine deaminase (e.g., TadA*8.20 or TadA*8.19)).

By “Adenosine Base Editor (ABE)” is meant a base editor comprising an adenosine deaminase.

By “Adenosine Base Editor (ABE) polynucleotide” is meant a polynucleotide encoding an ABE.

By “Adenosine Base Editor 8 (ABE8) polypeptide” or “ABE8” is meant a base editor as defined herein comprising an adenosine deaminase or adenosine deaminase variant comprising one or more of the alterations listed in Table 5B, one of the combinations of alterations listed in Table 5B, or an alteration at one or more of the amino acid positions listed in Table 5B, such alterations are relative to the following reference sequence: MSEVEFSHEYWMRHALTLAKRARDEREVPVGAVLVLNNRVIGEGWNRAIGLHDPTAHAEIMALR QGGLVMQNYRLIDATLYVTFEPCVMCAGAMIHSRIGRVVFGVRNAKTGAAGSLMDVLHYPGMNH RVEITEGILADECAALLCYFFRMPRQVFNAQKKAQSSTD (SEQ ID NO: 1), or a corresponding position in another adenosine deaminase. In embodiments, ABE8 comprises alterations at amino acids 82 and/or 166 of SEQ ID NO: 1 In some embodiments, ABE8 comprises further alterations, as described herein, relative to the reference sequence.

By “Adenosine Base Editor 8 (ABE8) polynucleotide” is meant a polynucleotide encoding an ABE8 polypeptide.

“Allogeneic,” as used herein, refers to cells that are genetically dissimilar and immunologically incompatible.

“Administering” is referred to herein as providing one or more compositions described herein to a patient or a subject.

By “agent” is meant any cellular therapeutic, small molecule chemical compound, antibody, nucleic acid molecule, or polypeptide, or functional fragments thereof. In an embodiment, the agent is an immune effector cell (e.g., T cell, NK cell) expressing B2M fused to HLA-A, -B, or -C. In an embodiment, the agent is an immune effector cell (e.g., T cell, NK cell) with endogenous B2M knockout and expressing a heterologous B2M transgene.

By “alteration” is meant a change in the level, structure, or activity of an analyte, gene or polypeptide as detected by standard art known methods such as those described herein. As used herein, an alteration includes a change (e.g. increase or reduction) in expression levels. In embodiments, the increase or reduction in expression levels is by 10%, 25%, 40%, 50% or greater. In some embodiments, an alteration includes an insertion, deletion, or substitution of a nucleobase or amino acid (by, e.g., genetic engineering).

By “ameliorate” is meant reduce, suppress, attenuate, diminish, arrest, or stabilize the development or progression of a disease.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

“Autologous,” as used herein, refers to cells from the same subject.

By “base editor (BE),” or “nucleobase editor polypeptide (NBE)” is meant an agent that binds a polynucleotide and has nucleobase modifying activity. In various embodiments, the base editor comprises a nucleobase modifying polypeptide (e.g., a deaminase) and a polynucleotide programmable nucleotide binding domain (e.g., Cas9 or Cpf1) in conjunction with a guide polynucleotide (e.g., guide RNA (gRNA)). Representative nucleic acid and protein sequences of base editors include those sequences having about or at least about 85% sequence identity to any base editor sequence provided in the sequence listing, such as those corresponding to SEQ ID NOs: 2-11.

By “BE4 cytidine deaminase (BE4) polypeptide,” is meant a base editor comprising a nucleic acid programmable DNA binding protein (napDNAbp) domain, a cytidine deaminase domain, and two uracil glycosylase inhibitor domains (UGIs). In embodiments, the napDNAbp is a Cas9n (D10A) polypeptide. Non-limiting examples of cytidine deaminase domains include rAPOBEC, ppAPOBEC, RrA3F, AmAPOBEC1, and SsAPOBEC3B.

By “BE4 cytidine deaminase (BE4) polynucleotide,” is meant a polynucleotide encoding a BE4 polypeptide.

By “base editing activity” is meant acting to chemically alter a base within a polynucleotide. In one embodiment, a first base is converted to a second base. In one embodiment, the base editing activity is cytidine deaminase activity, e.g., converting target C·G to T·A. In another embodiment, the base editing activity is adenosine or adenine deaminase activity, e.g., converting A·T to G·C.

By “beta-2 microglobulin (B2M; B2M) polypeptide” is meant a protein having at least about 85% amino acid sequence identity to one or more of the exemplary B2M polypeptide sequences provided below, or a functional fragment thereof having immunomodulatory activity. In embodiments, the B2M polypeptide contains a modification at one or more of positions selected from K58, W60, and W95 (e.g., a K58E, W60G, and/or W95G alteration) or a corresponding position(s), where the positions are numbered relative to the “B2M Reference Sequence” provided below. Exemplary human B2M polypeptide sequences are provided below and include the sequence corresponding to UniProt Accession No. P61769, the “B2M Reference Sequence,” and the B2M (K58E), B2M (K58E/W60G), B2M (W60G), and B2M (W95G) amino acid sequences.

>sp|P61769|B2MG_HUMAN Beta-2-microglobulin
OS = Homo sapiens OX = 9606 GN = B2M
PE = 1 SV = 1
(SEQ ID NO: 425)
MSRSVALAVLALLSLSGLEAIQRTPKIQVYSRHPAENGKSNFLNCYVSGFHPSDIEVDLLKNGE
RIEKVEHSDLSFSKDWSFYLLYYTEFTPTEKDEYACRVNHVTLSQPKIVKWDRDM.
>B2M Reference Sequence
(SEQ ID NO: 426)
IQRTPKIQVYSRHPAENGKSNFLNCYVSGFHPSDIEVDLLKNGERIEKVEHSDLSFSKDWSFYL
LYYTEFTPTEKDEYACRVNHVTLSQPKIVKWDRDM.
>B2M (K58E) amino acid sequence
(SEQ ID NO: 427)
MSRSVALAVLALLSLSGLEAIQRTPKIQVYSRHPAENGKSNFLNCYVSGFHPSDIEVDLLKNGE
RIEKVEHSDLSFSEDWSFYLLYYTEFTPTEKDEYACRVNHVTLSQPKIVKWDRDM.
>B2M (K58E/W60G) amino acid sequence
(SEQ ID NO: 428)
MSRSVALAVLALLSLSGLEAIQRTPKIQVYSRHPAENGKSNFLNCYVSGFHPSDIEVDLLKNGE
RIEKVEHSDLSFSKDGSFYLLYYTEFTPTEKDEYACRVNHVTLSQPKIVKWDRDM.
>B2M (W60G) amino acid sequence
(SEQ ID NO: 429)
MSRSVALAVLALLSLSGLEAIQRTPKIQVYSRHPAENGKSNFLNCYVSGFHPSDIEVDLLKNGE
RIEKVEHSDLSFSEDGSFYLLYYTEFTPTEKDEYACRVNHVTLSQPKIVKWDRDM.
>B2M (W95G) amino acid sequence
(SEQ ID NO: 430)
MSRSVALAVLALLSLSGLEAIQRTPKIQVYSRHPAENGKSNFLNCYVSGFHPSDIEVDLLKNGE
RIEKVEHSDLSFSKDWSFYLLYYTEFTPTEKDEYACRVNHVTLSQPKIVKGDRDM.

By “beta-2-microglobulin (B2M; B2M) polynucleotide” is meant a nucleic acid molecule encoding an β2M polypeptide, as well as the introns, exons, 3′ untranslated regions, 5′ untranslated regions, and regulatory sequences associated with its expression, or fragments thereof. The beta-2-microglobulin gene encodes a serum protein associated with the major histocompatibility complex. B2M is involved in non-self recognition by host CD8⁺ T cells.

Exemplary β2M polynucleotide sequences are provided below and include Genbank Accession No. DQ217933.1 and the B2M (WT), B2M (K58E), B2M (K58E/W60G), and B2M (W60G) nucleotide sequences.

>DQ217933.1 Homo sapiens beta-2-microglobin (β2M) gene, complete cds
(SEQ ID NO: 431)
CATGTCATAAATGGTAAGTCCAAGAAAAATACAGGTATTCCCCCCCAAAGAAAACTGTAAAATC
GACTTTTTTCTATCTGTACTGTTTTTTATTGGTTTTTAAATTGGTTTTCCAAGTGAGTAAATCA
GAATCTATCTGTAATGGATTTTAAATTTAGTGTTTCTCTGTGATGTAGTAAACAAGAAACTAGA
GGCAAAAATAGCCCTGTCCCTTGCTAAACTTCTAAGGCACTTTTCTAGTACAACTCAACACTAA
CATTTCAGGCCTTTAGTGCCTTATATGAGTTTTTAAAAGGGGGAAAAGGGAGGGAGCAAGAGTG
TCTTAACTCATACATTTAGGCATAACAATTATTCTCATATTTTAGTTATTGAGAGGGCTGGTAG
AAAAACTAGGTAAATAATATTAATAATTATAGCGCTTATTAAACACTACAGAACACTTACTATG
TACCAGGCATTGTGGGAGGCTCTCTCTTGTGCATTATCTCATTTCATTAGGTCCATGGAGAGTA
TTGCATTTTCTTAGTTTAGGCATGGCCTCCACAATAAAGATTATCAAAAGCCTAAAAATATGTA
AAAGAAACCTAGAAGTTATTTGTTGTGCTCCTTGGGGAAGCTAGGCAAATCCTTTCAACTGAAA
ACCATGGTGACTTCCAAGATCTCTGCCCCTCCCCATCGCCATGGTCCACTTCCTCTTCTCACTG
TTCCTCTTAGAAAAGATCTGTGGACTCCACCACCACGAAATGGCGGCACCTTATTTATGGTCAC
TTTAGAGGGTAGGTTTTCTTAATGGGTCTGCCTGTCATGTTTAACGTCCTTGGCTGGGTCCAAG
GCAGATGCAGTCCAAACTCTCACTAAAATTGCCGAGCCCTTTGTCTTCCAGTGTCTAAAATATT
AATGTCAATGGAATCAGGCCAGAGTTTGAATTCTAGTCTCTTAGCCTTTGTTTCCCCTGTCCAT
AAAATGAATGGGGGTAATTCTTTCCTCCTACAGTTTATTTATATATTCACTAATTCATTCATTC
ATCCATCCATTCGTTCATTCGGTTTACTGAGTACCTACTATGTGCCAGCCCCTGTTCTAGGGTG
GAAACTAAGAGAATGATGTACCTAGAGGGCGCTGGAAGCTCTAAAGCCCTAGCAGTTACTGCTT
TTACTATTAGTGGTCGTTTTTTTCTCCCCCCCGCCCCCCGACAAATCAACAGAACAAAGAAAAT
TACCTAAACAGCAAGGACATAGGGAGGAACTTCTTGGCACAGAACTTTCCAAACACTTTTTCCT
GAAGGGATACAAGAAGCAAGAAAGGTACTCTTTCACTAGGACCTTCTCTGAGCTGTCCTCAGGA
TGCTTTTGGGACTATTTTTCTTACCCAGAGAATGGAGAAACCCTGCAGGGAATTCCCAAGCTGT
AGTTATAAACAGAAGTTCTCCTTCTGCTAGGTAGCATTCAAAGATCTTAATCTTCTGGGTTTCC
GTTTTCTCGAATGAAAAATGCAGGTCCGAGCAGTTAACTGGCTGGGGCACCATTAGCAAGTCAC
TTAGCATCTCTGGGGCCAGTCTGCAAAGCGAGGGGGCAGCCTTAATGTGCCTCCAGCCTGAAGT
CCTAGAATGAGCGCCCGGTGTCCCAAGCTGGGGCGCGCACCCCAGATCGGAGGGCGCCGATGTA
CAGACAGCAAACTCACCCAGTCTAGTGCATGCCTTCTTAAACATCACGAGACTCTAAGAAAAGG
AAACTGAAAACGGGAAAGTCCCTCTCTCTAACCTGGCACTGCGTCGCTGGCTTGGAGACAGGTG
ACGGTCCCTGCGGGCCTTGTCCTGATTGGCTGGGCACGCGTTTAATATAAGTGGAGGCGTCGCG
CTGGCGGGCATTCCTGAAGCTGACAGCATTCGGGCCGAGATGTCTCGCTCCGTGGCCTTAGCTG
TGCTCGCGCTACTCTCTCTTTCTGGCCTGGAGGCTATCCAGCGTGAGTCTCTCCTACCCTCCCG
CTCTGGTCCTTCCTCTCCCGCTCTGCACCCTCTGTGGCCCTCGCTGTGCTCTCTCGCTCCGTGA
CTTCCCTTCTCCAAGTTCTCCTTGGTGGCCCGCCGTGGGGCTAGTCCAGGGCTGGATCTCGGGG
AAGCGGCGGGGTGGCCTGGGAGTGGGGAAGGGGGTGCGCACCCGGGACGCGCGCTACTTGCCCC
TTTCGGCGGGGAGCAGGGGAGACCTTTGGCCTACGGCGACGGGAGGGTCGGGACAAAGTTTAGG
GCGTCGATAAGCGTCAGAGCGCCGAGGTTGGGGGAGGGTTTCTCTTCCGCTCTTTCGCGGGGCC
TCTGGCTCCCCCAGCGCAGCTGGAGTGGGGGACGGGTAGGCTCGTCCCAAAGGCGCGGCGCTGA
GGTTTGTGAACGCGTGGAGGGGCGCTTGGGGTCTGGGGGAGGCGTCGCCCGGGTAAGCCTGTCT
GCTGCGGCTCTGCTTCCCTTAGACTGGAGAGCTGTGGACTTCGTCTAGGCGCCCGCTAAGTTCG
CATGTCCTAGCACCTCTGGGTCTATGTGGGGCCACACCGTGGGGAGGAAACAGCACGCGACGTT
TGTAGAATGCTTGGCTGTGATACAAAGCGGTTTCGAATAATTAACTTATTTGTTCCCATCACAT
GTCACTTTTAAAAAATTATAAGAACTACCCGTTATTGACATCTTTCTGTGTGCCAAGGACTTTA
TGTGCTTTGCGTCATTTAATTTTGAAAACAGTTATCTTCCGCCATAGATAACTACTATGGTTAT
CTTCTGCCTCTCACAGATGAAGAAACTAAGGCACCGAGATTTTAAGAAACTTAATTACACAGGG
GATAAATGGCAGCAATCGAGATTGAAGTCAAGCCTAACCAGGGCTTTTGCGGGAGCGCATGCCT
TTTGGCTGTAATTCGTGCATTTTTTTTTAAGAAAAACGCCTGCCTTCTGCGTGAGATTCTCCAG
AGCAAACTGGGCGGCATGGGCCCTGTGGTCTTTTCGTACAGAGGGCTTCCTCTTTGGCTCTTTG
CCTGGTTGTTTCCAAGATGTACTGTGCCTCTTACTTTCGGTTTTGAAAACATGAGGGGGTTGGG
CGTGGTAGCTTACGCCTGTAATCCCAGCACTTAGGGAGGCCGAGGCGGGAGGATGGCTTGAGGT
CCGTAGTTGAGACCAGCCTGGCCAACATGGTGAAGCCTGGTCTCTACAAAAAATAATAACAAAA
ATTAGCCGGGTGTGGTGGCTCGTGCCTGTGGTCCCAGCTGCTCCGGTGGCTGAGGCGGGAGGAT
CTCTTGAGCTTAGGCTTTTGAGCTATCATGGCGCCAGTGCACTCCAGCGTGGGCAACAGAGCGA
GACCCTGTCTCTCAAAAAAGAAAAAAAAAAAAAAAGAAAGAGAAAAGAAAAGAAAGAAAGAAGT
GAAGGTTTGTCAGTCAGGGGAGCTGTAAAACCATTAATAAAGATAATCCAAGATGGTTACCAAG
ACTGTTGAGGACGCCAGAGATCTTGAGCACTTTCTAAGTACCTGGCAATACACTAAGCGCGCTC
ACCTTTTCCTCTGGCAAAACATGATCGAAAGCAGAATGTTTTGATCATGAGAAAATTGCATTTA
ATTTGAATACAATTTATTTACAACATAAAGGATAATGTATATATCACCACCATTACTGGTATTT
GCTGGTTATGTTAGATGTCATTTTAAAAAATAACAATCTGATATTTAAAAAAAAATCTTATTTT
GAAAATTTCCAAAGTAATACATGCCATGCATAGACCATTTCTGGAAGATACCACAAGAAACATG
TAATGATGATTGCCTCTGAAGGTCTATTTTCCTCCTCTGACCTGTGTGTGGGTTTTGTTTTTGT
TTTACTGTGGGCATAAATTAATTTTTCAGTTAAGTTTTGGAAGCTTAAATAACTCTCCAAAAGT
CATAAAGCCAGTAACTGGTTGAGCCCAAATTCAAACCCAGCCTGTCTGATACTTGTCCTCTTCT
TAGAAAAGATTACAGTGATGCTCTCACAAAATCTTGCCGCCTTCCCTCAAACAGAGAGTTCCAG
GCAGGATGAATCTGTGCTCTGATCCCTGAGGCATTTAATATGTTCTTATTATTAGAAGCTCAGA
TGCAAAGAGCTCTCTTAGCTTTTAATGTTATGAAAAAAATCAGGTCTTCATTAGATTCCCCAAT
CCACCTCTTGATGGGGCTAGTAGCCTTTCCTTAATGATAGGGTGTTTCTAGAGAGATATATCTG
GTCAAGGTGGCCTGGTACTCCTCCTTCTCCCCACAGCCTCCCAGACAAGGAGGAGTAGCTGCCT
TTTAGTGATCATGTACCCTGAATATAAGTGTATTTAAAAGAATTTTATACACATATATTTAGTG
TCAATCTGTATATTTAGTAGCACTAACACTTCTCTTCATTTTCAATGAAAAATATAGAGTTTAT
AATATTTTCTTCCCACTTCCCCATGGATGGTCTAGTCATGCCTCTCATTTTGGAAAGTACTGTT
TCTGAAACATTAGGCAATATATTCCCAACCTGGCTAGTTTACAGCAATCACCTGTGGATGCTAA
TTAAAACGCAAATCCCACTGTCACATGCATTACTCCATTTGATCATAATGGAAAGTATGTTCTG
TCCCATTTGCCATAGTCCTCACCTATCCCTGTTGTATTTTATCGGGTCCAACTCAACCATTTAA
GGTATTTGCCAGCTCTTGTATGCATTTAGGTTTTGTTTCTTTGTTTTTTAGCTCATGAAATTAG
GTACAAAGTCAGAGAGGGGTCTGGCATATAAAACCTCAGCAGAAATAAAGAGGTTTTGTTGTTT
GGTAAGAACATACCTTGGGTTGGTTGGGCACGGTGGCTCGTGCCTGTAATCCCAACACTTTGGG
AGGCCAAGGCAGGCTGATCACTTGAAGTTGGGAGTTCAAGACCAGCCTGGCCAACATGGTGAAA
TCCCGTCTCTACTGAAAATACAAAAATTAACCAGGCATGGTGGTGTGTGCCTGTAGTCCCAGGA
ATCACTTGAACCCAGGAGGCGGAGGTTGCAGTGAGCTGAGATCTCACCACTGCACACTGCACTC
CAGCCTGGGCAATGGAATGAGATTCCATCCCAAAAAATAAAAAAATAAAAAAATAAAGAACATA
CCTTGGGTTGATCCACTTAGGAACCTCAGATAATAACATCTGCCACGTATAGAGCAATTGCTAT
GTCCCAGGCACTCTACTAGACACTTCATACAGTTTAGAAAATCAGATGGGTGTAGATCAAGGCA
GGAGCAGGAACCAAAAAGAAAGGCATAAACATAAGAAAAAAAATGGAAGGGGTGGAAACAGAGT
ACAATAACATGAGTAATTTGATGGGGGCTATTATGAACTGAGAAATGAACTTTGAAAAGTATCT
TGGGGCCAAATCATGTAGACTCTTGAGTGATGTGTTAAGGAATGCTATGAGTGCTGAGAGGGCA
TCAGAAGTCCTTGAGAGCCTCCAGAGAAAGGCTCTTAAAAATGCAGCGCAATCTCCAGTGACAG
AAGATACTGCTAGAAATCTGCTAGAAAAAAAACAAAAAAGGCATGTATAGAGGAATTATGAGGG
AAAGATACCAAGTCACGGTTTATTCTTCAAAATGGAGGTGGCTTGTTGGGAAGGTGGAAGCTCA
TTTGGCCAGAGTGGAAATGGAATTGGGAGAAATCGATGACCAAATGTAAACACTTGGTGCCTGA
TATAGCTTGACACCAAGTTAGCCCCAAGTGAAATACCCTGGCAATATTAATGTGTCTTTTCCCG
ATATTCCTCAGGTACTCCAAAGATTCAGGTTTACTCACGTCATCCAGCAGAGAATGGAAAGTCA
AATTTCCTGAATTGCTATGTGTCTGGGTTTCATCCATCCGACATTGAAGTTGACTTACTGAAGA
ATGGAGAGAGAATTGAAAAAGTGGAGCATTCAGACTTGTCTTTCAGCAAGGACTGGTCTTTCTA
TCTCTTGTACTACACTGAATTCACCCCCACTGAAAAAGATGAGTATGCCTGCCGTGTGAACCAT
GTGACTTTGTCACAGCCCAAGATAGTTAAGTGGGGTAAGTCTTACATTCTTTTGTAAGCTGCTG
AAAGTTGTGTATGAGTAGTCATATCATAAAGCTGCTTTGATATAAAAAAGGTCTATGGCCATAC
TACCCTGAATGAGTCCCATCCCATCTGATATAAACAATCTGCATATTGGGATTGTCAGGGAATG
TTCTTAAAGATCAGATTAGTGGCACCTGCTGAGATACTGATGCACAGCATGGTTTCTGAACCAG
TAGTTTCCCTGCAGTTGAGCAGGGAGCAGCAGCAGCACTTGCACAAATACATATACACTCTTAA
CACTTCTTACCTACTGGCTTCCTCTAGCTTTTGTGGCAGCTTCAGGTATATTTAGCACTGAACG
AACATCTCAAGAAGGTATAGGCCTTTGTTTGTAAGTCCTGCTGTCCTAGCATCCTATAATCCTG
GACTTCTCCAGTACTTTCTGGCTGGATTGGTATCTGAGGCTAGTAGGAAGGGCTTGTTCCTGCT
GGGTAGCTCTAAACAATGTATTCATGGGTAGGAACAGCAGCCTATTCTGCCAGCCTTATTTCTA
ACCATTTTAGACATTTGTTAGTACATGGTATTTTAAAAGTAAAACTTAATGTCTTCCTTTTTTT
TCTCCACTGTCTTTTTCATAGATCGAGACATGTAAGCAGCATCATGGAGGTAAGTTTTTGACCT
TGAGAAAATGTTTTTGTTTCACTGTCCTGAGGACTATTTATAGACAGCTCTAACATGATAACCC
TCACTATGTGGAGAACATTGACAGAGTAACATTTTAGCAGGGAAAGAAGAATCCTACAGGGTCA
TGTTCCCTTCTCCTGTGGAGTGGCATGAAGAAGGTGTATGGCCCCAGGTATGGCCATATTACTG
ACCCTCTACAGAGAGGGCAAAGGAACTGCCAGTATGGTATTGCAGGATAAAGGCAGGTGGTTAC
CCACATTACCTGCAAGGCTTTGATCTTTCTTCTGCCATTTCCACATTGGACATCTCTGCTGAGG
AGAGAAAATGAACCACTCTTTTCCTTTGTATAATGTTGTTTTATTCTTCAGACAGAAGAGAGGA
GTTATACAGCTCTGCAGACATCCCATTCCTGTATGGGGACTGTGTTTGCCTCTTAGAGGTTCCC
AGGCCACTAGAGGAGATAAAGGGAAACAGATTGTTATAACTTGATATAATGATACTATAATAGA
TGTAACTACAAGGAGCTCCAGAAGCAAGAGAGAGGGAGGAACTTGGACTTCTCTGCATCTTTAG
TTGGAGTCCAAAGGCTTTTCAATGAAATTCTACTGCCCAGGGTACATTGATGCTGAAACCCCAT
TCAAATCTCCTGTTATATTCTAGAACAGGGAATTGATTTGGGAGAGCATCAGGAAGGTGGATGA
TCTGCCCAGTCACACTGTTAGTAAATTGTAGAGCCAGGACCTGAACTCTAATATAGTCATGTGT
TACTTAATGACGGGGACATGTTCTGAGAAATGCTTACACAAACCTAGGTGTTGTAGCCTACTAC
ACGCATAGGCTACATGGTATAGCCTATTGCTCCTAGACTACAAACCTGTACAGCCTGTTACTGT
ACTGAATACTGTGGGCAGTTGTAACACAATGGTAAGTATTTGTGTATCTAAACATAGAAGTTGC
AGTAAAAATATGCTATTTTAATCTTATGAGACCACTGTCATATATACAGTCCATCATTGACCAA
AACATCATATCAGCATTTTTTCTTCTAAGATTTTGGGAGCACCAAAGGGATACACTAACAGGAT
ATACTCTTTATAATGGGTTTGGAGAACTGTCTGCAGCTACTTCTTTTAAAAAGGTGATCTACAC
AGTAGAAATTAGACAAGTTTGGTAATGAGATCTGCAATCCAAATAAAATAAATTCATTGCTAAC
CTTTTTCTTTTCTTTTCAGGTTTGAAGATGCCGCATTTGGATTGGATGAATTCCAAATTCTGCT
TGCTTGCTTTTTAATATTGATATGCTTATACACTTACACTTTATGCACAAAATGTAGGGTTATA
ATAATGTTAACATGGACATGATCTTCTTTATAATTCTACTTTGAGTGCTGTCTCCATGTTTGAT
GTATCTGAGCAGGTTGCTCCACAGGTAGCTCTAGGAGGGCTGGCAACTTAGAGGTGGGGAGCAG
AGAATTCTCTTATCCAACATCAACATCTTGGTCAGATTTGAACTCTTCAATCTCTTGCACTCAA
AGCTTGTTAAGATAGTTAAGCGTGCATAAGTTAACTTCCAATTTACATACTCTGCTTAGAATTT
GGGGGAAAATTTAGAAATATAATTGACAGGATTATTGGAAATTTGTTATAATGAATGAAACATT
TTGTCATATAAGATTCATATTTACTTCTTATACATTTGATAAAGTAAGGCATGGTTGTGGTTAA
TCTGGTTTATTTTTGTTCCACAAGTTAAATAAATCATAAAACTTGATGTGTTATCTCTTATATC
TCACTCCCACTATTACCCCTTTATTTTCAAACAGGGAAACAGTCTTCAAGTTCCACTTGGTAAA
AAATGTGAACCCCTTGTATATAGAGTTTGGCTCACAGTGTAAAGGGCCTCAGTGATTCACATTT
TCCAGATTAGGAATCTGATGCTCAAAGAAGTTAAATGGCATAGTTGGGGTGACACAGCTGTCTA
GTGGGAGGCCAGCCTTCTATATTTTAGCCAGCGTTCTTTCCTGCGGGCCAGGTCATGAGGAGTA
TGCAGACTCTAAGAGGGAGCAAAAGTATCTGAAGGATTTAATATTTTAGCAAGGAATAGATATA
CAATCATCCCTTGGTCTCCCTGGGGGATTGGTTTCAGGACCCCTTCTTGGACACCAAATCTATG
GATATTTAAGTCCCTTCTATAAAATGGTATAGTATTTGCATATAACCTATCCACATCCTCCTGT
ATACTTTAAATCATTTCTAGATTACTTGTAATACCTAATACAATGTAAATGCTATGCAAATAGT
TGTTATTGTTTAAGGAATAATGACAAGAAAAAAAAGTCTGTACATGCTCAGTAAAGACACAACC
ATCCCTTTTTTTCCCCAGTGTTTTTGATCCATGGTTTGCTGAATCCACAGATGTGGAGCCCCTG
GATACGGAAGGCCCGCTGTACTTTGAATGACAAATAACAGATTTAAA.
>B2M (WT) nucleotide sequence
(SEQ ID NO: 432)
ATGAGCCGGAGCGTGGCTCTGGCCGTGCTGGCCCTATTGTCTCTGAGCGGACTGGAAGCCATCC
AGAGAACCCCAAAGATCCAAGTGTACAGCAGACACCCTGCCGAGAACGGCAAGAGCAATTTCCT
GAACTGCTACGTGTCCGGCTTTCACCCCAGCGACATCGAGGTGGATCTGCTGAAGAACGGCGAG
AGAATCGAGAAGGTCGAACACAGCGATCTGAGCTTCAGCAAGGATTGGTCCTTCTACCTGCTGT
ACTACACCGAGTTCACCCCTACAGAGAAGGACGAGTACGCCTGCAGAGTGAACCACGTGACCCT
GAGCCAGCCTAAGATCGTGAAGTGGGATAGAGATATG.
>B2M (K58E) nucleotide sequence
(SEQ ID NO: 433)
ATGAGCCGGAGCGTGGCTCTGGCCGTGCTGGCCCTATTGTCTCTGAGCGGACTGGAAGCCATCC
AGAGAACCCCAAAGATCCAAGTGTACAGCAGACACCCTGCCGAGAACGGCAAGAGCAATTTCCT
GAACTGCTACGTGTCCGGCTTTCACCCCAGCGACATCGAGGTGGATCTGCTGAAGAACGGCGAG
AGAATCGAGAAGGTCGAACACAGCGATCTGAGCTTCAGCGAAGATTGGTCCTTCTACCTGCTGT
ACTACACCGAGTTCACCCCTACAGAGAAGGACGAGTACGCCTGCAGAGTGAACCACGTGACCCT
GAGCCAGCCTAAGATCGTGAAGTGGGATAGAGATATG.
>B2M (K58E/W60G) nucleotide sequence
(SEQ ID NO: 434)
ATGAGCCGGAGCGTGGCTCTGGCCGTGCTGGCCCTATTGTCTCTGAGCGGACTGGAAGCCATCC
AGAGAACCCCAAAGATCCAAGTGTACAGCAGACACCCTGCCGAGAACGGCAAGAGCAATTTCCT
GAACTGCTACGTGTCCGGCTTTCACCCCAGCGACATCGAGGTGGATCTGCTGAAGAACGGCGAG
AGAATCGAGAAGGTCGAACACAGCGATCTGAGCTTCAGCAAGGATGGATCCTTCTACCTGCTGT
ACTACACCGAGTTCACCCCTACAGAGAAGGACGAGTACGCCTGCAGAGTGAACCACGTGACCCT
GAGCCAGCCTAAGATCGTGAAGTGGGATAGAGATATG.
>B2M (W60G) nucleotide sequence
(SEQ ID NO: 435)
ATGAGCCGGAGCGTGGCTCTGGCCGTGCTGGCCCTATTGTCTCTGAGCGGACTGGAAGCCATCC
AGAGAACCCCAAAGATCCAAGTGTACAGCAGACACCCTGCCGAGAACGGCAAGAGCAATTTCCT
GAACTGCTACGTGTCCGGCTTTCACCCCAGCGACATCGAGGTGGATCTGCTGAAGAACGGCGAG
AGAATCGAGAAGGTCGAACACAGCGATCTGAGCTTCAGCGAAGATGGATCCTTCTACCTGCTGT
ACTACACCGAGTTCACCCCTACAGAGAAGGACGAGTACGCCTGCAGAGTGAACCACGTGACCCT
GAGCCAGCCTAAGATCGTGAAGTGGGATAGAGATATG.

The term “base editor system” refers to an intermolecular complex for editing a nucleobase of a target nucleotide sequence. In various embodiments, the base editor (BE) system comprises (1) a polynucleotide programmable nucleotide binding domain, a deaminase domain (e.g., cytidine deaminase or adenosine deaminase) for deaminating nucleobases in the target nucleotide sequence; and (2) one or more guide polynucleotides (e.g., guide RNA) in conjunction with the polynucleotide programmable nucleotide binding domain. In various embodiments, the base editor (BE) system comprises a nucleobase editor domain selected from an adenosine deaminase or a cytidine deaminase, and a domain having nucleic acid sequence specific binding activity. In some embodiments, the base editor system comprises (1) a base editor (BE) comprising a polynucleotide programmable DNA binding domain and a deaminase domain for deaminating one or more nucleobases in a target nucleotide sequence; and (2) one or more guide RNAs in conjunction with the polynucleotide programmable DNA binding domain. In some embodiments, the polynucleotide programmable nucleotide binding domain is a polynucleotide programmable DNA binding domain. In some embodiments, the base editor is a cytidine base editor (CBE). In some embodiments, the base editor is an adenine or adenosine base editor (ABE). In some embodiments, the base editor is an adenine or adenosine base editor (ABE) or a cytidine or cytosine base editor (CBE). In some embodiments, the base editor system (e.g., a base editor system comprising a cytidine deaminase) comprises a uracil glycosylase inhibitor or other agent or peptide (e.g., a uracil stabilizing protein such as provided in WO2022015969, the disclosure of which is incorporated herein by reference in its entirety for all purposes) that inhibits the inosine base excision repair system.

The term “Cas9” or “Cas9 domain” refers to an RNA guided nuclease comprising a Cas9 protein, or a fragment thereof (e.g., a protein comprising an active, inactive, or partially active DNA cleavage domain of Cas9, and/or the gRNA binding domain of Cas9). A Cas9 nuclease is also referred to sometimes as a casnl nuclease or a CRISPR (clustered regularly interspaced short palindromic repeat) associated nuclease.

By “chimeric antigen receptor” or “CAR” is meant a synthetic or engineered receptor comprising an extracellular antigen binding domain joined to one or more intracellular signaling domains (e.g., T cell signaling domain), wherein the CAR confers specificity for an antigen bound by the antigen binding domain onto an immune effector cell. In some cases, the intracellular signaling domain is a T cell signaling domain. In embodiments, the immune effector cell is a T cell, an NK cell, or a macrophage. In embodiments, the CAR is a SUPRA CAR, an anti-tag CAR, a TCR-CAR, or a TCR-like CAR (see, e.g., Guedan, et al. “Engineering and Design of Chimeric Antigen Receptors,” Methods and Clinical Development, 12:145-156 (2019); Poorebrahim, et al., “TCR-like CARs and TCR-CARs targeting neoepitopes: an emerging potential,” Cancer Gene Therapy, 28:581-589 (2021); and Minutolo, et al. “The Emergence of Universal Immune Receptor T Cell Therapy for Cancer,” Front Oncol., 9:176 (2019), the disclosures of which are incorporated herein by reference in their entireties for all purposes).

By “chimeric antigen receptor (CAR) T cell” or “CAR-T cell” is meant a T cell expressing a CAR that has antigen specificity determined by the antibody-derived targeting domain of the CAR. As used herein, “CAR-T cells” includes T cells, regulatory T cells (TREG), or NK cells. As used herein, the term “CAR-T cells” includes cells engineered to express a CAR or a T cell receptor (TCR, sometimes referred to as TCR-CARs or TCR-like CARs). Methods of making CARs (e.g., for treatment of cancer) are publicly available (see, e.g., Park et al., Trends Biotechnol., 29:550-557, 2011; Grupp et al., N Engl J Med., 368:1509-1518, 2013; Han et al., J. Hematol Oncol. 6:47, 2013; Haso et al., (2013) Blood, 121, 1165-1174; Mohseni, et al., (2020) Front. Immunol., 11, art. 1608, doi: 10.3389/fimmu.2020.01608; Eggenhuizen, et al. Int. J. Mol. Sci. (2020), 21:7015, doi: 10.3390/ijms21197015; Poorebrahim, et al., Cancer Gene Ther 28, 581-589 (2021), doi.org/10.1038/s41417-021-00307-7, PCT Pubs. WO2012/079000, WO2013/059593; and U.S. Pub. 2012/0213783, the disclosure of each of which is incorporated herein by reference herein in its entirety). The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids can be defined where amino acids within a group exchange preferentially with each other, and therefore resemble each other most in their impact on the overall protein structure (Schulz, G. E. and Schirmer, R. H., supra). Non-limiting examples of conservative mutations include amino acid substitutions of amino acids, for example, lysine for arginine and vice versa such that a positive charge can be maintained; glutamic acid for aspartic acid and vice versa such that a negative charge can be maintained; serine for threonine such that a free —OH can be maintained; and glutamine for asparagine such that a free —NH₂ can be maintained.

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids can be defined where amino acids within a group exchange preferentially with each other, and therefore resemble each other most in their impact on the overall protein structure (Schulz, G. E. and Schirmer, R. H., supra). Non-limiting examples of conservative mutations include amino acid substitutions of amino acids, for example, lysine for arginine and vice versa such that a positive charge can be maintained; glutamic acid for aspartic acid and vice versa such that a negative charge can be maintained; serine for threonine such that a free-OH can be maintained; and glutamine for asparagine such that a free —NH₂ can be maintained.

By “cluster of differentiation 4 (CD4) polypeptide” is meant a protein having at least about 85% amino acid sequence identity to GenBank accession No. AAB51309.1 (SEQ ID NO: 436), or a functional fragment thereof having immunomodulatory activity.

By “cluster of differentiation 4 (CD4) polynucleotide” is meant a nucleic acid molecule encoding a CD4 polypeptide, as well as the introns, exons, 3′ untranslated regions, 5′ untranslated regions, and regulatory sequences associated with its expression, or fragments thereof. An exemplary CD4 polynucleotide sequence is provided at Ensembl Accession No. ENSG00000010610.

By “cluster of differentiation 4 transmembrane (CD4TM) polypeptide” is meant a polypeptide with at least about 85% amino acid sequence identity to the amino acid sequence MALIVLGGVAGLLLFIGLGIFFCVRC (SEQ ID NO: 437), or a fragment thereof capable of anchoring a polypeptide linked thereto to a cell membrane.

By “CD4TM polynucleotide” is meant a nucleic acid molecule encoding a CD4TM polypeptide.

By “cluster of differentiation 47 (CD47) polypeptide” is meant a protein having at least about 85% amino acid sequence identity to NCBI Ref. Seq. accession No. NP_001768.1 (SEQ ID NO: 668), or a functional fragment thereof having immunomodulatory activity.

By “cluster of differentiation 47 (CD47) polynucleotide” is meant a nucleic acid molecule encoding a CD47 polypeptide, as well as the introns, exons, 3′ untranslated regions, 5′ untranslated regions, and regulatory sequences associated with its expression, or fragments thereof. An exemplary CD47 polynucleotide sequence is provided at Ensembl Accession No. ENSG00000196776.

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids can be defined where amino acids within a group exchange preferentially with each other, and therefore resemble each other most in their impact on the overall protein structure (Schulz, G. E. and Schirmer, R. H., supra). Non-limiting examples of conservative mutations include amino acid substitutions of amino acids, for example, lysine for arginine and vice versa such that a positive charge can be maintained; glutamic acid for aspartic acid and vice versa such that a negative charge can be maintained; serine for threonine such that a free —OH can be maintained; and glutamine for asparagine such that a free —NH₂ can be maintained.

The term “coding sequence” or “protein coding sequence” as used interchangeably herein refers to a segment of a polynucleotide that codes for a protein. Coding sequences can also be referred to as open reading frames. The region or sequence is bounded nearer the 5′ end by a start codon and nearer the 3′ end with a stop codon. Stop codons useful with the base editors described herein include the following: TAG, TAA, and TGA.

By “complex” is meant a combination of two or more molecules whose interaction relies on inter-molecular forces. Non-limiting examples of inter-molecular forces include covalent and non-covalent interactions. Non-limiting examples of non-covalent interactions include hydrogen bonding, ionic bonding, halogen bonding, hydrophobic bonding, van der Waals interactions (e.g., dipole-dipole interactions, dipole-induced dipole interactions, and London dispersion forces), and x-effects. In an embodiment, a complex comprises polypeptides, polynucleotides, or a combination of one or more polypeptides and one or more polynucleotides. In one embodiment, a complex comprises one or more polypeptides that associate to form a base editor (e.g., base editor comprising a nucleic acid programmable DNA binding protein, such as Cas9, and a deaminase) and a polynucleotide (e.g., a guide RNA). In an embodiment, the complex is held together by hydrogen bonds. It should be appreciated that one or more components of a base editor (e.g., a deaminase, or a nucleic acid programmable DNA binding protein) may associate covalently or non-covalently. As one example, a base editor may include a deaminase covalently linked to a nucleic acid programmable DNA binding protein (e.g., by a peptide bond). Alternatively, a base editor may include a deaminase and a nucleic acid programmable DNA binding protein that associate noncovalently (e.g., where one or more components of the base editor are supplied in trans and associate directly or via another molecule such as a protein or nucleic acid). In an embodiment, one or more components of the complex are held together by hydrogen bonds.

By “cytosine” or “4-Aminopyrimidin-2 (1H)-one” is meant a purine nucleobase with the molecular formula C₄H₅N₃O, having the structure

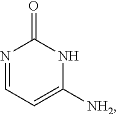

and corresponding to CAS No. 71-30-7.

By “cytidine” is meant a cytosine molecule attached to a ribose sugar via a glycosidic bond, having the structure

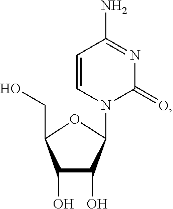

and corresponding to CAS No. 65-46-3. Its molecular formula is C₉H₁₃N₃O₅.

By “Cytidine Base Editor (CBE)” is meant a base editor comprising a cytidine deaminase.

By “Cytidine Base Editor (CBE) polynucleotide” is meant a polynucleotide encoding a CBE.

By “cytidine deaminase” or “cytosine deaminase” is meant a polypeptide or fragment thereof capable of deaminating cytidine or cytosine. In embodiments, the cytidine or cytosine is present in a polynucleotide. In one embodiment, the cytidine deaminase converts cytosine to uracil or 5-methylcytosine to thymine. The terms “cytidine deaminase” and “cytosine deaminase” are used interchangeably throughout the application. Petromyzon marinus cytosine deaminase 1 (PmCDA1) (SEQ ID NO: 13-14), Activation-induced cytidine deaminase (AICDA) (SEQ ID NOs: 15-21), and APOBEC (SEQ ID NOs: 12-61) are exemplary cytidine deaminases. Further exemplary cytidine deaminase (CDA) sequences are provided in the Sequence Listing as SEQ ID NOs: 62-66 and SEQ ID NOs: 67-189. Non-limiting examples of cytidine deaminases include those described in PCT/US20/16288, PCT/US2018/021878, 180802-021804/PCT, PCT/US2018/048969, and PCT/US2016/058344. By “cytosine deaminase activity” is meant catalyzing the deamination of cytosine or cytidine. In one embodiment, a polypeptide having cytosine deaminase activity converts an amino group to a carbonyl group. In an embodiment, a cytosine deaminase converts cytosine to uracil (i.e., C to U) or 5-methylcytosine to thymine (i.e., 5mC to T). In some embodiments, a cytosine deaminase as provided herein has increased cytosine deaminase activity (e.g., at least 10-fold, 20-fold, 30-fold, 40-fold, 50-fold, 60-fold, 70-fold, 80-fold, 90-fold, 100-fold or more) relative to a reference cytosine deaminase.

By “cytotoxic T lymphocyte-associated 4 (CTLA4) polypeptide” is meant a protein having at least about 85% amino acid sequence identity to NCBI Ref. Seq. accession No. NP_005205.2 (SEQ ID NO: 666), or a functional fragment thereof having immunomodulatory activity.

By “cytotoxic T lymphocyte-associated 4 (CTLA4) polynucleotide” is meant a nucleic acid molecule encoding a CTLA4 polypeptide, as well as the introns, exons, 3′ untranslated regions, 5′ untranslated regions, and regulatory sequences associated with its expression, or fragments thereof. An exemplary CTLA4 polynucleotide sequence is provided at Ensembl Accession No. ENSG00000163599.

The term “deaminase” or “deaminase domain,” as used herein, refers to a protein or fragment thereof that catalyzes a deamination reaction.

“Detect” refers to identifying the presence, absence or amount of the analyte to be detected. In one embodiment, a sequence alteration in a polynucleotide or polypeptide is detected. In another embodiment, the presence of indels is detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an enzyme linked immunosorbent assay (ELISA)), biotin, digoxigenin, or haptens.

By “disease” is meant any condition or disorder that damages or interferes with the normal function of a cell, tissue, or organ. In some embodiments, the disease is a cancer (e.g., a hematological cancer or a solid tumor). In some instances, the disease is a disease that can be treated using the modified allogeneic T cells of the disclosure.

By “dual editing activity” or “dual deaminase activity” is meant having adenosine deaminase and cytidine deaminase activity. In one embodiment, a base editor having dual editing activity has both A→G and C→T activity, wherein the two activities are approximately equal or are within about 10% or 20% of each other. In another embodiment, a dual editor has A→G activity that no more than about 10% or 20% greater than C→T activity. In another embodiment, a dual editor has A→G activity that is no more than about 10% or 20% less than C→T activity. In some embodiments, the adenosine deaminase variant has predominantly cytosine deaminase activity, and little, if any, adenosine deaminase activity. In some embodiments, the adenosine deaminase variant has cytosine deaminase activity, and no significant or no detectable adenosine deaminase activity.

By “effective amount” is meant the amount of an agent or active compound that is required to ameliorate the symptoms of a disease relative to an untreated patient or an individual without disease. In an embodiment, the agent is a CAR-T cell described herein (e.g., a CAR-T cell expressing a beta-2 microglobulin fused to an HLA-A, -B, or -C). The effective amount of active compound(s) or agent used to practice the present disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In one embodiment, an effective amount is the amount of a base editor of the disclosure sufficient to introduce an alteration in a gene of interest in a cell (e.g., a cell in vitro or in vivo). In one embodiment, an effective amount is the amount of a base editor required to achieve a therapeutic effect. Such therapeutic effect need not be sufficient to alter a pathogenic gene in all cells of a subject, tissue, or organ, but only to alter the pathogenic gene in about 1%, 5%, 10%, 25%, 50%, 75% or more of the cells present in a subject, tissue, or organ. In one embodiment, an effective amount is sufficient to ameliorate one or more symptoms of a disease.

The term “exonuclease” refers to a protein or polypeptide capable of removing successive nucleotides from either the 5′ or 3′ end of a polynucleotide.

The term “endonuclease” refers to a protein or polypeptide capable of catalyzing the cleavage of internal regions in a polynucleotide.

By “fragment” is meant a portion of a polypeptide or nucleic acid molecule. This portion contains, at least about 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids. In some embodiments, the fragment is a functional fragment.

In general, a “gene” is a region on the genome that is capable of being transcribed to an RNA that either has a regulatory function, a catalytic function, and/or encodes a protein. An eukaryotic gene typically has introns and exons, which may organize to produce different RNA splice variants that encode alternative versions of a mature protein. The skilled artisan will appreciate that the present disclosure encompasses all transcripts encoding a polypeptide of interest, including splice variants, allelic variants and transcripts that occur because of alternative promoter sites or alternative poly-adenylation sites. A “full-length” gene or RNA therefore encompasses any naturally occurring splice variants, allelic variants, other alternative transcripts, splice variants generated by recombinant technologies which bear the same function as the naturally occurring variants, and the resulting RNA molecules. In some embodiments, the fragment is a functional fragment.

“Graft versus host disease” (GVHD) refers to a pathological condition where transplanted cells of a donor generate an immune response against cells of the host.

By “guide polynucleotide” is meant a polynucleotide or polynucleotide complex that is specific for a target sequence and can form a complex with a polynucleotide programmable nucleotide binding domain protein (e.g., Cas9 or Cpf1). In an embodiment, the guide polynucleotide is a guide RNA (gRNA). gRNAs can exist as a complex of two or more RNAs, or as a single RNA molecule.

By “granzyme B (GZMB) polypeptide” is meant a protein having at least about 85% amino acid sequence identity to GenBank accession No. AAA75490.1 (SEQ ID NO: 438), or a functional fragment thereof having immunomodulatory activity.

By “granzyme B (GZMB) polynucleotide” is meant a nucleic acid molecule encoding an GZMB polypeptide, as well as the introns, exons, 3′ untranslated regions, 5′ untranslated regions, and regulatory sequences associated with its expression, or fragments thereof. An exemplary human GZMB polynucleotide sequence is provided at Ensembl Accession No. ENSG00000100453.

By “heterologous,” or “exogenous” is meant a polynucleotide or polypeptide that 1) has been experimentally incorporated to a polynucleotide or polypeptide sequence to which the polynucleotide or polypeptide is not normally found in nature; or 2) has been experimentally placed into a cell that does not normally comprise the polynucleotide or polypeptide. In some embodiments, “heterologous” means that a polynucleotide or polypeptide has been experimentally placed into a non-native context. In some embodiments, a heterologous polynucleotide or polypeptide is derived from a first species or host organism, and is incorporated into a polynucleotide or polypeptide derived from a second species or host organism. In some embodiments, the first species or host organism is different from the second species or host organism. In some embodiments the heterologous polynucleotide is DNA. In some embodiments the heterologous polynucleotide is RNA. “Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

“Host versus graft disease” (HVGD) refers to a pathological condition where the immune system of a host generates an immune response against transplanted cells of a donor.

By “Human Leukocyte Antigen-E (HLA-E) polypeptide” is meant a protein having at least about 85% amino acid sequence identity to NCBI Accession No. NP_005507.3 (SEQ ID NO: 629), or a fragment thereof having immunomodulatory activity.

By “Human Leukocyte Antigen-E (HLA-E) polynucleotide” is meant a nucleic acid molecule encoding an HLA-E polypeptide, as well as the introns, exons, 3′ untranslated regions, 5′ untranslated regions, and regulatory sequences associated with its expression, or fragments thereof. An exemplary HLA-E polynucleotide is provided at NCBI Accession No. NM_005516.6 (SEQ ID NO: 630). The HLA-E gene corresponds to Ensembl: ENSG00000116815.

By “interferon gamma (IFN-G) polypeptide” is meant a protein having at least about 85% amino acid sequence identity to GenBank accession No. CAA44325.1 (SEQ ID NO: 439), or a functional fragment thereof having immunomodulatory activity.

By “interferon gamma (IFN-G) polynucleotide” is meant a nucleic acid molecule encoding an IFN-G polypeptide, as well as the introns, exons, 3′ untranslated regions, 5′ untranslated regions, and regulatory sequences associated with its expression, or fragments thereof. An exemplary human IFN-G polynucleotide sequence is provided at Ensembl Accession No. ENSG00000111537.

By “major histocompatibility complex, class I, A (HLA-A) polypeptide” is meant an HLA class-I heavy chain polypeptide having at least about 85% amino acid sequence identity to GenBank Accession No. BAA07530.1, to HLA-A*02, and/or to HLA-A*03, which are provided below, or a fragment thereof having antigen presenting activity. In embodiments, the HLA-A polypeptide contains an amino acid alteration at one or more positions selected from D227, T228, and A245 (e.g., a D227K, T228A, and/or A245V alteration), or a corresponding position(s), where the positions are numbered relative to the “HLA-A*02” or “HLA-A*03” sequence provided below. In some cases, the HLA-A polypeptide contains one or more amino acid alterations within the alpha-3 domain (e.g., positions 183-274 numbered relative to the “HLA-A*02” or “HLA-A*03” sequence provided below, or corresponding positions).

>BAA07530.1 HLA-A [Homo sapiens] (signal peptide is in bold)
(SEQ ID NO: 440)
MAVMAPRTLVLLLSGALALTQTWAGSHSMRYFFTSVSRPGRGEPRFIAVGYVDDTQFVRFDSDA
ASQRMEPRAPWIEQEGPEYWDGETRKVKAHSQTHRVDLGTLRGYYNQSEAGSHTVQRMCGCDVG
SDWRFLRGYHQYAYDGKDYIALKEDLRSWTAADMAAQTTKHKWEAAHVAEQLRAYLEGTCVEWL
RRYLENGKETLQRTDAPKTHMTHHAVSDHEATLRCWALSFYPAEITLTWQRDGEDQTQDTELVE
TRPAGDGTFQKWAAVVVPSGQEQR.
>HLA-A*02 (HLA-A2)
(SEQ ID NO: 441)
GSHSMRYFFTSVSRPGRGEPRFIAVGYVDDTQFVRFDSDAASQRMEPRAPWIEQEGPEYWDGET
RKVKAHSQTHRVDLGTLRGYYNQSEAGSHTVQRMYGCDVGSDWRFLRGYHQYAYDGKDYIALKE
DLRSWTAADMAAQTTKHKWEAAHVAEQLRAYLEGTCVEWLRRYLENGKETLQRTDAPKTHMTHH
AVSDHEATLRCWALSFYPAEITLTWQRDGEDQTQDTELVETRPAGDGTFQKWAAVVVPSGQEQR
YTCHVQHEGLPKPLTLRWEPSSQPTIPIVGIIAGLVLFGAVITGAVVAAVMWRRKSSDRKGGSY
SQAASSDSAQGSDVSLTACKV.
>HLA-A*03
(SEQ ID NO: 442)
GSHSMRYFFTSVSRPGRGEPRFIAVGYVDDTQFVRFDSDAASQRMEPRAPWIEQEGPEYWDQET
RNVKAQSQTDRVDLGTLRGYYNQSEAGSHTIQIMYGCDVGSDGRFLRGYRQDAYDGKDYIALNE
DLRSWTAADMAAQITKRKWEAAHEAEQLRAYLDGTCVEWLRRYLENGKETLQRTDPPKTHMTHH
PISDHEATLRCWALGFYPAEITLTWQRDGEDQTQDTELVETRPAGDGTFQKWAAVVVPSGEEQR
YTCHVQHEGLPKPLTLRWELSSQPTIPIVGIIAGLVLLGAVITGAVVAAVMWRRKSSDRKGGSY
TQAASSDSAQGSDVSLTACKV.

By “major histocompatibility complex, class I, A (HLA-A) polynucleotide” is meant a nucleic acid molecule encoding an HLA-A polypeptide, as well as the introns, exons, 3′ untranslated regions, 5′ untranslated regions, and regulatory sequences associated with its expression, or fragments thereof. An exemplary HLA-A polynucleotide sequence is provided at Genbank Accession No. D38525.1, which is provided below.

>D38525.1:1-843 Homo sapiens HLA-A mRNA, complete cds, HLA-A null allele HLA-A*0215N ATGGCCGTCATGGCGCCCCGAACCCTCGTCCTGCTACTCTCGGGGGCTCTGGCCCTGACCCAGA CCTGGGCGGGCTCTCACTCCATGAGGTATTTCTTCACATCCGTGTCCCGGCCCGGCCGCGGGGA GCCCCGCTTCATCGCAGTGGGCTACGTGGACGACACGCAGTTCGTGCGGTTCGACAGCGACGCC GCGAGCCAGAGGATGGAGCCGCGGGCGCCGTGGATAGAGCAGGAGGGTCCGGAGTATTGGGACG GGGAGACACGGAAAGTGAAGGCCCACTCACAGACTCACCGAGTGGACCTGGGGACCCTGCGCGG CTACTACAACCAGAGCGAGGCCGGTTCTCACACCGTCCAGAGGATGTGTGGCTGCGACGTGGGG TCGGACTGGCGCTTCCTCCGCGGGTACCACCAGTACGCCTACGACGGCAAGGATTACATCGCCC TGAAAGAGGACCTGCGCTCTTGGACCGCGGCGGACATGGCAGCTCAGACCACCAAGCACAAGTG GGAGGCGGCCCATGTGGCGGAGCAGTTGAGAGCCTACCTGGAGGGCACGTGCGTGGAGTGGCTC CGCAGATACCTGGAGAACGGGAAGGAGACGCTGCAGCGCACGGACGCCCCCAAAACGCATATGA CTCACCACGCTGTCTCTGACCATGAAGCCACCCTGAGGTGCTGGGCCCTGAGCTTCTACCCTGC GGAGATCACACTGACCTGGCAGCGGGATGGGGAGGACCAGACCCAGGACACGGAGCTCGTGGAG ACCAGGCCTGCAGGGGATGGAACCTTCCAGAAGTGGGCGGCTGTGGTGGTGCCTTCTGGACAGG AGCAGAGATAA (SEQ ID NO: 443). The HLA-A gene corresponds to Ensemble ENSG00000206503.

By “major histocompatibility complex, class I, B (HLA-B) polypeptide” is meant an HLA class-I heavy chain polypeptide having at least about 85% amino acid sequence identity to GenBank Accession No. CAD30340.1, to HLA-B*57, and/or to HLA-B*44, which are provided below, or a fragment thereof having antigen presenting activity. In embodiments, the HLA-B polypeptide contains an amino acid alteration at one or more positions selected from D227, T228, and A245 (e.g., a D227K, T228A, and/or A245V alteration), or a corresponding position(s), where the positions are numbered relative to the “HLA-B*57” or “HLA-B*44” sequence provided below. In some cases, the HLA-B polypeptide contains one or more amino acid alterations within the alpha-3 domain (e.g., positions 183-274 numbered relative to the e “HLA-B*57” or “HLA-B*44” sequence provided below, or corresponding positions in another HLA-B polypeptide).

>CAD30340.1 HLA-B [Homo sapiens]
(SEQ ID NO: 444)
MRVTAPRTVLLLLSAALALTETWAGSHSMRYFHTAMSRPGRGEPRFITVGYVDDTLFVRFDSDA
TSPRKEPRAPWIEQEGPEYWDRETQISKTNTQTYRESLRNLRGYYNQSEAGSHTWQRMYGCDLG
PDGRLLRGYNQLAYDGKDYIALNEDLSSWTAADTAAQITQRKWEAARVAEQDRAYLEGLCVESL
RRYLENGKETLQRADPPKTHVTHHPISDHEATLRCWALGFYPAEITLTWQRDGEDQTQDTELVE
TRPAGDRTFQKWAAVVVPSGEEQRYTCHVQHEGLPKPLTLRWEPSSQSTIPIVGIVAGLAVLAV
VVIGAVVATVMCRRKSSGGKGGSYSQAASSDSAQGSDVSLTA.
>HLA-B*57
(SEQ ID NO: 445)
GSHSMRYFYTAMSRPGRGEPRFIAVGYVDDTQFVRFDSDAASPRMAPRAPWIEQEGPEYWDGET
RNMKASAQTYRENLRIALRYYNQSEAGSHIIQVMYGCDVGPDGRLLRGHDQSAYDGKDYIALNE
DLSSWTAADTAAQITQRKWEAARVAEQLRAYLEGLCVEWLRRYLENGKETLQRADPPKTHVTHH
PISDHEATLRCWALGFYPAEITLTWQRDGEDQTQDTELVETRPAGDRTFQKWAAVVVPSGEEQR
YTCHVQHEGLPKPLTLRWEPSSQSTVPIVGIVAGLAVLAVVVIGAVVAAVMCRRKSSGGKGGSY
SQAACSDSAQGSDVSLTA.
>HLA-B*44
(SEQ ID NO: 446)
GSHSMRYFYTAMSRPGRGEPRFITVGYVDDTLFVRFDSDATSPRKEPRAPWIEQEGPEYWDRET
QISKTNTQTYRENLRTALRYYNQSEAGSHIIQRMYGCDVGPDGRLLRGYDQDAYDGKDYIALNE
DLSSWTAADTAAQITQRKWEAARVAEQDRAYLEGLCVESLRRYLENGKETLQRADPPKTHVTHH
PISDHEVTLRCWALGFYPAEITLTWQRDGEDQTQDTELVETRPAGDRTFQKWAAVVVPSGEEQR
YTCHVQHEGLPKPLTLRWEPSSQSTVPIVGIVAGLAVLAVVVIGAVVAAVMCRRKSSGGKGGSY
SQAACSDSAQGSDVSLTA

By “major histocompatibility complex, class I, B (HLA-B) polynucleotide” is meant a nucleic acid molecule encoding an HLA-B polypeptide, as well as the introns, exons, 3′ untranslated regions, 5′ untranslated regions, and regulatory sequences associated with its expression, or fragments thereof. An exemplary HLA-B polynucleotide sequence is provided at Genbank Accession No. AJ458992.1, which is provided below.

>AJ458992.1:302-374,503-772, 1023-1298, 1872-2147,2252-2368,2810-2842,2949-2992 Homo sapiens HLA-B gene for MHC class I antigen, HLA-B*4501 allele, exons 1-7

(SEQ ID NO: 447)
ATGCGGGTCACGGCACCCCGAACCGTCCTCCTGCTGCTCTCGGCG
GCCCTGGCCCTGACCGAGACCTGGGCCGGCTCCCACTCCATGAGG
TATTTCCACACCGCCATGTCCCGGCCCGGCCGCGGGGAGCCCCGC
TTCATCACCGTGGGCTACGTGGACGACACGCTGTTCGTGAGGTTC
GACAGCGACGCCACGAGTCCGAGGAAGGAGCCGCGGGCGCCATGG
ATAGAGCAGGAGGGGCCGGAGTATTGGGACCGGGAGACACAGATC
TCCAAGACCAACACACAGACTTACCGAGAGAGCCTGCGGAACCTG
CGCGGCTACTACAACCAGAGCGAGGCCGGGTCTCACACTTGGCAG
AGGATGTATGGCTGCGACCTGGGGCCCGACGGGCGCCTCCTCCGC
GGGTATAACCAGTTAGCCTACGACGGCAAGGATTACATCGCCCTG
AACGAGGACCTGAGCTCCTGGACCGCGGCGGACACCGCGGCTCAG
ATCACCCAGCGCAAGTGGGAGGCGGCCCGTGTGGCGGAGCAGGAC
AGAGCCTACCTGGAGGGCCTGTGCGTGGAGTCGCTCCGCAGATAC
CTGGAGAACGGGAAGGAGACGCTGCAGCGCGCGGACCCCCCAAAG
ACACATGTGACCCACCACCCCATCTCTGACCATGAGGCCACCCTG
AGGTGCTGGGCCCTGGGCTTCTACCCTGCGGAGATCACACTGACC
TGGCAGCGGGATGGCGAGGACCAAACTCAGGACACCGAGCTTGTG
GAGACCAGACCAGCAGGAGATAGAACCTTCCAGAAGTGGGCAGCT
GTGGTGGTGCCTTCTGGAGAAGAGCAGAGATACACATGCCATGTA
CAGCATGAGGGGCTGCCGAAGCCCCTCACCCTGAGATGGGAGCCA
TCTTCCCAGTCCACCATCCCCATCGTGGGCATTGTTGCTGGCCTG
GCTGTCCTAGCAGTTGTGGTCATCGGAGCTGTGGTCGCTACTGTG
ATGTGTAGGAGGAAGAGCTCAGGTGGAAAAGGAGGGAGCTACTCT
CAGGCTGCGTCCAGCGACAGTGCCCAGGGCTCTGATGTGTCTCTC
ACAGCTTGA.

By “major histocompatibility complex, class I, C (HLA-C) polypeptide” is meant an HLA class-I heavy chain polypeptide having at least about 85% amino acid sequence identity to GenBank Accession No. BBO94058.1, to HLA-C*04, HLA-C*05, and/or to HLA-C*07, which are provided below, or a fragment thereof having antigen presenting activity. In embodiments, the HLA-C polypeptide contains an amino acid alteration at one or more positions selected from C1, D227, T228, and A245 (e.g., a CIG, A73T, D227K, T228A, and/or A245V alteration), or a corresponding position(s), where the positions are numbered relative to the “HLA-C*04,” “HLA-C*05”, or “HLA-C*07” sequence provided below. In some cases, the HLA-C polypeptide contains one or more amino acid alterations within the alpha-3 domain (e.g., positions 183-274 numbered relative to the “HLA-C*04,” “HLA-C*05”, or “HLA-C*07” sequence provided below, or corresponding positions in another HLA-C polypeptide).

>BB094058.1 MHC class I antigen, HLA-C alpha chain [Homo sapiens]
(SEQ ID NO: 448)
MRVMAPRTLILLLSGALALTETWACSHSMRYFYTAVSRPGRGEPRFIAVGYVDDTQFVRFDSDA
ASPRGEPRAPWVEQEGPEYWDRETQKYKRQAQADRVSLRNLRGYYNQSEAGSHTLQRMYGCDLG
PDGRLLRGYDQSAYDGKDYIALNEDLRSWTAADTAAQITQRKWEAAREAEQWRAYLEGTCVEWL
RRYLENGKETLQRAEHPKTHVTHHPVSDHEATLRCWALGFYPAEITLTWQRDGEDQTQDTELVE
TRPAGDGTFQKWAAVVVPSGEEQRYTCHVQHEGLPEPLTLRWEPSSQPTIPIMGIVAGLAVLAV
LAVLGAVMAVVMCRRKSSGGKGGSCSQAASSNSAQGSDESLIACKA.
>HLA-C*04
(SEQ ID NO: 449)
GSHSMRYFSTSVSWPGRGEPRFIAVGYVDDTQFVRFDSDAASPRGEPREPWVEQEGPEYWDRET
QKYKRQAQADRVNLRKLRGYYNQSEDGSHTLQRMFGCDLGPDGRLLRGYNQFAYDGKDYIALNE
DLRSWTAADTAAQITQRKWEAAREAEQRRAYLEGTCVEWLRRYLENGKETLQRAEHPKTHVTHH
PVSDHEATLRCWALGFYPAEITLTWQWDGEDQTQDTELVETRPAGDGTFQKWAAVVVPSGEEQR
YTCHVQHEGLPEPLTLRWKPSSQPTIPIVGIVAGLAVLAVLAVLGAMVAVVMCRRKSSGGKGGS
CSQAASSNSAQGSDESLIACKA.
>HLA-C*05
(SEQ ID NO: 450)
CSHSMRYFYTAVSRPGRGEPRFIAVGYVDDTQFVQFDSDAASPRGEPRAPWVEQEGPEYWDRET
QKYKRQAQTDRVNLRKLRGYYNQSEAGSHTLQRMYGCDLGPDGRLLRGYNQFAYDGKDYIALNE
DLRSWTAADKAAQITQRKWEAAREAEQRRAYLEGTCVEWLRRYLENGKKTLQRAEHPKTHVTHH
PVSDHEATLRCWALGFYPAEITLTWQRDGEDQTQDTELVETRPAGDGTFQKWAAVVVPSGEEQR
YTCHVQHEGLPEPLTLRWGPSSQPTIPIVGIVAGLAVLAVLAVLGAVMAVVMCRRKSSGGKGGS
CSQAASSNSAQGSDESLIACKA.
>HLA-C*07
(SEQ ID NO: 451)
CSHSMRYFDTAVSRPGRGEPRFISVGYVDDTQFVRFDSDAASPRGEPRAPWVEQEGPEYWDRET
QKYKRQAQADRVSLRNLRGYYNQSEDGSHTLQRMSGCDLGPDGRLLRGYDQSAYDGKDYIALNE
DLRSWTAADTAAQITQRKLEAARAAEQLRAYLEGTCVEWLRRYLENGKETLQRAEPPKTHVTHH
PLSDHEATLRCWALGFYPAEITLTWQRDGEDQTQDTELVETRPAGDGTFQKWAAVVVPSGQEQR
YTCHMQHEGLQEPLTLSWEPSSQPTIPIMGIVAGLAVLVVLAVLGAVVTAMMCRRKSSGGKGGS
CSQAACSNSAQGSDESLITCKA.

By “major histocompatibility complex, class I, C (HLA-C) polynucleotide” is meant a nucleic acid molecule encoding an HLA-C polypeptide, as well as the introns, exons, 3′ untranslated regions, 5′ untranslated regions, and regulatory sequences associated with its expression, or fragments thereof. An exemplary HLA-C polynucleotide sequence is provided at Genbank Accession No. LC508210.1, which is provided below.

>LC508210.1:1-73,204-473,720-995,1583-1858,1980-2099,2539-2571,2679-2726,2891-2895 Homo sapiens HLA-C gene for MHC class I antigen, HLA-C alpha chain, complete cds, HLA-C*12:02:02:01 variant

(SEQ ID NO: 452)
ATGCGGGTCATGGCGCCCCGAACCCTCATCCTGCTGCTCTCGGGA
GCCCTGGCCCTGACCGAGACCTGGGCCTGCTCCCACTCCATGAGG
TATTTCTACACCGCCGTGTCCCGGCCCGGCCGCGGAGAGCCCCGC
TTCATCGCAGTGGGCTACGTGGACGACACGCAGTTCGTGCGGTTC
GACAGCGACGCCGCGAGTCCAAGAGGGGAGCCGCGGGCGCCGTGG
GTGGAGCAGGAGGGGCCGGAGTATTGGGACCGGGAGACACAGAAG
TACAAGCGCCAGGCACAGGCTGACCGAGTGAGCCTGCGGAACCTG
CGCGGCTACTACAACCAGAGCGAGGCCGGGTCTCACACCCTCCAG
AGGATGTACGGCTGCGACCTGGGGCCCGACGGGCGCCTCCTCCGC
GGGTATGACCAGTCCGCCTACGACGGCAAGGATTACATCGCCCTG
AACGAGGACCTGCGCTCCTGGACCGCTGCGGACACGGCGGCTCAG
ATCACCCAGCGCAAGTGGGAGGCGGCCCGTGAGGCGGAGCAGTGG
AGAGCCTACCTGGAGGGCACGTGCGTGGAGTGGCTCCGCAGATAC
CTGGAGAACGGGAAGGAGACGCTGCAGCGCGCGGAACACCCAAAG
ACACACGTGACCCACCATCCCGTCTCTGACCATGAGGCCACCCTG
AGGTGCTGGGCCCTGGGCTTCTACCCTGCGGAGATCACACTGACC
TGGCAGCGGGATGGCGAGGACCAAACTCAGGACACCGAGCTTGTG
GAGACCAGGCCAGCAGGAGATGGAACCTTCCAGAAGTGGGCAGCT
GTGGTGGTGCCTTCTGGAGAAGAGCAGAGATACACGTGCCATGTG
CAGCACGAGGGGCTGCCAGAGCCCCTCACCCTGAGATGGGAGCCA
TCTTCCCAGCCCACCATCCCCATCATGGGCATCGTTGCTGGCCTG
GCTGTCCTGGCTGTCCTAGCTGTCCTAGGAGCTGTGATGGCTGTT
GTGATGTGTAGGAGGAAGAGCTCAGGTGGAAAAGGAGGGAGCTGC
TCTCAGGCTGCGTCCAGCAACAGTGCCCAGGGCTCTGATGAGTCT
CTCATCGCTTGTAAAGCCTGA.

By “immune cell” is meant a cell of the immune system capable of generating an immune response. Exemplary immune cell include, but are not limited to, T cells, NK cells, B cells, or hematopoietic stem cells.

By “immune effector cell” is meant a lymphocyte, once activated, capable of effecting an immune response upon a target cell. In some embodiments, immune effector cells are effector T cells. In some embodiments, the effector T cell is a naïve CD8⁺ T cell, a cytotoxic T cell, a natural killer T (NKT) cell, a natural killer (NK) cell, or a regulatory T (Treg) cell. In some embodiments, immune effector cells are effector NK cells. In some embodiments, the effector T cells are thymocytes, immature T lymphocytes, mature T lymphocytes, resting T lymphocytes, or activated T lymphocytes. In some embodiments the immune effector cell is a CD4⁺ CD8⁺ T cell or a CD4⁻ CD8⁻ T cell. In some embodiments the immune effector cell is a T helper cell. In some embodiments the T helper cell is a T helper 1 (Th1), a T helper 2 (Th2) cell, or a helper T cell expressing CD4 (CD4⁺ T cell).

By “immunomodulatory activity” is meant increasing, decreasing, or sustaining an immune response. In embodiments, the reduction in immune response is at least about 5%, 10%, 10%, 25%, 50%, 75%, 80%, 90%, 95% or 100%. In embodiments, a reduction in immune response is measured by detecting the expression, levels, or activity of granzyme B (GZMB), IFN-gamma, and TNF-alpha. In other embodiments, a reduction in immune response is measured by detecting a reduction in the killing of a target cell (e.g., modified immune cell) by an effector cell (e.g., CAR-T cell, T cell, NK cell).

By “increases” is meant a positive alteration of at least 10%, 25%, 50%, 75%, or 100%, or about 1.5 fold, about 2 fold, about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, about 50-fold, or about 100-fold.

The terms “inhibitor of base repair”, “base repair inhibitor”, “IBR” or their grammatical equivalents refer to a protein that is capable in inhibiting the activity of a nucleic acid repair enzyme, for example a base excision repair enzyme.

An “intein” is a fragment of a protein that is able to excise itself and join the remaining fragments (the exteins) with a peptide bond in a process known as protein splicing.

By “programmed cell death ligand 1 (PD-L1) polypeptide” is meant a protein having at least about 85% amino acid sequence identity to GenBank accession No. AAP13470.1 (SEQ ID NO: 667), or a functional fragment thereof having immunomodulatory activity.

By “programmed cell death ligand 1 (PD-L1) polynucleotide” is meant a nucleic acid molecule encoding a PD-L1 polypeptide, as well as the introns, exons, 3′ untranslated regions, 5′ untranslated regions, and regulatory sequences associated with its expression, or fragments thereof. An exemplary PD-L1 polynucleotide sequence is provided at Ensembl Accession No. ENSG00000120217.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid molecule that is free of the genes which, in the naturally-occurring genome of the organism from which the nucleic acid molecule of the disclosure is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the disclosure that has been separated from components that naturally accompany it. Typically, the polypeptide is isolated when it is at least 60%, by weight, free from the proteins and naturally-occurring organic molecules with which it is naturally associated. In some embodiments, the preparation is at least 75%, at least 90%, or at least 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term “linker”, as used herein, refers to a molecule that links two moieties. In one embodiment, the term “linker” refers to a covalent linker (e.g., covalent bond) or a non-covalent linker.

By “marker” is meant any protein, polynucleotide, or other analyte having an alteration in expression, level, structure, or activity that is associated with a disease or disorder.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, IRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O (6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The term “nuclear localization sequence,” “nuclear localization signal,” or “NLS” refers to an amino acid sequence that promotes import of a protein into the cell nucleus. Nuclear localization sequences are known in the art and described, for example, in Plank et al., International PCT application, PCT/EP2000/011690, filed Nov. 23, 2000, published as WO/2001/038547 on May 31, 2001, the contents of which are incorporated herein by reference for their disclosure of exemplary nuclear localization sequences. In other embodiments, the NLS is an optimized NLS described, for example, by Koblan et al., Nature Biotech. 2018 doi:10.1038/nbt.4172. In some embodiments, an NLS comprises the amino acid sequence

(SEQ ID NO: 190)
KRTADGSEFESPKKKRKV,
(SEQ ID NO: 191)
KRPAATKKAGQAKKKK,
(SEQ ID NO: 192)
KKTELQTTNAENKTKKL,
(SEQ ID NO: 193)
KRGINDRNFWRGENGRKTR,
(SEQ ID NO: 194)
RKSGKIAAIVVKRPRK,
(SEQ ID NO: 195)
PKKKRKV,
(SEQ ID NO: 196)
MDSLLMNRRKFLYQFKNVRWAKGRRETYLC,
(SEQ ID NO: 328)
PKKKRKVEGADKRTADGSEFESPKKKRKV,
or
(SEQ ID NO: 329)
RKSGKIAAIVVKRPRKPKKKRKV.

The term “nucleobase,” “nitrogenous base,” or “base,” used interchangeably herein, refers to a nitrogen-containing biological compound that forms a nucleoside, which in turn is a component of a nucleotide. The ability of nucleobases to form base pairs and to stack one upon another leads directly to long-chain helical structures such as ribonucleic acid (RNA) and deoxyribonucleic acid (DNA). Five nucleobases—adenine (A), cytosine (C), guanine (G), thymine (T), and uracil (U)—are called primary or canonical. Adenine and guanine are derived from purine, and cytosine, uracil, and thymine are derived from pyrimidine. DNA and RNA can also contain other (non-primary) bases that are modified. Non-limiting exemplary modified nucleobases can include hypoxanthine, xanthine, 7-methylguanine, 5,6-dihydrouracil, 5-methylcytosine (m5C), and 5-hydromethylcytosine. Hypoxanthine and xanthine can be created through mutagen presence, both of them through deamination (replacement of the amine group with a carbonyl group). Hypoxanthine can be modified from adenine. Xanthine can be modified from guanine. Uracil can result from deamination of cytosine. A “nucleoside” consists of a nucleobase and a five carbon sugar (either ribose or deoxyribose). Examples of a nucleoside include adenosine, guanosine, uridine, cytidine, 5-methyluridine (m5U), deoxyadenosine, deoxyguanosine, thymidine, deoxyuridine, and deoxycytidine. Examples of a nucleoside with a modified nucleobase includes inosine (I), xanthosine (X), 7-methylguanosine (m7G), dihydrouridine (D), 5-methylcytidine (m5C), and pseudouridine (Y). A “nucleotide” consists of a nucleobase, a five carbon sugar (either ribose or deoxyribose), and at least one phosphate group. Non-limiting examples of modified nucleobases and/or chemical modifications that a modified nucleobase may include are the following: pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine.

The term “nucleic acid programmable DNA binding protein” or “napDNAbp” may be used interchangeably with “polynucleotide programmable nucleotide binding domain” to refer to a protein that associates with a nucleic acid (e.g., DNA or RNA), such as a guide nucleic acid or guide polynucleotide (e.g., gRNA), that guides the napDNAbp to a specific nucleic acid sequence. In some embodiments, the polynucleotide programmable nucleotide binding domain is a polynucleotide programmable DNA binding domain. In some embodiments, the polynucleotide programmable nucleotide binding domain is a polynucleotide programmable RNA binding domain. In some embodiments, the polynucleotide programmable nucleotide binding domain is a Cas9 protein. A Cas9 protein can associate with a guide RNA that guides the Cas9 protein to a specific DNA sequence that is complementary to the guide RNA. In some embodiments, the napDNAbp is a Cas9 domain, for example a nuclease active Cas9, a Cas9 nickase (nCas9), or a nuclease inactive Cas9 (dCas9). Non-limiting examples of nucleic acid programmable DNA binding proteins include, Cas9 (e.g., dCas9 and nCas9), Cas12a/Cpf1, Cas12b/C2c1, Cas12c/C2c3, Cas12d/CasY, Cas12e/CasX, Cas12g, Cas12h, Cas12i, and Cas12j/CasΦ (Cas12j/Casphi). Non-limiting examples of Cas enzymes include Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (also known as Csn1 or Csx12), Cas10, Cas10d, Cas12a/Cpf1, Cas12b/C2c1, Cas12c/C2c3, Cas12d/CasY, Cas12e/CasX, Cas12g, Cas12h, Cas12i, Cas12j/CasΦ, Cpf1, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, homologues thereof, or modified or engineered versions thereof. Other nucleic acid programmable DNA binding proteins are also within the scope of this disclosure, although they may not be specifically listed in this disclosure. See, e.g., Makarova et al. “Classification and Nomenclature of CRISPR-Cas Systems: Where from Here?” CRISPR J. 2018 October; 1:325-336. doi: 10.1089/crispr.2018.0033; Yan et al., “Functionally diverse type V CRISPR-Cas systems” Science. 2019 Jan. 4; 363 (6422): 88-91. doi: 10.1126/science.aav7271, the entire contents of each are hereby incorporated by reference. Exemplary nucleic acid programmable DNA binding proteins and nucleic acid sequences encoding nucleic acid programmable DNA binding proteins are provided in the Sequence Listing as SEQ ID NOs: 197-245, 254-260, and 378.

The terms “nucleobase editing domain” or “nucleobase editing protein,” as used herein, refers to a protein or enzyme that can catalyze a nucleobase modification in RNA or DNA, such as cytosine (or cytidine) to uracil (or uridine) or thymine (or thymidine), and adenine (or adenosine) to hypoxanthine (or inosine) deaminations, as well as non-templated nucleotide additions and insertions. In some embodiments, the nucleobase editing domain is a deaminase domain (e.g., an adenine deaminase or an adenosine deaminase; or a cytidine deaminase or a cytosine deaminase).

As used herein, “obtaining” as in “obtaining an agent” includes synthesizing, purchasing, or otherwise acquiring the agent.

By “subject” or “patient” is meant a mammal, including, but not limited to, a human or non-human mammal. In embodiments, the mammal is a bovine, equine, canine, ovine, rabbit, rodent, nonhuman primate, or feline. In an embodiment, “patient” refers to a mammalian subject with a higher than average likelihood of developing a disease or a disorder. Exemplary patients can be humans, non-human primates, cats, dogs, pigs, cattle, cats, horses, camels, llamas, goats, sheep, rodents (e.g., mice, rabbits, rats, or guinea pigs) and other mammalians that can benefit from the therapies disclosed herein. Exemplary human patients can be male and/or female.

“Patient in need thereof” or “subject in need thereof” is referred to herein as a patient diagnosed with, at risk or having, predetermined to have, or suspected of having a disease or disorder.

The terms “pathogenic mutation”, “pathogenic variant”, “disease causing mutation”, “disease causing variant”, “deleterious mutation”, or “predisposing mutation” refers to a genetic alteration or mutation that is associated with a disease or disorder or that increases an individual's susceptibility or predisposition to a certain disease or disorder. In some embodiments, the pathogenic mutation comprises at least one wild-type amino acid substituted by at least one pathogenic amino acid in a protein encoded by a gene. In some embodiments, the pathogenic mutation is in a terminating region (e.g., stop codon). In some embodiments, the pathogenic mutation is in a non-coding region (e.g., intron, promoter, etc.).

The term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). The terms such as “excipient,” “carrier,” “pharmaceutically acceptable carrier,” “vehicle,” or the like are used interchangeably herein.

The term “pharmaceutical composition” means a composition formulated for pharmaceutical use. In some embodiments, the pharmaceutical composition further comprises a pharmaceutically acceptable carrier. In some embodiments, the pharmaceutical composition comprises additional agents (e.g., for specific delivery, increasing half-life, or other therapeutic compounds).

The terms “protein”, “peptide”, “polypeptide”, and their grammatical equivalents are used interchangeably herein, and refer to a polymer of amino acid residues linked together by peptide (amide) bonds. A protein, peptide, or polypeptide can be naturally occurring, recombinant, or synthetic, or any combination thereof.

The term “fusion protein” as used herein refers to a hybrid polypeptide which comprises protein domains from at least two different proteins.

The term “recombinant” as used herein in the context of proteins or nucleic acids refers to proteins or nucleic acids that do not occur in nature, but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

By “reduces” is meant a negative alteration of at least 10%, 25%, 50%, 75%, or 100%.

By “reference” is meant a standard or control condition. In one embodiment, the reference is a wild-type or healthy cell. In one embodiment, the reference is an unedited cell (e.g., an unmodified immune cell). In other embodiments and without limitation, a reference is an untreated cell that is not subjected to a test condition, or is subjected to placebo or normal saline, medium, buffer, and/or a control vector that does not harbor a polynucleotide of interest. In some cases, the reference is an unedited or wild type cell (e.g., a T cell). In some embodiments, the reference is a B2M KO T cell that does not encode a polypeptide or polypeptide variant of interest (e.g., an HLA class-I single-chain dimer or trimer of the present disclosure or a modified B2M polypeptide of the present disclosure) or encodes a polypeptide that lacks one or more amino acid alterations (e.g., a W60G, W95G, and/or K58E alteration to a B2M polypeptide and/or a D227K, T228A, and/or A245V alteration to an HLA-A, -B, or -C polypeptide).

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, about 100 nucleotides or about 300 nucleotides or any integer thereabout or therebetween. In some embodiments, a reference sequence is a wild-type sequence of a protein of interest. In other embodiments, a reference sequence is a polynucleotide sequence encoding a wild-type protein.

The term “RNA-programmable nuclease,” and “RNA-guided nuclease” refer to a nuclease that forms a complex with (e.g., binds or associates with) one or more RNA(s) that is not a target for cleavage. In some embodiments, an RNA-programmable nuclease, when in a complex with an RNA, may be referred to as a nuclease: RNA complex. Typically, the bound RNA(s) is referred to as a guide RNA (gRNA). In some embodiments, the RNA-programmable nuclease is the (CRISPR-associated system) Cas9 endonuclease, for example, Cas9 (Csn1) from Streptococcus pyogenes (e.g., SEQ ID NO: 197), Cas9 from Neisseria meningitidis (NmeCas9; SEQ ID NO: 208), Nme2Cas9 (SEQ ID NO: 209), Streptococcus constellatus (ScoCas9), or derivatives thereof (e.g., a sequence with at least about 85% sequence identity to a Cas9, such as Nme2Cas9 or spCas9).

The term “single nucleotide polymorphism (SNP)” is a variation in a single nucleotide that occurs at a specific position in the genome, where each variation is present to some appreciable degree within a population (e.g., >1%). SNPs can fall within coding regions of genes, non-coding regions of genes, or in the intergenic regions (regions between genes). In some embodiments, SNPs within a coding sequence do not necessarily change the amino acid sequence of the protein that is produced, due to degeneracy of the genetic code. SNPs in the coding region are of two types: synonymous and nonsynonymous SNPs. Synonymous SNPs do not affect the protein sequence, while nonsynonymous SNPs change the amino acid sequence of protein. The nonsynonymous SNPs are of two types: missense and nonsense. SNPs that are not in protein-coding regions can still affect gene splicing, transcription factor binding, messenger RNA degradation, or the sequence of noncoding RNA. Gene expression affected by this type of SNP is referred to as an eSNP (expression SNP) and can be upstream or downstream from the gene. A single nucleotide variant (SNV) is a variation in a single nucleotide without any limitations of frequency and can arise in somatic cells. A somatic single nucleotide variation can also be called a single-nucleotide alteration.

By “specifically binds” is meant a nucleic acid molecule, polypeptide, polypeptide/polynucleotide complex, compound, or molecule that recognizes and binds a target polypeptide and/or nucleic acid molecule of the disclosure, but which does not substantially recognize and bind other molecules in a sample, for example, a biological sample.

By “substantially identical” is meant a polypeptide or nucleic acid molecule exhibiting at least 50% identity to a reference amino acid sequence. In one embodiment, a reference sequence is a wild-type amino acid or nucleic acid sequence. In another embodiment, a reference sequence is any one of the amino acid or nucleic acid sequences described herein. In one embodiment, such a sequence is at least about 60%, 80%, 85%, 90%, 95%, 96%, 97%, 98%, 99%, 99.9%, or even 99.99%, identical at the amino acid level or nucleic acid level to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

Nucleic acid molecules useful in the methods of the disclosure include any nucleic acid molecule that encodes a polypeptide of the disclosure or a functional fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the disclosure include any nucleic acid molecule that encodes a polypeptide of the disclosure or a functional fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

By “split” is meant divided into two or more fragments.

A “split polypeptide” or “split protein” refers to a protein that is provided as an N-terminal fragment and a C-terminal fragment translated as two separate polypeptides from a nucleotide sequence(s). The polypeptides corresponding to the N-terminal portion and the C-terminal portion of the split protein may be spliced to form a “reconstituted” protein.

The term “target cell” refers to a cell that is acted on by another cell. In an embodiment, the target cell is a modified immune cell that is the target of the host immune system.

The term “target site” refers to a nucleotide sequence or nucleotide of interest within a nucleic acid molecule that is modified. In embodiments, the modification is deamination of a base. The deaminase can be a cytidine or an adenine deaminase. The fusion protein or base editing complex comprising a deaminase may comprise a dCas9-adenosine deaminase fusion protein, a Cas12b-adenosine deaminase fusion, or a base editor disclosed herein.

As used herein, the terms “treat,” treating,” “treatment,” and the like refer to reducing or ameliorating a disorder and/or symptoms associated therewith or obtaining a desired pharmacologic and/or physiologic effect. It will be appreciated that, although not precluded, treating a disorder or condition does not require that the disorder, condition or symptoms associated therewith be completely eliminated. In some embodiments, the effect is therapeutic, i.e., without limitation, the effect partially or completely reduces, diminishes, abrogates, abates, alleviates, reduces the intensity of, or cures a disease and/or adverse symptom attributable to the disease. In some embodiments, the effect is preventative, i.e., the effect protects or prevents an occurrence or reoccurrence of a disease or condition. To this end, the presently disclosed methods comprise administering a therapeutically effective amount of a composition as described herein.

By “tumor necrosis factor alpha (TNF-alpha) polypeptide” is meant a protein having at least about 85% amino acid sequence identity to GenBank accession No. CAA26669.1, which is provided below, or a functional fragment thereof having immunomodulatory activity.

>CAA26669.1 TNF-alpha [Homo sapiens]

(SEQ ID NO: 453)
MSTESMIRDVELAEEALPKKTGGPQGSRRCLF
LSLFSFLIVAGATTLFCLLHFGVIGPQREEFP
RDLSLISPLAQAVRSSSRTPSDKPVAHVVANP
QAEGQLQWLNRRANALLANGVELRDNQLVVPS
EGLYLIYSQVLFKGQGCPSTHVLLTHTISRIA
VSYQTKVNLLSAIKSPCQRETPEGAEAKPWYE
PIYLGGVFQLEKGDRLSAEINRPDYLDFAESG
QVYFGIIAL.

By “tumor necrosis factor alpha (TNF-alpha) polynucleotide” is meant a nucleic acid molecule encoding an TNF-alpha polypeptide, as well as the introns, exons, 3′ untranslated regions, 5′ untranslated regions, and regulatory sequences associated with its expression, or fragments thereof. An exemplary human TNF-alpha polynucleotide sequence is provided at Ensembl Accession No. ENSG00000232810.

By “uracil glycosylase inhibitor” or “UGI” is meant an agent that inhibits the uracil-excision repair system. Base editors comprising a cytidine deaminase convert cytosine to uracil, which is then converted to thymine through DNA replication or repair. In various embodiments, a uracil DNA glycosylase (UGI) prevent base excision repair which changes the U back to a C. In some instances, contacting a cell and/or polynucleotide with a UGI and a base editor prevents base excision repair which changes the U back to a C. An exemplary UGI comprises an amino acid sequence as follows:

>splP14739IUNGI_BPPB2 Uracil-DNA glycosylase inhibitor

(SEQ ID NO: 231)
MTNLSDIIEKETGKQLVIQESILMLPEEVEEVIG
NKPESDILVHTAYDESTDENVMLLTSDAPEYKPW
ALVIQDSNGENKIKML.

In some embodiments, the agent inhibiting the uracil-excision repair system is a uracil stabilizing protein (USP). See, e.g., WO 2022015969 A1, incorporated herein by reference.

As used herein, the term “vector” refers to a means of introducing a nucleic acid molecule into a cell, resulting in a transformed cell. Vectors include plasmids, transposons, phages, viruses, liposomes, lipid nanoparticles, and episomes.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All terms are intended to be understood as they would be understood by a person skilled in the art. Unless defined otherwise, all technical and scientific terms used herein have the same meaning as commonly understood by one of ordinary skill in the art to which the disclosure pertains

In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended. This wording indicates that specified elements, features, components, and/or method steps are present, but does not exclude the presence of other elements, features, components, and/or method steps. Any embodiments specified as “comprising” a particular component(s) or element(s) are also contemplated as “consisting of” or “consisting essentially of” the particular component(s) or element(s) in some embodiments. It is contemplated that any embodiment discussed in this specification can be implemented with respect to any method or composition of the present disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

### BRIEF DESCRIPTION OF THE DRAWINGS

FIG. 1 provides a schematic showing an embodiment of a method for generating HLA class-I attenuated allogeneic immune cells. First, allogeneic immune cells are edited by contacting the cells with a base editor and a guide RNA (gRNA) targeting an endogenous beta-2-microglobulin (B2M) polynucleotide in the cells to modify a nucleobase of the B2M polynucleotide, where modification of the nucleobase leads to ablation of surface-display of HLA class-I polypeptides on the cells. Then, surface-display of HLA class-I polypeptides is reconstituted so that expression and/or activity of HLA class-I polypeptides is attenuated relative to unedited cell expression. In various embodiments, this is done by 1) expressing B2M in the cells under the control of a promoter (e.g., an altered or heterologous promoter) that effects reduced levels (e.g., mRNA transcript and/or polypeptide levels) of B2M in the cells relative to levels in a wild type cell, and/or 2) expressing in the cells a B2M polypeptide containing an amino acid alteration(s) (e.g., W60G, W95G) that reduce the affinity of B2M for HLA class-I heavy chain polypeptides. In embodiments, expressing the altered B2M polypeptide in the cells involves modifying a B2M polynucleotide in the cells.

FIGS. 2A and 2B provide a schematic and a bar graph. FIG. 2A provides a schematic showing lentiviral constructs generated that contained polynucleotides encoding polypeptides containing from N-terminus to C-terminus: A) a CD4 extracellular domain based chimeric antigen receptor (CD4 CAR), B) a T2A self-cleaving peptide, and C) either i) a wild-type B2M polypeptide (i.e., B2M (WT)) or ii) a B2M polypeptide containing a W60G alteration (i.e., B2M (W60G)). Expression of the polypeptides was driven by a human EF1-alpha (EF1a) promoter. FIG. 2B provides a bar graph showing the frequency of lentiviral transduced cells expressing B2M (WT) or B2M (W60G). In FIG. 2B, “UTD” indicates untransduced cells.

FIGS. 3A-3C provide histograms and bar graphs demonstrating that when B2M expression was driven by an EF1a promoter, surface-display of reconstituted B2M (WT) was less than endogenous B2M in unmodified cells, and that the W60G alteration further reduced B2M surface-display levels relative to wild type cells. FIG. 3A provides flow cytometry histograms showing that B2M surface-display levels in unmodified T cells (unmodified) was higher than that observed in cells containing a B2M polynucleotide base-edited to knock out expression of endogenous B2M (“B2M edit”) or cells containing the B2M edit to knock out endogenous B2M expression and containing a polypeptide encoding B2M (WT) or B2M (W60G) under the control of the EF1a promoter to reconstitute B2M expression. FIG. 3B provides a bar graph showing a reduction, as measured using flow cytometry, in B2M geometric mean fluorescent intensity (gMFI) in CD4+ T cells containing the B2M edit (i.e., B2M knock-out cells (B2M KO cells)) or cells containing the B2M edit (B2M KO) and expressing B2M (WT) or B2M (W60G) under the control of the EF1a promoter. FIG. 3C provides a bar graph showing a reduction, as measured using flow cytometry, in B2M geometric mean fluorescent intensity (gMFI) in CD8+ T cells containing the B2M edit (i.e., B2M knock-out cells (B2M KO cells)) or cells containing the B2M edit (B2M KO) and expressing B2M (WT) or B2M (W60G) under the control of the EF1a promoter. In FIGS. 3B and 3C, the percent loss is measured relative to wild type cells.

FIGS. 4A and 4B provide histograms and a bar graph. FIG. 4A provides flow cytometry histograms showing that reconstituting B2M expression in cells containing the B2M edit (i.e., cells base edited to knock out endogenous expression of B2M) by expressing B2M (WT) or B2M (W60G) under the control of the EF1a promoter in the cells restored surface-display levels of HLA-A2 on the cells. The numbers above each histogram in FIG. 4A indicate the percent of total cells counted that had HLA-A2 surface-display levels falling within the range indicated by the horizontal line. FIG. 4B provides a bar graph showing the levels of HLA-A2 surface-display levels measured as percent geometric mean fluorescent intensity (gMFI) relative to wild type cells observed in cells containing the B2M edit and expressing B2M (WT) or B2M (W60G) under the control of the EF1a promoter to reconstitute B2M expression. Cells reconstituting B2M expression by expressing B2M (WT) or B2M (W60G) showed reduced levels of surface-display (i.e., surface-expression) of HLA-A2.

FIGS. 5A and 5B present flow cytometry contour plots and a plot showing that reconstituting B2M expression in T cells through expression of B2M (W60G) in the T cells protected the T cells from alloreactive T cell lysis. FIG. 5A provides a set of flow cytometry contour plots showing that B2M knock-out T cells reconstituting B2M expression through expression of B2M WT under the control of an EF1a promoter were more susceptible to alloreactive T cell lysis than were B2M knock-out T cells reconstituting B2M expression through expression of B2M (W60G) under the control of the EF1a promoter. Cell lysis was evaluated using a T cell mixed leukocyte reaction, where the effector cells (E) were mismatched (i.e., alloreactive) T cells and the target cells (T) were the B2M KO cells expressing B2M (W60G) or B2M (WT) to reconstitute B2M expression. Cell lysis was evaluated at effector to target cell ratios (E: T) of 0:1 and 20:1. The numbers within the plots of FIG. 5A indicate the percent of counted cells falling within the outlined region, which corresponds to unlysed cells. Cells were counted after 48 hours of co-culture using fluorescent antibodies targeting CD8 (x-axis) and CD4 (y-axis). FIG. 5B provides a plot showing percent cell lysis observed at the various E: T ratios indicated on the x-axis in T cell mixed leukocyte reactions similar to those described for FIG. 5A. Cells were counted after 48 hours of co-culture. In FIG. 5A “BV510” represents the fluorescent dye Brilliant Violet 510TM and “PE-Cy™7” represents a tandem fluorophore that can be excited by a broad range of lasers including, for example, 488 nm, 532 nm, and 561 nm.

FIG. 6 provides a schematic showing the generation of allogeneic immune cells expressing a beta-2-microglobulin (B2M) polypeptide containing an alteration (e.g., K58E) that abrogates CD8 binding. Endogenous expression of B2M in the cells is knocked out using base editing to modify the B2M gene in the cells and expression of B2M is reconstituted in the cells by contacting the cells with a polynucleotide encoding the B2M polypeptide containing the alteration that abrogates CD8 binding (i.e., B2M (K58E)). The base editor to the B2M gene is associated with a reduction or ablation of surface-display of HLA class-I polypeptides on the cells. The base editing is carried out by contacting the cells with a base editor and a guide RNA (gRNA) targeting an endogenous B2M gene. Reconstitution of B2M expression in the cells is associated with a reconstitution of surface-display of HLA class-I polypeptides on the cells. The B2M (K58E) polypeptide has a reduced affinity for CD8 present on alloreactive T cells relative to a wild type B2M polypeptide.

FIGS. 7A-7C provide schematics of polypeptides containing an HLA class-I heavy chain (HLA-X) domain and how the polypeptides can be used to reduce or abrogate alloreactive T cell CD8 binding. FIG. 7A provides a schematic showing an HLA class-I single-chain dimer containing from N-terminus to C-terminus: a signal peptide (light grey box), a beta-2-microglobulin (B2M) domain, a linker (black box), and an HLA class-I heavy chain (HLA-X) domain. FIG. 7B provides a schematic showing an HLA class-I single chain trimer containing from N-terminus to C-terminus: a signal peptide (light grey box), a cognate peptide (cPEP) domain, a linker (black box), a B2M domain, a linker (black box), and an HLA class-I heavy chain (HLA-X) domain. In embodiments, the B2M domain contains an amino acid alteration that reduces or abrogates CD8 binding (e.g., a K58E alteration). In FIGS. 7A-7C, HLA-X represents an HLA class-I heavy chain domain derived from an HLA-A, HLA-B, =HLA-C, or HLA-E polypeptide. In embodiments, the HLA-X domain contains one or more amino acid alterations that reduce or abrogate binding to CD8 (e.g., D227K, T228A, and/or A245V). FIG. 7C provides a schematic showing how, in some embodiments, expression of an HLA-X polypeptide and/or HLA-X domain-containing polypeptide (e.g., the HLA class-I single chain dimer or HLA class-I single chain trimer) of the present disclosure in a T cell reduces or eliminates alloreactive T cell CD8 binding.

FIG. 8 provides a collection of flow cytometry contour plots showing surface-expression levels of HLA-A2 single-chain dimer polypeptides in B2M knock-out T cells contacted with lentiviral vectors containing polynucleotides encoding HLA-A2 single-chain dimer polypeptides containing a wild type (WT) HLA-A2 domain or an HLA-A2 domain containing a D227K/T228A alteration, an A245V alteration, or a D227K/T228A/A246V alteration (i.e., HLA-A2 (WT), HLA-A2 (D227K/T228A), HLA-A2 (A245V), and HLA-A2 (D227K/T228A/A246V), respectively), as indicated above the plots, and a wild type (WT) B2M domain or a B2M (K58E) domain, as indicated to the left of the plots (see FIG. 7A for a schematic of HLA class-I single-chain dimers). The alterations to the HLA-A2 domain were within the alpha-3 domain of HLA-A2. All cells were base-edited to knock out endogenous expression of B2M. The numbers in each plot indicate the percent of total counted cells falling within each respective quadrant delimited by the two crossed lines. In FIG. 8, “EGFR” represents a truncated epidermal growth factor receptor (EGFR), APC represents “allophycocyanin”, and “PE” represents phycoerythrin.

FIGS. 9A-9C provide bar graphs demonstrating that alterations in the alpha-3 domain of HLA-A2 single-chain dimer polypeptides expressed by B2M knock-out T cells reduced cytokine production by alloreactive T cells co-cultured with the B2M knock-out T cells. B2M knock-out T cells expressing HLA-A2 single-chain dimers containing the B2M domain and/or HLA-A2 domain alterations indicated beneath the x-axis were co-cultured with alloreactive T cells for 24 hours, after which supernatants were harvested and secretion of granzyme B (GZMB) (FIG. 9A), IFN-gamma (FIG. 9B), and TNF-alpha (FIG. 9C) were measured. In each of FIGS. 9A-9C, the y-axis indicates analyte production levels as a percentage of maximum production measured for alloreactive T cells co-cultured for 24 hours with B2M knock-out T cells expressing HLA-A2 single-chain dimer polypeptides containing a WT B2M domain and a WT HLA-A2 domain (WT B2M/HLA-A2). In FIGS. 9A-9C is included a matrix indicating the sequence of the B2M (i.e., WT or K58E) and HLA-A2 (i.e., WT, D227K/T228A, D227K/T228A/A245V, or A245V) domains of each HLA-A2 single-chain dimer polypeptide corresponding to each bar.

FIGS. 10A and 10B provide flow cytometry contour plots and a plot showing results from a cytotoxicity assay in which allogeneic T cells base edited to knock-out endogenous expression of beta-2-microglobulin and expressing HLA-A2 single-chain dimer polypeptides containing the indicated B2M and HLA-A2 domains were co-cultured with HLA-A2 alloreactive T cells for 48 hours. Cells expressing an HLA-A2 single-chain dimer polypeptide containing an HLA-A2 domain with a D227K/T228A alteration showed the greatest levels of protection from alloreactive T cell lysis. FIG. 10A provides flow cytometry contour plots showing that cells B2M knock-out cells expressing an HLA-A2 single-chain dimer polypeptide containing a wild type B2M domain (B2M (WT)) and an HLA-A2 domain containing a D227K/T228A alteration (A*02 (D-K/T-A) showed greater protection from lysis by alloreactive T cells than B2M knock-out cells expressing an HLA-A2 single-chain dimer polypeptide containing a B2M (WT) domain and a wild type HLA-A2 domain (A*02 (WT)). The B2M knock-out cells expressing the HLA-A2 single-chain dimer polypeptides were co-cultured as target cells (T) in the presence of HLA-A2 alloreactive T cells at the indicated ratios for 48 hours prior to cell counts carried out using flow cytometry. The target allogeneic T cells (right portion of each plot) all expressed a truncated epidermal growth factor receptor (EGFR) and an HLA-A2 single-chain dimer polypeptide, and off-target cells did not express EGFR (left portion of each plot) or the single-chain dimer polypeptide. The numbers listed in the plots indicate the number of total counted cells falling within the corresponding portion of the figure delimited by the vertical line. In FIG. 10A, “PE” represents phycoerythrin and “BV605” represents the tandem fluorochrome BD Horizon Brilliant Violet™ 605. FIG. 10B provides a plot summarizing results from cytotoxicity assays carried out as described for FIG. 10A using B2M KO cells expressing the HLA-A2 single-chain dimer polypeptides indicated in the legend. The highest levels of lysis were observed for cells expressing HLA-A2 single-chain dimer polypeptides containing B2M (WT) and wild-type HLA-A2 (A*02) domains.

FIG. 11 provides a collection of flow cytometry contour plots demonstrating surface-expression of the indicated HLA class-I single-chain dimers and trimers (see Tables 8A and 22) in B2M knock-out T cells transduced with polynucleotides encoding the same. In FIG. 11 the numbers 246-259 in the upper-left quadrant correspond to the ID #'s listed in Tables 8A and 9. In FIG. 11, the numbers having one or two decimal places indicate the percent of total counted cells falling within the indicated quadrant, “UE” indicates unedited cells, and “B2M KO” indicates cells edited using base-editing to knock out expression of B2M, where neither the UE cells nor the B2M KO cells expressed any of the HLA class-I single-chain dimers or trimers. Base-editing was carried out using ABE8.20m and a guide RNA targeting the B2M gene. Cells were co-stained for B2M and EGFR, which is co-surface-expressed with HLA class-I heavy chain polypeptides (e.g., HLA-A, HLA-B, HLA-C, and HLA-E).

FIGS. 12A-12C provide plots demonstrating that HLA-B and HLA-C HLA class-I single-chain dimers and trimers inhibited natural killer (NK) cell degranulation in vitro relative to B2M KO T cells. B2M knock-out T cells expressing the indicated HLA class-I single-chain dimers and trimers (see Tables 8A and 9) were co-cultured with primary human natural killer (NK) cells at a 1:1 effector-to-target cell ratio for 6 hours. The frequency of degranulating (CD107a+) NK cells was measured by fluorescent antibody staining using an anti-CD107a BV650 antibody. The NK cells were also stained with antibodies against inhibitory receptors including CD158a (FIG. 12A), CD158b (FIG. 12B), and CD158e1 (FIG. 12C). The plots of FIGS. 12A-12C each respectively show frequency of degranulating (i.e., CD107a+) NK cells in the subsets CD158a+, CD158b+, and CD158e1+, respectively. The grey boxes in FIGS. 12A-12C show the min-max range for % CD107a+. The minimum % CD107a+ value corresponds to the value measured for NK cells falling within a CD158a+ (FIG. 12A), CD158b+ (FIG. 12B), or CD158e1+ (FIG. 12C) subset when cultured with unmodified HLA-ABC+ T cells, and the maximum % CD107a+value corresponds to value measured for NK cells falling within a CD158a+ (FIG. 12A), CD158b+ (FIG. 12B), or CD158e1+ (FIG. 12C) subset when cocultured with B2M knock-out T cells. In FIGS. 12A-12C, the numbers listed below the x-axis correspond to the ID #'s listed in Tables 8A and 9.

FIG. 13 provides a plot demonstrating that reconstitution of HLA-A, -B, or -C surface-expression in B2M knock-out T cells conferred protection against NK cell lysis relative to untransduced on-target B2M knock-out cells. HLA-A, -B, or -C surface-expression was reconstituted in B2M knock-out T cells by transducing the cells with polynucleotides encoding the indicated HLA class-I single-chain dimers and trimers indicated beneath the x-axis of the plot of FIG. 13 (see Tables 8A and 9), such that the cells surface-expressed the encoded polypeptides. B2M knock-out T cells or B2M knock-out T cells surface-expressing the indicated HLA class-I single-chain dimers and trimers were mixed evenly with unmodified HLA-A+, -B+, C+ T cells (off-target), and then co-cultured with primary human NK cells at a 1:1 and 0:1 effector-to-target ratio for 48 hours. Then, specific lysis of on-target cells was calculated by determining the residual frequency of on-target cells in the presence of effector natural killer (NK) cells (i.e., 1:1 effector-to-target ratio) relative to a no effector NK cell control (i.e., 0:1 effector-to-target ratio). The plot of FIG. 13 shows the frequency of specific lysis of on-target cells mediated by NK cells.

FIGS. 14A and 14B provide schematics depicting HLA class-I single-chain dimers and trimers covalently fused to effector domains (i.e., “functionalized” single-chain dimers and trimers). The effector domains (alternatively “modules”) inhibit alloreactive T cells and/or natural killer cells. FIG. 14A provides a schematic depicting HLA class-I single-chain dimers and trimers fused (e.g., at the N-terminus) to an extracellular domain (ECD) of an effector domain (e.g., a PD-L1 domain, a CTLA4 domain, an HLA-A, -B, -C, or -E domain, and/or a B2M domain). In embodiments the extracellular domain of the effector domain is fused to the N-terminus of a membrane-bound HLA class-I single-chain dimer or trimer via a linker sequence. FIG. 14B provides a schematic depicting HLA class-I single-chain dimers and trimers fused (e.g., at the C-terminus) to a membrane-bound effector domain (e.g., CD47), optionally via a liker sequence. In embodiments, the HLA class-I single-chain dimer or trimer does not contain a transmembrane domain (TMB) (i.e., ΔTMD).

### DETAILED DESCRIPTION

The present disclosure features genetically modified allogeneic cells (e.g., immune cells, such as T- or NK-cells), and methods for producing and using these modified cells (e.g., T cells or NK cells) for the treatment of disease (e.g., cancer, autoimmune disease, etc.).

The disclosure is based, at least in part, on the discovery described further in the Examples provided herein that modified cell resistance to lysis by alloreactive T cells or natural killer (NK) cells is increased by modifying the cells to knock out endogenous expression of beta-2-microglobulin (B2M) (e.g., through base editing of the B2M gene) and reconstituting B2M expression in the cells by:
1) Expressing in the cells a B2M polypeptide under the control of a promoter that reduces levels of B2M surface expression (e.g., a promoter that is weaker than the endogenous B2M promoter);
2) Expressing in the cells a B2M polypeptide containing an amino acid alteration(s) (e.g., W60G, W95G) that reduces the affinity of B2M for HLA class-I heavy chain polypeptides; and/or
3) Expressing in the cells an HLA class-I single-chain dimer or trimer of the present disclosure, where in some embodiments the single-chain dimer or trimer contains a B2M and/or HLA class I heavy chain domain containing alterations that reduce or eliminate binding to CD8 (e.g., a K58E alteration to the B2M domain and/or aD227K, T228A, and/or A245V alteration to the HLA class I heavy chain domain).

In some instances, the methods of the disclosure involve one or more of actions 1) through 3) listed above. In embodiments, the methods of the disclosure further involve knocking out expression of HLA-A and HLA-B and not of HLA-C in a cell. In some embodiments, knocking out expression of HLA-A and HLA-B and not of HLA-C involves contacting the cell with an appropriate base editor and a gRNA selected from TSBTx4193 and TSBTx4194, thereby knocking out expression of HLA-A and HLA-B and not of HLA-C. In some embodiments, knocking out expression of HLA-A and HLA-B and not of HLA-C involves contacting the cell with an appropriate base editor and a gRNA having a spacer sequence selected from the spacer sequence of TSBTx4193 or TSBTx4194, thereby knocking out expression of HLA-A and HLA-B and not of HLA-C.

### HLA Class-I Polypeptides

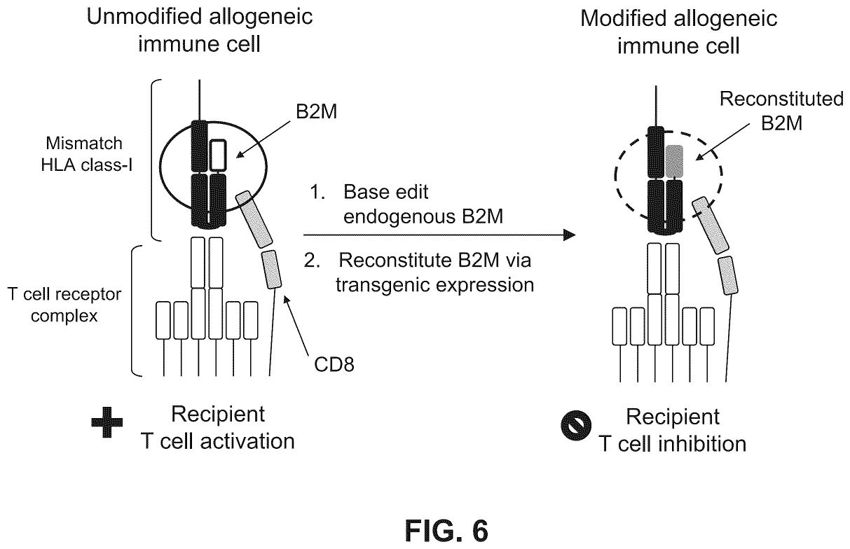

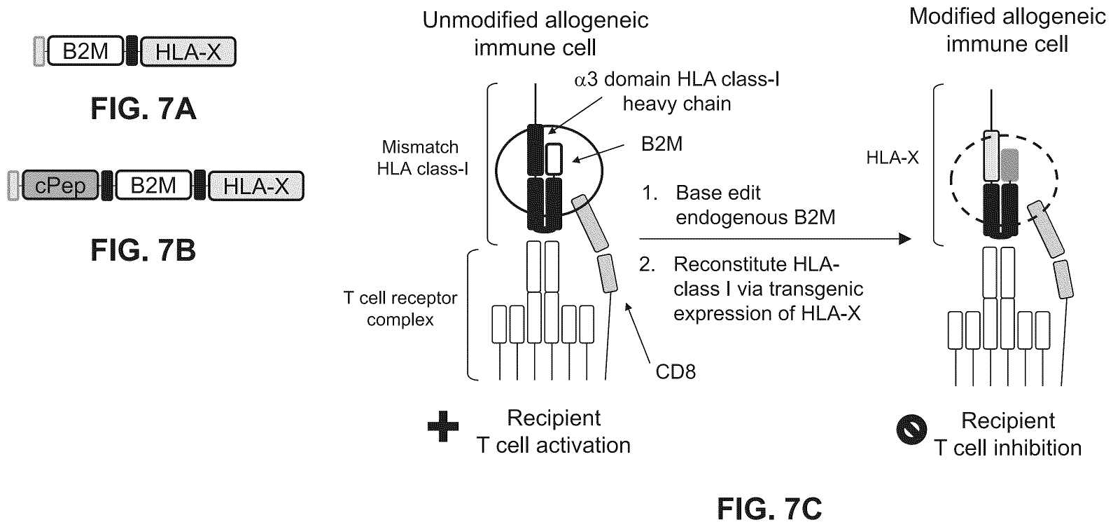

The HLA class-I polypeptides include the heavy chain class I polypeptides HLA-A, -B, and -C (see Sung Yoon Choo, “The HLA System: Genetics, Immunology, Clinical Testing, and Clinical Implications,” Yonsei Med J, 48:11-23 (2007), the disclosure of which is incorporated herein by reference in its entirety for all purposes). HLA class I molecules are expressed on the surface of almost all nucleated cells. The biological role of the HLA class I polypeptides is to present processed peptide antigens expressed within a cell to immune cells (e.g., T cells) and in immune cell activation by allogeneic cells (see, e.g., FIGS. 6 and 7).

HLA class I polypeptides contain glycosylated heavy chains encoded by the HLA class I genes (HLA-A, -B, and -C) and noncovalently bound extracellular beta-2-microglobulin (B2M). Human B2M is invariant and its gene maps to chromosome 15. The class I heavy chain has three extracellular domains (α₁, α₂, and α₃), a transmembrane region, and an intracytoplasmic domain. The α₁ and α2 domains contain variable amino acid sequences, and these domains determine the antigenic specificities of the HLA class I molecules. The α₃ and B2M domains together form immunoglobulin constant domain-like folds. The heavy chain α₁ and α₂ domains form a unique structure consisting of a platform of eight antiparallel β strands and two antiparallel α-helices on top of the platform. A groove is formed by the two α-helices and the β-pleated floor, and this is the binding site for processed peptide antigen. The class I peptide binding groove accommodates a processed peptide (e.g., a cognate peptide (cPep)) of 8 to 10 (predominantly nonamers) amino acid residues.

HLA-A and -B polypeptides can be grouped into superfamilies based upon various criteria, such as structure and/or peptide-binding specificity patterns. Non-limiting examples of HLA-A and -B polypeptides suitable for use in various aspects of the present disclosure include all known HLA-A and -B polypeptides falling within any known superfamily, such as those described in Harjanto, et al. “Clustering HLA Class I Superfamiiles Using Structural Interaction Patterns,” PLOS One, vol. 1, e86655; and/or Francisco, et al. “HLA supertype variation across populations: new insights into the role of natural selection in the evolution of HLA-A and HLA-B polymorphisms,” Immunogenetics, 67:651-663 (2015), the disclosures of which are incorporated herein by reference in their entireties for all purposes. Representative HLA-A superfamiles include A1, A2, A3, A24, and A6X. These HLA-A superfamilies each include the following alleles: A1 (A*0101, A*2601, A*2602, A*2603, A*2902, A*3002, A*8001), A2 (A*0201, A*0202, A*0203, A*0205, A*0206, A*0207, A*0211, A*0212, A*0216, A*0219), A3 (A*0301, A*0302, A*1101, A*3001, A*3101, A*3301, A*6801), A24 (A*2301, A*2402, A*2403), and A6X (A*6802, A*6901), where each superfamily name is followed in parenthesis by representative alleles falling within the superfamily. Representative HLA-B superfamiles include B7, B8, B27, B44, B58, and B62. These HLA-B superfamilies each include the following alleles: B7 (B*0702, B*3501, B*5101, B*5301, B*5401), B8 (B*0801, B*0802, B*0803), B27 (B*1402, B*2705, B*7301), B44 (B*4001, B*4002, B*4402, B*4403, B*4501), B58 (B*1516, B*1517, B*5701, B*5801), and B62 (B*1501, B*1502, B*1503, B*1509, B*3801, B*3901). where each superfamily name is followed in parenthesis by representative alleles falling within the superfamily.

### CAR-T Cell Therapies

Autologous, patient-derived chimeric antigen receptor-T cell (CAR-T) therapies have demonstrated remarkable efficacy in treating disease. While these products have led to significant clinical benefit for patients, the need to generate individualized therapies creates substantial manufacturing challenges and financial burdens. Allogeneic CAR-T therapies were developed as a potential solution to these challenges, having similar clinical efficacy profiles to autologous products while treating many patients with cells derived from a single healthy donor, thereby substantially reducing cost of goods and lot-to-lot variability. Allogeneic approaches are preferred over autologous cell preparation for a number of situations related to the uncertainty of engineering autologous T cells to express a CAR and finally achieving the desired cellular products for a transplant at the time of medical emergency.

However, for allogeneic T cells, or “off-the-shelf” T cells, it is important to carefully negotiate the host's reactivity to the CAR-T cells (HVGD), as well as the allogeneic T cell's potential hostility towards a host cell (GVHD). Additionally, adoptively transferred allogeneic CAR-T cells exhibit poor persistence in vivo due to recipient/host immune-mediated rejection mechanisms. Host-derived alloreactive T cells recognize allogeneic CAR-T cells as “non-self” by binding to peptide complexed with foreign Human Leukocyte Antigen (HLA) on the surface of CAR-T cells. The presence of surface peptide/HLA class-I negatively regulates Natural Killer (NK) cells by engaging inhibitory Killer Ig-Like Receptors (KIRs). As such, the absence of surface HLA on cells leaves them susceptible to NK cell-mediated lysis. Thus, generating HLA-deficient allogeneic CAR-T cells that are below the threshold to activate host-derived alloreactive T cell-mediated rejection and above the threshold to inhibit host-derived NK cells will likely improve allogeneic therapies.

Base editors (BEs) are a class of emerging gene editing reagents that enable highly efficient, user-defined modification of target genomic DNA without the creation of double-stranded breaks (DSBs). In contrast to a nuclease-only editing strategy, concurrent modification of one or more genetic loci by base editing produces highly efficient gene knock-outs with no detectable translocation events. Multiplex editing of genes is likely to be useful in the creation of CAR-T cell therapies with improved therapeutic properties. The methods described herein address known limitations of allogeneic immune cell (e.g., CAR-T cell) products and are a promising development towards the next generation of precision cell-based therapies.

The present disclosure provides modified allogeneic immune cells (e.g., T- or NK-cell) that are less susceptible to NK cell-mediated lysis and are able to overcome host-derived alloreactive T cell-mediated rejection. In some embodiments, the modified allogeneic immune cell described herein is an allogeneic modified CAR-T cell. In some embodiments, the CAR-T cell is an allogeneic T cell that expresses a desired CAR, and can be universally applicable, irrespective of the donor and the recipient's immunogenic compatibility. An allogenic immune cell may be derived from one or more donors. In certain embodiments, the allogenic immune cell is derived from a single human donor. For example, the allogenic T cell may be derived from PBMCs of a single healthy human donor. In certain embodiments, the allogenic immune cell is derived from multiple human donors. In some embodiments, an allogeneic immune cell may be generated, as described herein by using gene modification to introduce concurrent edits at one or more genetic loci. A modification, or concurrent modifications as described herein may be a genetic editing, such as a base editing, generated by a base editor. The base editor may be a C base editor or A base editor. As is discussed herein, base editing may be used to achieve a gene disruption, such that the gene is not expressed. A modification by base editing may be used to achieve a reduction in gene expression. In some embodiments base editor may be used to introduce a genetic modification such that the edited gene does not generate a structurally or functionally viable protein product. In some embodiments, a modification, such as the concurrent modifications described herein may comprise a genetic editing, such as base editing, such that the expression or functionality of the gene product is altered in any way. For example, the expression of the gene product may be enhanced or upregulated as compared to baseline expression levels. In some embodiments the activity or functionality of the gene product may be upregulated as a result of the base editing, or multiple base editing events acting in concert. In some embodiments, a base editor and sgRNAs that provide for multiplex editing are introduced in a single electroporation event, thereby reducing electroporation event associated toxicity. Any known methods for incorporation of exogenous genetic material into a cell may be used to replace electroporation, and such methods known in the art are contemplated for use in any of the methods described herein.

The present disclosure provides an alternative means of producing allogeneic immune cells by using base editing technology and/or a nuclease to reduce or eliminate surface HLA class-I expression and/or expression of an NK cell surface activating ligand (e.g., CD58, CD115, CD48, MICA, MICB, Nectin-2, and/or ULBP). In embodiments, base editing technology and/or a nuclease is used to reduce or eliminate activity and/or surface expression of a B2M, TAP1, TAP2, TAPBP, PDIA3, NLRC5, HLA-A, HLA-B, and/or HLA-C polypeptide. In some embodiments, at least one or more genes encoding proteins that form the peptide loading complex (PLC) (e.g., β2M, TAP1, TAP2, Tapasin) (“PLC genes”), or regulatory elements of such genes, are modified in an allogeneic immune cell with the base editing compositions and methods provided herein. In some embodiments, the PLC genes comprise or consist of B2M, TAP1, TAP2, and Tapasin. In some embodiments, the PLC genes are TAP1 and/or TAP2.

In some embodiments, the PLC genes (e.g., B2M, TAP1, TAP2, Tapasin), or regulatory elements thereof, are modified in an allogeneic immune cell in combination with one or more modifications in at least one additional gene sequence or regulatory element thereof. In some embodiments, the additional gene sequence or regulatory element is selected from TCRα Chain (TRAC), Cluster of Differentiation 58 (CD58), and Class II, Major Histocompatibility Complex Transactivator (CIITA). In some embodiments, one or more of β2M, TAP1, TAP2, and/or Tapasin encoding genes are modified in an allogeneic immune cell in combination with one or more modifications in TRAC, CD58, and/or CIITA encoding genes.

In some embodiments, PLC genes (e.g., B2M, TAP1, TAP2, Tapasin), or regulatory elements thereof, are modified in an allogeneic immune cell in combination with the overexpression of one or more inhibitory receptors. In some embodiments, the inhibitory receptors are selected from Human Leukocyte Antigen-E (HLA-E), Human Leukocyte Antigen-G (HLA-G), Programmed Death Ligand 1 (PD-L1), Cluster of Differentiation 47 (CD47), and/or Cluster of Differentiation 58. In some embodiments, one or more of β2M, TAP1, TAP2, and/or Tapasin are modified in an allogeneic immune cell in combination with the overexpression of one or more of HLA-E, HLA-G, PD-L1, CD47, and/or CD58. In some embodiments, one or more of β2M, TAP1, TAP2, Tapasin, and/or CD58 are modified in an allogeneic immune cell in combination with the overexpression of HLA-E, HLA-G, PD-L1, and/or CD47.

In some embodiments, at least one or more PLC genes (e.g., β2M, TAP1, TAP2, Tapasin, and/or CD58), or regulatory elements thereof, are modified in an allogeneic immune cell in combination with one or more modifications in at least one additional gene sequence or regulatory element thereof and with the overexpression of one or more inhibitory receptors. In some embodiments, one or more of 2M, TAP1, TAP2, and/or Tapasin, are modified in an allogeneic immune cell in combination with modifications in TRAC, CD58, and/or CIITA and with the overexpression of one or more of HLA-E, HLA-G, PD-L1, and/or CD47. In some embodiments, one or more of β2M, TAP1, TAP2, and/or Tapasin are modified in an allogeneic immune cell in combination with one or modifications in TRAC, CD58, and CIITA and with the overexpression of HLA-E, HLA-G, PD-L1, and CD47.

The modified immune cells and methods provided herein address known limitations of CAR-T therapy and is a promising development towards the next generation of precision cell-based therapies.

In embodiments, one or more genes are modified in an allogeneic immune cell so that the modified allogeneic immune cell has a reduced level of, lacks, or have virtually undetectable levels of beta-2-microglobulin and/or one or more of the following polypeptides relative to an unmodified immune cell: B cell leukemia/lymphoma 11b (Bcl11b); B cell leukemia/lymphoma 2 related protein Ald (Bcl2a1d); B cell leukemia/lymphoma 6 (Bcl6); butyrophilin-like 6 (Btn16); CD151 antigen (Cd151); chemokine (C-C motif) receptor 7 (Ccr7); discs large MAGUK scaffold protein 5 (Dlg5); erythropoietin (Epo); G protein-coupled receptor 18 (Gpr18); interferon alpha 15 (Ifna15); interleukin 6 signal transducer (Il6st); interleukin 7 receptor (I17r); Janus kinase 3 (Jak3); membrane associated ring-CH-type finger 7 (Marchf7); NCK associated protein 1 like (Nckap11); phospholipase A2, group IIF (Pla2g2f); runt related transcription factor 3 (Runx3); Signal-regulatory protein beta 1B (Sirpb1b); transforming growth factor, beta 1 (Tgfb1); tumor necrosis factor (ligand) superfamily, member 14 (Tnfsf14); tumor necrosis factor (ligand) superfamily, member 18 (Tnfsf18); tumor necrosis factor (ligand) superfamily, member 8 (Tnfsf8); zinc finger CCCH type containing 8 (Zc3h8); (Rac family small GTPase 2); (Slc4a1); 5-azacytidine induced gene 2 (Azi2); a disintegrin and metalloprotease domain 17 (Adam 17); a disintegrin and metalloprotease domain 8 (Adam8); Acetyl-CoA Acetyltransferase 1 (ACAT1); ACLY; adapter related protein complex 3 beta 1 sububit (Ap3b1); adapter related protein complex 3 delta 1 sububit (Ap3d1); adenosine A2a receptor (Adora2a); adenosine deaminase (Ada); adenosine kinase (Adk); adenosine regulating molecule 1 (Adrml); advanced glycosylation end product-specific receptor (Ager) allograft inflammatory factor 1 (Aif1); AKT1; AKT2; amyloid beta (A4) precursor protein-binding family B member 1 interacting protein (Apbblip); ankyrin repeat and LEM domain (Ankle1); annecin A1 (Anxal); arginase liver (Arg 1); arginase type II (Arg 2); AtPase Cu++ transporting, alpha polypeptide (Atp7a); autoimmune regulator (Aire); autophagy related 5 (Atg5); AXL; B and T Lymphocyte Associated (BTLA); B and T lymphocyte associated (Btla); B cell leukemia/lymphoma 10 (Bcl10); B cell leukemia/lymphoma 11a (Bcl11a); B cell leukemia/lymphoma 2 (Bcl2); B cell leukemia/lymphoma 3 (Bcl3); basic leucine zipper transcription factor, ATF-like (Batf); BCL2-associated X protein (Bax); BCL2L11; beta 2 microglobulin (B2m); BL2-associated agonist of cell dealth (Bad); BLIMP1; Bloom syndrome, RecQ like helicase (Blm); Bmi1 polycomb ring finger oncogene (Bmi1); Bone morphogenic protein 4 (Bmp4); Braf transforming gene (Braf); butyrophilin, subfamily 2, member A1 (Btn2a1); butyrophilin, subfamily 2, member A2 (Btn2a2); butyrophilin-like 1 (Btnl1); butyrophilin-like 2 (Btnl2); c-abl oncogene 1 (Abl1); c-abl oncogene 2 (Abl2); cadherin-like 26 (Cdh26); calcium channel, voltage dependent, beta 4 subunit (Cacnb4); CAMK2D; capping protein regulator and myosin 1 linker 2 (Carmil2); carcinoembryonic antigen-related cell adhesion molecule (Ceacam1); Casitas B-lineage lymphoma b (Cblb); CASP8; Caspase 3 (Casp3); caspase recruitment domain family member 11 (Card11); catenin (cadherin associated protein), beta 1 (Ctnnb1); caveolin 1 (Cav1); CBL-B; CCAAT/enhancer binding protein (C/EBP), beta (Cebpb); CCR10; CCR4; CCR5; CCR6; CCR9; CD103; CD11a; CD122; CD123; CD127; CD130; CD132; CD160 antigen (Cd160); CD161; CD19; CD1d1 antigen (Cd1d1); CD1d2 antigen (CD1d2); CD2 antigen (CD2); CD209e antigen (Cd209e); CD23; CD244 molecule A (Cd244a); CD24a antigen (Cd24a); CD27 antigen (CD27); CD274 antigen (Cd274); CD276 antigen (Cd276); CD28 antigen (Cd28); CD3 delta; CD3 epsilon; CD3 gamma; CD30; CD300A molecule (Cd300a); CD33; CD38; CD4 antigen (Cd4); CD40 ligand (Cd40lg); CD44 antigen (Cd44); CD46 antigen, complement regulatory protein (Cd46); CD47 antigen (Rh-related antigen, integrin-associated signal transducer) (Cd47); CD48 antigen (Cd48); CD5 antigen (Cd5); CD52; CD58; CD59b antigen (Cd59b); CD6 antigen (Cd6); CD69; CD7; CD70; CD74 antigen (Cd74); CD8; CD8 antigen (Cd8); CD80 antigen (Cd80); CD81 antigen (Cd81); CD82; CD83 antigen (Cd83); CD86; CD86 antigen (Cd86); CD8A; CD96; CD99; CDK4; CDK8; CDKN1B; chemokine (C motif) ligand 1 (Xcl1); chemokine (C-C motif) ligand 19 (Ccl19); chemokine (C-C motif) ligand 2 (Ccl2); chemokine (C-C motif) ligand 20 (Ccl20); chemokine (C-C motif) ligand 5 (Ccl5); chemokine (C-C motif) receptor 2 (Ccr2); chemokine (C-C motif) receptor 6 (Ccr6); chemokine (C-C motif) receptor 9 (Ccr9); chemokine (C-X-C motif) ligand 12 (Cxcl12); chemokine (C-X-C motif) receptor (Cxcr4); Chitinase 3 Like 1 (Chi311); cholinergic receptor, nicotinic, alpha polypeptide 7 (Chrna7); chromodomain helicase DNA binding protein 7 (Chd7); CLA; Class II Major Histocompatibility Complex Transactivator (CIITA); cleft lip and palate associated transmembrane protein 1 (Clptm1); Cluster of Differentiation 123 (CD123); Cluster of Differentiation 3 (CD3); Cluster of Differentiation 33 (CD33); Cluster of Differentiation 52 (CD52); Cluster of Differentiation 7 (CD7); Cluster of Differentiation 96 (CD96); coagulation factor II (thrombin) receptor-like 1 (F2rl1); coil-coil domain containing 88B (Ccdc88b); core-binding factor beta (Cbfb); coronin, actin binding protein 1A (Corola); coxsackie virus and adenovirus receptor (Cxadr); CS-1; CSF2CSK; c-src tyrosine kinase (Csk); C-type lectin domain family 2, member i (Clec2i); C-type lectin domain family 4, member a2 (Clec4a2); C-type lectin domain family 4, member d (Clec4d); C-type lectin domain family 4, member e (Clec4e); C-type lectin domain family 4, member f (Clec4f); C-type lectin domain family 4, member g (Clec4g); CUL3; CXCR3; cyclic GMP-AMP synthase (Cgas); cyclin D3 (Ccnd3); cyclin dependent kinase inhibitor 2A (Cdkn2a); cyclin-dependent kinase (Cdk6); CYLD lysine 63 deubiquitinase (Cyld); cysteine-rich protein 3 (Crip3); cytidine 5′-triphosphate synthase (Ctps); Cytochrome P450 Family 11 Subfamily A Member 1 (Cyp11a1); cytochrome P450, family 26, subfamily b, polypeptide (Cyp26b1); Cytokine Inducible SH2 Containing Protein (CISH); cytotoxic T lymphocyte-associated protein 2 alpha (Ctla2a); Cytotoxic T-Lymphocyte Associated Protein 4 (CTLA-4); DCK; dedicator of cytokinesis 2 (Dock2); dedicator of cytokinesis 8 (Dock8); delta like canonical Notch ligand 4 (Dll14); deltex 1, E3 ubiquitin ligase (Dtx1); deoxyhypusine synthase (Dhps); DGKA; DGKZ; DHX37; dicer 1, ribonuclease type III (Dicer1); dipeptidylpeptidase 4 (Dpp4); discs large MAGUK scaffold protein 1 (Dlg1); DnaJ heat shock protein family (Hsp40) member A3 (Dnaja3); dolichyl-di-phosphooligosaccharide-protein glycotransferase (Ddost); double homeobox B-like 1 (Duxbl1); drosha, ribonuclease type III (Drosha); dual specificity phosphatase 10 (Dusp10); dual specificity phosphatase 22 (Dusp22); dual specificity phosphatase 3 (Dusp3); E74-like factor 4 (Elf4); early growth response 1 (Egr1); early growth response 3 (Egr3); ELOB (TCEB2); ENTPD1 (CD39); eomesodermin (Eomes); Eph receptor B4 (Ephb4); Eph receptor B6 (Ephb6); ephrin B1 (Efnb1); ephrin B2 (Efnb2); ephrin B3 (Efnb3); Epstein-Barr virus induced gene 3 (Ebi3); erb-b2 receptor tyrosine kinase (Erbb2); eukaryotic translation initiation factor 2 alpha kinase 4 (Eif2ak4); FADD; family with sequence similarity 49, member B (Fam49b); Fanconi anemia, complementation group A (Fanca); Fanconi anemia, complementation group D2 (Fancd2); Fas (TNF receptor superfamily member 6) (Fas); Fas (TNFRSF6)-associated via death domain (Fadd); Fas Cell Surface Death Receptor (FAS); Fc receptor, IgE, high affinity I, gamma polypeptide (Fcer1g); fibrinogen-like protein 1 (Fgl1); fibrinogen-like protein 2 (Fgl2); FK506 binding protein 1a (Fkbp1a); FK506 binding protein 1b ((Fkbp1b); flotillin 2 (Flot2); FMS-like tyrosine kinase (Flt3); forkhead box J1 (Foxj1); forkhead box N1 (Foxn1); forkhead box P1 (Foxp1); forkhead box P3 (Foxp3); frizzled class receptor 5 (Fzd5); frizzled class receptor 7 (Fzd7); frizzled class receptor 8 (Fzd8); fucosyltransferase 7 (Fut7); Fyn proto-oncogene (Fyn); gap junction protein, alpha 1 (Gja1); GATA binding protein 3 (GATA3); GCN2 kinase (IDO pathway); gelsolin (Gsn); GLI-Kruppel family member GLI3 (Gli3); glycerol-3-phosphate acyltransferase, mitochondrial (Gpam); growth arrest and DNA-damage-inducible 45 gamma (Gadd45g); GTPase, IMAP family member 1 (Gimap1); HITET2; H2.0-like homeobox (Hlx); haematopoietic 1 (hem1); HCLS1 binding protein 3 (Hs1bp3); heat shock 105 kDa/110 kDa protein 1 (Hsph1); heat shock protein 1 (chaperonin) (Hspd1); heat shock protein 90, alpha (cytosolic), class A member 1 (Hsp90aal); hematopoietic SH2 domain containing (Hsh2d); hepatitis A virus cellular receptor 2 (Havcr2); hes family bHLH transcription factor 1 (Hes1); histocompatibility 2, class II antigen A, alpha (H2-Aa); histocompatibility 2, class II antigen A, beta 1 (H2-Ab1); histocompatibility 2, class II, locus DMa (H2-DMa); histocompatibility 2, M region locus 3 (H3-M3); histocompatibility 2, O region alpha locus (H2-Oa); histocompatibility 2, T region locus 23 (H2-T23); HLA-DR; homeostatic iron regulator (Hfe); icos ligand (Icosl); IKAROS family zinc finger 1 (Ikzf1); IL10; IL10RA; IL2 inducible T cell kinase (Itk); IL6R; Indian hedgehog (Ihh); indoleamine 2,3-dioxygenase 1 (Ido1); inducible T cell co-stimulator (Icos); inositol 1,4,5-trisphosphate 3-kinase B (Itpkb); insulin II (Ins2); insulin-like growth factor 1 (Igf1); insulin-like growth factor 2 (Igf2); insulin-like growth factor binding protein 2 (Igfbp2); integrin alpha L (Itgal); integrin alpha M (Itgam); integrin alpha V (Itgav); integrin alpha X (Itgax); integrin beta 2 (Itgb2); integrin, alpha D (Itgad); intercellular adhesion molecule 1 (Icam1); interferon (alpha and beta) receptor 1 (Ifnar1); interferon alpha 1 (Ifna1); interferon alpha 11 (Ifna11); interferon alpha 12 (Ifna12); interferon alpha 13 (Ifna13); interferon alpha 14 (Ifna14); interferon alpha 16 (Ifna16); interferon alpha 2 (Ifna2); interferon alpha 4 (Ifna4); interferon alpha 5 (Ifna5); interferon alpha 6 (Ifna6); interferon alpha 7 (Ifna7); interferon alpha 9 (Ifna9); interferon alpha B (Ifnab); interferon beta 1 (Ifnb1); interferon gamma (Ifng); interferon kappa (Ifnk); interferon regulatory factor 1 (Irf1); interferon regulatory factor 4 (Irf4); interferon zeta (Ifnz); interleukin 1 beta (Il1b; interleukin 1 family, member 8 (Il1f8); interleukin 1 receptor-like 2 (Il1r12); interleukin 12 receptor, beta1 (Il12rb1); interleukin 12a (Il12a); interleukin 12b (Il12b); interleukin 15 (Il15); interleukin 18 (Il18); interleukin 18 receptor 1 (Il18r1); interleukin 2 (112); interleukin 2 receptor, alpha chain (Il2ra); interleukin 2 receptor, gamma chain (Il2rg); interleukin 20 receptor beta (I120rb); interleukin 21 (I121); interleukin 23, alpha subunit p19 (Il23a); interleukin 27 (I127); interleukin 4 (Il4); interleukin 4 receptor, alpha (Il4ra); interleukin 6 (I16); interleukin 7 (I17); IRF8; itchy, E3 ubiquitin protein ligase (Itch); jagged 2 (Jag2); jumonji domain containing 6 (Jmjd6); JUNB; junction adhesion molecule like 9 (Jam9); K (lysine) acetyltransferase 2A (Kat2a); KDEL (Lys-Asp-Glu-Leu) endoplasmic reticulum protein retention receptor 1 (Kdelr1); KIT proto-oncogene receptor tyrosine kinase (Kit); LAG-3; LAIR-1 (CD305); LDHA; lectin, galactose binding, soluble 1 (Lgals1); lectin, galactose binding, soluble 3 (Lgals3); lectin, galactose binding, soluble 8 (Lgals8); lectin, galactose binding, soluble 9 (Lgals9); leptin (Lep); leptin receptor (Lepr); leucine rich repeat containing 32 (Lrrc32); leukocyte immunoglobulin-like receptor, subfamily B, member 4A (Lilrb4a); LFNG O-fucosylpeptide 3-beta-N-acetylglucosaminyltransferase (Lfng); LIF; ligase IV, DNA, ATP-dependent (Lig4); LIM domain only 1 (Lmo1); limb region 1 like (Lmbr1); linker for activation of T cells (Lat); lymphocyte antigen 9 (Ly9); lymphocyte cytosolic protein 1 (Lcp1); lymphocyte protein tyrosine kinase (Lck); lymphocyte transmembrane adaptor 1 (Lax1); lymphocyte-activation gene 3 (Lag3); lymphoid enhancer binding factor 1 (Lef1); LYN; lysyl oxidase-like 3 (Lox13); MAD1 mitotic arrest deficient 1-like 1 (Mad111); MALTI paracaspase (Malt1); MAP4K4; MAPK14; MCJ; mechanistic target of rapamycin kinase (Mtor); MEF2D; Methylation-Controlled J Protein (MCJ); methyltransferase like 3 (Mett13); MGAT5; MHC I like leukocyte 2 (Mill2); midkine (Mdk); mitogen-activated protein kinase 8 interacting protein 1 (Mapk8ip10); moesin (Msn); myelin protein zero-like 2 (Mpzl2); myeloblastosis oncogene (Myb); myosin, heavy polypeptide 9, non-muscle (Myh9); Nedd4 family interacting protein 1 (Ndfip1); neural precursor cell expressed, developmentally down-regulated 4 (Nedd4); NFATc1; NFATC2; NFATC4; NFKB activating protein (Nkap); nicastrin (Ncstn); NK2 homeobox 3 (Nkx2-3); NLR family, CARD domain containing 3 (Nlrc3); NLR family, pyrin domain containing 3 (Nlrp3); non-catalytic region of tyrosine kinase adaptor protein 1 (Nck1); non-catalytic region of tyrosine kinase adaptor protein 2 (Nck2); non-homologous end joining factor 1 (Nhej1); non-SMC condensin II complex, subunit H2 (Ncaph2); Notch-regulated ankyrin repeat protein (Nrarp); NT5E (CD73); nuclear factor of activated T cells, cytoplasmic, calcineurin dependent (Nfatc3); nuclear factor of kappa light polypeptide gene enhancer in B cells inhibitor, delta (Nfkbid); nuclear receptor co-repressor 1 (Ncor1); Nuclear Receptor Subfamily 4 Group A Member 1 (NR4A1); Nuclear Receptor Subfamily 4 Group A Member 2 (NR4A2); Nuclear Receptor Subfamily 4 Group A Member 3 (NR4A3); ODC1; OTU domain containing 5 (Otud5); OTULINL (FAM105A); paired box 1 (Pax1); PDCD1 (PD1; PD-1); PDIA3; pellino 1 (Peli1); peroxiredoxin 2 (Prdx2); PHD1 (EGLN2); PHD2 (EGLN1); PHD3 (EGLN3); phosphodiesterase 5A, cGMP-specific (Pde5a); phosphoinositide-3-kinase regulatory subunit (Pik3r6); phospholipase A2, group IIA (Pla2g2a); phospholipase A2, group IID (Pla2g2d); phospholipase A2, group IIE (Pla2g2e); phosphoprotein associated with glycosphingolipid microdomains 1 (Pag1); PIK3CD; PIKFYVE; POZ (BTB) and AT hook containing zinc finger 1 (Patz1); PPARa; PPARd; PR domain containing 1, with ZNF domain (Prdm1); presenilin 1 (Psen1); presenilin 2 (Psen2); PRKACA; PRKC, apoptosis, WT1, regulator (Pawr); programmed cell death 1 ligand 2 (Pdcd11g2); prosaposin (Psap); prostaglandin E receptor 4 (subtype EP4) (Ptger4); protein kinase C, theta 2 (Prkcq); protein kinase C, zeta (Prkcz); protein kinase, cAMP dependent regulatory, type I, alpha (Prkarla); protein kinase, DNA activated, catalytic polypeptide (Prkdc); protein phosphatase 3, catalytic subunit, beta isoform (Ppp3cb); protein tyrosine phosphatase, non-receptor type 2 (Ptpn2); protein tyrosine phosphatase, non-receptor type 22 (lymphoid) (Ptpn22); protein tyrosine phosphatase, non-receptor type 6 (Ptpn6); protein tyrosine phosphatase, receptor type, C (Ptprc); PTEN; PTPN11; purine-nucleoside phosphorylase (Pnp); purinergic receptor P2X, ligand-gated ion channel, 7 (P2rx7); PVR Related Immunoglobulin Domain Containing (PVRIG; CD112R); PYD and CARD domain containing 7 (Pycard); RAB27A, member RAS oncogene family (Rab27a); RAB29, member RAS oncogene family (Rab29); radical S-adenosyl methionine domain containing 2 (Rsad2); RAR-related orphan receptor alpha (Rora); RAR-related orphan receptor gamma (Ror); RAS guanyl releasing protein 1 (Rasgrp1); ras homolog family member A (Rhoa); ras homolog family member H (Rhoh); RAS protein activator like 3 (Rasal3); RASA2; receptor (TNFRSF)-interacting serine-threonine kinase 2 (Ripk2); recombination activating gene 1 (Rag1); recombination activating gene 2 (Rag2); Regulatory Factor X Associated Ankyrin Containing Protein (RFXANK); RHO family interacting cell polarization regulator 2 (Ripor2); ribosomal protein L22 (Rpl 22); ribosomal protein S6 (Rps6); RING CCCH (C3H) domains 1 (Rc3h1); ring finger and CCCH-type zinc finger domains 2 (Rc3h2); RNF2; runt related transcription factor 1 (Runx1); runt related transcription factor 2 (Runx2); SAM and SH3 domain containing 3 (Sash3); schlafen 1; Selectin P Ligand/P-Selectin Glycoprotein Ligand-1 (SELPG/PSGL1) polypeptide; selenoprotein K (Selenok); sema domain immunoglobulin domain (Ig), transmembrane domain (TM) and short cytoplasmic domain, (semaphorin) 4A (Sema4a); serine/threonine kinase 11 (Stk11); SH3 domain containing ring finger 1 (Sh3rf1); SHP1; sialophorin (Spn); SIGLEC15; signal transducer and activator of transcription 3 (Stat3); signal transducer and activator of transcription 5A (Stat5A); signal transducer and activator of transcription 5B (Stat5B); signal-regulatory protein alpha (Sirpa); Signal-regulatory protein beta 1A (Sirpb1a); Signal-regulatory protein beta 1C (Sirpb1c); SLA; SLAM family member 6 (Slamf6); SLAMF7; SMAD family member 3 (Smad3); SMAD family member 7 (Smad7); SMARCA4; solute carrier family 11 (proton-coupled divalent metal ion transporters), member 1 (Slc11a1); solute carrier family 4 (anion exchanger), member 1; solute carrier family 46, member 2 (Slc46a2); sonic hedgehog (Shh); SOS Ras/Rac guanine nucleotide exchange factor 1 (Sos1); SOS Ras/Rac guanine nucleotide exchange factor 2 (Sos2); special AT-rich sequence binding protein 1 (Satb1); spleen tyrosine kinase (Syk); Sprouty RTK Signaling Antagonist 1 (Spry1); Sprouty RTK Signaling Antagonist 2 (Spry2); squamous cell carcinoma antigen recognized by T cells (Sart1); src homology 2 domain-containing transforming protein B (Shb); Src-like-adaptor 2 (Sla2); SRY (sex determining region Y)-box 4 (Sox4); STK4; suppression inducing transmembrane adaptor 1 (Sit1); suppressor of cytokine signaling 1 (Socs1); suppressor of cytokine signaling 5 (Socs5); suppressor of cytokine signaling 6 (Socs6); surfactant associated protein D (Sftpd); SUV39; syndecan 4 (Sdc4); syntaxin 11 (Stx11); T Cell Immunoglobulin Mucin 3 (Tim-3); T cell immunoreceptor with Ig and ITIM domains (Tigit); T cell receptor alpha joining 18 (Traj18); T Cell Receptor Beta Constant 1 (TRBC1); T Cell Receptor Beta Constant 2 (TRBC2); T cell, immune regulator 1, ATPase, H+ transporting, lysosomal V0 protein A3 (Tcirg1); T cell-interacting, activating receptor on myeloid cells 1 (Tarm1); T-box 21 (Tbx21); TCR; TCR alpha; TCR beta; TCR complex gene sequence; Tet Methylcytosine Dioxygenase 2 (TET2); TGFbRII; TGFbRII (TGFBR2); three prime repair exonuclease 1 (Trex1); thymocyte selection associated (Themis); thymus cell antigen 1, theta (Thyl); TMEM222; TNF receptor-associated factor 6 (Traf6); TNFAIP3; TNFRSF10B; TNFRSF8 (CD30); TOX; TOX2; TRAC; transformation related protein 53 (Trp53); Transforming Growth Factor Beta Receptor II (TGFbRII); transforming growth factor, beta receptor II (Tgfbr2); transmembrane 131 like (Tmem1311); transmembrane protein 98 (Tmem98); triggering receptor expressed on myeloid cells-like 2 (Trem12); TSC complex subunit 1 (Tsc1); tumor necrosis factor (ligand) superfamily, member 11 (Tnfsf11); tumor necrosis factor (ligand) superfamily, member 13b (Tnfsf13b); tumor necrosis factor (ligand) superfamily, member 4 (Tnfsf4); tumor necrosis factor (ligand) superfamily, member 9 (Tnfsf9); tumor necrosis factor receptor superfamily, member 13c (Tnfrsf13c); tumor necrosis factor receptor superfamily, member 4 (Tnfrsf4); tumor necrosis factor, alpha-induced protein 8-like 2 (Tnfalp812); twisted gastrulation BMP signaling modulator 1 (Twsg1); UBASH3A; vanin 1 (Vnn1); vascular cell adhesion molecule 1 (Vcam1); VHL; v-maf musculoaponeurotic fibrosarcoma oncogene family, protein B (avian) (Mafb); V-set and immunoglobulin domain containing 4 (Vsig4); V-Set Immunoregulatory Receptor (VISTA); WD repeat and FYVE domain containing 4 (Wdfy4); wingless-type MMTV integration site family, member 1 (Wnt1); wingless-type MMTV integration site family, member 4 (Wnt4); WNT signaling pathway regulator (Apc); WW domain containing E3 ubiquitin protein ligase 1 (Wwp1); XBP1; YAP1; ZAP70; ZC3H12A; zfp35; zinc finger and BTB domain containing 1 (Zbtb1); zinc finger and BTB domain containing 7B (Zbtb7B); zinc finger CCCH type containing 12A (Zc3h12a); zinc finger CCCH type containing 12D (Zc3h12d); zinc finger E-box binding homeobox 1 (Zeb1); zinc finger protein 36, C3H type (Zfp36); zinc finger protein 36, C3H type-like 1 (Zfp36L1); zinc finger protein 36, C3H type-like 2 (Zfp36L2); and zinc finger protein 683 (Zfp683).

### HLA Single Chain Dimers and Trimers

The present disclosure provides human leukocyte antigen (HLA) single chain dimers and trimers (e.g., HLA class-I single-chain dimers and trimers). In some embodiments, the HLA single chain dimers comprise an HLA-A, -B, -C, and/or -E domain (see, e.g., Table 8A and Example 2) and a beta-2-microglobulin domain. In some instances, the HLA single chain trimers comprise an HLA-A, -B, -C, and/or -E domain (see, e.g., Table 8A and Example 2), a beta-2-microglobulin domain, and a cognate peptide (cPep). In some embodiments, the HLA construct is membrane-bound (e.g., the HLA single-chain dimer or trimer contains a transmembrane domain, such as a transmembrane domain derived from CD4) or the HLA single-chain dimer or trimer is secreted by a cell. Expression of the HLA class-I single-chain dimers or trimers in an immune cell (e.g., a β2M knock-out (KO) immune cell) results in increased resistance to lysis by alloreactive T cells. In some instances, a modified immune cell expressing an HLA single chain dimer or trimer includes modifications to reduce or eliminate endogenous expression of β2M.

In some instances, the HLA heavy chain domain (e.g., HLA-C) contains an A73T amino acid alteration. In some embodiments, the A73T alteration broadens the number of NK cell inhibitory receptors to which an HLA heavy chain domain (e.g., HLA-C) binds.

The various domains of an HLA construct can be connected by linkers, such as those provided herein. The length of the linkers may be elongated or truncated by about or at least about 1, 2, 3, 4, 5, 6, 7, 8, 9, 10, 15, 20, 25, 30, 35, 40, 45, or 50 amino acids. In various instances, the linker is a Gly/Ser-linker (GS-linker). The length of the linkers may be about, at least about, or no more than about 1, 2, 3, 4, 5, 6, 7, 8, 9, 10, 15, 20, 25, 30, 35, 40, 45, 50, 75, or 100 amino acids.

In some instances, the HLA construct comprises an N-terminal signal peptide (e.g., a β2M signal peptide). Any signal peptide known in the art and suitable for secretion and/or membrane-localization of a polypeptide is suitable in the HLA single chain dimers or trimers provided herein.

In some instances, the HLA single chain dimer or trimer contains a transmembrane domain (e.g., a Type I or Type II transmembrane domain; a CD4 transmembrane domain; an HLA heavy chain transmembrane domain, such as a heterologous HLA heavy chain transmembrane domain), optionally at an N-terminal or C-terminal portion thereof. In some embodiments, the HLA single chain dimer or trimer contains a Type II transmembrane domain. In some instances, the HLA-A, -B, -C, or -E domain is derived from an HLA-A, -B, -C, or -E polypeptide from which a transmembrane domain has been deleted. In some instances, the HLA single-chain dimer or trimer contains a wild-type HLA heavy chain transmembrane domain. As described in Chou and Elrod, Proteins: Structure, Function, and Genetics 34:137-153 (1999), a Type I membrane protein is a single-pass transmembrane protein having an extracellular (or luminal) N-terminus and a cytoplasmic C terminus for a cell (or organelle) membrane, and a Type I membrane protein is a single-pass transmembrane protein having an extracellular (or luminal) C-terminus and a cytoplasmid N-terminus for a cell (or organelle) membrane.

In embodiments, an HLA construct contains any one or more of the domains described in Table 8A and/or in Example 2, functional fragments thereof, or extensions thereof, where the functional fragment may correspond to an N-terminal and/or C-terminal truncation by about, at least about, and/or no more than about 1, 2, 3, 4, 5, 6, 7, 8, 9, 10, 15, 20, or 25 amino acids, and where the extension may correspond to an N-terminal and/or C-terminal extension by about, at least about, and/or no more than about 1, 2, 3, 4, 5, 6, 7, 8, 9, 10, 15, 20, or 25 amino acids.

In some embodiments, the HLA single-chain dimer contains one of the following domain arrangements from N-terminus to C-terminus: signal peptide-β2M domain-linker-HLA heavy chain (HLA-X) domain; signal peptide-β2M domain-linker-HLA-X domain-transmembrane domain; transmembrane domain-HLA-X domain-linker-β2M domain; transmembrane domain-β2M domain-HLA-X domain; where HLA-X is an HLA-A, HLA-B, HLA-C, or HLA-E domain. In some embodiments, the HLA single-chain trimer contains one of the following domain arrangements from N-terminus to C-terminus: signal peptide-cPep-linker-β2M domain-linker-HLA-X domain; signal peptide-cPep-linker-β2M domain-linker-HLA-X domain-transmembrane domain; where HLA-X is an HLA-A, HLA-B, HLA-C, or HLA-E domain). In some instances, any one of these domain arrangements can be modified to not include any β2M domain.

Non-limiting examples of cognate peptide (cPep) amino acid sequences include those listed in Tables A and 8A. In some cases, the cPep assists in trimerization of a HLA single-chain trimer. In various instances, trimerization of the HLA single-chain trimer increases inhibition of lysis of a cell by an NK cell.

TABLE A
Representative cPep amino acid sequences.

| | SEQ ID | |
|---|---|---|
| Sequence | NO: | Protein of Origin |
| IIDKSGSTV | 631 | AP-3 complex subunit mu-1 |
| VGDKPVSNF | 632 | Protein unc-119 homolog B |
| AVDGSGTKF | 633 | Ribosome biogenesis protein WDR12 |
| IVDRGSTNL | 634 | Alpha-adducin |
| YVDEHGTRL | 635 | Proteasome subunit beta type-8 |
| GSDPRVTQL | 636 | RILP-like protein 2 |
| IVDRPVTLV | 637 | NADH dehydrogenase [ubiquinone] 1 beta subcomplex |
| | | subunit 10 |
| IVDKSGRTL | 638 | Methionine synthase |
| AGDDAPRAV | 639 | β-Actin or POTE ankyrin domain family member F |
| VSDOANHVL | 640 | Kinesin-like protein KIF21A |
| ASDHAPHTL | 641 | CAD protein |
| VGDPHTVTV | 642 | Bromodomain-containing protein 8 |
| KSDERPVHI | 643 | E3 ubiquitin-protein ligase TRIM33 |
| ASDDGTVRI | 644 | WD repeat-containing protein 26 |
| SIDKTGENF | 645 | 40S ribosomal protein S4, X isoform X isoform |
| TSDDVAKEF | 646 | Protein unc-13 homolog C |
| SADSKPIDV | 647 | Aminoacyl tRNA synthase complex-interacting |
| | | multifunctional protein 1 |
| KLDETGNSL | 648 | DNA topoisomerase 2-alpha |
| MADRGEARL | 649 | RecQ-mediated genome instability protein 2 |
| AADFEIGHF | 650 | Nucleosome assembly protein 1-like 1 |
| NADGKIISL | 651 | Bifunctional glutamate/proline-tRNA ligase |
| AADGKGVVV | 652 | 60S ribosomal protein L28 |
| AADKIHSSF | 653 | Plasminogen activator inhibitor 2 |
| NADAIVVKL | 654 | Staphylococcal nuclease domain-containing protein 1 |
| NADTVSSKL | 655 | AP-2 complex subunit beta |
| SAEKAPVSV | 656 | Transforming acidic coiled-coil-containing protein 1 |
| QGDAVVLKI | 657 | Immunoglobulin superfamily member 8 precursor |
| AADHYSQQM | 658 | Guanylate-binding protein 4 |

The transmembrane domain traverses a cell's lipid bilayer cellular membrane. In some embodiments, this domain is derived from a receptor (e.g., an antigen receptor) having a transmembrane domain, while in other embodiments, this domain is synthetic. In some cases, the transmembrane domain is an HLA-A, -B, -C, or -E transmembrane domain (e.g., a wild-type HLA-A, -B. -C, or -E transmembrane domain). In some embodiments, the transmembrane domain may be derived from a non-human transmembrane domain and, in some embodiments, humanized. By “humanized” is meant having the sequence of the nucleic acid encoding the transmembrane domain optimized such that it is more reliably or efficiently expressed in a human subject. In some embodiments, the transmembrane domain is derived from another transmembrane protein expressed in a human immune effector cell. Examples of such proteins include, but are not limited to, subunits of the T cell receptor (TCR) complex, PD1, or any of the Cluster of Differentiation proteins (e.g., CD4), or other proteins, that are expressed in the immune effector cell and that have a transmembrane domain. In some embodiments, the transmembrane domain will be synthetic, and such sequences will comprise many hydrophobic residues.

Transmembrane domains for use in the disclosed HLA constructs can include at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. In some embodiments, the transmembrane domain is derived from CD4, CD8a, CD28 and CD35.

In embodiments, the HLA single-chain dimers and trimers further comprise an effector domain that functions to improve inhibition of alloreactive T cells and/or NK cells. The HLA single chain dimers and trimers fused to the effector domain can be referred to as “functionalized” single-chain dimers or trimers. In some cases, the effector domain is fused to the N-terminus or C-terminus of a HLA single-chain dimer or trimer. Non-limiting examples of effector domains include PD-L1 or CTLA4 extracellular domains or a CD47 polypeptide. Further non-limiting examples of effector domains include additional single-chain dimers or trimers, which in some instances do not contain any transmembrane domain. In some instances, the effector domain is a membrane-bound domain or a transmembrane domain. If the effector domain is a transmembrane domain or contains a transmembrane domain, it can be advantageous to delete a transmembrane domain from the HLA single-chain dimer or trimer to which the effector domain is fused (e.g., delete a transmembrane domain from the HLA heavy-chain domain). In some cases, the effector domain is fused to the single-chain dimer or trimer via a linker peptide.

In some embodiments, the fusion polypeptide further comprises a tag or marker (e.g., a fluorescent protein, such as green fluorescent protein, a His tag, or a FLAG tag). Non-limiting examples of peptide tags include an ALFA-tag, an AviTag, a C-tag, a Calmodulin-tag, an iCap Tag™, a polyglutamate tag, a polyarginine tag, an E-tag, a FLAG-tag, an HA-tag, a His-tag, a Myc-tag, an NE-tag, a RHO1D4-tag, an S-tag, an SBP-tag, a Softag 1, a Softag 3, a Spot-tag, a Strep-tag, a T7-tag, a Ty-tag, a TC-tag, a V5-tag, a VSV-tag, an Xpress-tag, Isopeptag, Spy Tag, SnoopTag, DogTag, SdyTag, BCCP, a glutathione-S-transferase-tag, a GFP-tag, a HaloTag, a SNAP-tag, a CLIP-tag, a HUH-tag, a maltose-binding protein-tag, a Nus-tag, a thioredoxin-tag, an Fc-tag, and a carbohydrate recognition domain tag.

### Modified Cells

The present disclosure provides cells (e.g., T- or NK-cells, or any cell suitable for use in regenerative medicine, solid-organ transplant, or treatment of an autoimmune disease) modified using nucleobase editors and/or nucleases described herein. The modified immune cells may express chimeric antigen receptors (CARs) (e.g., CAR-T cells). In embodiments, the modified immune cells express an HLA single-chain dimer or trimer construct (e.g., those described above, listed in Table 8A, and/or described in Example 2). Modification of immune cells to express a chimeric antigen receptor can enhance an immune cell's immunoreactive activity, wherein the chimeric antigen receptor has an affinity for an epitope on an antigen, wherein the antigen is associated with an altered fitness of an organism. For example, the chimeric antigen receptor can have an affinity for an epitope on a protein expressed in a diseased cell. Because the CAR-T cells can act independently of major histocompatibility complex (MHC), activated CAR-T cells can kill the diseased cell expressing the antigen. The direct action of the CAR-T cell evades defensive mechanisms that have evolved in response to MHC presentation of antigens to immune cells. Some embodiments comprise autologous immune cell immunotherapy, wherein immune cells are obtained from a subject having a disease or altered fitness characterized by cancerous or otherwise altered cells expressing a surface marker. The obtained immune cells are genetically modified to express a chimeric antigen receptor and are effectively redirected against specific antigens. Thus, in some embodiments, immune cells are obtained from a subject in need of CAR-T immunotherapy. In some embodiments, these autologous immune cells are cultured and modified shortly after they are obtained from the subject. In other embodiments, the autologous cells are obtained and then stored for future use. This practice may be advisable for individuals who may be undergoing parallel treatment that will diminish immune cell counts in the future. Some embodiments comprise allogeneic immune cell immunotherapy. In allogeneic immune cell immunotherapy, immune cells are obtained from a donor other than the subject who will be receiving treatment. In some embodiments, immune cells are obtained from a healthy subject or donor and are genetically modified to express a chimeric antigen receptor and are effectively redirected against specific antigens. The immune cells, after modification to express a chimeric antigen receptor (CAR), are administered to a subject for treating a disease. In some embodiments, immune cells to be modified to express a chimeric antigen receptor (CAR) can be obtained from pre-existing stock cultures of immune cells.

Non-limiting examples of cells suitable for use in embodiments of the disclosure include, but are not limited to, the following cell types or progenitors thereof: bone cells (e.g., osteoblasts, osteoclasts, osteocytes, and lining cells), cartilage cells (e.g., chondrocytes), endothelial cells, epithelial cells, fat cells (e.g., white or brown adipocytes), muscle cells (e.g., skeletal, cardiac, or smooth muscle cells), nerve cells (e.g., neurons or neuroglial cells), platelets, red blood cells (e.g., erythrocytes), sex cells (e.g., spermatozoa or ova), skin cells (e.g., keratinocytes, melanocytes, Merkel cells, or Langerhans cells), stem cells (e.g., embryonic stem cells or adult stem cells), and white blood cells (e.g., granulocytes such as neutrophils, eosinophils, or basophils, or agranulocytes such as monocytes or lymphocytes). Non-limiting examples of cells suitable for use in embodiments of the disclosure also include, but are not limited to, the following cell types or progenitors thereof: bone marrow cells, vascular endothelial cells, hepatocytes, neurons, glia, bronchial endothelial cells, epidermal cells, respiratory interstitial cells, adipocytes, and dermal fibroblasts. Non-limiting examples of cells suitable for use in embodiments of the disclosure also include, but are not limited to, the following cell types or progenitors thereof: Endothelial cells, Adrenergic neural cells, Alpha cells, Ameloblast, Anterior lens epithelial cells, Apocrine sweat gland cells, Astrocytes, Auditory inner hair cells of organ of Corti, Auditory outer hair cells of organ of Corti, B cells, Bartholin's gland cells, basal cells (stem cells), Basal cells of olfactory epithelium, Basket cells, Basophil granulocyte, Beta cells, Betz cells, Bone marrow reticular tissue fibroblasts, Border cells of organ of Corti, Boundary cells, Bowman's gland cells, Brown fat cells, Brunner's gland cells, Brunner's gland cells, Bulbourethral gland cells, Bushy cells, Cajal-Retzius cells, Cardiac muscle cells, Cartwheel cells, cells of the Zona fasciculata, cells of the Zona glomerulosa, cells of the Zona reticularis, Cementoblast cells, Centroacinar cells, Ceruminous gland cells, Chandelier cells, Chemoreceptor glomus cells of carotid body cells, Chief cells, Cholinergic neuron cells, Chromaffin cells, Club cells, Cold-sensitive primary sensory neurons, Connective tissue macrophage, Corneal fibroblasts, Corpus luteum cells, Cortical hair shaft cells, Corticotropes, Crystallin-containing lens fiber cells, Cuticular hair shaft cells, Cytotoxic T cells, D cells, Delta cells, Dendritic cells, Double-bouquet cells, Duct cells, Eccrine sweat gland clear cells, Eccrine sweat gland dark cells, Efferent ducts cells, Elastic cartilage chondrocyte, Enteric glial cells, Enterochromaffin cells, Enterochromaffin-like cells, Eosinophil granulocyte, Epidermal basal cells, Epidermal Langerhans cells, Epididymal basal cells, Epididymal principal cells, Epithelial reticular cells, Epsilon cells, Erythrocytes, Fibrocartilage chondrocyte, Fork neurons, Foveolar cells, G cells, Gall bladder epithelial cells, Gland of Littre cells, Gland of Moll cells in eyelid, Golgi cells, Gonadotropes, Granule cells, Granulosa cells, Granulosa lutein cells, Grid cells, Head direction cells, Heat-sensitive primary sensory neurons, Helper T cells, Hematopoietic stem cells and committed progenitors for the blood and immune system (various types), Henle's layer hair root sheath cells, Hensen's cells of organ of Corti, Hepatic stellate cells (Ito cells), Huxley's layer hair root sheath cells, Hyaline cartilage chondrocytes, Hyalocytes, I cells, Inner hair cells of vestibular system of ear, Inner phalangeal cells of organ of Corti, Inner pillar cells of organ of Corti, Insulated goblet cells, Intercalated cells, Intercalated duct cells, Intercalated duct cells, Intermediate skeletal muscle cells, Interstitial kidney cells, Intestinal brush border cells (with microvilli), Juxtaglomerular cells, K cells, Keratinocyte, Kidney distal tubule cells, L cells, Lacrimal gland cells, Lactiferous duct cells, Lactotropes, Leydig cells, Liver lipocyte, Loop of Henle thin segment cells, Loose connective tissue fibroblasts, Lugaro cells, Macula densa cells, Magnocellsular neurosecretory cells, Mammary gland cells, Martinotti cells, Mast cells, Medium spiny neurons, Medullary hair shaft cells, Megakaryocyte, Melanocyte, Melanotropes, Merkel cells of epidermis, Mesangial cells, Microglial cells, Mo cells (or M cells), Monocytes, Myoepithelial cells, Myosatellite cells, N cells, Natural killer cells, Natural killer T cells, Neurogliaform cells, Neutrophil granulocytes, Nuclear bag cells, Nuclear chain cells, Nucleus pulposus cells, Odontoblasts, Olfactory ensheathing cells, Olfactory epithelium supporting cells, Olfactory receptor neurons, Oligodendrocytes, Oogoniums/Oocytes, Organ of Corti interdental epithelial cells, Osteoblasts/osteocytes, Osteoclasts, Osteoprogenitor cells, Other nonepithelial fibroblasts, Outer hair cells of vestibular system of ear, Outer phalangeal cells of organ of Corti, Outer pillar cells of the organ of Corti, Outer root sheath hair cells, Oxyphil cells, Pain-sensitive primary sensory neurons, Pancreatic acinar cells, Pancreatic stellate cells, Paneth cells, Parafollicular cells, Parathyroid chief cells, Parietal cells, Parietal epithelial cells, Parvocellsular neurosecretory cells, Peptidergic neural cells (various types), Peripolar cells, Photoreceptor blue-sensitive cone cells of eye, Photoreceptor green-sensitive cone cells of eye, Photoreceptor red-sensitive cone cells of eye, Photoreceptor rod cells, Pituicytes, Place cells, Planum semilunar epithelial cells of vestibular system of ear, Plasma cells, Platelets if considered distinct cells, currently there's debate on the subject, Podocyte, PP cells (gamma cells), Principal cells, Proprioceptive primary sensory neurons, Prostate gland cells, Proximal tubule brush border cells, Purkinje cells, Purkinje fiber cells, Red skeletal muscle cells (slow twitch), Regulatory T cells, Renshaw cells, Retina horizontal cells, S cells, SA node cells, Salivary gland mucous cells, Salivary gland serous cells, Satellite glial cells, Schwann cells, Sebaceous gland cells, Seminal vesicle cells, Sertoli cells, Smooth muscle cells, Somatotropes, Speed cells, Spermatids, Spermatocytes, Spermatogonium cells, Spermatozoon cells, Spindle neurons, Starburst amacrine cells, Stellate cells, Stellate cells, Striated duct cells, Surface epithelial cells, Tanycytes, Taste bud supporting cells, Taste receptor cells of taste bud, Tendon fibroblasts, Theca Interna cells, Theca lutein cells, Thyroid epithelial cells, Thyrotropes, Touch-sensitive primary sensory neurons, Transitional epithelium, Trichocytes, Type I pneumocytes, Type II pneumocytes, Unipolar brush cells, Uterus endometrium cells, Vestibular apparatus supporting cells, Von Ebner's gland cells, White fat cells, and White skeletal muscle cells (fast twitch). In some embodiments, a cell of the disclosure is a universal cell, hepatocyte, islet cell (e.g., pancreatic islet or islet of Langerhans), induced pluripotent stem cell (iPSC), mesenchymal stem cell, neural stem cell, fibroblast, nerve cell, muscle cell, epithelial cell, or multi-potent cell. In some cases, the cell is suitable for use in and/or is associated with tissue regeneration.

Immune cells and/or immune effector cells can be isolated or purified from a sample collected from a subject or a donor using standard techniques known in the art. For example, immune effector cells can be isolated or purified from a whole blood sample by lysing red blood cells and removing peripheral mononuclear blood cells by centrifugation. The immune effector cells can be further isolated or purified using a selective purification method that isolates the immune effector cells based on cell-specific markers such as CD25, CD3, CD4, CD8, CD28, CD45RA, or CD45RO. In one embodiment, CD4⁺ is used as a marker to select T cells. In one embodiment, CD8⁺ is used as a marker to select T cells. In one embodiment, CD4⁺ and CD8⁺ are used as a marker to select regulatory T cells.

In another embodiment, the present disclosure provides T cells that have been modified (e.g., through base editing) to knock-out expression of beta-2-microglobulin.

In another embodiment, the present disclosure provides donor T cells selected ex vivo to recognize minor histocompatibility antigens expressed on recipient hematopoietic cells, thereby minimizing the risk of graft-versus-host disease (GVHD), which is the main cause of morbidity and mortality after transplantation (Warren et al., Blood 2010; 115 (19): 3869-3878).

Another technique for isolating or purifying immune effector cells is flow cytometry. In fluorescence activated cell sorting a fluorescently labelled antibody with affinity for an immune effector cell marker is used to label immune effector cells in a sample. A gating strategy appropriate for the cells expressing the marker is used to segregate the cells. For example, T lymphocytes can be separated from other cells in a sample by using, for example, a fluorescently labeled antibody specific for an immune effector cell marker (e.g., CD4, CD8, CD28, CD45) and corresponding gating strategy. In one embodiment, a CD4 gating strategy is employed. In one embodiment, a CD8 gating strategy is employed. In one embodiment, a CD4 and CD8 gating strategy is employed. In some embodiments, a gating strategy for other markers specific to an immune effector cell is employed instead of, or in combination with, the CD4 and/or CD8 gating strategy.

The immune effector cells contemplated in the present disclosure are effector T cells. In some embodiments, the effector T cell is a naïve CD8⁺ T cell, a cytotoxic T cell, a natural killer T (NKT) cell, a natural killer (NK) cell, or a regulatory T (Treg) cell. In some embodiments, the effector T cells are thymocytes, immature T lymphocytes, mature T lymphocytes, resting T lymphocytes, or activated T lymphocytes. In some embodiments the immune effector cell is a CD4+CD8⁺ T cell or a CD4-CD8-T cell. In some embodiments the immune effector cell is a T helper cell. In some embodiments the T helper cell is a T helper 1 (Th1), a T helper 2 (Th2) cell, or a helper T cell expressing CD4 (CD4+ T cell). In some embodiments, immune effector cells are effector NK cells. In some embodiments, the immune effector cell is any other subset of T cells. The modified immune effector cell may express, in addition to the chimeric antigen receptor (CAR), an exogenous cytokine, a different chimeric receptor, or any other agent that would enhance immune effector cell signaling or function. For example, co-expression of the chimeric antigen receptor and a cytokine may enhance the CAR-T cell's ability to lyse a target cell.

Chimeric antigen receptors (CARs) as contemplated in the present disclosure may comprise an extracellular binding domain, a transmembrane domain, and an intracellular domain. Binding of an antigen to the extracellular binding domain can activate the CAR-T cell and generate an effector response, which includes CAR-T cell proliferation, cytokine production, and other processes that lead to the death of the antigen expressing cell. Exemplary CARs include those described in Maldini, et al. “Dual CD4-based CAR-T cells with distinct costimulatory domains mitigate HIV pathogenesis in vivo,” Nat. Med. 26:1776-1787 (2020); Maldini, et al. “HIV-Resistant and HIV-Specific CAR-Modified CD4+ T Cells Mitigate HIV Disease Progression and Confer CD4+ T Cell Help In Vivo,” Mol. Ther. 28:1585-1599 (2020); and Leibman, et al. “Supraphysiologic control over HIV-1 replication mediated by CD8 T cells expressing a re-engineered CD4-based chimeric antigen receptor,” PLOS Pathog. 13 (10): e1006613 (2017), the entire disclosure of each of which is incorporated herein by reference in its entirety for all purposes.

In some embodiments of the present disclosure, the chimeric antigen receptor further comprises a linker. In some embodiments, the linker is a (GGGGS)ₙ linker (SEQ ID NO: 247). In some embodiments, the linker is a (GGGGS)₃ linker (SEQ ID NO: 478). In some embodiments, a CAR of the present disclosure includes a leader peptide sequence (e.g., N-terminal to the antigen binding domain). An exemplary leader peptide amino acid sequence is:

(SEQ ID NO: 479)
METDTLLLWVLLLWVPGSTG.

Provided herein are also nucleic acids that encode the chimeric antigen receptors (CARs) described herein. In some embodiments, the nucleic acid molecule is isolated or purified. Delivery of the nucleic acid molecules ex vivo can be accomplished using methods known in the art. For example, immune cells obtained from a subject may be transformed with a nucleic acid vector encoding the chimeric antigen receptor. The vector may then be used to transform recipient immune cells so that these cells will then express the chimeric antigen receptor. Efficient means of transforming immune cells include transfection and transduction. Such methods are well known in the art. For example, applicable methods for delivery the nucleic acid molecule encoding the chimeric antigen receptor (and the nucleic acid(s) encoding the base editor) can be found in International Application No. PCT/US2009/040040 and U.S. Pat. Nos. 8,450,112; 9,132,153; and 9,669,058, each of which is incorporated herein in its entirety. Additionally, those methods and vectors described herein for delivering the nucleic acid encoding the base editor are applicable to delivering the nucleic acid encoding the chimeric antigen receptor.

Some aspects of the present disclosure provide for immune cells comprising a chimeric antigen receptor (CAR) and an altered endogenous gene that provides increased resistance to lysis by immune effector cells (e.g., alloreactive T cells and/or natural killer cells), resistance to fratricide, enhances immune cell function, resistance to immunosuppression or inhibition, or a combination thereof. In some embodiments, the altered endogenous gene may be created by base editing. In some embodiments, the base editing may reduce or attenuate the gene expression. In some embodiments, the base editing may reduce or attenuate the gene activation. In some embodiments, the base editing may reduce or attenuate the functionality of the gene product. In some other embodiments, the base editing may activate or enhance the gene expression. In some embodiments, the base editing may increase the functionality of the gene product. In some embodiments, the altered endogenous gene may be modified or edited in an exon, an intron, an exon-intron injunction, or a regulatory element thereof. The modification may be edit to a single nucleobase in a gene or a regulatory element thereof. The modification may be in a exon, more than one exons, an intron, or more than one introns, or a combination thereof. The modification may be in an open reading frame of a gene. The modification may be in an untranslated region of the gene, for example, a 3′-UTR or a 5′-UTR. In some embodiments, the modification is in a regulatory element of an endogenous gene. In some embodiments, the modification is in a promoter, an enhancer, an operator, a silencer, an insulator, a terminator, a transcription initiation sequence, a translation initiation sequence (e.g. a Kozak sequence), or any combination thereof.

Allogeneic immune cells expressing an endogenous immune cell receptor, as well as a chimeric antigen receptor (CAR) may recognize and attack host cells, a circumstance termed graft versus host disease (GVHD). The alpha component of the immune cell receptor complex is encoded by the TRAC gene, and in some embodiments, this gene is edited such that the alpha subunit of the TCR complex is nonfunctional or absent. Because this subunit is necessary for endogenous immune cell signaling, editing this gene can reduce the risk of graft versus host disease caused by allogeneic immune cells.

In some embodiments, editing of genes and/or expression of an HLA class-I single chain dimer or trimer of the present disclosure to provide increased resistance to lysis by immune effector cells (e.g., alloreactive T cells or natural killer cells), fratricide resistance, enhance the function of the immune cell and/or to reduce immunosuppression or inhibition can occur in the immune cell before the cell is transformed to express a chimeric antigen receptor (CAR). In other aspects, editing of genes and/or expression of an HLA class-I single chain dimer or trimer to increase resistance to lysis by immune effector cells (e.g., alloreactive T cells or natural killer cells), provide fratricide resistance, enhance the function of the immune cell or to reduce immunosuppression or inhibition can occur in a CAR-T cell, i.e., after the immune cell has been transformed to express a chimeric antigen receptor (CAR).

In some embodiments, the immune cell may comprise one or more edited genes, one or more regulatory elements thereof, or combinations thereof, wherein expression of the edited gene is either knocked out or knocked down. In some embodiments, the immune cell may comprise one or more edited genes, one or more regulatory elements thereof, or combinations thereof, wherein expression of the edited gene is increased. In some embodiments, the immune cell may comprise a chimeric antigen receptor (CAR) and one or more edited genes, one or more regulatory elements thereof, or combinations thereof, wherein expression of the edited gene is either knocked out or knocked down. In some embodiments, the immune cell may comprise a chimeric antigen receptor (CAR) and one or more edited genes, one or more regulatory elements thereof, or combinations thereof, wherein expression of the edited gene is increased.

In some embodiments, the modified immune cells of the present disclosure have reduced or inactivated surface HLA class-I expression relative to a reference cell. In some embodiments, the modified immune cells have increased resistance to lysis by immune effector cells (e.g., alloreactive T cells or natural killer cells) relative to a reference cell. In some embodiments, the modified immune cells cells have increased fratricide resistance relative to a reference cell. In some embodiments, the modified immune cells have reduced immunogenicity relative to a reference cell. In some embodiments, the modified immune cells have increased anti-neoplasia activity relative to a reference cell. In some embodiments, the modified immune cells have increased T- and/or NK-cell resistance relative to a reference cell. The one or more genes may be edited by base editing. In some embodiments the one or more genes are directed to components of the peptide loading complex (PLC) or regulatory components thereof. In some embodiments the one or more genes may be selected from a group consisting of: β2M, TAP1, TAP2, Tapasin, and CD58. In some the genes may be edited by base editing and or using a nuclease (e.g., Cas12b). In some embodiments, the one or more genes are selected from CD58, CD115, CD48, MICA, MICB, Nectin-2, ULBP, β2M, TAP1, TAP2, TAPBP, PDIA3, NLRC5, HLA-A, HLA-B, and/or HLA-C. In some embodiments, one or more additional genes may be edited using a base editor or nuclease. In some embodiments, the one or more additional genes may be selected from TRAC, and CIITA. In some embodiments, the one or more additional genes edited may be selected from HLA-E, HLA-G, PD-L1, and CD47. In some embodiments, one or more of β2M, TAP1, TAP2, Tapasin, and/or CD58 are edited in combination with edits in each of HLA-E, HLA-G, PD-L1, and CD47.

In some embodiments, an immune cell comprises a chimeric antigen receptor, an HLA class-I single chain dimer or trimer, and/or one or more edited genes, a regulatory element thereof, or combinations thereof. An edited gene may be an immune response regulation gene, an immunogenic gene, a checkpoint inhibitor gene, a gene involved in immune responses, a cell surface marker, e.g. a T cell surface marker, or any combination thereof. In some embodiments, an immune cell comprises a chimeric antigen receptor and an edited gene that is associated with activated T cell proliferation, alpha-beta T cell activation, gamma-delta T cell activation, positive regulation of T cell proliferation, negative regulation of T-helper cell proliferation or differentiation, or their regulatory elements thereof, or combinations thereof. In some embodiments, the edited gene may be a checkpoint inhibitor gene, for example, such as a PD1 gene, a PDC1 gene, or a member related to or regulating the pathway of their formation or activation.

In some embodiments, provided herein is an immune cell with an edited gene in the peptide loading complex (PLC) or a regulatory element thereof, such that the immune cell does not express or expresses at reduced levels surface HLA class-I peptides. In embodiments, the immune cell expresses an altered β2M polypeptide that effects reduced levels of surface HLA class-I peptide expression relative to a reference cell. In some embodiments, provided herein is an immune cell with an edited gene in the peptide loading complex (PLC) or a regulatory element thereof, such that the immune cell has increased resistance to lysis by alloreactive T cells and/or other immune effector cells. In some embodiments, the immune cell comprises an edited gene in the peptide loading complex (PLC) or a regulatory element thereof, and additionally, at least one other edited gene.

In some embodiments, provided herein is an immune cell (e.g., T- or NK-cell) with an edited 82M gene, such that the immune cell does not express an endogenous functional Beta-2-microglobulin. In some embodiments, provided herein is an immune cell with an edited 82M gene, such that the immune cell does not express or expresses at reduced levels surface HLA class-I peptides. In some embodiments, provided herein is an immune cell with an edited β2M gene, such that the immune cell has increased resistance to lysis by an effector cell (e.g., an alloreactive T cell or a natural killer cell). In some embodiments, the immune cell comprises an edited β2M gene, and additionally, at least one other edited gene.

In some embodiments, each edited gene may comprise a single base edit. In some embodiments, each edited gene may comprise multiple base edits at different regions of the gene. In some embodiments, a single modification event (such as electroporation), may introduce one or more gene edits. In some embodiments at least one, two, three, four, five, six, seven, eight, nine, ten, eleven, twelve, thirteen, fourteen, fifteen, sixteen, seventeen, eighteen, nineteen, twenty or more edits may be introduced in one or more genes simultaneously. In some embodiments, an immune cell, including but not limited to any immune cell comprising an edited gene selected from any of the aforementioned gene edits, can be edited to generate mutations in other genes that enhance the CAR-T's function or reduce immunosuppression or inhibition of the cell.

### Editing of Target Polynucleotides in Immune Cells

In general, base editing is carried out to induce therapeutic changes (e.g., knocking out of expression of beta-2-microglobulin and/or another polypeptide(s) listed herein) in the genome of a cell (e.g., immune cell (e.g., T- or NK-cell)). Base editing can be carried out in vitro or in vivo. In various embodiments, base editing can be used to introduce a stop codon to a gene or to disrupt a splice motif (e.g., a acceptor site, or a splice donor site). In some embodiments, cells (e.g., immune cell (e.g., T- or NK-cell)) are collected from a subject or a donor. In some embodiments, base editing is carried out to induce therapeutic changes in the genome of an immune cell (e.g., T- or NK-cell). In some embodiments, base editing is carried out to induce therapeutic changes in the genome of an allogeneic immune cell (e.g., T- or NK-cell) of a subject. In some embodiments, base editing is carried out to induce therapeutic changes in the genome of an allogeneic CAR-T cell.

To produce the gene edits described above, immune cells are collected from a subject and contacted with one or more guide RNAs and a nucleobase editor polypeptide comprising a nucleic acid programmable DNA binding protein (napDNAbp) and a cytidine deaminase or adenosine deaminase, or comprising one or more deaminases with cytidine deaminase and/or adenosine deaminase activity (e.g., a “dual deaminase” which has cytidine and adenosine deaminase activity). In some embodiments, cells to be edited are contacted with at least one nucleic acid, wherein the at least one nucleic acid encodes one or more guide RNAs and a nucleobase editor polypeptide comprising a nucleic acid programmable DNA binding protein (napDNAbp) and a cytidine deaminase. In some embodiments, the gRNA comprises nucleotide analogs. In some instances, the gRNA is added directly to a cell. These nucleotide analogs can inhibit degradation of the gRNA from cellular processes. Tables 1 and 2 provide representative target sequences and spacer sequences to be used for gRNAs, as well as representative gRNA sequences.

In some embodiments, immune cells (e.g., T- or NK-cell) of the present disclosure, are contacted with one or more guide RNAs and a nucleobase editor polypeptide comprising a nucleic acid programmable DNA binding protein (napDNAbp) (e.g., Cas9) domain and a deaminase (e.g., cytidine deaminase and/or adenosine deaminase) domain. In some embodiments, immune cells (e.g., T- or NK-cell) of the present disclosure, are contacted with one or more guide RNAs and a nucleobase editor polypeptide comprising a nucleic acid programmable DNA binding protein (napDNAbp) (e.g., Cas9) domain and an adenosine deaminase domain. In some embodiments, immune cells (e.g., T- or NK-cell) of the present disclosure, are contacted with one or more guide RNAs and a nucleobase editor polypeptide comprising a nucleic acid programmable DNA binding protein (napDNAbp) (e.g., Cas9) domain and a cytidine deaminase domain. In some embodiments, immune cells (e.g., T- or NK-cell) of the present disclosure, are contacted with one or more guide RNAs and a nucleobase editor polypeptide comprising a nucleic acid programmable DNA binding protein (napDNAbp) (e.g., Cas9) domain and an adenosine/cytidine deaminase domain. In some embodiments, the at least one nucleic acid molecule encoding one or more guide RNAs and a nucleobase editor polypeptide is delivered to cells by one or more vectors (e.g., AAV vector).

In some embodiments, one or more vectors (e.g., AAV vector) comprise at least one nucleic acid molecule encoding one or more guide RNAs and a nucleobase editor polypeptide, which comprises a nucleic acid programmable DNA binding protein (napDNAbp) (e.g., Cas9) domain and a deaminase (e.g., cytidine deaminase and/or adenosine deaminase) domain. In some embodiments, one or more vectors (e.g., AAV vector) comprise at least one nucleic acid molecule encoding one or more guide RNAs, which direct a nucleobase editor polypeptide to edit a site in the genome of a cell (e.g., immune cell (e.g., T- or NK-cell)).

The present disclosure provides one or more guide RNAs that direct a nucleobase editor polypeptide to edit a site in the genome of the cell (e.g., immune cell (e.g., T- or NK-cell)). In some embodiments, the present disclosure provides guide RNAs that target components of the peptide loading complex (PLC) (e.g., β2M, TAP1, TAP2, Tapasin) and/or CD58 in an immune cell (e.g., T- or NK-cell). In some embodiments, the present disclosure provides guide RNAs that target β2M, TAP1, TAP2, Tapasin, and/or CD58. In some embodiments, the gRNA comprises nucleotide analogs. These nucleotide analogs can inhibit degradation of the gRNA from cellular processes. Exemplary guide spacer sequences, gRNA sequences, and target sequences are provided in the following Tables 1 and 2. A non-limiting example of a gRNA scaffold sequence includes the following:

(SEQ ID NO: 626)
GUUUUAGAGCUAGAAAUAGCAAGUUAAAAUAA
GGCUAGUCCGUUAUCAACUUGAAAAAGUGGCA
CCGAGUCGGUGCUUUU.

In various instances, it is advantageous for a spacer sequence to include a 5′ and/or a 3′ “G” nucleotide. In some cases, for example, any spacer sequence or guide polynucleotide provided herein comprises or further comprises a 5′ “G”, where, in some embodiments, the 5′ “G” is or is not complementary to a target sequence. In some embodiments, the 5′ “G” is added to a spacer sequence that does not already contain a 5′ “G.” For example, it can be advantageous for a guide RNA to include a 5′ terminal “G” when the guide RNA is expressed under the control of a U6 promoter or the like because the U6 promoter prefers a “G” at the transcription start site (see Cong, L. et al. “Multiplex genome engineering using CRISPR/Cas systems. Science 339:819-823 (2013) doi: 10.1126/science.1231143). In some cases, a 5′ terminal “G” is added to a guide polynucleotide that is to be expressed under the control of a promoter, but is optionally not added to the guide polynucleotide if or when the guide polynucleotide is not expressed under the control of a promoter.

Exemplary guide RNA and spacer sequences suitable for use in methods of the disclosure include those listed in PCT/US22/75021, filed Aug. 16, 2022, the disclosure of which is incorporated herein in its entirety for all purposes.

TABLE 1
Exemplary Guide RNAs.

| | | | | | | Guide |
|---|---|---|---|---|---|---|
| | | | | | | with |
| | | | | | | Guide |
| | | | | Guide | | Scaffold |
| | | Base | Spacer | SEQ ID | | SEQ ID |
| Guide No. | Gene | Editor | Sequence | NO: | gRNA sequence | NO: |
| TSBTx760 | B2M | ABE | CUUACCCC | 537 | CUUACCCCACUUAACUAU | 577 |
| | | | ACUUAACU | | CUGUUUUAGAGCUAGAA | |
| | | | AUCU | | AUAGCAAGUUAAAAUAA | |
| | | | | | GGCUAGUCCGUUAUCAAC | |
| | | | | | UUGAAAAAGUGGCACCGA | |
| | | | | | GUCGGUGCUUUU | |
| Guide 417 | B2M | CBE | ACAGCCC | 538 | ACAGCCCAAGAUAGUUAAG | 578 |
| (b2m_315) | | | AAGAUAG | | UGUUUUAGAGCUAGAAAUA | |
| | | | UUAAGU | | GCAAGUUAAAAUAAGGCUA | |
| | | | | | GUCCGUUAUCAACUUGAAA | |
| | | | | | AAGUGGCACCGAGUCGGUG | |
| | | | | | CUUUU | |
| Guide 418 | B2M | CBE | CAGCCCA | 539 | CAGCCCAAGAUAGUUAAGU | 579 |
| (b2m_316) | | | AGAUAGU | | GGUUUUAGAGCUAGAAAUA | |
| | | | UAAGUG | | GCAAGUUAAAAUAAGGCUA | |
| | | | | | GUCCGUUAUCAACUUGAAA | |
| | | | | | AAGUGGCACCGAGUCGGUG | |
| | | | | | CUUUU | |
| Guide 431 | B2M | CBE, | CAGUAAG | 540 | CAGUAAGUCAACUUCAAUG | 580 |
| (b2m_629) | | ABE | UCAACUU | | UGUUUUAGAGCUAGAAAUA | |
| | | | CAAUGU | | GCAAGUUAAAAUAAGGCUA | |
| | | | | | GUCCGUUAUCAACUUGAAA | |
| | | | | | AAGUGGCACCGAGUCGGUG | |
| | | | | | CUUUU | |
| b2m_825 | B2M | CBE | ACUCACG | 541 | ACUCACGCUGGAUAGCCUC | 581 |
| (TSBTx84 | | | CUGGAUA | | CGUUUUAGAGCUAGAAAUA | |
| 5) | | | GCCUCC | | GCAAGUUAAAAUAAGGCUA | |
| | | | | | GUCCGUUAUCAACUUGAAA | |
| | | | | | AAGUGGCACCGAGUCGGUG | |
| | | | | | CUUUU | |
| b2m_630 | B2M | CBE | AAGUCAA | 542 | AAGUCAACUUCAAUGUCGG | 582 |
| | | | CUUCAAU | | AGUUUUAGAGCUAGAAAUA | |
| | | | GUCGGA | | GCAAGUUAAAAUAAGGCUA | |
| | | | | | GUCCGUUAUCAACUUGAAA | |
| | | | | | AAGUGGCACCGAGUCGGUG | |
| | | | | | CUUUU | |
| b2m_227 | B2M | CBE | GGAUUUG | 543 | GGAUUUGCCUAGCUUCCCC | 583 |
| | | | CCUAGCU | | AGUUUUAGAGCUAGAAAUA | |
| | | | UCCCCA | | GCAAGUUAAAAUAAGGCUA | |
| | | | | | GUCCGUUAUCAACUUGAAA | |
| | | | | | AAGUGGCACCGAGUCGGUG | |
| | | | | | CUUUU | |
| b2m_596 | B2M | CBE | UCGAUCU | 544 | UCGAUCUAUGAAAAAGACA | 584 |
| | | | AUGAAAA | | GGUUUUAGAGCUAGAAAUA | |
| | | | AGACAG | | GCAAGUUAAAAUAAGGCUA | |
| | | | | | GUCCGUUAUCAACUUGAAA | |
| | | | | | AAGUGGCACCGAGUCGGUG | |
| | | | | | CUUUU | |
| TSBTx418 | HLA- | ABE, | UGACGGC | 545 | UGACGGCCAUCCUCGGCGU | 585 |
| 6 (846) | A | CBE | CAUCCUC | | CGUUUUAGAGCUAGAAAUA | |
| | | | GGCGUC | | GCAAGUUAAAAUAAGGCUA | |
| | | | | | GUCCGUUAUCAACUUGAAA | |
| | | | | | AAGUGGCACCGAGUCGGUG | |
| | | | | | CUUUU | |
| TSBTx418 | HLA- | ABE, | GACGGCC | 546 | GACGGCCAUCCUCGGCGUC | 586 |
| 7 (847) | A | CBE | AUCCUCG | | UGUUUUAGAGCUAGAAAUA | |
| | | | GCGUCU | | GCAAGUUAAAAUAAGGCUA | |
| | | | | | GUCCGUUAUCAACUUGAAA | |
| | | | | | AAGUGGCACCGAGUCGGUG | |
| | | | | | CUUUU | |
| TSBTx418 | HLA- | ABE, | ACGGCCA | 547 | ACGGCCAUCCUCGGCGUCU | 587 |
| 8 (848) | A | CBE | UCCUCGG | | GGUUUUAGAGCUAGAAAUA | |
| | | | CGUCUG | | GCAAGUUAAAAUAAGGCUA | |
| | | | | | GUCCGUUAUCAACUUGAAA | |
| | | | | | AAGUGGCACCGAGUCGGUG | |
| | | | | | CUUUU | |
| TSBTx418 | HLA- | ABE, | CGCACUC | 548 | CGCACUCACCCGCCCAGGU | 588 |
| 9 (849) | A | CBE | ACCCGCC | | CGUUUUAGAGCUAGAAAUA | |
| | | | CAGGUC | | GCAAGUUAAAAUAAGGCUA | |
| | | | | | GUCCGUUAUCAACUUGAAA | |
| | | | | | AAGUGGCACCGAGUCGGUG | |
| | | | | | CUUUU | |
| TSBTx419 | HLA- | ABE, | GCACUCA | 549 | GCACUCACCCGCCCAGGUC | 589 |
| 0 (850) | A | CBE | CCCGCCC | | UGUUUUAGAGCUAGAAAUA | |
| | | | AGGUCU | | GCAAGUUAAAAUAAGGCUA | |
| | | | | | GUCCGUUAUCAACUUGAAA | |
| | | | | | AAGUGGCACCGAGUCGGUG | |
| | | | | | CUUUU | |
| TSBTx419 | HLA- | ABE | CCCAGGC | 550 | CCCAGGCUCGCACUCCAUG | 590 |
| 1 (851) | A | | UCGCACU | | GUUUUAGAGCUAGAAAUAG | |
| | | | CCAUG | | CAAGUUAAAAUAAGGCUAG | |
| | | | | | UCCGUUAUCAACUUGAAAA | |
| | | | | | AGUGGCACCGAGUCGGUGC | |
| | | | | | UUUU | |
| TSBTx419 | HLA- | ABE, | GUCACUC | 551 | GUCACUCACCGGCCUCGCU | 591 |
| 2 (852) | A, | CBE | ACCGGCC | | CGUUUUAGAGCUAGAAAUA | |
| | HLA- | | UCGCUC | | GCAAGUUAAAAUAAGGCUA | |
| | B, | | | | GUCCGUUAUCAACUUGAAA | |
| | HLA-C | | | | AAGUGGCACCGAGUCGGUG | |
| | | | | | CUUUU | |
| TSBTx419 | HLA- | ABE, | CCUUACC | 552 | CCUUACCCCAUCUCAGGGU | 592 |
| 3 (853) | A, | CBE | CCAUCUC | | GGUUUUAGAGCUAGAAAUA | |
| | HLA- | | AGGGUG | | GCAAGUUAAAAUAAGGCUA | |
| | B, | | | | GUCCGUUAUCAACUUGAAA | |
| | HLA-C | | | | AAGUGGCACCGAGUCGGUG | |
| | | | | | CUUUU | |
| TSBTx419 | HLA- | ABE, | CUUACCC | 553 | CUUACCCCAUCUCAGGGUG | 593 |
| 4 (854) | A, | CBE | CAUCUCA | | AGUUUUAGAGCUAGAAAUA | |
| | HLA- | | GGGUGA | | GCAAGUUAAAAUAAGGCUA | |
| | B, | | | | GUCCGUUAUCAACUUGAAA | |
| | HLA-C | | | | AAGUGGCACCGAGUCGGUG | |
| | | | | | CUUUU | |
| TSBTx419 | HLA- | CBE | GGCUCUG | 554 | GGCUCUGGGAAAAGAGGGG | 594 |
| 5 (855) | A | | GGAAAAG | | AGUUUUAGAGCUAGAAAUA | |
| | | | AGGGGA | | GCAAGUUAAAAUAAGGCUA | |
| | | | | | GUCCGUUAUCAACUUGAAA | |
| | | | | | AAGUGGCACCGAGUCGGUG | |
| | | | | | CUUUU | |
| TSBTx419 | HLA- | ABE | CUUCCCA | 555 | CUUCCCACAGAUAGAAAAG | 595 |
| 6 (856) | A | | CAGAUAG | | GGUUUUAGAGCUAGAAAUA | |
| | | | AAAAGG | | GCAAGUUAAAAUAAGGCUA | |
| | | | | | GUCCGUUAUCAACUUGAAA | |
| | | | | | AAGUGGCACCGAGUCGGUG | |
| | | | | | CUUUU | |
| TSBTx419 | HLA- | ABE | UUCCCAC | 556 | UUCCCACAGAUAGAAAAGG | 596 |
| 7 (857) | A | | AGAUAGA | | AGUUUUAGAGCUAGAAAUA | |
| | | | AAAGGA | | GCAAGUUAAAAUAAGGCUA | |
| | | | | | GUCCGUUAUCAACUUGAAA | |
| | | | | | AAGUGGCACCGAGUCGGUG | |
| | | | | | CUUUU | |
| TSBTx419 | HLA- | ABE | CCCAGGC | 557 | CCCAGGCAGUGACAGUGCC | 597 |
| 8 (858) | A | | AGUGACA | | CGUUUUAGAGCUAGAAAUA | |
| | | | GUGCCC | | GCAAGUUAAAAUAAGGCUA | |
| | | | | | GUCCGUUAUCAACUUGAAA | |
| | | | | | AAGUGGCACCGAGUCGGUG | |
| | | | | | CUUUU | |
| TSBTx419 | HLA- | ABE | CCAGGCA | 558 | CCAGGCAGUGACAGUGCCC | 598 |
| 9 (859) | A | | GUGACAG | | AGUUUUAGAGCUAGAAAUA | |
| | | | UGCCCA | | GCAAGUUAAAAUAAGGCUA | |
| | | | | | GUCCGUUAUCAACUUGAAA | |
| | | | | | AAGUGGCACCGAGUCGGUG | |
| | | | | | CUUUU | |
| TSBTx420 | HLA- | ABE | GACCCGC | 559 | GACCCGCAUCUCGGCGUCU | 599 |
| 0 (860) | B, | | AUCUCGG | | GGUUUUAGAGCUAGAAAUA | |
| | HLA-C | | CGUCUG | | GCAAGUUAAAAUAAGGCUA | |
| | | | | | GUCCGUUAUCAACUUGAAA | |
| | | | | | AAGUGGCACCGAGUCGGUG | |
| | | | | | CUUUU | |
| TSBTx420 | HLA-B | ABE, | GCACUCA | 560 | GCACUCACCGGCCCAGGUC | 600 |
| 1 (861) | | CBE | CCGGCCC | | UGUUUUAGAGCUAGAAAUA | |
| | | | AGGUCU | | GCAAGUUAAAAUAAGGCUA | |
| | | | | | GUCCGUUAUCAACUUGAAA | |
| | | | | | AAGUGGCACCGAGUCGGUG | |
| | | | | | CUUUU | |
| TSBTx420 | HLA-B | CBE | UGGGAGC | 561 | UGGGAGCCUGGGGGCGAGG | 601 |
| 2 (862) | | | CUGGGGG | | AGUUUUAGAGCUAGAAAUA | |
| | | | CGAGGA | | GCAAGUUAAAAUAAGGCUA | |
| | | | | | GUCCGUUAUCAACUUGAAA | |
| | | | | | AAGUGGCACCGAGUCGGUG | |
| | | | | | CUUUU | |
| TSBTx420 | HLA-B | CBE | GGGAGCC | 562 | GGGAGCCUGGGGGCGAGGA | 602 |
| 3 (863) | | | UGGGGGC | | GGUUUUAGAGCUAGAAAUA | |
| | | | GAGGAG | | GCAAGUUAAAAUAAGGCUA | |
| | | | | | GUCCGUUAUCAACUUGAAA | |
| | | | | | AAGUGGCACCGAGUCGGUG | |
| | | | | | CUUUU | |
| TSBTx420 | HLA- | ABE | CCCCAGG | 563 | CCCCAGGCUCCCACUCCAU | 603 |
| 4 (864) | B, | | CUCCCAC | | GGUUUUAGAGCUAGAAAUA | |
| | HLA-C | | UCCAUG | | GCAAGUUAAAAUAAGGCUA | |
| | | | | | GUCCGUUAUCAACUUGAAA | |
| | | | | | AAGUGGCACCGAGUCGGUG | |
| | | | | | CUUUU | |
| TSBTx420 | HLA- | ABE, | UUACCCC | 564 | UUACCCCAUCUCAGGGUGA | 604 |
| 5 (865) | B, | CBE | AUCUCAG | | GGUUUUAGAGCUAGAAAUA | |
| | HLA-C | | GGUGAG | | GCAAGUUAAAAUAAGGCUA | |
| | | | | | GUCCGUUAUCAACUUGAAA | |
| | | | | | AAGUGGCACCGAGUCGGUG | |
| | | | | | CUUUU | |
| TSBTx420 | HLA- | ABE | UUCCCAC | 565 | UUCCCACAGGUGGAAAAGG | 605 |
| 6 (866) | B, | | AGGUGGA | | AGUUUUAGAGCUAGAAAUA | |
| | HLA-C | | AAAGGA | | GCAAGUUAAAAUAAGGCUA | |
| | | | | | GUCCGUUAUCAACUUGAAA | |
| | | | | | AAGUGGCACCGAGUCGGUG | |
| | | | | | CUUUU | |
| TSBTx420 | HLA-C | ABE, | UGACCCG | 566 | UGACCCGCAUCUCGGCGUC | 606 |
| 7 (867) | | CBE | CAUCUCG | | UGUUUUAGAGCUAGAAAUA | |
| | | | GCGUCU | | GCAAGUUAAAAUAAGGCUA | |
| | | | | | GUCCGUUAUCAACUUGAAA | |
| | | | | | AAGUGGCACCGAGUCGGUG | |
| | | | | | CUUUU | |
| TSBTx420 | HLA-C | ABE, | GCACUCA | 567 | GCACUCACAGGCCCAGGUC | 607 |
| 8 (868) | | CBE | CAGGCCC | | UGUUUUAGAGCUAGAAAUA | |
| | | | AGGUCU | | GCAAGUUAAAAUAAGGCUA | |
| | | | | | GUCCGUUAUCAACUUGAAA | |
| | | | | | AAGUGGCACCGAGUCGGUG | |
| | | | | | CUUUU | |
| TSBTx420 | HLA-C | ABE, | CACAGGC | 568 | CACAGGCCCAGGUCUCGGU | 608 |
| 9 (869) | | CBE | CCAGGUC | | CGUUUUAGAGCUAGAAAUA | |
| | | | UCGGUC | | GCAAGUUAAAAUAAGGCUA | |
| | | | | | GUCCGUUAUCAACUUGAAA | |
| | | | | | AAGUGGCACCGAGUCGGUG | |
| | | | | | CUUUU | |
| TSBTx421 | HLA-C | CBE | AGACCCU | 569 | AGACCCUGGCCCCGCCCCC | 609 |
| 0 (870) | | | GGCCCCG | | GGUUUUAGAGCUAGAAAUA | |
| | | | CCCCCG | | GCAAGUUAAAAUAAGGCUA | |
| | | | | | GUCCGUUAUCAACUUGAAA | |
| | | | | | AAGUGGCACCGAGUCGGUG | |
| | | | | | CUUUU | |
| TSBTx421 | HLA-C | CBE | GGCUCUG | 570 | GGCUCUGGGAAAGGAGGGG | 610 |
| 1 (871) | | | GGAAAGG | | AGUUUUAGAGCUAGAAAUA | |
| | | | AGGGGA | | GCAAGUUAAAAUAAGGCUA | |
| | | | | | GUCCGUUAUCAACUUGAAA | |
| | | | | | AAGUGGCACCGAGUCGGUG | |
| | | | | | CUUUU | |
| TSBTx421 | HLA-C | ABE | CCCAGCC | 571 | CCCAGCCAGCAACAGUGCC | 611 |
| 2 (872) | | | AGCAACA | | CGUUUUAGAGCUAGAAAUA | |
| | | | GUGCCC | | GCAAGUUAAAAUAAGGCUA | |
| | | | | | GUCCGUUAUCAACUUGAAA | |
| | | | | | AAGUGGCACCGAGUCGGUG | |
| | | | | | CUUUU | |
| TSBTx416 | HLA-C | Cas12b | GGACCGG | 572 | GUUCUGUCUUUUGGUCAGG | 612 |
| 7 (873) | | | GAGACAC | | ACAACCGUCUAGCUAUAAG | |
| | | | AGAAGUA | | UGCUGCAGGGUGUGAGAAA | |
| | | | C | | CUCCUAUUGCUGGACGAUG | |
| | | | | | UCUCUUACGAGGCAUUAGC | |
| | | | | | ACGGACCGGGAGACACAGA | |
| | | | | | AGUAC | |
| TSBTx416 | HLA- | Cas12b | CAUCGCC | 573 | GUUCUGUCUUUUGGUCAGG | 613 |
| 8 (874) | C, | | CUGAACG | | ACAACCGUCUAGCUAUAAG | |
| | HLA- | | AGGACCU | | UGCUGCAGGGUGUGAGAAA | |
| | B, | | G | | CUCCUAUUGCUGGACGAUG | |
| | HLA- | | | | UCUCUUACGAGGCAUUAGC | |
| | A | | | | ACCAUCGCCCUGAACGAGG | |
| | | | | | ACCUG | |
| TSBTx416 | HLA-C | Cas12b | CAUCGCC | 574 | GUUCUGUCUUUUGGUCAGG | 614 |
| 9 (875) | | | CUGAACG | | ACAACCGUCUAGCUAUAAG | |
| | | | AGGAUCU | | UGCUGCAGGGUGUGAGAAA | |
| | | | G | | CUCCUAUUGCUGGACGAUG | |
| | | | | | UCUCUUACGAGGCAUUAGC | |
| | | | | | ACCAUCGCCCUGAACGAGG | |
| | | | | | AUCUG | |
| TSBTx417 | HLA-B | Cas12b | UUGCUGG | 575 | GUUCUGUCUUUUGGUCAGG | 615 |
| 0 (876) | | | CCUGGCU | | ACAACCGUCUAGCUAUAAG | |
| | | | GUCCUAG | | UGCUGCAGGGUGUGAGAAA | |
| | | | C | | CUCCUAUUGCUGGACGAUG | |
| | | | | | UCUCUUACGAGGCAUUAGC | |
| | | | | | ACUUGCUGGCCUGGCUGUC | |
| | | | | | CUAGC | |
| TSBTx416 | HLA- | Cas12b | CAUCGCC | 576 | GUUCUGUCUUUUGGUCAGG | 616 |
| 8 (877) | A | | CUGAACG | | ACAACCGUCUAGCUAUAAG | |
| | | | AGGACCU | | UGCUGCAGGGUGUGAGAAA | |
| | | | G | | CUCCUAUUGCUGGACGAUG | |
| | | | | | UCUCUUACGAGGCAUUAGC | |
| | | | | | ACCAUCGCCCUGAACGAGG | |
| | | | | | ACCUG | |

TABLE 2
Exemplary Spacers.

| | | | SEQ | |
|---|---|---|---|---|
| ID | Target | Spacer Sequence | ID NO | Editor |
| TSBTx760 | B2M | CUUACCCCACUUAACUAU | 617 | ABE |
| | | CU | | |
| sgRNA990 | HLA | UCCUUACCCCAUCUCAGG | 618 | ABE/CBE/CABE |
| | class-I | GUG | | |
| sgRNA991 | HLA | CUCCUUACCCCAUCUCAG | 619 | ABE/CBE/CABE |
| | class-I | GGUG | | |
| sgRNA992 | HLA | CUUACCCCAUCUCAGGGU | 620 | ABE/CBE/CABE |
| | class-I | G | | |
| sgRNA993 | HLA | UUACCCCAUCUCAGGGUG | 621 | ABE/CBE/CABE |
| | class-I | | | |
| sgRNA994 | HLA | CCUUACCCCAUCUCAGGG | 622 | ABE/CBE/CABE |
| | class-I | UGA | | |
| sgRNA995 | HLA | UCACCCUGAGAUGGGGUA | 623 | ABE/CBE/CABE |
| | class-I | AGGA | | |
| sgRNA996 | HLA | UUACCCCAUCUCAGGGUG | 624 | ABE/CBE/CABE |
| | class-I | A | | |
| sgRNA997 | HLA | UACCCCAUCUCAGGGUGA | 625 | ABE/CBE/CABE |
| | class-I | | | |

### Nucleobase Editors

Useful in the methods and compositions described herein are nucleobase editors that edit, modify or alter a target nucleotide sequence of a polynucleotide. Nucleobase editors described herein typically include a polynucleotide programmable nucleotide binding domain and a nucleobase editing domain (e.g., adenosine deaminase, cytidine deaminase, or a dual deaminase). A polynucleotide programmable nucleotide binding domain, when in conjunction with a bound guide polynucleotide (e.g., gRNA), can specifically bind to a target polynucleotide sequence and 10 thereby localize the base editor to the target nucleic acid sequence desired to be edited.

### Polynucleotide Programmable Nucleotide Binding Domain

Polynucleotide programmable nucleotide binding domains bind polynucleotides (e.g., RNA, DNA). A polynucleotide programmable nucleotide binding domain of a base editor can itself comprise one or more domains (e.g., one or more nuclease domains). In some embodiments, the nuclease domain of a polynucleotide programmable nucleotide binding domain comprises an endonuclease or an exonuclease.

Disclosed herein are base editors comprising a polynucleotide programmable nucleotide binding domain comprising all or a portion (e.g., a functional portion) of a CRISPR protein (i.e., a base editor comprising as a domain all or a portion (e.g., a functional portion) of a CRISPR protein (e.g., a Cas protein), also referred to as a “CRISPR protein-derived domain” of the base editor). A CRISPR protein-derived domain incorporated into a base editor can be modified compared to a wild-type or natural version of the CRISPR protein. A CRISPR protein-derived domain can comprise one or more mutations, insertions, deletions, rearrangements and/or recombinations relative to a wild-type or natural version of the CRISPR protein.

Cas proteins that can be used herein include class 1 and class 2. Non-limiting examples of Cas proteins include Cas1, Cas1B, Cas2, Cas3, Cas4, Cas5, Cas5d, Cas5t, Cas5h, Cas5a, Cas6, Cas7, Cas8, Cas9 (also known as Csn1 or Csx12), Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, Cas12a/Cpf1, Cas12b/C2c1 (e.g., SEQ ID NO: 232), Cas12c/C2c3, Cas12d/CasY, Cas12e/CasX, Cas12g, Cas12h, Cas12i, and Cas12j/CasΦ, CARF, DinG, homologues thereof, or modified versions thereof. A CRISPR enzyme can direct cleavage of one or both strands at a target sequence, such as within a target sequence and/or within a complement of a target sequence. For example, a CRISPR enzyme can direct cleavage of one or both strands within about 1, 2, 3, 4, 5, 6, 7, 8, 9, 10, 15, 20, 25, 50, 100, 200, 500, or more base pairs from the first or last nucleotide of a target sequence.

A vector that encodes a CRISPR enzyme that is mutated to with respect to a corresponding wild-type enzyme such that the mutated CRISPR enzyme lacks the ability to cleave one or both strands of a target polynucleotide containing a target sequence can be used. A Cas protein (e.g., Cas9, Cas12) or a Cas domain (e.g., Cas9, Cas12) can refer to a polypeptide or domain with at least or at least about 50%, 60%, 70%, 80%, 90%, 91%, 92%, 93%, 94%, 95%, 96%, 97%, 98%, 99%, or 100% sequence identity and/or sequence homology to a wild-type exemplary Cas polypeptide or Cas domain. Cas (e.g., Cas9, Cas12) can refer to the wild-type or a modified form of the Cas protein that can comprise an amino acid change such as a deletion, insertion, substitution, variant, mutation, fusion, chimera, or any combination thereof. In some embodiments, a CRISPR protein-derived domain of a base editor can include all or a portion (e.g., a functional portion) of Cas9 from Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquis (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); Neisseria meningitidis (NCBI Ref: YP_002342100.1), Streptococcus pyogenes, or Staphylococcus aureus.

Some aspects of the disclosure provide high fidelity Cas9 domains. High fidelity Cas9 domains are known in the art and described, for example, in Kleinstiver, B. P., et al. “High-fidelity CRISPR-Cas9 nucleases with no detectable genome-wide off-target effects.” Nature 529, 490-495 (2016); and Slaymaker, I. M., et al. “Rationally engineered Cas9 nucleases with improved specificity.” Science 351, 84-88 (2015); the entire contents of each of which are incorporated herein by reference. An Exemplary high fidelity Cas9 domain is provided in the Sequence Listing as SEQ ID NO: 233.

In some embodiments, any of the Cas9 fusion proteins or complexes provided herein comprise one or more of a D10A, N497X, a R661X, a Q695X, and/or a Q926X mutation, or a corresponding mutation in any of the amino acid sequences provided herein, wherein X is any amino acid.

Typically, Cas9 proteins, such as Cas9 from S. pyogenes (spCas9), require a “protospacer adjacent motif (PAM)” or PAM-like motif, which is a 2-6 base pair DNA sequence immediately following the DNA sequence targeted by the Cas9 nuclease in the CRISPR bacterial adaptive immune system. The presence of an NGG PAM sequence is required to bind a particular nucleic acid region, where the “N” in “NGG” is adenosine (A), thymidine (T), or cytosine (C), and the G is guanosine. In some embodiments, any of the fusion proteins or complexes provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

In some embodiments, the napDNAbp is a circular permutant (e.g., SEQ ID NO: 238).

In some embodiments, the polynucleotide programmable nucleotide binding domain comprises a nickase domain. Herein the term “nickase” refers to a polynucleotide programmable nucleotide binding domain comprising a nuclease domain that is capable of cleaving only one strand of the two strands in a duplexed nucleic acid molecule (e.g., DNA). For example, where a polynucleotide programmable nucleotide binding domain comprises a nickase domain derived from Cas9, the Cas9-derived nickase domain can include a D10A mutation and a histidine at position 840. In another example, a Cas9-derived nickase domain comprises an H840A mutation, while the amino acid residue at position 10 remains a D.

In some embodiments, a Cas9 nuclease has an inactive (e.g., an inactivated) DNA cleavage domain, that is, the Cas9 is a nickase, referred to as an “nCas9” protein (for “nickase” Cas9; SEQ ID NO: 201). The Cas9 nickase may be a Cas9 protein that is capable of cleaving only one strand of a duplexed nucleic acid molecule (e.g., a duplexed DNA molecule). In some embodiments the Cas9 nickase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the Cas9 nickases provided herein. Additional suitable Cas9 nickases will be apparent to those of skill in the art based on this disclosure and knowledge in the field and are within the scope of this disclosure.

Also provided herein are base editors comprising a polynucleotide programmable nucleotide binding domain which is catalytically dead (i.e., incapable of cleaving a target polynucleotide sequence). For example, in the case of a base editor comprising a Cas9 domain, the Cas9 can comprise both a D10A mutation and an H840A mutation. In further embodiments, a catalytically dead polynucleotide programmable nucleotide binding domain comprises a point mutation (e.g., D10A or H840A) as well as a deletion of all or a portion (e.g., a functional portion) of a nuclease domain. dCas9 domains are known in the art and described, for example, in Qi et al., “Repurposing CRISPR as an RNA-guided platform for sequence-specific control of gene expression.” Cell. 2013; 152 (5): 1173-83, the entire contents of which are incorporated herein by reference.

The term “protospacer adjacent motif (PAM)” or PAM-like motif refers to a 2-6 base pair DNA sequence immediately following the DNA sequence targeted by a nucleic acid programmable DNA binding protein. In some embodiments, the PAM can be a 5′ PAM (i.e., located upstream of the 5′ end of the protospacer). In other embodiments, the PAM can be a 3′ PAM (i.e., located downstream of the 5′ end of the protospacer). The PAM sequence can be any PAM sequence known in the art. Suitable PAM sequences include, but are not limited to, NGG, NGA, NGC, NGN, NGT, NGTT, NGCG, NGAG, NGAN, NGNG, NGCN, NGCG, NGTN, NNGRRT, NNNRRT, NNGRR (N), TTTV, TYCV, TYCV, TATV, NNNNGATT, NNAGAAW, or NAAAAC. Y is a pyrimidine; N is any nucleotide base; W is A or T.

A base editor provided herein can comprise a CRISPR protein-derived domain that is capable of binding a nucleotide sequence that contains a canonical or non-canonical protospacer adjacent motif (PAM) sequence.

In some embodiments, the PAM is an “NRN” PAM where the “N” in “NRN” is adenine (A), thymine (T), guanine (G), or cytosine (C), and the R is adenine (A) or guanine (G); or the PAM is an “NYN” PAM, wherein the “N” in NYN is adenine (A), thymine (T), guanine (G), or cytosine (C), and the Y is cytidine (C) or thymine (T), for example, as described in R. T. Walton et al., 2020, Science, 10.1126/science.aba8853 (2020), the entire contents of which are incorporated herein by reference.

Several PAM variants are described in Table 3 below.

TABLE 3
Cas9 proteins and corresponding PAM sequences.
N is A, C, T, or G; and V is A, C, or G.

| Variant | PAM |
|---|---|
| spCas9 | NGG |
| spCas9-VRQR | NGA |
| spCas9-VRER | NGCG |
| xCas9 (sp) | NGN |
| saCas9 | NNGRRT |
| saCas9-KKH | NNNRRT |
| spCas9-MQKSER | NGCG |
| spCas9-MQKSER | NGCN |
| spCas9-LRKIQK | NGTN |
| spCas9-LRVSQK | NGTN |
| spCas9-LRVSQL | NGTN |
| spCas9-MQKFRAER | NGC |
| Cpf1 | 5′ (TTTV) |
| SpyMac | 5′-NAA-3′ |

In some embodiments, the PAM is NGC. In some embodiments, the NGC PAM is recognized by a Cas9 variant. In some embodiments, the NGC PAM Cas9 variant includes one or more amino acid substitutions selected from D1135M, S1136Q, G1218K, E1219F, A1322R, D1332A, R1335E, and T1337R (collectively termed “MQKFRAER”) of spCas9 (SEQ ID No: 197), or a corresponding mutation in another Cas9. In some embodiments, the Cas9 variant contains one or more amino acid substitutions selected from D1135V, G1218R, R1335Q, and T1337R (collectively termed VRQR) of spCas9 (SEQ ID No: 197), or a corresponding mutation in another Cas9. In some embodiments, the Cas9 variant contains one or more amino acid substitutions selected from D1135V, G1218R, R1335E, and T1337R (collectively termed VRER) of spCas9 (SEQ ID No: 197), or a corresponding mutation in another Cas9. In some embodiments, the Cas9 variant contains one or more amino acid substitutions selected from E782K, N968K, and R1015H (collectively termed KHH) of saCas9 (SEQ ID NO: 218). In some embodiments, the Cas9 variant includes one or more amino acid substitutions selected from D1135M, S1136Q, G1218K, E1219S, R1335E, and T1337R (collectively termed “MQKSER”) of spCas9 (SEQ ID No: 197), or a corresponding mutation in another Cas9. In some embodiments, the Cas9 variant includes one or more amino acid substitutions selected from D1135M, S1136Q, G1218K, E1219S, R1335E, and T1337R (collectively termed “MQKSER”) of spCas9 (SEQ ID No: 197), or a corresponding mutation in another Cas9.

In some embodiments, a CRISPR protein-derived domain of a base editor comprises all or a portion (e.g., a functional portion) of a Cas9 protein with a canonical PAM sequence (NGG). In other embodiments, a Cas9-derived domain of a base editor can employ a non-canonical PAM sequence. Such sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); R. T. Walton et al. “Unconstrained genome targeting with near-PAMless engineered CRISPR-Cas9 variants” Science 10.1126/science.aba8853 (2020); Hu et al. “Evolved Cas9 variants with broad PAM compatibility and high DNA specificity,” Nature, 2018 Apr. 5, 556 (7699), 57-63; Miller et al., “Continuous evolution of SpCas9 variants compatible with non-G PAMs” Nat. Biotechnol., 2020 April; 38 (4): 471-481; the entire contents of each are hereby incorporated by reference.

Fusion Proteins or Complexes Comprising a NapDNAbp and a Cytidine Deaminase and/or Adenosine Deaminase

Some aspects of the disclosure provide fusion proteins or complexes comprising a Cas9 domain or other nucleic acid programmable DNA binding protein (e.g., Cas12) and one or more cytidine deaminase, adenosine deaminase, or cytidine adenosine deaminase domains. It should be appreciated that the Cas9 domain may be any of the Cas9 domains or Cas9 proteins (e.g., dCas9 or nCas9) provided herein. In some embodiments, any of the Cas9 domains or Cas9 proteins (e.g., dCas9 or nCas9) provided herein may be fused with any of the cytidine deaminases and/or adenosine deaminases provided herein. The domains of the base editors disclosed herein can be arranged in any order.

In some embodiments, the fusion proteins or complexes comprising a cytidine deaminase or adenosine deaminase and a napDNAbp (e.g., Cas9 or Cas12 domain) do not include a linker sequence. In some embodiments, a linker is present between the cytidine or adenosine deaminase and the napDNAbp. In some embodiments, cytidine or adenosine deaminase and the napDNAbp are fused via any of the linkers provided herein. For example, in some embodiments the cytidine or adenosine deaminase and the napDNAbp are fused via any of the linkers provided herein.

It should be appreciated that the fusion proteins or complexes of the present disclosure may comprise one or more additional features. For example, in some embodiments, the fusion protein or complex may comprise inhibitors, cytoplasmic localization sequences, export sequences, such as nuclear export sequences, or other localization sequences, as well as sequence tags that are useful for solubilization, purification, or detection of the fusion proteins or complexes. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein or complex comprises one or more His tags.

Exemplary, yet nonlimiting, fusion proteins are described in International PCT Application Nos. PCT/US2017/045381, PCT/US2019/044935, and PCT/US2020/016288, each of which is incorporated herein by reference for its entirety.

Fusion Proteins or Complexes with Internal Insertions

Provided herein are fusion proteins or complexes comprising a heterologous polypeptide fused to a nucleic acid programmable nucleic acid binding protein, for example, a napDNAbp. The heterologous polypeptide can be fused to the napDNAbp at a C-terminal end of the napDNAbp, an N-terminal end of the napDNAbp, or inserted at an internal location of the napDNAbp. In some embodiments, the heterologous polypeptide is a deaminase (e.g., cytidine or adenosine deaminase) or a functional fragment thereof. For example, a fusion protein can comprise a deaminase flanked by an N-terminal fragment and a C-terminal fragment of a Cas9 or Cas12 (e.g., Cas12b/C2c1), polypeptide.

The deaminase can be a circular permutant deaminase. In some embodiments, the deaminase is a circular permutant TadA, circularly permutated at amino acid residue 116, 136, or 65 as numbered in a TadA reference sequence.

The fusion protein or complexes can comprise more than one deaminase. The fusion protein or complex can comprise, for example, 1, 2, 3, 4, 5 or more deaminases. The deaminases in a fusion protein or complex can be adenosine deaminases, cytidine deaminases, or a combination thereof.

In some embodiments, the napDNAbp in the fusion protein or complex contains a Cas9 polypeptide or a fragment thereof. The Cas9 polypeptide can be a variant Cas9 polypeptide. The Cas9 polypeptide can be a circularly permuted Cas9 protein.

The heterologous polypeptide (e.g., deaminase) can be inserted in the napDNAbp (e.g., Cas9 or Cas12 (e.g., Cas12b/C2c1)) at a suitable location, for example, such that the napDNAbp retains its ability to bind the target polynucleotide and a guide nucleic acid. A deaminase (e.g., adenosine deaminase, cytidine deaminase, or adenosine deaminase and cytidine deaminase (dual deaminase)) can be inserted into a napDNAbp without compromising function of the deaminase (e.g., base editing activity) or the napDNAbp (e.g., ability to bind to target nucleic acid and guide nucleic acid).

In some embodiments, the deaminase (e.g., adenosine deaminase, cytidine deaminase, or adenosine deaminase and cytidine deaminase) is inserted in regions of the Cas9 polypeptide comprising higher than average B-factors (e.g., higher B factors compared to the total protein or the protein domain comprising the disordered region). Cas9 polypeptide positions comprising a higher than average B-factor can include, for example, residues 768, 792, 1052, 1015, 1022, 1026, 1029, 1067, 1040, 1054, 1068, 1246, 1247, and 1248 as numbered in the above Cas9 reference sequence. Cas9 polypeptide regions comprising a higher than average B-factor can include, for example, residues 792-872, 792-906, and 2-791 as numbered in the above Cas9 reference sequence.

In some embodiments, a heterologous polypeptide (e.g., deaminase) is inserted in a flexible loop of a Cas9 polypeptide. The flexible loop portions can be selected from the group consisting of 530-537, 569-570, 686-691, 943-947, 1002-1025, 1052-1077, 1232-1247, or 1298-1300 as numbered in the above Cas9 reference sequence, or a corresponding amino acid residue in another Cas9 polypeptide. The flexible loop portions can be selected from the group consisting of: 1-529, 538-568, 580-685, 692-942, 948-1001, 1026-1051, 1078-1231, or 1248-1297 as numbered in the above Cas9 reference sequence, or a corresponding amino acid residue in another Cas9 polypeptide.

A heterologous polypeptide (e.g., adenine deaminase) can be inserted into a Cas9 polypeptide region corresponding to amino acid residues: 1017-1069, 1242-1247, 1052-1056, 1060-1077, 1002-1003, 943-947, 530-537, 568-579, 686-691, 1242-1247, 1298-1300, 1066-1077, 1052-1056, or 1060-1077 as numbered in the above Cas9 reference sequence, or a corresponding amino acid residue in another Cas9 polypeptide.

A heterologous polypeptide (e.g., adenine deaminase) can be inserted in place of a deleted region of a Cas9 polypeptide. The deleted region can correspond to an N-terminal or C-terminal portion of the Cas9 polypeptide. Exemplary internal fusions base editors are provided in Table 4A below:

TABLE 4A
Insertion loci in Cas9 proteins

| BE ID | Modification | Other ID |
|---|---|---|
| IBE001 | Cas9 TadA ins 1015 | ISLAY01 |
| IBE002 | Cas9 TadA ins 1022 | ISLAY02 |
| IBE003 | Cas9 TadA ins 1029 | ISLAY03 |
| IBE004 | Cas9 TadA ins 1040 | ISLAY04 |
| IBE005 | Cas9 TadA ins 1068 | ISLAY05 |
| IBE006 | Cas9 TadA ins 1247 | ISLAY06 |
| IBE007 | Cas9 TadA ins 1054 | ISLAY07 |
| IBE008 | Cas9 TadA ins 1026 | ISLAY08 |
| IBE009 | Cas9 TadA ins 768 | ISLAY09 |
| IBE020 | delta HNH TadA 792 | ISLAY20 |
| IBE021 | N-term fusion single TadA helix truncated 165-end | ISLAY21 |
| IBE029 | TadA-Circular Permutant116 ins1067 | ISLAY29 |
| IBE031 | TadA- Circular Permutant 136 ins1248 | ISLAY31 |
| IBE032 | TadA- Circular Permutant 136ins 1052 | ISLAY32 |
| IBE035 | delta 792-872 TadA ins | ISLAY35 |
| IBE036 | delta 792-906 TadA ins | ISLAY36 |
| IBE043 | TadA-Circular Permutant 65 ins1246 | ISLAY43 |
| IBE044 | TadA ins C-term truncate2 791 | ISLAY44 |

A heterologous polypeptide (e.g., deaminase) can be inserted within a structural or functional domain of a Cas9 polypeptide. A heterologous polypeptide (e.g., deaminase) can be inserted between two structural or functional domains of a Cas9 polypeptide. A heterologous polypeptide (e.g., deaminase) can be inserted in place of a structural or functional domain of a Cas9 polypeptide, for example, after deleting the domain from the Cas9 polypeptide. The structural or functional domains of a Cas9 polypeptide can include, for example, RuvC I, RuvC II, RuvC III, Rec1, Rec2, PI, or HNH.

A fusion protein can comprise a linker between the deaminase and the napDNAbp polypeptide. The linker can be a peptide or a non-peptide linker. For example, the linker can be an XTEN, (GGGS)ₙ (SEQ ID NO: 246), SGGSSGGS (SEQ ID NO: 330), (GGGGS)ₙ (SEQ ID NO: 247), (G)ₙ, (EAAAK)n (SEQ ID NO: 248), (GGS)ₙ, SGSETPGTSESATPES (SEQ ID NO: 249). In some embodiments, the fusion protein comprises a linker between the N-terminal Cas9 fragment and the deaminase. In some embodiments, the fusion protein comprises a linker between the C-terminal Cas9 fragment and the deaminase. In some embodiments, the N-terminal and C-terminal fragments of napDNAbp are connected to the deaminase with a linker. In some embodiments, the N-terminal and C-terminal fragments are joined to the deaminase domain without a linker. In some embodiments, the fusion protein comprises a linker between the N-terminal Cas9 fragment and the deaminase but does not comprise a linker between the C-terminal Cas9 fragment and the deaminase. In some embodiments, the fusion protein comprises a linker between the C-terminal Cas9 fragment and the deaminase but does not comprise a linker between the N-terminal Cas9 fragment and the deaminase.

In some embodiments, the napDNAbp in the fusion protein or complex is a Cas12 polypeptide, e.g., Cas12b/C2c1, or a functional fragment thereof capable of associating with a nucleic acid (e.g., a gRNA) that guides the Cas12 to a specific nucleic acid sequence. The Cas12 polypeptide can be a variant Cas12 polypeptide. In other embodiments, the N- or C-terminal fragments of the Cas12 polypeptide comprise a nucleic acid programmable DNA binding domain or a RuvC domain. In other embodiments, the fusion protein contains a linker between the Cas12 polypeptide and the catalytic domain. In other embodiments, the amino acid sequence of the linker is GGSGGS (SEQ ID NO: 250) or GSSGSETPGTSESATPESSG (SEQ ID NO: 251). In other embodiments, the linker is a rigid linker. In other embodiments of the above aspects, the linker is encoded by GGAGGCTCTGGAGGAAGC (SEQ ID NO: 252) or GGCTCTTCTGGATCTGAAACACCTGGCACAAGCGAGAGCGCCACCCCTGAGAGCTCTGGC (SEQ ID NO: 253).

In other embodiments, the fusion protein or complex contains a nuclear localization signal (e.g., a bipartite nuclear localization signal). In other embodiments, the amino acid sequence of the nuclear localization signal is MAPKKKRKVGIHGVPAA (SEQ ID NO: 261). In other embodiments of the above aspects, the nuclear localization signal is encoded by the following sequence:

ATGGCCCCAAAGAAGAAGCGGAAGGTCGGTATCCACGGAGTCCCAGCAGCC (SEQ ID NO: 262). In other embodiments, the Cas12b polypeptide contains a mutation that silences the catalytic activity of a RuvC domain. In other embodiments, the Cas12b polypeptide contains D574A, D829A and/or D952A mutations.

In some embodiments, the fusion protein or complex comprises a napDNAbp domain (e.g., Cas12-derived domain) with an internally fused nucleobase editing domain (e.g., all or a portion (e.g., a functional portion) of a deaminase domain, e.g., an adenosine deaminase domain). In some embodiments, the napDNAbp is a Cas12b. In some embodiments, the base editor comprises a BhCas12b domain with an internally fused TadA*8 domain inserted at the loci provided in Table 4B below.

TABLE 4B
Insertion loci in Cas12b proteins

| | Insertion site | Inserted between aa |
|---|---|---|
| BhCas12b | | |
| position 1 | 153 | PS |
| position 2 | 255 | KE |
| position 3 | 306 | DE |
| position 4 | 980 | DG |
| position 5 | 1019 | KL |
| position 6 | 534 | FP |
| position 7 | 604 | KG |
| position 8 | 344 | HF |
| BvCas12b | | |
| position 1 | 147 | PD |
| position 2 | 248 | GG |
| position 3 | 299 | PE |
| position 4 | 991 | GE |
| position 5 | 1031 | KM |
| AaCas12b | | |
| position 1 | 157 | PG |
| position 2 | 258 | VG |
| position 3 | 310 | DP |
| position 4 | 1008 | GE |
| position 5 | 1044 | GK |

In some embodiments, the base editing system described herein is an ABE with TadA inserted into a Cas9. Polypeptide sequences of relevant ABEs with TadA inserted into a Cas9 are provided in the attached Sequence Listing as SEQ ID NOs: 263-308.

Exemplary, yet nonlimiting, fusion proteins are described in International PCT Application Nos. PCT/US2020/016285 and U.S. Provisional Application Nos. 62/852,228 and 62/852,224, the contents of which are incorporated by reference herein in their entireties.

### A to G Editing

In some embodiments, a base editor described herein comprises an adenosine deaminase domain. Such an adenosine deaminase domain of a base editor can facilitate the editing of an adenine (A) nucleobase to a guanine (G) nucleobase by deaminating the A to form inosine (I), which exhibits base pairing properties of G. In some embodiments, an A-to-G base editor further comprises an inhibitor of inosine base excision repair, for example, a uracil glycosylase inhibitor (UGI) domain or a catalytically inactive inosine specific nuclease. Without wishing to be bound by any particular theory, the UGI domain or catalytically inactive inosine specific nuclease can inhibit or prevent base excision repair of a deaminated adenosine residue (e.g., inosine), which can improve the activity or efficiency of the base editor.

A base editor comprising an adenosine deaminase can act on any polynucleotide, including DNA, RNA and DNA-RNA hybrids. In an embodiment an adenosine deaminase domain of a base editor comprises all or a portion (e.g., a functional portion) of an ADAT comprising one or more mutations which permit the ADAT to deaminate a target A in DNA. For example, the base editor can comprise all or a portion (e.g., a functional portion) of an ADAT from Escherichia coli (EcTadA) comprising one or more of the following mutations: D108N, A106V, D147Y, E155V, L84F, H123Y, 1156F, or a corresponding mutation in another adenosine deaminase. Exemplary ADAT homolog polypeptide sequences are provided in the Sequence Listing as SEQ ID NOs: 1 and 309-315.

The adenosine deaminase can be derived from any suitable organism (e.g., E. coli). In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenine deaminase is a naturally-occurring adenosine deaminase that includes one or more mutations corresponding to any of the mutations provided herein (e.g., mutations in ecTadA). The corresponding residue in any homologous protein can be identified by e.g., sequence alignment and determination of homologous residues. The mutations in any naturally-occurring adenosine deaminase (e.g., having homology to ecTadA) that correspond to any of the mutations described herein (e.g., any of the mutations identified in ecTadA) can be generated accordingly.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any of the adenosine deaminases provided herein. It should be appreciated that adenosine deaminases provided herein may include one or more mutations (e.g., any of the mutations provided herein). The disclosure provides any deaminase domains with a certain percent identify plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the adenosine deaminases provided herein.

It should be appreciated that any of the mutations provided herein (e.g., based on a TadA reference sequence, such as TadA*7.10 (SEQ ID NO: 1)) can be introduced into other adenosine deaminases, such as E. coli TadA (ecTadA), S. aureus TadA (saTadA), or other adenosine deaminases (e.g., bacterial adenosine deaminases). In some embodiments, the TadA reference sequence is TadA*7.10 (SEQ ID NO: 1). It would be apparent to the skilled artisan that additional deaminases may similarly be aligned to identify homologous amino acid residues that can be mutated as provided herein. Thus, any of the mutations identified in a TadA reference sequence can be made in other adenosine deaminases (e.g., ecTada) that have homologous amino acid residues. It should also be appreciated that any of the mutations provided herein can be made individually or in any combination in a TadA reference sequence or another adenosine deaminase.

In some embodiments, the adenosine deaminase comprises an alteration or set of alterations selected from those listed in Tables 5A-5E below:

TABLE 5A
Adenosine Deaminase Variants. Residue positions in the E. coli TadA variant
(TadA*) are indicated.

| | 23 | 26 | 36 | 37 | 48 | 49 | 51 | 72 | 84 | 87 | 106 | 108 | 123 | 125 | 142 | 146 | 147 | 152 | 155 | 156 | 157 | 161 |
|---|---|---|---|---|---|---|---|---|---|---|---|---|---|---|---|---|---|---|---|---|---|---|
| TadA*0.1 | W | R | H | N | P | | R | N | L | S | A | D | H | G | A | S | D | R | E | I | K | K |
| TadA*0.2 | W | R | H | N | P | | R | N | L | S | A | D | H | G | A | S | D | R | E | I | K | K |
| TadA*1.1 | W | R | H | N | P | | R | N | L | S | A | N | H | G | A | S | D | R | E | I | K | K |
| TadA*1.2 | W | R | H | N | P | | R | N | L | S | V | N | H | G | A | S | D | R | E | I | K | K |
| TadA*2.1 | W | R | H | N | P | | R | N | L | S | V | N | H | G | A | S | Y | R | V | I | K | K |
| TadA*2.2 | W | R | H | N | P | | R | N | L | S | V | N | H | G | A | S | Y | R | V | I | K | K |
| TadA*2.3 | W | R | H | N | P | | R | N | L | S | V | N | H | G | A | S | Y | R | V | I | K | K |
| TadA*2.4 | W | R | H | N | P | | R | N | L | S | V | N | H | G | A | S | Y | R | V | I | K | K |
| TadA*2.5 | W | R | H | N | P | | R | N | L | S | V | N | H | G | A | S | Y | R | V | I | K | K |
| TadA*2.6 | W | R | H | N | P | | R | N | T | S | V | N | H | G | A | S | Y | R | V | I | K | K |
| TadA*2.7 | W | R | H | N | P | | R | N | L | S | V | N | H | G | A | S | Y | R | V | I | K | K |
| TadA*2.8 | W | R | H | N | P | | R | N | L | S | V | N | H | G | A | S | Y | R | V | I | K | K |
| TadA*2.9 | W | R | H | N | P | | R | N | L | S | V | N | H | G | A | S | Y | R | V | I | K | K |
| TadA*2.10 | W | R | H | N | P | | R | N | L | S | V | N | H | G | A | S | Y | R | V | I | K | K |
| TadA*2.11 | W | R | H | N | P | | R | N | L | S | V | N | H | G | A | S | Y | R | V | I | K | K |
| TadA*2.12 | W | R | H | N | P | | R | N | L | S | V | N | H | G | A | S | Y | R | V | I | K | K |
| TadA*3.1 | W | R | H | N | P | | R | N | F | S | V | N | Y | G | A | S | Y | R | V | F | K | K |
| TadA*3.2 | W | R | H | N | P | | R | N | F | S | V | N | Y | G | A | S | Y | R | V | F | K | K |
| TadA*3.3 | W | R | H | N | P | | R | N | F | S | V | N | Y | G | A | S | Y | R | V | F | K | K |
| TadA*3.4 | W | R | H | N | P | | R | N | F | S | V | N | Y | G | A | S | Y | R | V | F | K | K |
| TadA*3.5 | W | R | H | N | P | | R | N | F | S | V | N | Y | G | A | S | Y | R | V | F | K | K |
| TadA*3.6 | W | R | H | N | P | | R | N | F | S | V | N | Y | G | A | S | Y | R | V | F | K | K |
| TadA*3.7 | W | R | H | N | P | | R | N | F | S | V | N | Y | G | A | S | Y | R | V | F | K | K |
| TadA*3.8 | W | R | H | N | P | | R | N | F | S | V | N | Y | G | A | S | Y | R | V | F | K | K |
| TadA*4.1 | W | R | H | N | P | | R | N | L | S | V | N | H | G | N | S | Y | R | V | I | K | K |
| TadA*4.2 | W | G | H | N | P | | R | N | L | S | V | N | H | G | N | S | Y | R | V | I | K | K |
| TadA*4.3 | W | R | H | N | P | | R | N | F | S | V | N | Y | G | N | S | Y | R | V | F | K | K |
| TadA*5.1 | W | R | L | N | P | | L | N | F | S | V | N | Y | G | A | C | Y | R | V | F | N | K |
| TadA*5.2 | W | R | H | S | P | | R | N | F | S | V | N | Y | G | A | S | Y | R | V | F | K | T |
| TadA*5.3 | W | R | L | N | P | | L | N | I | S | V | N | Y | G | A | C | Y | R | V | F | N | K |
| TadA*5.4 | W | R | H | S | P | | R | N | F | S | V | N | Y | G | A | S | Y | R | V | F | K | T |
| TadA*5.5 | W | R | L | N | P | | L | N | F | S | V | N | Y | G | A | C | Y | R | V | F | N | K |
| TadA*5.6 | W | R | L | N | P | | L | N | F | S | V | N | Y | G | A | C | Y | R | V | F | N | K |
| TadA*5.7 | W | R | L | N | P | | L | N | F | S | V | N | Y | G | A | C | Y | R | V | F | N | K |
| TadA*5.8 | W | R | L | N | P | | L | N | F | S | V | N | Y | G | A | C | Y | R | V | F | N | K |
| TadA*5.9 | W | R | L | N | P | | L | N | F | S | V | N | Y | G | A | C | Y | R | V | F | N | K |
| TadA*5.10 | W | R | L | N | P | | L | N | F | S | V | N | Y | G | A | C | Y | R | V | F | N | K |
| TadA*5.11 | W | R | L | N | P | | L | N | F | S | V | N | Y | G | A | C | Y | R | V | F | N | K |
| TadA*5.12 | W | R | L | N | P | | L | N | F | S | V | N | Y | G | A | C | Y | R | V | F | N | K |
| TadA*5.13 | W | R | H | N | P | | L | D | F | S | V | N | Y | A | A | S | Y | R | V | F | K | K |
| TadA*5.14 | W | R | H | N | S | | L | N | F | C | V | N | Y | G | A | S | Y | R | V | F | K | K |
| TadA*6.1 | W | R | H | N | S | | L | N | F | S | V | N | Y | G | N | S | Y | R | V | F | K | K |
| TadA*6.2 | W | R | H | N | T | V | L | N | F | S | V | N | Y | G | N | S | Y | R | V | F | N | K |
| TadA*6.3 | W | R | L | N | S | | L | N | F | S | V | N | Y | G | A | C | Y | R | V | F | N | K |
| TadA*6.4 | W | R | L | N | S | | L | N | F | S | V | N | Y | G | N | C | Y | R | V | F | N | K |
| TadA*6.5 | W | R | L | N | T | V | L | N | F | S | V | N | Y | G | A | C | Y | R | V | F | N | K |
| TadA*6.6 | W | R | L | N | T | V | L | N | F | S | V | N | Y | G | N | C | Y | R | V | F | N | K |
| TadA*7.1 | W | R | L | N | A | | L | N | F | S | V | N | Y | G | A | C | Y | R | V | F | N | K |
| TadA*7.2 | W | R | L | N | A | | L | N | F | S | V | N | Y | G | N | C | Y | R | V | F | N | K |
| TadA*7.3 | L | R | L | N | A | | L | N | F | S | V | N | Y | G | A | C | Y | R | V | F | N | K |
| TadA*7.4 | R | R | L | N | A | | L | N | F | S | V | N | Y | G | A | C | Y | R | V | F | N | K |
| TadA*7.5 | W | R | L | N | A | | L | N | F | S | V | N | Y | G | A | C | Y | H | V | F | N | K |
| TadA*7.6 | W | R | L | N | A | | L | N | I | S | V | N | Y | G | A | C | Y | P | V | F | N | K |
| TadA*7.7 | L | R | L | N | A | | L | N | F | S | V | N | Y | G | A | C | Y | P | V | F | N | K |
| TadA*7.8 | L | R | L | N | A | | L | N | F | S | V | N | Y | G | N | C | Y | R | V | F | N | K |
| TadA*7.9 | L | R | L | N | A | | L | N | F | S | V | N | Y | G | N | C | Y | P | V | F | N | K |
| TadA*7.10 | R | R | L | N | A | | L | N | F | S | V | N | Y | G | A | C | Y | P | V | F | N | K |

TABLE 5B
TadA*8 Adenosine Deaminase Variants. Residue positions in the E. coli TadA
variant (TadA*) are indicated. Alterations are referenced to TadA*7.10 (first row).

| | 23 | 36 | 48 | 51 | 76 | 82 | 84 | 106 | 108 | 123 | 146 | 147 | 152 | 154 | 155 | 156 | 157 | 166 |
|---|---|---|---|---|---|---|---|---|---|---|---|---|---|---|---|---|---|---|
| TadA*7.10 | R | L | A | L | I | V | F | V | N | Y | C | Y | P | Q | V | F | N | T |
| TadA*8.1 | | | | | | | | | | | | T | | | | | | |
| TadA*8.2 | | | | | | | | | | | | R | | | | | | |
| TadA*8.3 | | | | | | | | | | | | | | S | | | | |
| TadA*8.4 | | | | | | | | | | H | | | | | | | | |
| TadA*8.5 | | | | | | S | | | | | | | | | | | | |
| TadA*8.6 | | | | | | | | | | | | | | | | | | R |
| TadA*8.7 | | | | | | | | | | | | | | R | | | | |
| TadA*8.8 | | | | | | | | | | H | | R | | R | | | | |
| TadA*8.9 | | | | | Y | | | | | | | R | | R | | | | |
| TadA*8.10 | | | | | | | | | | | | R | | R | | | | R |
| TadA*8.11 | | | | | | | | | | | | T | | R | | | | |
| TadA*8.12 | | | | | | | | | | | | T | | S | | | | |
| TadA*8.13 | | | | | Y | | | | | H | | R | | R | | | | |
| TadA*8.14 | | | | | Y | S | | | | | | | | | | | | |
| TadA*8.15 | | | | | | S | | | | | | R | | | | | | |
| TadA*8.16 | | | | | | S | | | | H | | R | | | | | | |
| TadA*8.17 | | | | | | S | | | | | | | | R | | | | |
| TadA*8.18 | | | | | | S | | | | H | | | | R | | | | |
| TadA*8.19 | | | | | | S | | | | H | | R | | R | | | | |
| TadA*8.20 | | | | | Y | S | | | | H | | R | | R | | | | |
| TadA*8.21 | | | | | | | | | | | | R | | S | | | | |
| TadA*8.22 | | | | | | S | | | | | | | | S | | | | |
| TadA*8.23 | | | | | | S | | | | H | | | | | | | | |
| TadA*8.24 | | | | | | S | | | | H | | T | | | | | | |

TABLE 5C
TadA*9 Adenosine Deaminase Variants. Alterations are referenced to TadA*7.10.
Additional details of TadA*9 adenosine deaminases are described in
International PCT Application No. PCT/US2020/049975, which is incorporated
herein by reference in its entirety for all purposes.

| TadA*9 Description | Alterations |
|---|---|
| TadA*9.1 | E25F, V82S, Y123H, T133K, Y147R, Q154R |
| TadA*9.2 | E25F, V82S, Y123H, Y147R, Q154R |
| TadA*9.3 | V82S, Y123H, P124W, Y147R, Q154R |
| TadA*9.4 | L51W, V82S, Y123H, C146R, Y147R, Q154R |
| TadA*9.5 | P54C, V82S, Y123H, Y147R, Q154R |
| TadA*9.6 | Y73S, V82S, Y123H, Y147R, Q154R |
| TadA*9.7 | N38G, V82T, Y123H, Y147R, Q154R |
| TadA*9.8 | R23H, V82S, Y123H, Y147R, Q154R |
| TadA*9.9 | R21N, V82S, Y123H, Y147R, Q154R |
| TadA*9.10 | V82S, Y123H, Y147R, Q154R, A158K |
| TadA*9.11 | N72K, V82S, Y123H, D139L, Y147R, Q154R, |
| TadA*9.12 | E25F, V82S, Y123H, D139M, Y147R, Q154R |
| TadA*9.13 | M70V, V82S, M94V, Y123H, Y147R, Q154R |
| TadA*9.14 | Q71M, V82S, Y123H, Y147R, Q154R |
| TadA*9.15 | E25F, V82S, Y123H, T133K, Y147R, Q154R |
| TadA*9.16 | E25F, V82S, Y123H, Y147R, Q154R |
| TadA*9.17 | V82S, Y123H, P124W, Y147R, Q154R |
| TadA*9.18 | L51W, V82S, Y123H, C146R, Y147R, Q154R |
| TadA*9.19 | P54C, V82S, Y123H, Y147R, Q154R |
| TadA*9.2 | Y73S, V82S, Y123H, Y147R, Q154R |
| TadA*9.21 | N38G, V82T, Y123H, Y147R, Q154R |
| TadA*9.22 | R23H, V82S, Y123H, Y147R, Q154R |
| TadA*9.23 | R21N, V82S, Y123H, Y147R, Q154R |
| TadA*9.24 | V82S, Y123H, Y147R, Q154R, A158K |
| TadA*9.25 | N72K, V82S, Y123H, D139L, Y147R, Q154R, |
| TadA*9.26 | E25F, V82S, Y123H, D139M, Y147R, Q154R |
| TadA*9.27 | M70V, V82S, M94V, Y123H, Y147R, Q154R |
| TadA*9.28 | Q71M, V82S, Y123H, Y147R, Q154R |
| TadA*9.29 | E25F_I76Y_V82S_Y123H_Y147R_Q154R |
| TadA*9.30 | I76Y_V82T_Y123H_Y147R_Q154R |
| TadA*9.31 | N38G_I76Y_V82S_Y123H_Y147R_Q154R |
| TadA*9.32 | N38G_I76Y_V82T_Y123H_Y147R_Q154R |
| TadA*9.33 | R23H_I76Y_V82S_Y123H_Y147R_Q154R |
| TadA*9.34 | P54C_I76Y_V82S_Y123H_Y147R_Q154R |
| TadA*9.35 | R21N_I76Y_V82S_Y123H_Y147R_Q154R |
| TadA*9.36 | I76Y_V82S_Y123H_D138M_Y147R_Q154R |
| TadA*9.37 | Y72S_I76Y_V82S_Y123H_Y147R_Q154R |
| TadA*9.38 | E25F_I76Y_V82S_Y123H_Y147R_Q154R |
| TadA*9.39 | I76Y_V82T_Y123H_Y147R_Q154R |
| TadA*9.40 | N38G_I76Y_V82S_Y123H_Y147R_Q154R |
| TadA*9.41 | N38G_I76Y_V82T_Y123H_Y147R_Q154R |
| TadA*9.42 | R23H_I76Y_V82S_Y123H_Y147R_Q154R |
| TadA*9.43 | P54C_I76Y_V82S_Y123H_Y147R_Q154R |
| TadA*9.44 | R21N_I76Y_V82S_Y123H_Y147R_Q154R |
| TadA*9.45 | I76Y_V82S_Y123H_D138M_Y147R_Q154R |
| TadA*9.46 | Y72S_I76Y_V82S_Y123H_Y147R_Q154R |
| TadA*9.47 | N72K_V82S, Y123H, Y147R, Q154R |
| TadA*9.48 | Q71M_V82S, Y123H, Y147R, Q154R |
| TadA*9.49 | M70V, V82S, M94V, Y123H, Y147R, Q154R |
| TadA*9.50 | V82S, Y123H, T133K, Y147R, Q154R |
| TadA*9.51 | V82S, Y123H, T133K, Y147R, Q154R, A158K |
| TadA*9.52 | M70V, Q71M, N72K, V82S, Y123H, Y147R, Q154R |
| TadA*9.53 | N72K_V82S, Y123H, Y147R, Q154R |
| TadA*9.54 | Q71M_V82S, Y123H, Y147R, Q154R |
| TadA*9.55 | M70V, V82S, M94V, Y123H, Y147R, Q154R |
| TadA*9.56 | V82S, Y123H, T133K, Y147R, Q154R |
| TadA*9.57 | V82S, Y123H, T133K, Y147R, Q154R, A158K |
| TadA*9.58 | M70V, Q71M, N72K, V82S, Y123H, Y147R, Q154R |

In some embodiments, the adenosine deaminase comprises a TadA*8.20 adenosine deaminase variant further comprising an F149Y amino acid alteration. In some embodiments, the adenosine deaminase comprises a TadA*8.20 adenosine deaminase variant further comprising the amino acid alterations R147D, F149Y, T166I, and D167N (TadA*8.10+). In some embodiments, the adenosine deaminase comprises a TadA*8.20 adenosine deaminase variant further comprising the amino acid alterations S82T and F149Y (TadA*9v1). In some embodiments, the adenosine deaminase comprises a TadA*8.20 adenosine deaminase variant further comprising the amino acid alterations Y147D, F149Y, T166I, D167N and S82T (TadA*9v2).

In some embodiments, the adenosine deaminase comprises one or more of MII, S2A, S2E, V4D, V4E, V4M, F6S, H8E, H8Y, E9Y, M12S, R13H, R13I, R13Y, T17L, T17S, L18A, L18E, A19N, R21N, K20K, K20R, R21A, G22P, W23D, R23H, W23G, W23Q, W23L, W23R, D24E, D24G, E25F, E25M, E25D, E25A, E25G, E25R, E25V, E25S, E25Y, R26D, R26E, R26G, R26N, R26Q, R26C, R26L, R26K, R26W, E27V, E27D, P29V, V30G, L34S, L34V, L36H, H36L, H36N, N37N, N37T, N37S, N38G, N38R, W45A, W45L, W45N, N46N, R46W, R46F, R46Q, R46M, R47A, R47Q, R47F, R47K, R47P, R47W, R47M, P48T, P48L, P48A, P48I, P48S, 149G, 149H, 149V, 149F, 149H, G50L, R51H, R51L, R51N, L51W, R51Y, H52D, H52Y, D53P, P54C, P54T, A55H, T55A, A56E, A56S, E59A, E59G, E59I, E59Q, E59W, M61A, M61I, M61L, M61V, L63S, L63V, Q65V, G66C, G67D, G67L, G67V, L68Q, M70H, M70Q, L84F, M70V, M70L, E70A, M70V, Q71M, Q7IN, Q71L, Q71R, N72A, N72K, N72S, N72D, N72Y, Y73G, Y73I, Y73K, Y73R, Y73S, R74A, R74Q, R74G, R74K, R74L, R74N, I76D, I76F, 1761, 176N, I76T, I76Y, D77G, A78I, T79M, L80M, L80Y, V82A, V82S, V82G, V82T, L84E, L84F, L84Y, E85K, E85G, E85P, E85S, S87C, S87L, S87V, V88A, V88M, C90S, A91A, A91G, A91S, A91V, A91T, G92T, A93I, M94A, M94V, M94L, M94I, M94H, 195S, 195G, 195L, 195H, 195V, H96A, H96L, H96R, H96S, S97C, S97G, S971, S97M, S97R, S97S, R98K, R98I, R98N, R98Q, G100R, G100V, R101V, R101R, V102A, V102F, V102I, V102V, D103A, F104G, D104N, F104V, F104I, F104L, A106T, V106Q, V106F, V106W, V106M, A106A, A106Q, A106F, A106G, A106W, A106M, A106V, A106R, R107C, R107G, R107P, R107K, R107A, R107N, R107W, R107H, R107S, D108N, D108F, D108G, D108V, D108A, D108Y, D108H, D108I, D108K, D108L, D108M, D108Q, N108Q, N108F, N108W, N108M, N108K, D108K, D108F, D108M, D108Q, D108R, D108W, D108S, A109H, A109K, A109R, A109S, A109T, A109V, K110G, K110H, K110I, K110R, K110T, T111A, T111G, T111H, T111R, G112A, A114G, A114H, A114V, G115S, L117M, L117N, L117V, M118D, M118G, M118K, M118N, M118V, D119L, D119N, D119S, D119V, V120H, V120L, H122H, H122N, H122P, H122R, H122S, H122Y, H123C, H123G, H123P, H123V, H123Y, Y123H, P124G, P124I, P124L, P124W, G125H, G125I, G125A, G125M, G125K, M126D, M126H, M126K, M126I, M126N, M1260, M126S, M126Y, N127H, N127S, N127D, N127K, N127R, H128R, R129H, R129Q, R129V, R129I, R129E, R129V, 11321, 1132F, T133V, T133E, T133G, T133K, E134A, E134E, E134G, E134I, G135G, G135V, 1136G, I136L, 1136T, 1137A, 1137D, 1137E, L137M, 1137S, A138D, A138E, A138G, S138A, A138N, A138S, A138T, A138V, A138Y, D139E, D139I, D139C, D139L, D139M, E140A, E140C, E140L, E140R, A142N, A142D, A142G, A142A, A142L, A142S, A142T, A142N, A142S, A142V, A143D, A143E, A143G, A143D, A143G, A143E, A143L, A143W, A143M, A143S, A143Q, A143R, C146R, S146A, S146C, S146D, S146F, S146R, S146T, D147D, D147L, D147F, D147G, D147Y, Y147T, Y147R, Y147D, D147R, F148L, F148F, F148R, F148Y, F149C, F149M, F149R, F149Y, M151F, M151P, M151R, M151V, R152C, R152F, R152H, R152P, R152R, R153C, R153Q, R153R, R153V, Q154E, Q154H, Q154M, Q154R, Q154L, Q154S, Q154V, E155F, E155G, E155I, E155K, E155P, E155V, E155D, 1156A, 1156F, 1156D, 1156K, 1156N, 1156R, 1156Y, E157A, E157F, E157I, E157P, E157T, E157V, N157K, K157N, K157R, A158Q, A158K, A158V, Q159F, Q159K, Q159L, Q159N, K160A, K160S, K160E, K160K, K160N, K161I, K161A, K161N, K161Q, K161S, K161T, A162D, A162Q, R162H, R162P, A162S, Q163G, Q163H, Q163N, Q163R, S164I, S164R, S164Y, S165A, S165D, S165I, S165T, S165Y, T166D, T166K, T166I, T166N, T166P, T166R, D167S and/or D167N mutation in a TadA reference sequence (e.g., TadA*7.10, ecTadA, or TadA8e), and any alternative mutation at the corresponding position, or any substitution from R26, W23, E27, H36, R47, P48, R51, H52, R74, I76, V82, V88, M94, 195, H96, A106, D108, A109, K110, T111, A114, D119, H122, H123, M126, N127, A142, S146, D147, F149, R152, Q154, E155, 1156, E157, K161, T166, and/or D167, with respect to a TadA reference sequence, or a substitution of 2-50 amino acids in a TadA reference sequence, which may be selected from W23R, E27D, H36L, R47K, P48A, R51H, R51L, I76F, I76Y, V82S, A106V, D108G, A109S, K110R, T111H, A114V, D119N, H122R, H122N, H123Y, M126I, N127K, S146C, D147R, R152P, Q154R, E155V, 1156F,K157N, K161N, T166I, and D167N, or one or more corresponding mutations in another adenosine deaminase. Additional mutations are described in U.S. Patent Application Publication No. 2022/0307003 A1 and International Patent Application Publications No. WO 2023/288304 A2 and WO 2023/034959 A2, the disclosures of which are incorporated herein by reference in their entirety for all purposes.

In embodiments, a variant of TadA*7.10 comprises one or more alterations selected from any of those alterations provided herein.

In particular embodiments, an adenosine deaminase heterodimer comprises a TadA*8 domain and an adenosine deaminase domain selected from Staphylococcus aureus (S. aureus) TadA, Bacillus subtilis (B. subtilis) TadA, Salmonella typhimurium (S. typhimurium) TadA, Shewanella putrefaciens (S. putrefaciens) TadA, Haemophilus influenzae F3031 (H. influenzae) TadA, Caulobacter crescentus (C. crescentus) TadA, Geobacter sulfurreducens (G. sulfurreducens) TadA, or TadA*7.10.

In some embodiments, the TadA*8 is a variant as shown in Table 5D. Table 5D shows certain amino acid position numbers in the TadA amino acid sequence and the amino acids present in those positions in the TadA-7.10 adenosine deaminase. Table 5D also shows amino acid changes in TadA variants relative to TadA-7.10 following phage-assisted non-continuous evolution (PANCE) and phage-assisted continuous evolution (PACE), as described in M. Richter et al., 2020, Nature Biotechnology, doi.org/10.1038/s41587-020-0453-z, the entire contents of which are incorporated by reference herein. In some embodiments, the TadA*8 is TadA*8a, TadA*8b, TadA*8c, TadA*8d, or TadA*8e. In some embodiments, the TadA*8 is TadA*8e. In one embodiment, an adenosine deaminase is a TadA*8 that comprises or consists essentially of SEQ ID NO: 316 or a fragment thereof having adenosine deaminase activity.

TABLE 5D
Select TadA*8 Variants

| | | TadA amino acid number | | | | | | | | | |
|---|---|---|---|---|---|---|---|---|---|---|---|
| | TadA | 26 | 88 | 109 | 111 | 119 | 122 | 147 | 149 | 166 | 167 |
| | TadA-7.10 | R | V | A | T | D | H | Y | F | T | D |
| PANCE 1 | | | | | R | | | | | | |
| PANCE 2 | | | | S/T | R | | | | | | |
| PACE | TadA-8a | C | | S | R | N | N | D | Y | I | N |
| | TadA-8b | | A | S | R | N | N | | Y | I | N |
| | TadA-8c | C | | S | R | N | N | | Y | I | N |
| | TadA-8d | | A | | R | N | | | Y | | |
| | TadA-8e | | | S | R | N | N | D | Y | I | N |

In some embodiments, the TadA variant is a variant as shown in Table 5E. Table 5E shows certain amino acid position numbers in the TadA amino acid sequence and the amino acids present in those positions in the TadA*7.10 adenosine deaminase. In some embodiments, the TadA variant is MSP605, MSP680, MSP823, MSP824, MSP825, MSP827, MSP828, or MSP829. In some embodiments, the TadA variant is MSP828. In some embodiments, the TadA variant is MSP829.

TABLE 5E
TadA Variants

| | TadA Amino Acid Number | | | | | | | |
|---|---|---|---|---|---|---|---|---|
| Variant | 36 | 76 | 82 | 147 | 149 | 154 | 157 | 167 |
| TadA-7.10 | L | I | V | Y | F | Q | N | D |
| MSP605 | | | G | T | | S | | |
| MSP680 | | Y | G | T | | S | | |
| MSP823 | H | | G | T | | S | K | |
| MSP824 | | | G | D | Y | S | | N |
| MSP825 | H | | G | D | Y | S | K | N |
| MSP827 | H | Y | G | T | | S | K | |
| MSP828 | | Y | G | D | Y | S | | N |
| MSP829 | H | Y | G | D | Y | S | K | N |

In particular embodiments, the fusion proteins or complexes comprise a single (e.g., provided as a monomer) TadA* (e.g., TadA*8 or TadA*9). Throughout the present disclosure, an adenosine deaminase base editor that comprises a single TadA* domain is indicates using the terminology ABEm or ABE #m, where “#” is an identifying number (e.g., ABE8.20m), where “m” indicates “monomer.” In some embodiments, the TadA* is linked to a Cas9 nickase. In some embodiments, the fusion proteins or complexes of the disclosure comprise as a heterodimer of a wild-type TadA (TadA(wt)) linked to a TadA*. Throughout the present disclosure, an adenosine deaminase base editor that comprises a single TadA* domain and a TadA (wt) domain is indicates using the terminology ABEd or ABE #d, where “#” is an identifying number (e.g., ABE8.20d), where “d” indicates “dimer.” In other embodiments, the fusion proteins or complexes of the disclosure comprise as a heterodimer of a TadA*7.10 linked to a TadA*. In some embodiments, the base editor is ABE8 comprising a TadA* variant monomer. In some embodiments, the base editor is ABE comprising a heterodimer of a TadA* and a TadA (wt). In some embodiments, the base editor is ABE comprising a heterodimer of a TadA* and TadA*7.10. In some embodiments, the base editor is ABE comprising a heterodimer of a TadA*. In some embodiments, the TadA* is selected from Tables 5A-5E.

In some embodiments, the adenosine deaminase is expressed as a monomer. In other embodiments, the adenosine deaminase is expressed as a heterodimer. In some embodiments, the deaminase or other polypeptide sequence lacks a methionine, for example when included as a component of a fusion protein. This can alter the numbering of positions. However, the skilled person will understand that such corresponding mutations refer to the same mutation.

Any of the mutations provided herein and any additional mutations (e.g., based on the ecTadA amino acid sequence) can be introduced into any other adenosine deaminases. Any of the mutations provided herein can be made individually or in any combination in a TadA reference sequence or another adenosine deaminase (e.g., ecTadA).

Details of A to G nucleobase editing proteins are described in International PCT Application No. PCT/US2017/045381 (WO2018/027078) and Gaudelli, N. M., et al., “Programmable base editing of A·T to G·C in genomic DNA without DNA cleavage” Nature, 551, 464-471 (2017), the entire contents of which are hereby incorporated by reference.

### C to T Editing

In some embodiments, a base editor disclosed herein comprises a fusion protein or complex comprising cytidine deaminase capable of deaminating a target cytidine (C) base of a polynucleotide to produce uridine (U), which has the base pairing properties of thymine. In some embodiments, for example where the polynucleotide is double-stranded (e.g., DNA), the uridine base can then be substituted with a thymidine base (e.g., by cellular repair machinery) to give rise to a C: G to a T: A transition. In other embodiments, deamination of a C to U in a nucleic acid by a base editor cannot be accompanied by substitution of the U to a T.

The deamination of a target C in a polynucleotide to give rise to a U is a non-limiting example of a type of base editing that can be executed by a base editor described herein. In another example, a base editor comprising a cytidine deaminase domain can mediate conversion of a cytosine (C) base to a guanine (G) base. For example, a U of a polynucleotide produced by deamination of a cytidine by a cytidine deaminase domain of a base editor can be excised from the polynucleotide by a base excision repair mechanism (e.g., by a uracil DNA glycosylase (UDG) domain), producing an abasic site. The nucleobase opposite the abasic site can then be substituted (e.g., by base repair machinery) with another base, such as a C, by for example a translesion polymerase. Although it is typical for a nucleobase opposite an abasic site to be replaced with a C, other substitutions (e.g., A, G or T) can also occur.

Accordingly, in some embodiments a base editor described herein comprises a deamination domain (e.g., cytidine deaminase domain) capable of deaminating a target C to a U in a polynucleotide. Further, as described below, the base editor can comprise additional domains which facilitate conversion of the U resulting from deamination to, in some embodiments, a T or a G. For example, a base editor comprising a cytidine deaminase domain can further comprise a uracil glycosylase inhibitor (UGI) domain to mediate substitution of a U by a T, completing a C-to-T base editing event. In another example, the base editor can comprise a uracil stabilizing protein as described herein. In another example, a base editor can incorporate a translesion polymerase to improve the efficiency of C-to-G base editing, since a translesion polymerase can facilitate incorporation of a C opposite an abasic site (i.e., resulting in incorporation of a G at the abasic site, completing the C-to-G base editing event).

A base editor comprising a cytidine deaminase as a domain can deaminate a target C in any polynucleotide, including DNA, RNA and DNA-RNA hybrids.

In some embodiments, a cytidine deaminase of a base editor comprises all or a portion (e.g., a functional portion) of an apolipoprotein B mRNA editing complex (APOBEC) family deaminase. APOBEC is a family of evolutionarily conserved cytidine deaminases. Members of this family are C-to-U editing enzymes. The N-terminal domain of APOBEC like proteins is the catalytic domain, while the C-terminal domain is a pseudocatalytic domain. More specifically, the catalytic domain is a zinc dependent cytidine deaminase domain and is important for cytidine deamination. APOBEC family members include APOBEC1, APOBEC2, APOBEC3A, APOBEC3B, APOBEC3C, APOBEC3D (“APOBEC3E” now refers to this), APOBEC3F, APOBEC3G, APOBEC3H, APOBEC4, and Activation-induced (cytidine) deaminase.

Other exemplary deaminases that can be fused to Cas9 according to aspects of this disclosure are provided below. In embodiments, the deaminases are activation-induced deaminases (AID). It should be understood that, in some embodiments, the active domain of the respective sequence can be used, e.g., the domain without a localizing signal (nuclear localization sequence, without nuclear export signal, cytoplasmic localizing signal).

Some aspects of the present disclosure are based on the recognition that modulating the deaminase domain catalytic activity of any of the fusion proteins or complexes described herein, for example by making point mutations in the deaminase domain, affect the processivity of the fusion proteins (e.g., base editors) or complexes. For example, mutations that reduce, but do not eliminate, the catalytic activity of a deaminase domain within a base editing fusion protein or complexes can make it less likely that the deaminase domain will catalyze the deamination of a residue adjacent to a target residue, thereby narrowing the deamination window. The ability to narrow the deamination window can prevent unwanted deamination of residues adjacent to specific target residues, which can reduce or prevent off-target effects.

In some embodiments, an APOBEC deaminase incorporated into a base editor can comprise one or more mutations selected from the group consisting of H121R, H122R, R126A, R126E, R118A, W90A, W90Y, and R132E of rAPOBEC1; D316R, D317R, R320A, R320E, R313A, W285A, W285Y, and R326E of hAPOBEC3G; and any alternative mutation at the corresponding position, or one or more corresponding mutations in another APOBEC deaminase.

A number of modified cytidine deaminases are commercially available, including, but not limited to, SaBE3, SaKKH-BE3, VQR-BE3, EQR-BE3, VRER-BE3, YE1-BE3, EE-BE3, YE2-BE3, and YEE-BE3, which are available from Addgene (plasmids 85169, 85170, 85171, 85172, 85173, 85174, 85175, 85176, 85177). In some embodiments, a deaminase incorporated into a base editor comprises all or a portion (e.g., a functional portion) of an APOBEC1 deaminase.

In some embodiments, the fusion proteins or complexes of the disclosure comprise one or more cytidine deaminase domains. In some embodiments, the cytidine deaminases provided herein are capable of deaminating cytosine or 5-methylcytosine to uracil or thymine. In some embodiments, the cytidine deaminases provided herein are capable of deaminating cytosine in DNA. The cytidine deaminase may be derived from any suitable organism. In some embodiments, the cytidine deaminase is a naturally-occurring cytidine deaminase that includes one or more mutations corresponding to any of the mutations provided herein. One of skill in the art will be able to identify the corresponding residue in any homologous protein, e.g., by sequence alignment and determination of homologous residues. Accordingly, one of skill in the art would be able to generate mutations in any naturally-occurring cytidine deaminase that corresponds to any of the mutations described herein. In some embodiments, the cytidine deaminase is from a prokaryote. In some embodiments, the cytidine deaminase is from a bacterium. In some embodiments, the cytidine deaminase is from a mammal (e.g., human).

In some embodiments, the cytidine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the cytidine deaminase amino acid sequences set forth herein. It should be appreciated that cytidine deaminases provided herein may include one or more mutations (e.g., any of the mutations provided herein). Some embodiments provide a polynucleotide molecule encoding the cytidine deaminase nucleobase editor polypeptide of any previous aspect or as delineated herein. In some embodiments, the polynucleotide is codon optimized.

In embodiments, a fusion protein of the disclosure comprises two or more nucleic acid editing domains.

Details of C to T nucleobase editing proteins are described in International PCT Application No. PCT/US2016/058344 (WO2017/070632) and Komor, A. C., et al., “Programmable editing of a target base in genomic DNA without double-stranded DNA cleavage” Nature 533, 420-424 (2016), the entire contents of which are hereby incorporated by reference.

### Cytidine Adenosine Base Editors (CABEs)

In some embodiments, a base editor described herein comprises an adenosine deaminase variant that has increased cytidine deaminase activity. Such base editors may be referred to as “cytidine adenosine base editors (CABEs)” or “cytosine base editors derived from TadA* (CBE-Ts),” and their corresponding deaminase domains may be referred to as “TadA* acting on DNA cytosine (T_{AD}C)” domains. In some instances, an adenosine deaminase variant has both adenine and cytosine deaminase activity (i.e., is a dual deaminase). In some embodiments, the adenosine deaminase variants deaminate adenine and cytosine in DNA. In some embodiments, the adenosine deaminase variants deaminate adenine and cytosine in single-stranded DNA. In some embodiments, the adenosine deaminase variants deaminate adenine and cytosine in RNA. In some embodiments, the adenosine deaminase variant predominantly deaminates cytosine in DNA and/or RNA (e.g., greater than 30%, 40%, 50%, 60%, 70%, 80%, 90%, 95%, or 99% of all deaminations catalyzed by the adenosine deaminase variant, or the number of cytosine deaminations catalyzed by the variant is about or at least about 2-fold, 3-fold, 4-fold, 5-fold, 6-fold, 7-fold, 8-fold, 9-fold, 10-fold, 25-fold, 50-fold, 75-fold, 100-fold, 500-fold, or 1,000-fold greater than the number adenine deaminations catalyzed by the variant). In some embodiments, the adenosine deaminase variant has approximately equal cytosine and adenosine deaminase activity (e.g., the two activities are within about 10% or 20% of each other). In some embodiments, the adenosine deaminase variant has predominantly cytosine deaminase activity, and little, if any, adenosine deaminase activity. In some embodiments, the adenosine deaminase variant has cytosine deaminase activity, and no significant or no detectable adenosine deaminase activity. In some embodiments, the target polynucleotide is present in a cell in vitro or in vivo. In some embodiments, the cell is a bacteria, yeast, fungi, insect, plant, or mammalian cell.

In some embodiments, the CABE comprises a bacterial TadA deaminase variant (e.g., ecTadA). In some embodiments, the CABE comprises a truncated TadA deaminase variant. In some embodiments, the CABE comprises a fragment of a TadA deaminase variant. In some embodiments, the CABE comprises a TadA*8.20 variant.

In some embodiments, an adenosine deaminase variant of the disclosure is a TadA adenosine deaminase comprising one or more alterations that increase cytosine deaminase activity (e.g., at least about 10-fold, 20-fold, 30-fold, 40-fold, 50-fold, 60-fold, 70-fold or more increase) while maintaining adenosine deaminase activity (e.g., at least about 30%, 40%, 50% or more of the activity of a reference adenosine deaminase (e.g., TadA*8.20 or TadA*8.19)). In some instances, the adenosine deaminase variant comprises one or more alterations that increase cytosine deaminase activity (e.g., at least about 10-fold, 20-fold, 30-fold, 40-fold, 50-fold, 60-fold, 70-fold or more increase) relative to the activity of a reference adenosine deaminase and comprise undetectable adenosine deaminase activity or adenosine deaminase activity that is less than 30%, 20%, 10%, or 5% of that of a reference adenosine deaminase. In some embodiments, the reference adenosine deaminase is TadA*8.20 or TadA*8.19.

In some embodiments, the adenosine deaminase variant is an adenosine deaminase comprising two or more alterations at an amino acid position selected from the group consisting of 2, 4, 6, 8, 13, 17, 23, 27, 29, 30, 47, 48, 49, 67, 76, 77, 82, 84, 96, 100, 107, 112, 114, 115, 118, 119, 122, 127, 142, 143, 147, 149, 158, 159, 162 165, 166, and 167, of an amino acid sequence having at least about 70%, 75%, 80%, 85%, 90%, 95%, 98%, 99% or greater identity to SEQ ID NO: 1, or a corresponding alteration in another deaminase.

In some embodiments, the adenosine deaminase variant is an adenosine deaminase comprising one or more alterations selected from the group consisting of S2H, V4K, V4S, V4T, V4Y, F6G, F6H, F6Y, H8Q, R13G, T17A, T17W, R23Q, E27C, E27G, E27H, E27K, E27Q, E27S, E27G, P29A, P29G, P29K, V30F, V30I, R47G, R47S, A48G, 149K, 149M, 149N, 149Q, I49T, G67W, I76H, I76R, I76W, Y76H, Y76R, Y76W, F84A, F84M, H96N, G100A, G100K, T111H, G112H, A114C, G115M, M118L, H122G, H122R, H122T, N127I, N127K, N127P, A142E, R147H, A158V, Q159S, A162C, A162N, A162Q, and S165P of an amino acid sequence having at least about 70%, 75%, 80%, 85%, 90%, 95%, 98%, 99% or greater identity to SEQ ID NO: 1, or a corresponding alteration in another deaminase.

In some embodiments, the adenosine deaminase variant is an adenosine deaminase comprising an amino acid alteration or combination of amino acid alterations selected from those listed in any of Tables 6A-6F.

The residue identity of exemplary adenosine deaminase variants that are capable of deaminating adenine and/or cytidine in a target polynucleotide (e.g., DNA) is provided in Tables 6A-6F below. Further examples of adenosine deaminase variants include the following variants of 1.17 (see Table 6A): 1.17+E27H; 1.17+E27K; 1.17+E27S; 1.17+E27S+I49K; 1.17+E27G; 1.17+I49N; 1.17+E27G+149N; and 1.17+E27Q. In some embodiments, any of the amino acid alterations provided herein are substituted with a conservative amino acid. Additional mutations known in the art can be further added to any of the adenosine deaminase variants provided herein.

In some embodiments, the base editor systems comprising a CABE provided herein have at least about a 30%, 40%, 50%, 60%, 70% or more C to T editing activity in a target polynucleotide (e.g., DNA). In some embodiments, a base editor system comprising a CABE as provided herein has an increased C to T base editing activity (e.g., increased at least about 30-fold, 40-fold, 50-fold, 60-fold, 70-fold or more) relative to a reference base editor system comprising a reference adenosine deaminase (e.g., TadA*8.20 or TadA*8.19).

TABLE 6A
Adenosine Deaminase Variants. Mutations are indicated with reference to
TadA*8.20. “S” indicates “Surface,” and “NAS” indicates “Near Active Site.”

| location in structure | N/A | S h1 | S h1 | S h1 | NAS | NAS | NAS | NAS | | S | |
|---|---|---|---|---|---|---|---|---|---|---|---|
| Amino Acid No. (*START Met is AA#1) | 2 | 8 | 13 | 17 | 27 | 47 | 48 | 49 | 67 | 76 | 77 |
| TadA*8.20 | S | H | R | T | E | R | A | I | G | Y | D |
| TadA*8.19 | | | | | | | | | | I | |
| 1.1 | | | | | H | | | | | I | |
| 1.2 | | | | | H | | | K | | I | |
| 1.3 | | | | | S | | | K | | I | |
| 1.4 | | | | | S | | | K | | I | |
| 1.5 | | | | | K | | | | | | |
| 1.6 | | | | | K | | | | | | |
| 1.7 | | | | | H | | | | | I | |
| 1.8 | | | | | S | | | K | W | | |
| 1.9 | | | | | | | | T | W | | |
| 1.10 | | | | | C | | | | | I | |
| 1.11 | | | G | | Q | | | | | | |
| 1.12 | | | | A | H | | | M | | I | |
| 1.13 | | | | | | | | Q | | I | |
| 1.14 | H | | | | | | | K | | I | |
| 1.15 | | | | | | S | | | | | |
| 1.16 | | Q | | | | | | Q | | I | |
| 1.17 | | | | A | | | G | | | | |
| 1.18 | | | | | G | | | | | | |
| 1.19 | | | | | G | | | N | | | |
| 1.20 | | | | | G | | | | | | G |

TABLE 6A
Adenosine Deaminase Variants. Mutations are indicated with reference to
TadA*8.20. “I” indicates “Internal,” “S” indicates “Surface,” and “NAS” indicates “Near Active Site.”

| location in | I | NAS | | NAS | S | | S | S | S | | S | |
|---|---|---|---|---|---|---|---|---|---|---|---|---|
| structure | | | | | | | | | | | | |
| Amino Acid | 82 | 84 | 96 | 107 | 112 | 115 | 118 | 119 | 127 | 142 | 162 | 165 |
| No. (*START | | | | | | | | | | | | |
| Met is AA#1) | | | | | | | | | | | | |
| TadA*8.20 | S | F | H | R | G | G | M | D | N | A | A | S |
| TadA*8.19 | | | | | | | | | | | | |
| 1.1 | | M | | | | | | | | | | |
| 1.2 | | | | | | | | | | | | |
| 1.3 | | | | | | | | | | | | |
| 1.4 | | | | | | | | | | | N | |
| 1.5 | | | | | | | | | | | | |
| 1.6 | | | | | | | | N | | | | |
| 1.7 | | | | | | | | | | | | |
| 1.8 | | | | | | | | | | | | |
| 1.9 | | | N | | | | | | | | | |
| 1.10 | | | | | | | | N | | | | |
| 1.11 | | | | | | | | | K | | | |
| 1.12 | | | | | | | L | | | | | |
| 1.13 | | | | | | M | | | | | | |
| 1.14 | | | | | H | | | | | | | |
| 1.15 | | | | C | | | | | | | | |
| 1.16 | | | | | | | | | | | | |
| 1.17 | T | | | | | | | | | E | | |
| 1.18 | | | | | | | | | | | | |
| 1.19 | | | | | | | | | | | | |
| 1.20 | | | | | | | | | | | | P |

TABLE 6B
Adenosine deaminase variants. Mutations are indicated
with reference to TadA*8.20.

| Position No. | 27 | 29 | 30 | 49 | 82 | 84 | 107 | 112 | 115 | 142 |
|---|---|---|---|---|---|---|---|---|---|---|
| TadA*8.20 | E | P | V | I | S | F | R | G | G | A |
| Alterations | | | | | | | | | | |
| Evaluated | G/S/H | G/A/K | I/L/F | K | T | L/A | C | H | M | E |
| S1.1 | S | | | K | T | | | | | |
| S1.2 | S | | | K | T | | C | | | |
| S1.3 | S | | | K | T | | | H | | |
| S1.4 | S | | | K | T | | | | M | |
| S1.5 | S | | | K | T | | | | | E |
| S1.6 | S | | | K | T | | C | H | | |
| S1.7 | S | | | K | T | | C | | M | |
| S1.8 | S | | | K | T | | C | | | E |
| S1.9 | S | | | K | T | | | H | | E |
| S1.10 | S | | | K | T | | | | M | E |
| S1.11 | S | | | K | T | | C | H | M | E |
| S1.12 | S | | I | K | T | | | | | |
| S1.13 | S | | I | K | T | | C | | | |
| S1.14 | S | | I | K | T | | | H | | |
| S1.15 | S | | I | K | T | | | | M | |
| S1.16 | S | | I | K | T | | | | | E |
| S1.17 | S | | I | K | T | | C | H | | |
| S1.18 | S | | I | K | T | | C | | M | |
| S1.19 | S | | I | K | T | | C | | | E |
| S1.20 | S | | I | K | T | | | H | | E |
| S1.21 | S | | I | K | T | | | | M | E |
| S1.22 | S | | I | K | T | | C | H | M | E |
| S1.23 | S | | L | K | T | | | | | |
| S1.24 | S | | L | K | T | | C | | | |
| S1.25 | S | | L | K | T | | | H | | |
| S1.26 | S | | L | K | T | | | | M | |
| S1.27 | S | | L | K | T | | | | | E |
| S1.28 | S | | L | K | T | | C | H | | |
| S1.29 | S | | L | K | T | | C | | M | |
| S1.30 | S | | L | K | T | | C | | | E |
| S1.31 | S | | L | K | T | | | H | | E |
| S1.32 | S | | L | K | T | | | | M | E |
| S1.33 | S | | L | K | T | | C | H | M | E |
| S1.34 | S | | F | K | T | A | | | | |
| S1.35 | S | | F | K | T | A | C | | | |
| S1.36 | S | | F | K | T | A | | H | | |
| S1.37 | S | | F | K | T | A | | | M | |
| S1.38 | S | | F | K | T | A | | | | E |
| S1.39 | S | | F | K | T | A | C | H | | |
| S1.40 | S | | F | K | T | A | C | | M | |
| S1.41 | S | | F | K | T | A | C | | | E |
| S1.42 | S | | F | K | T | A | | H | | E |
| S1.43 | S | | F | K | T | A | | | M | E |
| S1.44 | S | | F | K | T | A | C | H | M | E |
| S1.45 | S | | | K | T | L | | | | |
| S1.46 | S | | | K | T | L | C | | | |
| S1.47 | S | | | K | T | L | | H | | |
| S1.48 | S | | | K | T | L | | | M | |
| S1.49 | S | | | K | T | L | | | | E |
| S1.50 | S | | | K | T | L | C | H | | |
| S1.51 | S | | | K | T | L | C | | M | |
| S1.52 | S | | | K | T | L | C | | | E |
| S1.53 | S | | | K | T | L | | H | | E |
| S1.54 | S | | | K | T | L | | | M | E |
| S1.55 | S | | | K | T | L | C | H | M | E |
| S1.56 | S | | I | K | T | L | | | | |
| S1.57 | S | | I | K | T | L | C | | | |
| S1.58 | S | | I | K | T | L | | H | | |
| S1.59 | S | | I | K | T | L | | | M | |
| S1.60 | S | | I | K | T | L | | | | E |
| S1.61 | S | | I | K | T | L | C | H | | |
| S1.62 | S | | I | K | T | L | C | | M | |
| S1.63 | S | | I | K | T | L | C | | | E |
| S1.64 | S | | I | K | T | L | | H | | E |
| S1.65 | S | | I | K | T | L | | | M | E |
| S1.66 | S | | I | K | T | L | C | H | M | E |
| S1.67 | S | G | | K | T | | | | | |
| S1.68 | S | G | | K | T | | C | | | |
| S1.69 | S | G | | K | T | | | H | | |
| S1.70 | S | G | | K | T | | | | M | |
| S1.71 | S | G | | K | T | | | | | E |
| S1.72 | S | G | | K | T | | C | H | | |
| S1.73 | S | G | | K | T | | C | | M | |
| S1.74 | S | G | | K | T | | C | | | E |
| S1.75 | S | G | | K | T | | | H | | E |
| S1.76 | S | G | | K | T | | | | M | E |
| S1.77 | S | G | | K | T | | C | H | M | E |
| S1.78 | | G | | K | T | | | | | |
| S1.79 | | G | | K | T | | C | | | |
| S1.80 | | G | | K | T | | | H | | |
| S1.81 | | G | | K | T | | | | M | |
| S1.82 | | G | | K | T | | | | | E |
| S1.83 | | G | | K | T | | C | H | | |
| S1.84 | | G | | K | T | | C | | M | |
| S1.85 | | G | | K | T | | C | | | E |
| S1.86 | | G | | K | T | | | H | | E |
| S1.87 | | G | | K | T | | | | M | E |
| S1.88 | | G | | K | T | | C | H | M | E |
| S1.89 | | K | | K | T | | | | | |
| S1.90 | | K | | K | T | | C | | | |
| S1.91 | | K | | K | T | | | H | | |
| S1.92 | | K | | K | T | | | | M | |
| S1.93 | | K | | K | T | | | | | E |
| S1.94 | | K | | K | T | | C | H | | |
| S1.95 | | K | | K | T | | C | | M | |
| S1.96 | | K | | K | T | | C | | | E |
| S1.97 | | K | | K | T | | | H | | E |
| S1.98 | | K | | K | T | | | | M | E |
| S1.99 | | K | | K | T | | C | H | M | E |
| S1.100 | | K | I | K | T | | | | | |
| S1.101 | | K | I | K | T | | C | | | |
| S1.102 | | K | I | K | T | | | H | | |
| S1.103 | | K | I | K | T | | | | M | |
| S1.104 | | K | I | K | T | | | | | E |
| S1.105 | | K | I | K | T | | C | H | | |
| S1.106 | | K | I | K | T | | C | | M | |
| S1.107 | | K | I | K | T | | C | | | E |
| S1.108 | | K | I | K | T | | | H | | E |
| S1.109 | | K | I | K | T | | | | M | E |
| S1.110 | | K | I | K | T | | C | H | M | E |
| S1.111 | | K | | K | T | L | | | | |
| S1.112 | | K | | K | T | L | C | | | |
| S1.113 | | K | | K | T | L | | H | | |
| S1.114 | | K | | K | T | L | | | M | |
| S1.115 | | K | | K | T | L | | | | E |
| S1.116 | | K | | K | T | L | C | H | | |
| S1.117 | | K | | K | T | L | C | | M | |
| S1.118 | | K | | K | T | L | C | | | E |
| S1.119 | | K | | K | T | L | | H | | E |
| S1.120 | | K | | K | T | L | | | M | E |
| S1.121 | | K | | K | T | L | C | H | M | E |
| S1.122 | | K | I | K | T | L | | | | |
| S1.123 | | K | I | K | T | L | C | | | |
| S1.124 | | K | I | K | T | L | | H | | |
| S1.125 | | K | I | K | T | L | | | M | |
| S1.126 | | K | I | K | T | L | | | | E |
| S1.127 | | K | I | K | T | L | C | H | | |
| S1.128 | | K | I | K | T | L | C | | M | |
| S1.129 | | K | I | K | T | L | C | | | E |
| S1.130 | | K | I | K | T | L | | H | | E |
| S1.131 | | K | I | K | T | L | | | M | E |
| S1.132 | | K | I | K | T | L | C | H | M | E |
| S1.133 | G | | | K | T | | | | | |
| S1.134 | G | | | K | T | | C | | | |
| S1.135 | G | | | K | T | | | H | | |
| S1.136 | G | | | K | T | | | | M | |
| S1.137 | G | | | K | T | | | | | E |
| S1.138 | G | | | K | T | | C | H | | |
| S1.139 | G | | | K | T | | C | | M | |
| S1.140 | G | | | K | T | | C | | | E |
| S1.141 | G | | | K | T | | | H | | E |
| S1.142 | G | | | K | T | | | | M | E |
| S1.143 | G | | | K | T | | C | H | M | E |
| S1.144 | H | | | K | T | | | | | |
| S1.145 | H | | | K | T | | C | | | |
| S1.146 | H | | | K | T | | | H | | |
| S1.147 | H | | | K | T | | | | M | |
| S1.148 | H | | | K | T | | | | | E |
| S1.149 | H | | | K | T | | C | H | | |
| S1.150 | H | | | K | T | | C | | M | |
| S1.151 | H | | | K | T | | C | | | E |
| S1.152 | H | | | K | T | | | H | | E |
| S1.153 | H | | | K | T | | | | M | E |
| S1.154 | H | | | K | T | | C | H | M | E |
| S1.155 | S | | | | T | | | | | |
| S1.156 | S | | | | T | | C | | | |
| S1.157 | S | | | | T | | | H | | |
| S1.158 | S | | | | T | | | | M | |
| S1.159 | S | | | | T | | | | | E |
| S1.160 | S | | | | T | | C | H | | |
| S1.161 | S | | | | T | | C | | M | |
| S1.162 | S | | | | T | | C | | | E |
| S1.163 | S | | | | T | | | H | | E |
| S1.164 | S | | | | T | | | | M | E |
| S1.165 | S | | | | T | | C | H | M | E |
| S1.166 | | A | | | T | | | | | |
| S1.167 | | A | | | T | | C | | | |
| S1.168 | | A | | | T | | | H | | |
| S1.169 | | A | | | T | | | | M | |
| S1.170 | | A | | | T | | | | | E |
| S1.171 | | A | | | T | | C | H | | |
| S1.172 | | A | | | T | | C | | M | |
| S1.173 | | A | | | T | | C | | | E |
| S1.174 | | A | | | T | | | H | | |
| S1.175 | | A | | | T | | | | M | E |
| S1.176 | | A | | | T | | C | H | M | E |
| S1.177 | S | | I | | T | | | | | |
| S1.178 | S | | I | | T | | C | | | |
| S1.179 | S | | I | | T | | | H | | |
| S1.180 | S | | I | | T | | | | M | |
| S1.181 | S | | I | | T | | | | | E |
| S1.182 | S | | I | | T | | C | H | | |
| S1.183 | S | | I | | T | | C | | M | |
| S1.184 | S | | I | | T | | C | | | E |
| S1.185 | S | | I | | T | | | H | | E |
| S1.186 | S | | I | | T | | | | M | E |
| S1.187 | S | | I | | T | | C | H | M | E |
| S1.188 | | A | I | | T | L | | | | |
| S1.189 | | A | I | | T | L | C | | | |
| S1.190 | | A | I | | T | L | | H | | |
| S1.191 | | A | I | | T | L | | | M | |
| S1.192 | | A | I | | T | L | | | | E |
| S1.193 | | A | I | | T | L | C | H | | |
| S1.194 | | A | I | | T | L | C | | M | |
| S1.195 | | A | I | | T | L | C | | | E |
| S1.196 | | A | I | | T | L | | H | | E |
| S1.197 | | A | I | | T | L | | | M | E |
| S1.198 | | A | I | | T | L | C | H | M | E |
| S1.199 | S | A | L | K | T | L | C | H | M | E |

TABLE 6C
Adenosine deaminase variants. Mutations are indicated with reference to variant 1.2 (Table 6A).

| | | Residue identity (START Met is amino | | | | | | | | |
|---|---|---|---|---|---|---|---|---|---|---|
| | | acid #1) | | | | | | | | |
| Variant Name | Alternative Variant Names | 4 | 6 | 17 | 23 | 76 | 77 | 100 | 111 | 114 |
| Reference | 1.2 (see Table 6A) | V | F | T | R | I | D | G | T | A |
| TadAC2.1 | pDKL-135; 2.1 | K | | | | | | | | C |
| TadAC2.2 | pDKL-136; 2.2 | K | | | | | G | | | |
| TadAC2.3 | pDKL-137; 2.3 | | Y | | | | | A | | |
| TadAC2.4 | pDKL-138; 2.4 | T | | | | R | | | | |
| TadAC2.5 | pDKL-139; 2.5 | | Y | | | W | | | | |
| TadAC2.6 | pDKL-140; 2.6 | | Y | | | | | | | |
| TadAC2.7 | pDKL-141; 2.7 | | Y | | | | | | | C |
| TadAC2.8 | pDKL-142; 2.8 | | Y | | | | | | | |
| TadAC2.9 | pDKL-143; 2.9 | K | | | | W | | | | |
| TadAC2.10 | pDKL-144; 2.10 | | G | | | R | | K | | |
| TadAC2.11 | pDKL-145; 2.11 | | H | | | | | | | |
| TadAC2.12 | pDKL-146; 2.12 | | | | | | | | | C |
| TadAC2.13 | pDKL-147; 2.13 | | Y | | | H | | | | |
| TadAC2.14 | pDKL-148; 2.14 | | | | | | | | | |
| TadAC2.15 | pDKL-149; 2.15 | | | | Q | R | | | | |
| TadAC2.16 | pDKL-150; 2.16 | | | | | H | | | | |
| TadAC2.17 | pDKL-151; 2.17 | | Y | | | | | | H | |
| TadAC2.18 | pDKL-152; 2.18 | | | | | W | | | | |
| TadAC2.19 | pDKL-153; 2.19 | | | | | | | | H | |
| TadAC2.20 | pDKL-154; 2.20 | | | | | | | | | |
| TadAC2.21 | pDKL-155; 2.21 | | Y | | | R | | | | |
| TadAC2.22 | pDKL-156; 2.22 | | | W | | H | | | | |
| TadAC2.23 | pDKL-157; 2.23 | S | | | | Y | | | | |
| TadAC2.24 | pDKL-158; 2.24 | | | | | | | | | |
| | | Residue identity (START Met is | | | | | | | | |
| | | amino acid #1) | | | | | | | | |
| Variant Name | Alternative Variant Names | 119 | 122 | 127 | 143 | 147 | 158 | 159 | 162 | 166 |
| Reference | 1.2 (see Table 6A) | D | H | N | A | R | A | Q | A | T |
| TadAC2.1 | pDKL-135; 2.1 | | | | | | | | | |
| TadAC2.2 | pDKL-136; 2.2 | | | | | | | | | |
| TadAC2.3 | pDKL-137; 2.3 | | R | | | | | | | |
| TadAC2.4 | pDKL-138; 2.4 | | G | | | | | | | |
| TadAC2.5 | pDKL-139; 2.5 | | | | | | | | | |
| TadAC2.6 | pDKL-140; 2.6 | N | | | | | | | | |
| TadAC2.7 | pDKL-141; 2.7 | | | | | | | | | |
| TadAC2.8 | pDKL-142; 2.8 | | | | | | | | | |
| TadAC2.9 | pDKL-143; 2.9 | | T | | | | | | | |
| TadAC2.10 | pDKL-144; 2.10 | | | | | | | | | |
| TadAC2.11 | pDKL-145; 2.11 | | N | | | | | | | |
| TadAC2.12 | pDKL-146; 2.12 | | | | | | | | | |
| TadAC2.13 | pDKL-147; 2.13 | | R | | | | | | | I |
| TadAC2.14 | pDKL-148; 2.14 | | | P | | | | | | |
| TadAC2.15 | pDKL-149; 2.15 | | | | | | | | | |
| TadAC2.16 | pDKL-150; 2.16 | | R | | | | V | | | |
| TadAC2.17 | pDKL-151; 2.17 | | | | | | | | | |
| TadAC2.18 | pDKL-152; 2.18 | | | | | | | | | |
| TadAC2.19 | pDKL-153; 2.19 | | G | | | | | | C | |
| TadAC2.20 | pDKL-154; 2.20 | | | | E | | | | | |
| TadAC2.21 | pDKL-155; 2.21 | | | | | | | | | |
| TadAC2.22 | pDKL-156; 2.22 | | G | | | | V | | | |
| TadAC2.23 | pDKL-157; 2.23 | | | | E | | | S | | |
| TadAC2.24 | pDKL-158; 2.24 | | | I | | | | | Q | |

TABLE 6D
Adenosine deaminase variants. Mutations are indicated with reference to
TadA*8.20.

| AA Positions | 6 | 27 | 49 | 76 | 77 | 82 | 107 | 112 | 114 | 115 | 119 | 122 | 127 | 142 | 143 |
|---|---|---|---|---|---|---|---|---|---|---|---|---|---|---|---|
| TadA*8.20 | F | E | I | Y | D | S | R | G | A | G | D | H | N | A | A |
| S1.154 | F | H | K | Y | D | T | C | H | | M | | | | E | |
| Alterations from Table 6C | Y | | | W | G | | | | C | | N | G | P | | E |
| S2.1 | Y | H | K | W | | | C | H | | M | | | | E | |
| S2.2 | Y | H | K | | G | T | C | H | | M | | | | E | |
| S2.3 | Y | H | K | | | T | C | H | C | M | | | | | |
| S2.4 | Y | H | K | | | T | C | H | | M | N | | | | |
| S2.5 | Y | H | K | | | T | C | H | | M | | G | | E | |
| S2.6 | Y | H | K | | | T | C | H | | M | | | P | E | |
| S2.7 | Y | H | K | | | T | C | H | | M | | | | E | E |
| S2.8 | Y | H | K | | | T | C | H | | M | | | | A | E |
| S2.9 | Y | H | K | W | G | T | C | H | | M | | | | E | |
| S2.10 | Y | H | K | W | | T | C | H | C | M | | | | E | |
| S2.11 | Y | H | K | W | | T | C | H | | M | N | | | E | |
| S2.12 | Y | H | K | W | | T | C | H | | M | | G | | E | |
| S2.13 | Y | H | K | W | | T | C | H | | M | | | P | E | |
| S2.14 | Y | H | K | W | | T | C | H | | M | | | | E | E |
| S2.15 | Y | H | K | W | | T | C | H | | M | | | | A | E |
| S2.16 | Y | H | K | | G | T | C | H | C | M | | | | E | |
| S2.17 | Y | H | K | | G | T | C | H | | M | N | | | E | |
| S2.18 | Y | H | K | | G | T | C | H | | M | | G | | E | |
| S2.19 | Y | H | K | | G | T | C | H | | M | | | P | E | |
| S2.20 | Y | H | K | | G | T | C | H | | M | | | | E | E |
| S2.21 | Y | H | K | | G | T | C | H | | M | | | | A | E |
| S2.22 | Y | H | K | | | T | C | H | C | M | N | | | E | |
| S2.23 | Y | H | K | | | T | C | H | C | M | | G | | E | |
| S2.24 | Y | H | K | | | T | C | H | C | M | | | P | E | |
| S2.25 | Y | H | K | | | T | C | H | | M | N | G | | E | |
| S2.26 | Y | H | K | | | T | C | H | | M | N | | P | E | |
| S2.27 | Y | H | K | | | T | C | H | | M | | G | P | E | |
| S2.28 | Y | H | K | W | G | T | C | H | C | M | | | | E | |
| S2.29 | Y | H | K | W | G | T | C | H | | M | N | | | E | |
| S2.30 | Y | H | K | W | G | T | C | H | | M | | G | | E | |
| S2.31 | Y | H | K | W | G | T | C | H | | M | | | P | E | |
| S2.32 | Y | H | K | W | G | T | C | H | | M | | | | E | E |
| S2.33 | Y | H | K | W | G | T | C | H | | M | | | | E | E |
| S2.34 | Y | H | K | W | | T | C | H | C | M | N | | | E | |
| S2.35 | Y | H | K | W | | T | C | H | C | M | | G | | E | |
| S2.36 | Y | H | K | W | | T | C | H | C | M | | | P | E | |
| S2.37 | Y | H | K | W | | T | C | H | C | M | | | | E | E |
| S2.38 | Y | H | K | W | | T | C | H | C | M | | | | E | E |
| S2.39 | Y | H | K | W | | T | C | H | | M | N | G | | E | |
| S2.40 | Y | H | K | W | | T | C | H | | M | N | | P | E | |
| S2.41 | Y | H | K | M | | T | C | H | | M | | G | P | E | |
| S2.42 | Y | H | K | W | | T | C | H | | M | N | G | | E | |
| S2.43 | Y | H | K | W | | T | C | H | C | M | N | | P | E | |
| S2.44 | Y | H | K | W | | T | C | H | C | M | | G | P | E | |
| S2.45 | Y | H | K | W | G | T | C | H | C | M | N | | | E | |
| S2.46 | Y | H | K | W | G | T | C | H | C | M | | G | | E | |
| S2.47 | Y | H | K | W | G | T | C | H | C | M | | | P | E | |
| S2.48 | Y | H | K | W | G | T | C | H | C | M | | | | E | E |
| S2.49 | Y | H | K | W | G | T | C | H | C | M | | | | A | E |
| S2.50 | Y | H | K | W | G | T | C | H | C | M | N | G | | E | |
| S2.51 | Y | H | K | W | G | T | C | H | C | M | N | | P | E | |
| S2.52 | Y | H | K | W | G | T | C | H | C | M | | G | | E | |
| S2.53 | Y | H | K | W | | T | C | H | C | M | N | G | P | E | E |
| S2.54 | Y | H | K | W | | T | C | H | C | M | N | G | P | A | E |
| S2.55 | Y | H | K | W | G | T | C | H | C | M | N | G | P | E | E |
| S2.56 | Y | H | K | W | G | T | C | H | C | M | N | G | P | A | E |

TABLE 6E
Hybrid constructs. Mutations are indicated with reference to TadA*7.10.

| | TadA amino acid subsitutions | | | | | | | | | | | |
|---|---|---|---|---|---|---|---|---|---|---|---|---|
| | 76 | 82 | 109 | 111 | 119 | 122 | 123 | 147 | 149 | 154 | 166 | 167 |
| TadA*7.10 | I | V | A | T | D | H | Y | Y | F | Q | T | D |
| TadA*8e | | | S | R | N | N | | D | Y | | I | N |
| TadA*8.20 | Y | S | | | | | H | R | | R | | |
| TadA*8.17 | | S | | | | | | | | R | | |
| pNMG-B878 | Y | S | | | | | H | D | | R | | |
| pNMG-B879 | Y | S | | | | | H | R | Y | R | | |
| pNMG-B880 | Y | S | | | | | H | R | | R | I | |
| pNMG-B881 | Y | S | | | | | H | R | | R | | N |
| pNMG-B882 | Y | S | | | | | H | D | Y | R | I | N |
| pNMG-B883 | Y | S | | R | N | | H | R | | R | | |
| pNMG-B884 | Y | S | S | R | N | N | H | R | | R | | |
| pNMG-B885 | Y | S | S | | | | H | R | | R | | |
| pNMG-B886 | Y | S | | R | | | H | R | | R | | |
| pNMG-B887 | Y | S | | | N | | H | R | | R | | |
| pNMG-B888 | Y | S | | | | N | H | R | | R | | |
| pNMG-B889 | Y | S | S | R | | | H | R | | R | | |
| pNMG-B890 | Y | S | | | N | N | H | R | | R | | |
| pNMG-B891 | Y | S | S | R | N | N | H | D | Y | R | I | N |

TABLE 6F
Base editor variants. Mutations are indicated with reference to TadA*8.19/8.20.

| AA positions: | 17 | 27 | 48 | 49 | 76 | 82 | 84 | 118 | 142 | 147 | 149 | 166 | 167 |
|---|---|---|---|---|---|---|---|---|---|---|---|---|---|
| ABE8.19 m/8.20 m | T | E | A | I | Y/I | S | F | M | A | Y | F | T | D |
| 1.1 + 8e(B879) | | H | | | I | | M | | | | Y | | |
| 1.2 + 8e(B879) | | H | | K | I | | | | | | Y | | |
| 1.12 + 8e(B879) | A | H | | M | I | | | L | | | Y | | |
| 1.17 + 8e(B879) | A | | G | | | T | | | E | | Y | | |
| 1.18 + 8e(B879) | | G | | | | | | | | | Y | | |
| 1.19 + 8e(B879) | | G | | N | | | | | | | Y | | |
| 1.1 + 8e(B882) | | H | | | I | | M | | | D | Y | I | N |
| 1.2 + 8e(B882) | | H | | K | I | | | | | D | Y | I | N |
| 1.12 + 8e(B882) | A | H | | M | I | | | L | | D | Y | I | N |
| 1.17 + 8e(B882) | A | | G | | | T | | | E | D | Y | I | N |
| 1.18 + 8e(B882) | | G | | | | | | | | D | Y | I | N |
| 1.19 + 8e(B882) | | G | | N | | | | | | D | Y | I | N |

### Guide Polynucleotides

A polynucleotide programmable nucleotide binding domain, when in conjunction with a bound guide polynucleotide (e.g., gRNA), can specifically bind to a target polynucleotide sequence (i.e., via complementary base pairing between bases of the bound guide nucleic acid and bases of the target polynucleotide sequence) and thereby localize the base editor to the target nucleic acid sequence desired to be edited. In some embodiments, the target polynucleotide sequence comprises single-stranded DNA or double-stranded DNA. In some embodiments, the target polynucleotide sequence comprises RNA. In some embodiments, the target polynucleotide sequence comprises a DNA-RNA hybrid.

In an embodiment, a guide polynucleotide described herein can be RNA or DNA. In one embodiment, the guide polynucleotide is a gRNA.

In some embodiments, the guide polynucleotide is at least one single guide RNA (“sgRNA” or “gRNA”). In some embodiments, a guide polynucleotide comprises two or more individual polynucleotides, which can interact with one another via for example complementary base pairing (e.g., a dual guide polynucleotide, dual gRNA). For example, a guide polynucleotide can comprise a CRISPR RNA (crRNA) and a trans-activating CRISPR RNA (tracrRNA) or can comprise one or more trans-activating CRISPR RNA (tracrRNA).

A guide polynucleotide may include natural or non-natural (or unnatural) nucleotides (e.g., peptide nucleic acid or nucleotide analogs). In some cases, the targeting region of a guide nucleic acid sequence (e.g., a spacer) can be at least 15, 16, 17, 18, 19, 20, 21, 22, 23, 24, 25, 26, 27, 28, 29, or 30 nucleotides in length.

In some embodiments, the methods described herein can utilize an engineered Cas protein. A guide RNA (gRNA) is a short synthetic RNA composed of a scaffold sequence necessary for Cas-binding and a user-defined ˜20 nucleotide spacer that defines the genomic target to be modified. Exemplary gRNA scaffold sequences are provided in the sequence listing as SEQ ID NOs: 317-327 and 669. Thus, a skilled artisan can change the genomic target of the Cas protein specificity is partially determined by how specific the gRNA targeting sequence is for the genomic target compared to the rest of the genome. In embodiments, the spacer is about 10, 11, 12, 13, 14, 15, 16, 17, 18, 19, 20, 22, 23, 24, 25, or more nucleotides in length. The spacer of a gRNA can be or can be about 19, 20, or 21 nucleotides in length.

A gRNA or a guide polynucleotide can target any exon or intron of a gene target. In some embodiments, a composition comprises multiple gRNAs that all target the same exon or multiple gRNAs that target different exons. An exon and/or an intron of a gene can be targeted. A gRNA or a guide polynucleotide can target a nucleic acid sequence of about 20 nucleotides or less than about 20 nucleotides (e.g., at least about 5, 10, 15, 16, 17, 18, 19, 20, 21, 22, 23, 24, 25, 30 nucleotides), or anywhere between about 1-100 nucleotides (e.g., 5, 10, 15, 16, 17, 18, 19, 20, 21, 22, 23, 24, 25, 30, 40, 50, 60, 70, 80, 90, 100). A target nucleic acid sequence can be or can be about 20 bases immediately 5′ of the first nucleotide of the PAM. A gRNA can target a nucleic acid sequence. A target nucleic acid can be at least or at least about 1-10, 1-20, 1-30, 1-40, 1-50, 1-60, 1-70, 1-80, 1-90, or 1-100 nucleotides.

The guide polynucleotides can comprise standard ribonucleotides, modified ribonucleotides (e.g., pseudouridine), ribonucleotide isomers, and/or ribonucleotide analogs.

In some embodiments, a base editor system may comprise multiple guide polynucleotides, e.g., gRNAs. For example, the gRNAs may target to one or more target loci (e.g., at least 1 gRNA, at least 2 gRNA, at least 5 gRNA, at least 10 gRNA, at least 20 gRNA, at least 30 g RNA, at least 50 gRNA) comprised in a base editor system. The multiple gRNA sequences can be tandemly arranged and may be separated by a direct repeat.

### Modified Polynucleotides

To enhance expression, stability, and/or genomic/base editing efficiency, and/or reduce possible toxicity, the base editor-coding sequence (e.g., mRNA) and/or the guide polynucleotide (e.g., gRNA) can be modified to include one or more modified nucleotides and/or chemical modifications, e.g. using pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), =constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine. Chemically protected gRNAs can enhance stability and editing efficiency in vivo and ex vivo. Methods for using chemically modified mRNAs and guide RNAs are known in the art and described, for example, by Jiang et al., Chemical modifications of adenine base editor mRNA and guide RNA expand its application scope. Nat Commun 11, 1979 (2020). doi.org/10.1038/s41467-020-15892-8, Callum et al., N1-Methylpseudouridine substitution enhances the performance of synthetic mRNA switches in cells, Nucleic Acids Research, Volume 48, Issue 6, 6 Apr. 2020, Page e35, and Andries et al., Journal of Controlled Release, Volume 217, 10 Nov. 2015, Pages 337-344, each of which is incorporated herein by reference in its entirety.

In some embodiments, the guide polynucleotide comprises one or more modified nucleotides at the 5′ end and/or the 3′ end of the guide. In some embodiments, the guide polynucleotide comprises two, three, four or more modified nucleosides at the 5′ end and/or the 3′ end of the guide. In some embodiments, the guide polynucleotide comprises two, three, four or more modified nucleosides at the 5′ end and/or the 3′ end of the guide.

In some embodiments, the guide comprises at least about 50%-75% modified nucleotides. In some embodiments, the guide comprises at least about 85% or more modified nucleotides. In some embodiments, at least about 1-5 nucleotides at the 5′ end of the gRNA are modified and at least about 1-5 nucleotides at the 3′ end of the gRNA are modified. In some embodiments, at least about 3-5 contiguous nucleotides at each of the 5′ and 3′ termini of the gRNA are modified. In some embodiments, at least about 20% of the nucleotides present in a direct repeat or anti-direct repeat are modified. In some embodiments, at least about 50% of the nucleotides present in a direct repeat or anti-direct repeat are modified. In some embodiments, at least about 50-75% of the nucleotides present in a direct repeat or anti-direct repeat are modified. In some embodiments, at least about 100 of the nucleotides present in a direct repeat or anti-direct repeat are modified. In some embodiments, at least about 20% or more of the nucleotides present in a hairpin present in the gRNA scaffold are modified. In some embodiments, at least about 50% or more of the nucleotides present in a hairpin present in the gRNA scaffold are modified. In some embodiments, the guide comprises a variable length spacer. In some embodiments, the guide comprises a 20-40 nucleotide spacer. In some embodiments, the guide comprises a spacer comprising at least about 20-25 nucleotides or at least about 30-35 nucleotides. In some embodiments, the spacer comprises modified nucleotides. In some embodiments, the guide comprises two or more of the following:
at least about 1-5 nucleotides at the 5′ end of the gRNA are modified and at least about 1-5 nucleotides at the 3′ end of the gRNA are modified;
at least about 20% of the nucleotides present in a direct repeat or anti-direct repeat are modified;
at least about 50-75% of the nucleotides present in a direct repeat or anti-direct repeat are modified;
at least about 20% or more of the nucleotides present in a hairpin present in the gRNA scaffold are modified;
a variable length spacer; and
a spacer comprising modified nucleotides.

In embodiments, the gRNA contains numerous modified nucleotides and/or chemical modifications (“heavy mods”). Such heavy mods can increase base editing ˜2 fold in vivo or in vitro. In embodiments, the gRNA comprises 2′-O-methyl or phosphorothioate modifications. In an embodiment, the gRNA comprises 2′-O-methyl and phosphorothioate modifications. In an embodiment, the modifications increase base editing by at least about 2 fold.

A guide polynucleotide can comprise one or more modifications to provide a nucleic acid with a new or enhanced feature. A guide polynucleotide can comprise a nucleic acid affinity tag. A guide polynucleotide can comprise synthetic nucleotide, synthetic nucleotide analog, nucleotide derivatives, and/or modified nucleotides.

A gRNA or a guide polynucleotide can also be modified by 5′ adenylate, 5′ guanosine-triphosphate cap, 5′ N7-Methylguanosine-triphosphate cap, 5′ triphosphate cap, 3′ phosphate, 3′ thiophosphate, 5′ phosphate, 5′ thiophosphate, Cis-Syn thymidine dimer, trimers, C12 spacer, C3 spacer, C6 spacer, dSpacer, PC spacer, rSpacer, Spacer 18, Spacer 9, 3′-3′ modifications, 2′-O-methyl thioPACE (MSP), 2′-O-methyl-PACE (MP), and constrained ethyl (S-cEt), 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′ DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′-deoxyribonucleoside analog purine, 2′-deoxyribonucleoside analog pyrimidine, ribonucleoside analog, 2′-O-methyl ribonucleoside analog, sugar modified analogs, wobble/universal bases, fluorescent dye label, 2′-fluoro RNA, 2′-O-methyl RNA, methylphosphonate, phosphodiester DNA, phosphodiester RNA, phosphothioate DNA, phosphorothioate RNA, UNA, pseudouridine-5′-triphosphate, 5′-methylcytidine-5′-triphosphate, or any combination thereof.

In some cases, a phosphorothioate enhanced RNA gRNA can inhibit RNase A, RNase T1, calf serum nucleases, or any combinations thereof. These properties can allow the use of PS-RNA gRNAs to be used in applications where exposure to nucleases is of high probability in vivo or in vitro. For example, phosphorothioate (PS) bonds can be introduced between the last 3-5 nucleotides at the 5′- or 3′-end of a gRNA which can inhibit exonuclease degradation. In some cases, phosphorothioate bonds can be added throughout an entire gRNA to reduce attack by endonucleases.

### Fusion Proteins or Complexes Comprising a Nuclear Localization Sequence (NLS)

In some embodiments, the fusion proteins or complexes provided herein further comprise one or more (e.g., 2, 3, 4, 5) nuclear targeting sequences, for example a nuclear localization sequence (NLS). In one embodiment, a bipartite NLS is used. In some embodiments, a NLS comprises an amino acid sequence that facilitates the importation of a protein, that comprises an NLS, into the cell nucleus (e.g., by nuclear transport). In some embodiments, the NLS is fused to the N-terminus or the C-terminus of the fusion protein. In some embodiments, the NLS is fused to the C-terminus or N-terminus of an nCas9 domain or a dCas9 domain. In some embodiments, the NLS is fused to the N-terminus or C-terminus of the Cas12 domain. In some embodiments, the NLS is fused to the N-terminus or C-terminus of the cytidine or adenosine deaminase. In some embodiments, the NLS is fused to the fusion protein via one or more linkers. In some embodiments, the NLS is fused to the fusion protein without a linker. In some embodiments, the NLS comprises an amino acid sequence of any one of the NLS sequences provided or referenced herein. Additional nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences.

In some embodiments, the NLS is present in a linker or the NLS is flanked by linkers, for example described herein. A bipartite NLS comprises two basic amino acid clusters, which are separated by a relatively short spacer sequence (hence bipartite-2 parts, while monopartite NLSs are not). The NLS of nucleoplasmin, KR [PAATKKAGQA] KKKK (SEQ ID NO: 191), is the prototype of the ubiquitous bipartite signal: two clusters of basic amino acids, separated by a spacer of about 10 amino acids. The sequence of an exemplary bipartite NLS follows:

(SEQ ID NO: 328)
PKKKRKVEGADKRTADGSEFESPKKKRKV.

In some embodiments, any of the fusion proteins or complexes provided herein comprise an NLS comprising the amino acid sequence EGADKRTADGSEFESPKKKRKV (amino acids 8 to 29 of SEQ ID NO 328). In some embodiments, any of the adenosine base editors provided herein, for example ABE Variant A, ABE Variant B, ABE Variant C, ABE Variant D, ABE Variant E, ABE Variant F, ABE Variant G, ABE Variant H, ABE Variant I, ABE Variant J, ABE Variant K, or ABE Variant D comprise an NLS comprising the amino acid sequence EGADKRTADGSEFESPKKKRKV (amino acids 8 to 29 of SEQ ID NO: 328). In some embodiments, the NLS is at a C-terminal portion of the adenosine base editor. In some embodiments, the NLS is at the C-terminus of the adenosine base editor.

### Additional Domains

A base editor described herein can include any domain which helps to facilitate the nucleobase editing, modification or altering of a nucleobase of a polynucleotide. In some embodiments, a base editor comprises a polynucleotide programmable nucleotide binding domain (e.g., Cas9), a nucleobase editing domain (e.g., deaminase domain), and one or more additional domains. In some embodiments, the additional domain can facilitate enzymatic or catalytic functions of the base editor, binding functions of the base editor, or be inhibitors of cellular machinery (e.g., enzymes) that could interfere with the desired base editing result. In some embodiments, a base editor comprises a nuclease, a nickase, a recombinase, a deaminase, a methyltransferase, a methylase, an acetylase, an acetyltransferase, a transcriptional activator, or a transcriptional repressor domain.

In some embodiments, a base editor comprises an uracil glycosylase inhibitor (UGI) domain. In some cases, a base editor is expressed in a cell in trans with a UGI polypeptide. In some embodiments, cellular DNA repair response to the presence of U: G heteroduplex DNA can be responsible for a reduction in nucleobase editing efficiency in cells. In such embodiments, uracil DNA glycosylase (UDG) can catalyze removal of U from DNA in cells, which can initiate base excision repair (BER), mostly resulting in reversion of the U: G pair to a C: G pair. In such embodiments, BER can be inhibited in base editors comprising one or more domains that bind the single strand, block the edited base, inhibit UGI, inhibit BER, protect the edited base, and/or promote repairing of the non-edited strand. Thus, this disclosure contemplates a base editor fusion protein or complex comprising a UGI domain and/or a uracil stabilizing protein (USP) domain.

### Base Editor System

Provided herein are systems, compositions, and methods for editing a nucleobase using a base editor system. In some embodiments, the base editor system comprises (1) a base editor (BE) comprising a polynucleotide programmable nucleotide binding domain and a nucleobase editing domain (e.g., a deaminase domain) for editing the nucleobase; and (2) a guide polynucleotide (e.g., guide RNA) in conjunction with the polynucleotide programmable nucleotide binding domain. In some embodiments, the base editor system is a cytidine base editor (CBE) or an adenosine base editor (ABE). In some embodiments, the polynucleotide programmable nucleotide binding domain is a polynucleotide programmable DNA or RNA binding domain. In some embodiments, the nucleobase editing domain is a deaminase domain. In some embodiments, a deaminase domain can be a cytidine deaminase or an cytosine deaminase. In some embodiments, a deaminase domain can be an adenine deaminase or an adenosine deaminase. In some embodiments, the adenosine base editor can deaminate adenine in DNA. In some embodiments, the base editor is capable of deaminating a cytidine in DNA.

Use of the base editor system provided herein comprises the steps of: (a) contacting a target nucleotide sequence of a polynucleotide (e.g., double- or single stranded DNA or RNA) of a subject with a base editor system comprising a nucleobase editor (e.g., an adenosine base editor or a cytidine base editor) and a guide polynucleotide (e.g., gRNA), wherein the target nucleotide sequence comprises a targeted nucleobase pair; (b) inducing strand separation of said target region; (c) converting a first nucleobase of said target nucleobase pair in a single strand of the target region to a second nucleobase; and (d) cutting no more than one strand of said target region, where a third nucleobase complementary to the first nucleobase base is replaced by a fourth nucleobase complementary to the second nucleobase. It should be appreciated that in some embodiments, step (b) is omitted. In some embodiments, said targeted nucleobase pair is a plurality of nucleobase pairs in one or more genes. In some embodiments, the base editor system provided herein is capable of multiplex editing of a plurality of nucleobase pairs in one or more genes. In some embodiments, the plurality of nucleobase pairs is located in the same gene. In some embodiments, the plurality of nucleobase pairs is located in one or more genes, wherein at least one gene is located in a different locus.

The components of a base editor system (e.g., a deaminase domain, a guide RNA, and/or a polynucleotide programmable nucleotide binding domain) may be associated with each other covalently or non-covalently. For example, in some embodiments, the deaminase domain can be targeted to a target nucleotide sequence by a polynucleotide programmable nucleotide binding domain, optionally where the polynucleotide programmable nucleotide binding domain is complexed with a polynucleotide (e.g., a guide RNA). In some embodiments, a polynucleotide programmable nucleotide binding domain can be fused or linked to a deaminase domain. In some embodiments, a polynucleotide programmable nucleotide binding domain can target a deaminase domain to a target nucleotide sequence by non-covalently interacting with or associating with the deaminase domain. For example, in some embodiments, the nucleobase editing component (e.g., the deaminase component) comprises an additional heterologous portion or domain that is capable of interacting with, associating with, or capable of forming a complex with a corresponding heterologous portion, antigen, or domain that is part of a polynucleotide programmable nucleotide binding domain and/or a guide polynucleotide (e.g., a guide RNA) complexed therewith. In some embodiments, the polynucleotide programmable nucleotide binding domain, and/or a guide polynucleotide (e.g., a guide RNA) complexed therewith, comprises an additional heterologous portion or domain that is capable of interacting with, associating with, or capable of forming a complex with a corresponding heterologous portion, antigen, or domain that is part of a nucleobase editing domain (e.g., the deaminase component). In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion is capable of binding to a polynucleotide linker. An additional heterologous portion may be a protein domain. In some embodiments, an additional heterologous portion comprises a polypeptide, such as a 22 amino acid RNA-binding domain of the lambda bacteriophage antiterminator protein N (N22p), a 2G12 IgG homodimer domain, an ABI, an antibody (e.g. an antibody that binds a component of the base editor system or a heterologous portion thereof) or fragment thereof (e.g. heavy chain domain 2 (CH2) of IgM (MHD2) or IgE (EHD2), an immunoglobulin Fc region, a heavy chain domain 3 (CH3) of IgG or IgA, a heavy chain domain 4 (CH4) of IgM or IgE, an Fab, an Fab2, miniantibodies, and/or ZIP antibodies), a barnase-barstar dimer domain, a Bcl-xL domain, a Calcineurin A (CAN) domain, a Cardiac phospholamban transmembrane pentamer domain, a collagen domain, a Com RNA binding protein domain (e.g. SfMu Com coat protein domain, and SfMu Com binding protein domain), a Cyclophilin-Fas fusion protein (CyP-Fas) domain, a Fab domain, an Fc domain, a fibritin foldon domain, an FK506 binding protein (FKBP) domain, an FKBP binding domain (FRB) domain of mTOR, a foldon domain, a fragment X domain, a GAI domain, a GID1 domain, a Glycophorin A transmembrane domain, a GyrB domain, a Halo tag, an HIV Gp41 trimerisation domain, an HPV45 oncoprotein E7 C-terminal dimer domain, a hydrophobic polypeptide, a K Homology (KH) domain, a Ku protein domain (e.g., a Ku heterodimer), a leucine zipper, a LOV domain, a mitochondrial antiviral-signaling protein CARD filament domain, an MS2 coat protein domain (MCP), a non-natural RNA aptamer ligand that binds a corresponding RNA motif/aptamer, a parathyroid hormone dimerization domain, a PP7 coat protein (PCP) domain, a PSD95-Dlgl-zo-1 (PDZ) domain, a PYL domain, a SNAP tag, a SpyCatcher moiety, a SpyTag moiety, a streptavidin domain, a streptavidin-binding protein domain, a streptavidin binding protein (SBP) domain, a telomerase Sm7 protein domain (e.g. Sm7 homoheptamer or a monomeric Sm-like protein), and/or fragments thereof. In embodiments, an additional heterologous portion comprises a polynucleotide (e.g., an RNA motif), such as an MS2 phage operator stem-loop (e.g., an MS2, an MS2 C-5 mutant, or an MS2 F-5 mutant), a non-natural RNA motif, a PP7 operator stem-loop, an SfMu phate Com stem-loop, a steril alpha motif, a telomerase Ku binding motif, a telomerase Sm7 binding motif, and/or fragments thereof. Non-limiting examples of additional heterologous portions include polypeptides with at least about 85% sequence identity to any one or more of SEQ ID NOs: 380, 382, 384, 386-388, or fragments thereof. Non-limiting examples of additional heterologous portions include polynucleotides with at least about 85% sequence identity to any one or more of SEQ ID NOs: 379, 381, 383, 385, or fragments thereof.

In some instances, components of the base editing system are associated with one another through the interaction of leucine zipper domains (e.g., SEQ ID NOs: 387 and 388). In some cases, components of the base editing system are associated with one another through polypeptide domains (e.g., FokI domains) that associate to form protein complexes containing about, at least about, or no more than about 1, 2 (i.e., dimerize), 3, 4, 5, 6, 7, 8, 9, 10 polypeptide domain units, optionally the polypeptide domains may include alterations that reduce or eliminate an activity thereof.

In some instances, components of the base editing system are associated with one another through the interaction of multimeric antibodies or fragments thereof (e.g., IgG, IgD, IgA, IgM, IgE, a heavy chain domain 2 (CH2) of IgM (MHD2) or IgE (EHD2), an immunoglobulin Fc region, a heavy chain domain 3 (CH3) of IgG or IgA, a heavy chain domain 4 (CH4) of IgM or IgE, an Fab, and an Fab2). In some instances, the antibodies are dimeric, trimeric, or tetrameric. In embodiments, the dimeric antibodies bind a polypeptide or polynucleotide component of the base editing system.

In some cases, components of the base editing system are associated with one another through the interaction of a polynucleotide-binding protein domain(s) with a polynucleotide(s). In some instances, components of the base editing system are associated with one another through the interaction of one or more polynucleotide-binding protein domains with polynucleotides that are self-complementary and/or complementary to one another so that complementary binding of the polynucleotides to one another brings into association their respective bound polynucleotide-binding protein domain(s).

In some instances, components of the base editing system are associated with one another through the interaction of a polypeptide domain(s) with a small molecule(s) (e.g., chemical inducers of dimerization (CIDs), also known as “dimerizers”). Non-limiting examples of CIDs include those disclosed in Amara, et al., “A versatile synthetic dimerizer for the regulation of protein-protein interactions,” PNAS, 94:10618-10623 (1997); and Voß, et al. “Chemically induced dimerization: reversible and spatiotemporal control of protein function in cells,” Current Opinion in Chemical Biology, 28:194-201 (2015), the disclosures of each of which are incorporated herein by reference in their entireties for all purposes. In some embodiments, the base editor inhibits base excision repair (BER) of the edited strand. In some embodiments, the base editor protects or binds the non-edited strand. In some embodiments, the base editor comprises UGI activity or USP activity. In some embodiments, the base editor comprises a catalytically inactive inosine-specific nuclease.

The base editors of the present disclosure can comprise any domain, feature or amino acid sequence which facilitates the editing of a target polynucleotide sequence. For example, in some embodiments, the base editor comprises a nuclear localization sequence (NLS). In some embodiments, an NLS of the base editor is localized between a deaminase domain and a polynucleotide programmable nucleotide binding domain. In some embodiments, an NLS of the base editor is localized C-terminal to a polynucleotide programmable nucleotide binding domain.

Protein domains included in the fusion protein can be a heterologous functional domain. Non-limiting examples of protein domains which can be included in the fusion protein include a deaminase domain (e.g., cytidine deaminase and/or adenosine deaminase), a uracil glycosylase inhibitor (UGI) domain, epitope tags, and reporter gene sequences.

In some embodiments, the adenosine base editor (ABE) can deaminate adenine in DNA. In some embodiments, ABE is generated by replacing APOBEC1 component of BE3 with natural or engineered E. coli TadA, human ADAR2, mouse ADA, or human ADAT2. In some embodiments, ABE comprises an evolved TadA variant. In some embodiments, the base editor is ABE8.1, which comprises or consists essentially of the following sequence or a fragment thereof having adenosine deaminase activity: SEQ ID NO: 331. Other ABE8 sequences are provided in the attached sequence listing (SEQ ID NOs: 332-354).

In some embodiments, the base editor includes an adenosine deaminase variant comprising an amino acid sequence, which contains alterations relative to an ABE 7*10 reference sequence, as described herein. The term “monomer” as used in Table 7 refers to a monomeric form of TadA*7.10 comprising the alterations described. The term “heterodimer” as used in Table 7 refers to the specified wild-type E. coli TadA adenosine deaminase fused to a TadA*7.10 comprising the alterations as described.

TABLE 7
Adenosine Deaminase Base Editor Variants

| | Adenosine | |
|---|---|---|
| ABE | Deaminase | Adenosine Deaminase Description |
| ABE-605m | MSP605 | monomer_TadA*7.10 + V82G + Y147T + Q154S |
| ABE-680m | MSP680 | monomer_TadA*7.10 + I76Y + V82G + Y147T + Q154S |
| ABE-823m | MSP823 | monomer_TadA*7.10 + L36H + V82G + Y147T + Q154S + |
| | | N157K |
| ABE-824m | MSP824 | monomer_TadA*7.10 + V82G + Y147D + F149Y + Q154S + |
| | | D167N |
| ABE-825m | MSP825 | monomer_TadA*7.10 + L36H + V82G + Y147D + F149Y + |
| | | Q154S + N157K + D167N |
| ABE-827m | MSP827 | monomer_TadA*7.10 + L36H + I76Y + V82G + Y147T + Q154S + |
| | | N157K |
| ABE-828m | MSP828 | monomer_TadA*7.10 + I76Y + V82G + Y147D + F149Y + Q154S + |
| | | D167N |
| ABE-829m | MSP829 | monomer_TadA*7.10 + L36H + I76Y + V82G + Y147D + F149Y + |
| | | Q154S + N157K + D167N |
| ABE-605d | MSP605 | heterodimer_(WT) + (TadA*7.10 + V82G + Y147T + Q154S) |
| ABE-680d | MSP680 | heterodimer_(WT) + (TadA*7.10 + I76Y + V82G + Y147T + |
| | | Q154S) |
| ABE-823d | MSP823 | heterodimer_(WT) + (TadA*7.10 + L36H + V82G + Y147T + |
| | | Q154S + N157K) |
| ABE-824d | MSP824 | heterodimer_(WT) + (TadA*7.10 + V82G + Y147D + F149Y + |
| | | Q154S + D167N) |
| ABE-825d | MSP825 | heterodimer_(WT) + (TadA*7.10 + L36H + V82G + Y147D + |
| | | F149Y + Q154S + N157K + D167N) |
| ABE-827d | MSP827 | heterodimer_(WT) + (TadA*7.10 + L36H + I76Y + V82G + Y147T + |
| | | Q154S + N157K) |
| ABE-828d | MSP828 | heterodimer_(WT) + (TadA*7.10 + I76Y + V82G + Y147D + |
| | | F149Y + Q154S + D167N) |
| ABE-829d | MSP829 | heterodimer_(WT) + (TadA*7.10 + L36H + I76Y + V82G + Y147D + |
| | | F149Y + Q154S + N157K + D167N) |

In some embodiments, the base editor comprises a domain comprising all or a portion (e.g., a functional portion) of a uracil glycosylase inhibitor (UGI) or a uracil stabilizing protein (USP) domain.

### Linkers

In certain embodiments, linkers may be used to link any of the peptides or peptide domains of the disclosure. The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.).

In some embodiments, any of the fusion proteins provided herein, comprise a cytidine or adenosine deaminase and a Cas9 domain that are fused to each other via a linker. Various linker lengths and flexibilities between the cytidine or adenosine deaminase and the Cas9 domain can be employed (e.g., ranging from very flexible linkers of the form (GGGS)ₙ (SEQ ID NO: 246), (GGGGS)ₙ (SEQ ID NO: 247), and (G)ₙ to more rigid linkers of the form (EAAAK)ₙ (SEQ ID NO: 248), (SGGS)ₙ (SEQ ID NO: 355), SGSETPGTSESATPES (SEQ ID NO: 249) (see, e.g., Guilinger J P, et al. Fusion of catalytically inactive Cas9 to FokI nuclease improves the specificity of genome modification. Nat. Biotechnol. 2014; 32 (6): 577-82; the entire contents are incorporated herein by reference) and (XP)n) in order to achieve the optimal length for activity for the cytidine or adenosine deaminase nucleobase editor. In some embodiments, n is 1, 2, 3, 4, 5, 6, 7, 8, 9, 10, 11, 12, 13, 14, or 15. In some embodiments, the linker comprises a (GGS)n motif, wherein n is 1, 3, or 7. In some embodiments, cytidine deaminase or adenosine deaminase and the Cas9 domain of any of the fusion proteins provided herein are fused via a linker comprising the amino acid sequence SGSETPGTSESATPES (SEQ ID NO: 249), which can also be referred to as the XTEN linker.

In some embodiments, the domains of the base editor are fused via a linker that comprises the amino acid sequence of:

(SEQ ID NO: 356)
SGGSSGSETPGTSESATPESSGGS,
(SEQ ID NO: 357)
SGGSSGGSSGSETPGTSESATPESSGGSSGGS,
or
(SEQ ID NO: 358)
GGSGGSPGSPAGSPTSTEEGTSESATPESGPGTSTEPSEGSAPGSPAG
SPTSTEEGTSTEPSEGSAPGTSTEPSEGSAPGTSESATPESGPGSEPA
TSGGSGGS.

In some embodiments, domains of the base editor are fused via a linker comprising the amino acid sequence SGSETPGTSESATPES (SEQ ID NO: 249), which may also be referred to as the XTEN linker. In some embodiments, a linker comprises the amino acid sequence SGGS (SEQ ID NO: 355). In some embodiments, the linker is 24 amino acids in length. In some embodiments, the linker comprises the amino acid sequence SGGSSGGSSGSETPGTSESATPES (SEQ ID NO: 359). In some embodiments, the linker is 40 amino acids in length. In some embodiments, the linker comprises the amino acid sequence: SGGSSGGSSGSETPGTSESATPESSGGSSGGSSGGSSGGS (SEQ ID NO: 360). In some embodiments, the linker is 64 amino acids in length. In some embodiments, the linker comprises the amino acid sequence: SGGSSGGSSGSETPGTSESATPESSGGSSGGSSGGSSGGSSGSETPGTSESATPESSGGSSGGS (SEQ ID NO: 361). In some embodiments, the linker is 92 amino acids in length. In some embodiments, the linker comprises the amino acid sequence:

(SEQ ID NO: 362)
PGSPAGSPTSTEEGTSESATPESGPGTSTEPSEGSAPGSPAGSPTSTE
EGTSTEPSEGSAPGTSTEPSEGSAPGTSESATPESGPGSEPATS.

In some embodiments, a linker comprises a plurality of proline residues and is 5-21, 5-14, 5-9, 5-7 amino acids in length, e.g., PAPAP (SEQ ID NO: 363), PAPAPA (SEQ ID NO: 364), PAPAPAP (SEQ ID NO: 365), PAPAPAPA (SEQ ID NO: 366), P (AP) 4 (SEQ ID NO: 367), P (AP) 7 (SEQ ID NO: 368), P (AP) 10 (SEQ ID NO: 369) (see, e.g., Tan J, Zhang F, Karcher D, Bock R. Engineering of high-precision base editors for site-specific single nucleotide replacement. Nat Commun. 2019 Jan. 25; 10 (1): 439; the entire contents are incorporated herein by reference). Such proline-rich linkers are also termed “rigid” linkers.
Nucleic Acid Programmable DNA Binding Proteins with Guide RNAs

Provided herein are compositions and methods for base editing in cells. Further provided herein are compositions comprising a guide polynucleotide sequence, e.g., a guide RNA sequence, or a combination of 2, 3, 4, 5, 6, 7, 8, 9, 10, 11, 12, 13, 14, 15, 16, 17, 18, 19, 20, or more guide RNAs as provided herein. In some embodiments, a composition for base editing as provided herein further comprises a polynucleotide that encodes a base editor, e.g., a C-base editor or an A-base editor. For example, a composition for base editing may comprise a mRNA sequence encoding a BE, a BE4, an ABE, and a combination of one or more guide RNAs as provided. A composition for base editing may comprise a base editor polypeptide and a combination of one or more of any guide RNAs provided herein. Such a composition may be used to effect base editing in a cell through different delivery approaches, for example, electroporation, nucleofection, viral transduction or transfection. In some embodiments, the composition for base editing comprises an mRNA sequence that encodes a base editor and a combination of one or more guide RNA sequences provided herein for electroporation.

Some aspects of this disclosure provide systems comprising any of the fusion proteins or complexes provided herein, and a guide RNA bound to a nucleic acid programmable DNA binding protein (napDNAbp) domain (e.g., a Cas9 (e.g., a dCas9, a nuclease active Cas9, or a Cas9 nickase) or Cas12) of the fusion protein or complex. These complexes are also termed ribonucleoproteins (RNPs). In some embodiments, the guide nucleic acid (e.g., guide RNA) is from 15-100 nucleotides long and comprises a sequence of at least 10 contiguous nucleotides that is complementary to a target sequence. In some embodiments, the target sequence is a DNA sequence. In some embodiments, the target sequence is an RNA sequence. In some embodiments, the target sequence is a sequence in the genome of a bacteria, yeast, fungi, insect, plant, or animal. In some embodiments, the target sequence is a sequence in the genome of a human. In some embodiments, the 3′ end of the target sequence is immediately adjacent to a canonical PAM sequence (NGG). In some embodiments, the 3′ end of the target sequence is immediately adjacent to a non-canonical PAM sequence (e.g., a sequence listed in Table 3 or 5′-NAA-3′). In some embodiments, the guide nucleic acid (e.g., guide RNA) is complementary to a sequence in a gene of interest (e.g., a gene associated with a disease or disorder).

Some aspects of this disclosure provide methods of using the fusion proteins, or complexes provided herein. For example, some aspects of this disclosure provide methods comprising contacting a DNA molecule with any of the fusion proteins or complexes provided herein, and with at least one guide RNA, wherein the guide RNA is about 15-100 nucleotides long and comprises a sequence of at least 10 contiguous nucleotides that is complementary to a target sequence.

The domains of the base editor disclosed herein can be arranged in any order.

A defined target region can be a deamination window. A deamination window can be the defined region in which a base editor acts upon and deaminates a target nucleotide. In some embodiments, the deamination window is within a 2, 3, 4, 5, 6, 7, 8, 9, or 10 base regions. In some embodiments, the deamination window is 5, 6, 7, 8, 9, 10, 11, 12, 13, 14, 15, 16, 17, 18, 19, 20, 21, 22, 23, 24, or 25 bases upstream of the PAM.

The base editors of the present disclosure can comprise any domain, feature or amino acid sequence which facilitates the editing of a target polynucleotide sequence.

### Methods of Using Fusion Proteins or Complexes Comprising a Cytidine or Adenosine Deaminase and a Cas9 Domain

Some aspects of this disclosure provide methods of using the fusion proteins, or complexes provided herein. For example, some aspects of this disclosure provide methods comprising contacting a DNA molecule with any of the fusion proteins or complexes provided herein, and with at least one guide RNA described herein.

In some embodiments, a fusion protein or complex of the disclosure is used for editing a target gene of interest. In particular, a cytidine deaminase or adenosine deaminase nucleobase editor described herein is capable of making multiple mutations within a target sequence. These mutations may affect the function of the target. For example, when a cytidine deaminase or adenosine deaminase nucleobase editor is used to target a regulatory region the function of the regulatory region is altered and the expression of the downstream protein is reduced or eliminated.

### Base Editor Efficiency

In some embodiments, the purpose of the methods provided herein is to alter a gene and/or gene product via gene editing. The nucleobase editing proteins provided herein can be used for gene editing-based human therapeutics in vitro or in vivo. It will be understood by the skilled artisan that the nucleobase editing proteins provided herein, e.g., the fusion proteins or complexes comprising a polynucleotide programmable nucleotide binding domain (e.g., Cas9) and a nucleobase editing domain (e.g., an adenosine deaminase domain or a cytidine deaminase domain) can be used to edit a nucleotide from A to G or C to T.

Advantageously, base editing systems as provided herein provide genome editing without generating double-strand DNA breaks, without requiring a donor DNA template, and without inducing an excess of stochastic insertions and deletions as CRISPR may do. In some embodiments, the present disclosure provides base editors that efficiently generate an intended mutation, such as a STOP codon, in a nucleic acid (e.g., a nucleic acid within a genome of a subject) without generating a significant number of unintended mutations, such as unintended point mutations.

The base editors of the disclosure advantageously modify a specific nucleotide base encoding a protein without generating a significant proportion of indels (i.e., insertions or deletions). Such indels can lead to frame shift mutations within a coding region of a gene.

In some embodiments, the base editors provided herein are capable of generating a ratio of intended mutations to indels (i.e., intended point mutations: unintended point mutations) that is greater than 1:1. In some embodiments, the base editors provided herein are capable of generating a ratio of intended mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more. The number of intended mutations and indels may be determined using any suitable method.

In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor. In some embodiments, any of the base editors provided herein can limit the formation of indels at a region of a nucleic acid to less than 1%, less than 1.5%, less than 2%, less than 2.5%, less than 3%, less than 3.5%, less than 4%, less than 4.5%, less than 5%, less than 6%, less than 7%, less than 8%, less than 9%, less than 10%, less than 12%, less than 15%, or less than 20%.

Base editing is often referred to as a “modification”, such as, a genetic modification, a gene modification and modification of the nucleic acid sequence and is clearly understandable based on the context that the modification is a base editing modification. A base editing modification is therefore a modification at the nucleotide base level, for example as a result of the deaminase activity discussed throughout the disclosure, which then results in a change in the gene sequence and may affect the gene product.

In some embodiments, the modification, e.g., single base edit results in about or at least about a 10%, 15%, 20%, 25%, 30%, 35%, 40%, 45%, 50%, 55%, 60%, 65%, 70%, 75%, 80%, 85%, 90%, 95%, 99%, or 100% reduction, or reduction to an undetectable level, of the gene targeted expression.

The disclosure provides adenosine deaminase variants (e.g., ABE8 variants) that have increased efficiency and specificity. In particular, the adenosine deaminase variants described herein are more likely to edit a desired base within a polynucleotide and are less likely to edit bases that are not intended to be altered (e.g., “bystanders”).

In some embodiments, any of the base editing system comprising one of the ABE8 base editor variants described herein has reduced bystander editing or mutations by at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 99% compared to a base editor system comprising an ABE7 base editor, e.g., ABE7.10.

In some embodiments, any of the ABE8 base editor variants described herein has higher base editing efficiency compared to the ABE7 base editors. In some embodiments, any of the ABE8 base editor variants described herein have at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99%, 100%, 105%, 110%, 115%, 120%, 125%, 130%, 135%, 140%, 145%, 150%, 155%, 160%, 165%, 170%, 175%, 180%, 185%, 190%, 195%, 200%, 210%, 220%, 230%, 240%, 250%, 260%, 270%, 280%, 290%, 300%, 310%, 320%, 330%, 340%, 350%, 360%, 370%, 380%, 390%, 400%, 450%, or 500% higher base editing efficiency compared to an ABE7 base editor, e.g., ABE7.10.

The ABE8 base editor variants described herein may be delivered to a host cell via a plasmid, a vector, a LNP complex, or an mRNA. In some embodiments, any of the ABE8 base editor variants described herein is delivered to a host cell as an mRNA.

In some embodiments, the method described herein, for example, the base editing methods has minimum to no off-target effects. In some embodiments, the method described herein, for example, the base editing methods, has minimal to no chromosomal translocations.

In some embodiments, the base editing method described herein results in about 91%, 92%, 93%, 94%, 95%, 96%, 97%, 98%, 99% or 100% of a cell population that have been successfully edited.

In some embodiments, the percent of viable cells in a cell population following a base editing intervention is greater than at least 60%, 70%, 80%, or 90% of the starting cell population at the time of the base editing event. In some embodiments, the percent of viable cells in a cell population following editing is about 70%. In some embodiments, the percent of viable cells in a cell population following editing is about 75%. In some embodiments, the percent of viable cells in a cell population following editing is about 80%. In some embodiments, the percent of viable cells in a cell population as described above is about 85%. In some embodiments, the percent of viable cells in a cell population as described above is about 90%, or about 91%, 92%, 93%, 94% 95%, 96%, 97%, 98%, 99%, or 100% of the cells in the population at the time of the base editing event.

In embodiments, the cell population is a population of cells contacted with a base editor, complex, or base editor system of the present disclosure.

The number of intended mutations and indels can be determined using any suitable method, for example, as described in International PCT Application Nos. PCT/US2017/045381 (WO2018/027078) and PCT/US2016/058344 (WO2017/070632); Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); and Komor, A. C., et al., “Improved base excision repair inhibition and bacteriophage Mu Gam protein yields C: G-to-T: A base editors with higher efficiency and product purity” Science Advances 3: eaao4774 (2017); the entire contents of which are hereby incorporated by reference.

In some embodiments, to calculate indel frequencies, sequencing reads are scanned for exact matches to two 10-bp sequences that flank both sides of a window in which indels can occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively. In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor.

### Multiplex Editing

In some embodiments, the base editor system provided herein is capable of multiplex editing of a plurality of nucleobase pairs in one or more genes or polynucleotide sequences. In some embodiments, the plurality of nucleobase pairs is located in the same gene or in one or more genes, wherein at least one gene is located in a different locus. In some embodiments, the multiplex editing comprises one or more guide polynucleotides. In some embodiments, the multiplex editing comprises one or more base editor systems. In some embodiments, the multiplex editing comprises one or more base editor systems with a single guide polynucleotide or a plurality of guide polynucleotides. In some embodiments, the multiplex editing comprises one or more guide polynucleotides with a single base editor system. It should be appreciated that the characteristics of the multiplex editing using any of the base editors as described herein can be applied to any combination of methods using any base editor provided herein. It should also be appreciated that the multiplex editing using any of the base editors as described herein can comprise a sequential editing of a plurality of nucleobase pairs.

In some embodiments, the base editor system capable of multiplex editing of a plurality of nucleobase pairs in one or more genes comprises one of ABE7, ABE8, and/or ABE9 base editors.

### Expression of Fusion Proteins or Complexes in a Host Cell

Fusion proteins or complexes of the disclosure comprising a deaminase may be expressed in virtually any host cell of interest, including but not limited to bacteria, yeast, fungi, insects, plants, and animal cells using routine methods known to the skilled artisan. For example, a DNA encoding an adenosine deaminase of the disclosure can be cloned by designing suitable primers for the upstream and downstream of CDS based on the cDNA sequence. The cloned DNA may be directly, or after digestion with a restriction enzyme when desired, or after addition of a suitable linker and/or a nuclear localization signal, ligated with a DNA encoding one or more additional components of a base editing system. The base editing system is translated in a host cell to form a complex.

A polynucleotide encoding a polypeptide described herein can be obtained by chemically synthesizing the polynucleotide, or by connecting synthesized partly overlapping oligo short chains by utilizing the PCR method and the Gibson Assembly method to construct a polynucleotide (e.g., DNA) encoding the full length thereof. The advantage of constructing a full-length polynucleotide by chemical synthesis or a combination of PCR method or Gibson Assembly method is that the codons to be used can be selected in according to the host into which the polynucleotide is to be introduced. In the expression from a heterologous DNA molecule, the protein expression level is expected to increase by converting the DNA sequence thereof to a codon highly frequently used in the host organism. Codon use data for a host cell (e.g., codon use data available at kazusa.or.jp/codon/index.html) can be used to guide codon optimization for a polynucleotide sequence encoding a polypeptide. Codons having low use frequency in the host may be converted to a codon coding the same amino acid and having high use frequency.

An expression vector containing a polynucleotide encoding a nucleic acid sequence-recognizing module and/or a nucleic acid base converting enzyme can be produced, for example, by linking the DNA to the downstream of a promoter in a suitable expression vector.

As the expression vector, Escherichia coli-derived plasmids (e.g., pBR322, pBR325, pUC12, pUC13); Bacillus subtilis-derived plasmids (e.g., pUB110, pTP5, pC194); yeast-derived plasmids (e.g., pSH19, pSH15); insect cell expression plasmids (e.g., pFast-Bac); animal cell expression plasmids (e.g., pA1-11, pXT1, pRc/CMV, pRc/RSV, pcDNAI/Neo); bacteriophages such as .lambda phage and the like; insect virus vectors such as baculovirus and the like (e.g., BmNPV, AcNPV); animal virus vectors such as retrovirus, vaccinia virus, adenovirus and the like, and the like are used.

Regarding the promoter to be used, any promoter appropriate for a host to be used for gene expression can be used. In a conventional method using double-stranded breaks, since the survival rate of the host cell sometimes reduces markedly due to the toxicity, it is desirable to increase the number of cells by the start of the induction by using an inductive promoter. However, since sufficient cell proliferation can also be afforded by expressing the nucleic acid-modifying enzyme complex of the present disclosure, a constitutive promoter can be used without limitation.

For example, when the host is an animal cell, an SR.alpha. promoter, SV40 promoter, LTR promoter, cytomegalovirus (CMV) promoter, Rous sarcoma virus (RSV) promoter, Moloney mouse leukemia virus (MoMuLV), LTR, herpes simplex virus thymidine kinase (HSV-TK) promoter, and the like can be used. Of these, CMV promoter, SR.alpha. promoter and the like may be used.

When the host is Escherichia coli, a trp promoter, lac promoter, recA promoter, .lamda.P.sub.L promoter, lpp promoter, T7 promoter, and the like can be used.

When the host is in the genus Bacillus, the SPO1 promoter, SPO2 promoter, penP promoter, and the like can be used.

When the host is a yeast, the Gal1/10 promoter, PHO5 promoter, PGK promoter, GAP promoter, ADH promoter, and the like can be used.

When the host is an insect cell, the polyhedrin promoter, P10 promoter, and the like can be used.

When the host is a plant cell, the CaMV35S promoter, CaMV19S promoter, NOS promoter, and the like can be used.

Expression vectors for use in the present disclosure, besides those mentioned above, can comprise an enhancer, a splicing signal, a terminator, a poly A addition signal, a selection marker such as drug resistance gene, an auxotrophic complementary gene and the like, a replication origin, and the like can be used.

An RNA encoding a protein domain described herein can be prepared by, for example, in vitro transcription of a nucleic acid sequence encoding any of the fusion proteins or complexes disclosed herein.

A fusion protein or complex of the disclosure can be intracellularly expressed by introducing into the cell an expression vector comprising a nucleic acid sequence encoding the fusion protein or complex.

Host cells of interest, include but are not limited to bacteria, yeast, fungi, insects, plants, and animal cells. For example, a host cell may comprise bacteria from the genus Escherichia, such as Escherichia coli K12.cndot.DH1 [Proc. Natl. Acad. Sci. USA, 60, 160 (1968)], Escherichia coli JM103 [Nucleic Acids Research, 9, 309 (1981)], Escherichia coli JA221 [Journal of Molecular Biology, 120, 517 (1978)], Escherichia coli HB101 [Journal of Molecular Biology, 41, 459 (1969)], Escherichia coli C600 [Genetics, 39, 440 (1954)] and the like.

A host cell may comprise bacteria from the genus Bacillus, for example Bacillus subtilis M1114 [Gene, 24, 255 (1983)], Bacillus subtilis 207-21 [Journal of Biochemistry, 95, 87 (1984)] and the like.

A host cell may be a yeast cell. Examples of yeast cells include Saccharomyces cerevisiae AH22, AH22R.sup.-, NA87-11A, DKD-5D, 20B-12, Schizosaccharomyces pombe NCYC1913, NCYC2036, Pichia pastoris KM71 and the like.

When the viral delivery methods utilize the virus AcNPV, cells from a cabbage armyworm larva-derived established line (Spodoptera frugiperda cell; Sf cell), MG1 cells derived from the mid-intestine of Trichoplusia ni, High Five™ cells derived from an ovary of Trichoplusia ni, Mamestra brassicae-derived cells, Estigmena acrea-derived cells and the like can be used. When the virus is BmNPV, cells of Bombyx mori-derived established line (Bombyx mori N cell; BmN cell) and the like are used. As the Sf cell, for example, Sf9 cell (ATCC CRL1711), Sf21 cell [all above, In Vivo, 13, 213-217 (1977)] and the like are used.

An insect can be any insect, for example, larva of Bombyx mori, Drosophila, cricket, and the like [Nature, 315, 592 (1985)].

Animal cells contemplated in the present disclosure include, but are not limited to, cell lines such as monkey COS-7 cells, monkey Vero cells, Chinese hamster ovary (CHO) cells, dhfr gene-deficient CHO cells, mouse L cells, mouse AtT-20 cells, mouse myeloma cells, rat GH3 cells, human FL cells and the like, pluripotent stem cells such as iPS cells, ES cells derived humans and other mammals, and primary cultured cells prepared from various tissues. Furthermore, zebrafish embryo, Xenopus oocyte, and the like can also be used.

Plant cells are also contemplated in the present disclosure. Plant cells include, but are not limited to, suspended cultured cells, callus, protoplast, leaf segment, root segment and the like prepared from various plants (e.g., grain such as rice, wheat, corn, and the like; product crops such as tomato, cucumber, eggplant and the like; garden plants such as carnations, Eustoma russellianum, and the like; and other plants such as tobacco, Arabidopsis thaliana and the like) are used.

All the above-mentioned host cells may be haploid (monoploid), or polyploid (e.g., diploid, triploid, tetraploid, etc.). Using conventional methods, mutations, in principle, introduced into only one homologous chromosome produce a heterogenous cell. Therefore, the desired phenotype is not expressed unless the mutation is dominant. For recessive mutations, acquiring a homozygous cell can be inconvenient due to labor and time requirements. In contrast, according to the present disclosure, since a mutation can be introduced into any allele on the homologous chromosome in the genome, the desired phenotype can be expressed in a single generation even in the case of recessive mutation, thereby solving the problem associated with conventional mutagenesis methods.

An expression vector can be introduced by a known method (e.g., the lysozyme method, the competent method, the PEG method, the CaCl₂) coprecipitation method, electroporation, microinjection, particle gun method, lipofection, Agrobacterium-mediated delivery, etc.) according to the kind of the host.

Escherichia coli can be transformed according to the methods described in, for example, Proc. Natl. Acad. Sci. USA, 69, 2110 (1972), Gene, 17, 107 (1982).

The genus Bacillus can be introduced into a vector according to the methods described in, for example, Molecular & General Genetics, 168, 111 (1979).

A yeast can be introduced into a vector according to the methods described in, for example, Methods in Enzymology, 194, 182-187 (1991), Proc. Natl. Acad. Sci. USA, 75, 1929 (1978).

An insect cell and an insect can be introduced into a vector according to the methods described in, for example, Bio/Technology, 6, 47-55 (1988).

A vector can be introduced into an animal cell according to the methods described in, for example, Cell Engineering additional volume 8, New Cell Engineering Experiment Protocol, 263-267 (1995) (published by Shujunsha), and Virology, 52, 456 (1973).

A cell comprising a vector can be cultured according to a known method according to the kind of the host. For example, when Escherichia coli or genus Bacillus is cultured, a liquid medium may be used as a medium to be used for the culture. The medium may contain a carbon source, nitrogen source, inorganic substance and the like necessary for the growth of the transformant. Examples of the carbon source include glucose, dextrin, soluble starch, sucrose and the like; examples of the nitrogen source include inorganic or organic substances such as ammonium salts, nitrate salts, corn steep liquor, peptone, casein, meat extract, soybean cake, potato extract and the like; and examples of the inorganic substance include calcium chloride, sodium dihydrogen phosphate, magnesium chloride and the like. The medium may contain yeast extract, vitamins, growth promoting factor and the like. The pH of the medium is between about 5 about 8 in embodiments.

As a medium for culturing Escherichia coli, for example, M9 medium containing glucose, casamino acid [Journal of Experiments in Molecular Genetics, 431-433, Cold Spring Harbor Laboratory, New York 1972] can be used. Where necessary, for example, agents such as 3B-indolylacrylic acid may be added to the medium to ensure an efficient function of a promoter. Escherichia coli is cultured at generally about 15 to about 43° C. Where necessary, aeration and stirring may be performed.

The genus Bacillus is cultured at generally about 30 to about 40° C. Where necessary, aeration and stirring may be performed.

Examples of the medium for culturing yeast include Burkholder minimum medium [Proc. Natl. Acad. Sci. USA, 77, 4505 (1980)], SD medium containing 0.5% casamino acid [Proc. Natl. Acad. Sci. USA, 81, 5330 (1984)] and the like. The pH of the medium may be between about 5 to about 8. The culture is performed at generally about 20° C. to about 35° C. Where necessary, aeration and stirring may be performed.

As a medium for culturing an insect cell or insect, for example, Grace's Insect Medium [Nature, 195, 788 (1962)] containing an additive such as inactivated 10% bovine serum and the like as appropriate and the like are used. The pH of the medium is may be between about 6.2 to about 6.4. The culture is performed at generally about 27° C. Where necessary, aeration and stirring may be performed.

As a medium for culturing an animal cell, for example, minimum essential medium (MEM) containing about 5 to about 20% of fetal bovine serum [Science, 122, 501 (1952)], Dulbecco's modified Eagle medium (DMEM) [Virology, 8, 396 (1959)], RPMI 1640 medium [The Journal of the American Medical Association, 199, 519 (1967)], 199 medium [Proceeding of the Society for the Biological Medicine, 73, 1 (1950)] and the like are used. The pH of the medium may be between about 6 to about 8. The culture is performed at generally about 30° C. to about 40° C. Where necessary, aeration and stirring may be performed.

As a medium for culturing a plant cell, for example, MS medium, LS medium, B5 medium and the like are used. The pH of the medium may be between about 5-about 8. The culture is performed at generally about 20° C. to about 30° C. Where necessary, aeration and stirring may be performed.

When a higher eukaryotic cell, such as animal cell, insect cell, plant cell and the like is used as a host cell, a polynucleotide encoding a base editing system of the present disclosure (e.g., comprising an adenosine deaminase variant) is introduced into a host cell under the regulation of an inducible promoter (e.g., metallothionein promoter (induced by heavy metal ion), heat shock protein promoter (induced by heat shock), Tet-ON/Tet-OFF system promoter (induced by addition or removal of tetracycline or a derivative thereof), steroid-responsive promoter (induced by steroid hormone or a derivative thereof) etc.), the induction substance is added to the medium (or removed from the medium) at an appropriate stage to induce expression of the nucleic acid-modifying enzyme complex, culture is performed for a given period to carry out a base editing and, introduction of a mutation into a target gene, transient expression of the base editing system can be realized.

Prokaryotic cells such as Escherichia coli and the like can utilize an inducible promoter. Examples of the inducible promoter include, but are not limited to, lac promoter (induced by IPTG), cspA promoter (induced by cold shock), araBAD promoter (induced by arabinose) and the like.

Alternatively, the above-mentioned inductive promoter can also be utilized as a vector removal mechanism when higher eukaryotic cells, such as animal cell, insect cell, plant cell and the like are used as a host cell. That is, a vector is mounted with a replication origin that functions in a host cell, and a nucleic acid encoding a protein necessary for replication (e.g., SV40 on and large T antigen, oriP and EBNA-1 etc. for animal cells), of the expression of the nucleic acid encoding the protein is regulated by the above-mentioned inducible promoter. As a result, while the vector is autonomously replicable in the presence of an induction substance, when the induction substance is removed, autonomous replication is not available, and the vector naturally falls off along with cell division (autonomous replication is not possible by the addition of tetracycline and doxycycline in Tet-OFF system vector).

### Delivery Systems

### Nucleic Acid-Based Delivery of Base Editor Systems

Nucleic acid molecules encoding a base editor system according to the present disclosure can be administered to subjects or delivered into cells in vitro or in vivo by art-known methods or as described herein. For example, a base editor system comprising a deaminase (e.g., cytidine or adenine deaminase) can be delivered by vectors (e.g., viral or non-viral vectors), or by naked DNA, DNA complexes, lipid nanoparticles, or a combination of the aforementioned compositions. A base editor system may be delivered to a cell using any methods available in the art including, but not limited to, physical methods (e.g., electroporation, particle gun, calcium phosphate transfection), viral methods, non-viral methods (e.g., liposomes, cationic methods, lipid nanoparticles, polymeric nanoparticles), or biological non-viral methods (e.g., attenuated bacterial, engineered bacteriophages, mammalian virus-like particles, biological liposomes, erythrocyte ghosts, exosomes).

Nanoparticles, which can be organic or inorganic, are useful for delivering a base editor system or component thereof. Nanoparticles are well known in the art and any suitable nanoparticle can be used to deliver a base editor system or component thereof, or a nucleic acid molecule encoding such components. In one example, organic (e.g., lipid and/or polymer) nanoparticles are suitable for use as delivery vehicles in certain embodiments of this disclosure.

Non-limiting examples of lipid nanoparticles suitable for use in the methods of the present disclosure include those described in International Patent Application Publications No. WO2022140239, WO2022140252, WO2022140238, WO2022159421, WO2022159472, WO2022159475, WO2022159463, WO2021113365, and WO2021141969, the disclosures of each of which is incorporated herein by reference in its entirety for all purposes.

### Viral Vectors

A base editor described herein can be delivered with a viral vector. In some embodiments, a base editor disclosed herein can be encoded on a nucleic acid that is contained in a viral vector. In some embodiments, one or more components of the base editor system can be encoded on one or more viral vectors.

Viral vectors can include lentivirus (e.g., HIV and FIV-based vectors), Adenovirus (e.g., AD100), Retrovirus (e.g., Maloney murine leukemia virus, MML-V), herpesvirus vectors (e.g., HSV-2), and Adeno-associated viruses (AAVs), or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses can be based on or extrapolated to an average 70 kg individual (e.g., a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific base editing, the expression of the base editor and optional guide nucleic acid can be driven by a cell-type specific promoter.

Viral vectors can be selected based on the application. For example, for in vivo gene delivery, AAV can be advantageous over other viral vectors. In some embodiments, AAV allows low toxicity, which can be due to the purification method not requiring ultra-centrifugation of cell particles that can activate the immune response. In some embodiments, AAV allows low probability of causing insertional mutagenesis because it doesn't integrate into the host genome. Adenoviruses are commonly used as vaccines because of the strong immunogenic response they induce. Packaging capacity of the viral vectors can limit the size of the base editor that can be packaged into the vector.

AAV has a packaging capacity of about 4.5 Kb or 4.75 Kb including two 145 base inverted terminal repeats (ITRs). This means disclosed base editor as well as a promoter and transcription terminator can fit into a single viral vector. Constructs larger than 4.5 or 4.75 Kb can lead to significantly reduced virus production. For example, SpCas9 is quite large, the gene itself is over 4.1 Kb, which makes it difficult for packing into AAV. Therefore, embodiments of the present disclosure include utilizing a disclosed base editor which is shorter in length than conventional base editors. In some examples, the base editors are less than 4 kb. Disclosed base editors can be less than 4.5 kb, 4.4 kb, 4.3 kb, 4.2 kb, 4.1 kb, 4 kb, 3.9 kb, 3.8 kb, 3.7 kb, 3.6 kb, 3.5 kb, 3.4 kb, 3.3 kb, 3.2 kb, 3.1 kb, 3 kb, 2.9 kb, 2.8 kb, 2.7 kb, 2.6 kb, 2.5 kb, 2 kb, or 1.5 kb. In some embodiments, the disclosed base editors are 4.5 kb or less in length.

An AAV can be AAV1, AAV2, AAV5, AAV6 or any combination thereof. One can select the type of AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. A tabulation of certain AAV serotypes as to these cells can be found in Grimm, D. et al, J. Virol. 82:5887-5911 (2008)).

In some embodiments, lentiviral vectors are used to transduce a cell of interest with a polynucleotide encoding a base editor or base editor system as provided herein. Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

In another embodiment, minimal non-primate lentiviral vectors based on the equine infectious anemia virus (EIAV) are also contemplated. In another embodiment, RetinoStat®, an equine infectious anemia virus-based lentiviral gene therapy vector that expresses angiostatic proteins endostatin and angiostatin that is contemplated to be delivered via a subretinal injection. In another embodiment, use of self-inactivating lentiviral vectors are contemplated.

Any RNA of the systems, for example a guide RNA or a base editor-encoding mRNA, can be delivered in the form of RNA. Base editor-encoding mRNA can be generated using in vitro transcription. For example, nuclease mRNA can be synthesized using a PCR cassette containing the following elements: T7 promoter, optional kozak sequence (GCCACC), nuclease sequence, and 3′ UTR such as a 3′ UTR from beta globin-polyA tail. The cassette can be used for transcription by T7 polymerase. Guide polynucleotides (e.g., gRNA) can also be transcribed using in vitro transcription from a cassette containing a T7 promoter, followed by the sequence “GG”, and guide polynucleotide sequence.

### Non-Viral Platforms for Gene Transfer

Non-viral platforms for introducing a heterologous polynucleotide into a cell of interest are known in the art.

For example, the disclosure provides a method of inserting a heterologous polynucleotide into the genome of a cell using a Cas9 or Cas12 (e.g., Cas12b) ribonucleoprotein complex (RNP)-DNA template complex where an RNP including a Cas9 or Cas12 nuclease domain and a guide RNA, wherein the guide RNA specifically hybridizes to a target region of the genome of the cell, and wherein the Cas nuclease domain cleaves the target region to create an insertion site in the genome of the cell. A DNA template is then used to introduce a heterologous polynucleotide. In embodiments, the DNA template is a double-stranded or single-stranded DNA template, wherein the size of the DNA template is about 200 nucleotides or is greater than about 200 nucleotides, wherein the 5′ and 3′ ends of the DNA template comprise nucleotide sequences that are homologous to genomic sequences flanking the insertion site. In some embodiments, the DNA template is a single-stranded circular DNA template. In embodiments, the molar ratio of RNP to DNA template in the complex is from about 3:1 to about 100:1.

In some embodiments, the DNA template is a linear DNA template. In some examples, the DNA template is a single-stranded DNA template. In certain embodiments, the single-stranded DNA template is a pure single-stranded DNA template. In some embodiments, the single stranded DNA template is a single-stranded oligodeoxynucleotide (ssODN).

In other embodiments, a single-stranded DNA (ssDNA) can produce efficient homology-directed repair (HDR) with minimal off-target integration. In one embodiment, an ssDNA phage is used to efficiently and inexpensively produce long circular ssDNA (cssDNA) donors. These cssDNA donors serve as efficient HDR templates when used with Cas9 or Cas12 (e.g., Cas12a, Cas12b), with integration frequencies superior to linear ssDNA (IssDNA) donors.

In some embodiments, a heterologous polynucleotide may be inserted into the genome of a cell using a transposable element such as a transposon, as described, for example, in Tipanee, et al. Human Gene Therapy, November 2017, 1087-1104, DOI: 10.1089/hum.2017.128. Transposable elements are divided into two categories: retrotransposons and DNA transposons. Transposable elements can alter the genome of the host cells through insertions, duplications, deletions, and translocations. Retrotransposons are described as mobile elements that employ an RNA intermediate that is first reverse transcribed into a complementary single-stranded (c) DNA strand by a reverse transcriptase encoded by the retrotransposon. Subsequently, the single-stranded DNA is converted into a double-stranded DNA that then integrates into the host genome. This so-called “replicative mechanism” yields several new copies of retrotransposons expanding throughout the target genome over evolutionary time. Retrotransposons are categorized into many subtypes according to the DNA sequences of the long terminal repeats and its open reading frames. Retrotransposons are employed to enable transgene integration into the target cell DNA, in some cases relying on adenoviral delivery. Alternatively, DNA transposons translocate via a “non-replicative mechanism,” whereby two Terminal Inverted Repeats (TIRs) are recognized and cleaved by a transposase enzyme, releasing the cognate DNA transposons with free DNA ends. The excised DNA transposons then integrate into a new genomic region where target sites are recognized and cut by the same transposase. This cut-and-paste mechanism usually duplicates DNA target sites upon insertion, leaving target site duplications (TSDs). Non-limiting examples of transposons include the Sleeping Beauty (SB) transposon, the piggyBac (PB) transposon, and Tol2 transposable elements.

### Inteins

Inteins (intervening protein) are auto-processing domains found in a variety of diverse organisms, which carry out a process known as protein splicing.

Non-limiting examples of inteins include any intein or intein-pair known in the art, which include a synthetic intein based on the dnaE intein, the Cfa-N (e.g., split intein-N) and Cfa-C (e.g., split intein-C) intein pair, has been described (e.g., in Stevens et al., J Am Chem Soc. 2016 Feb. 24; 138 (7): 2162-5, incorporated herein by reference), and DnaE. Non-limiting examples of pairs of inteins that may be used in accordance with the present disclosure include: Cfa DnaE intein, Ssp GyrB intein, Ssp DnaX intein, Ter DnaE3 intein, Ter Thy X intein, Rma DnaB intein and Cne Prp8 intein (e.g., as described in U.S. Pat. No. 8,394,604, incorporated herein by reference). Exemplary nucleotide and amino acid sequences of inteins are provided in the Sequence Listing at SEQ ID NOs: 370-377 and 389-424. Inteins suitable for use in embodiments of the present disclosure and methods for use thereof are described in U.S. Pat. No. 10,526,401, International Patent Application Publication No. WO 2013/045632 or WO 2020/051561, and in U.S. Patent Application Publication No. US 2020/0055900, the full disclosures of which are incorporated herein by reference in their entireties by reference for all purposes.

Intein-N and intein-C may be fused to the N-terminal portion of a split Cas9 and the C-terminal portion of the split Cas9, respectively, for the joining of the N-terminal portion of the split Cas9 and the C-terminal portion of the split Cas9. For example, in some embodiments, an intein-N is fused to the C-terminus of the N-terminal portion of the split Cas9, i.e., to form a structure of N—[N-terminal portion of the split Cas9]-[intein-N]—C. In some embodiments, an intein-C is fused to the N-terminus of the C-terminal portion of the split Cas9, i.e., to form a structure of N-[intein-C]—[C-terminal portion of the split Cas9]-C. In embodiments, a base editor is encoded by two polynucleotides, where one polynucleotide encodes a fragment of the base editor fused to an intein-N and another polynucleotide encodes a fragment of the base editor fused to an intein-C. Methods for designing and using inteins are known in the art and described, for example by WO2014004336, WO2017132580, WO2013045632A1, US20150344549, and US20180127780, each of which is incorporated herein by reference in their entirety.

In some embodiments, an ABE was split into N- and C-terminal fragments at Ala, Ser, Thr, or Cys residues within selected regions of SpCas9. These regions correspond to loop regions identified by Cas9 crystal structure analysis.

The N-terminus of each fragment is fused to an intein-N and the C-terminus of each fragment is fused to an intein C at amino acid positions S303, T310, T313, S355, A456, S460, A463, T466, S469, T472, T474, C574, S577, A589, and S590, referenced to SEQ ID NO: 197.

### Pharmaceutical Compositions

In some aspects, the present disclosure provides a pharmaceutical composition comprising any of the cells, polynucleotides, vectors, base editors, base editor systems, guide polynucleotides, fusion proteins, complexes, or the fusion protein-guide polynucleotide complexes described herein.

The pharmaceutical compositions of the present disclosure can be prepared in accordance with known techniques. See, e.g., Remington, The Science And Practice of Pharmacy (21st ed. 2005). In general, the cell, or population thereof is admixed with a suitable carrier prior to administration or storage, and in some embodiments, the pharmaceutical composition further comprises a pharmaceutically acceptable carrier. Suitable pharmaceutically acceptable carriers generally comprise inert substances that aid in administering the pharmaceutical composition to a subject, aid in processing the pharmaceutical compositions into deliverable preparations, or aid in storing the pharmaceutical composition prior to administration. Pharmaceutically acceptable carriers can include agents that can stabilize, optimize or otherwise alter the form, consistency, viscosity, pH, pharmacokinetics, solubility of the formulation. Such agents include buffering agents, wetting agents, emulsifying agents, diluents, encapsulating agents, and skin penetration enhancers. For example, carriers can include, but are not limited to, saline, buffered saline, dextrose, arginine, sucrose, water, glycerol, ethanol, sorbitol, dextran, sodium carboxymethyl cellulose, and combinations thereof.

In some embodiments, the pharmaceutical composition is formulated for delivery to a subject. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., the site of a neoplasia). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In some embodiments, any of the fusion proteins, gRNAs, and/or complexes described herein are provided as part of a pharmaceutical composition. In some embodiments, the pharmaceutical composition comprises any of the fusion proteins or complexes provided herein.

In some embodiments pharmaceutical composition comprises a gRNA, a nucleic acid programmable DNA binding protein, a cationic lipid, and a pharmaceutically acceptable excipient. In embodiments, pharmaceutical compositions comprise a lipid nanoparticle and a pharmaceutically acceptable excipient. In embodiments, the lipid nanoparticle contains a gRNA, a base editor, a complex, a base editor system, or a component thereof of the present disclosure, and/or one or more polynucleotides encoding the same. Pharmaceutical compositions can optionally comprise one or more additional therapeutically active substances.

The compositions, as described above, can be administered in effective amounts. The effective amount will depend upon the mode of administration, the particular condition being treated, and the desired outcome. It may also depend upon the stage of the condition, the age and physical condition of the subject, the nature of concurrent therapy, if any, and like factors well-known to the medical practitioner. For therapeutic applications, it is that amount sufficient to achieve a medically desirable result.

In some embodiments, compositions in accordance with the present disclosure can be used for treatment of any of a variety of diseases, disorders, and/or conditions.

### Methods of Treatment

Some aspects of the present disclosure provide methods of treating a subject in need, the method comprising administering to a subject in need an effective therapeutic amount of a pharmaceutical composition as described herein. More specifically, the methods of treatment include administering to a subject in need thereof one or more pharmaceutical compositions comprising one or more cells having at least one edited gene. In other embodiments, the methods of the disclosure comprise expressing or introducing into a cell a base editor polypeptide and one or more guide RNAs capable of targeting a nucleic acid molecule encoding at least one polypeptide.

One of ordinary skill in the art would recognize that multiple administrations of the pharmaceutical compositions contemplated in particular embodiments may be required to affect the desired therapy. For example, a composition may be administered to the subject 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

Administration of the pharmaceutical compositions contemplated herein may be carried out using conventional techniques including, but not limited to, infusion, transfusion, or parenterally. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally.

### Kits

The disclosure provides kits for the treatment of a disease or disorder (e.g., a neoplasia, such as a tumor or cancer) in a subject. In some embodiments, the kit includes a polypeptide of the disclosure (e.g., a base editor, beta-2-microglobulin, or a HLA class-I single-chain dimer or trimer) and/or a polynucleotide encoding the same. In some embodiments, the kit further includes a base editor system or a polynucleotide encoding a base editor system, wherein the base editor polypeptide system a nucleic acid programmable DNA binding protein (napDNAbp), a deaminase, and a guide RNA. In some embodiments, the napDNAbp is Cas9 or Cas12. In some embodiments, the polynucleotide encoding the base editor is a mRNA sequence. In some embodiments, the deaminase is a cytidine deaminase or an adenosine deaminase. In some embodiments, the kit comprises an edited cell and instructions regarding the use of the cell.

The kits may further comprise written instructions for using a polynucleotide, polypeptide, base editor, base editor system and/or edited cell as described herein. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit comprises instructions in the form of a label or separate insert (package insert) for suitable operational parameters. In yet another embodiment, the kit comprises one or more containers with appropriate positive and negative controls or control samples, to be used as standard(s) for detection, calibration, or normalization. The kit can further comprise a second container comprising a pharmaceutically-acceptable buffer, such as (sterile) phosphate-buffered saline, Ringer's solution, or dextrose solution. It can further include other materials desirable from a commercial and user standpoint, including other buffers, diluents, filters, needles, syringes, and package inserts with instructions for use.

The practice of embodiments of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing embodiments of the disclosure. Particularly useful techniques for particular embodiments will be discussed in the sections that follow.

The following examples are put forth so as to provide those of ordinary skill in the art with a complete disclosure and description of how to make and use the assay, screening, and therapeutic methods of the disclosure, and are not intended to limit the scope of what the inventors regard as their invention.

### EXAMPLES

### Example 1: HLA-Class I Attenuated Allogeneic Immune Cells Showed Improved Protection from Lysis by Immune Effector Cells

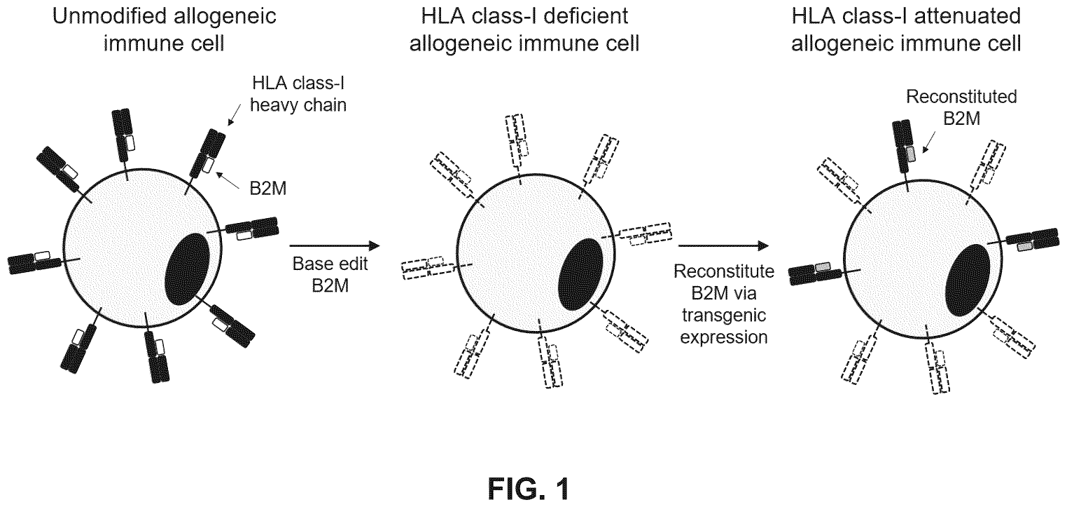

Experiments were undertaken to develop allogeneic T cells with improved resistance to lysis by alloreactive T cells by modifying allogeneic T cells to have attenuated surface-expression of HLA class-I polypeptides (see FIG. 1). Throughout the present Example, the allogeneic T cells were isolated from a leukopak blood collection from a de-identified healthy human donor. To prepare the allogeneic T cells with attenuated surface-expression of the HLA class-I polypeptides, HLA-A, HLA-B, and HLA-C, the allogeneic T cells were first edited to knock out endogenous expression of beta-2-microglobulin (B2M) by contacting the cells with a base editor (ABE8.20m) and a guide RNA (gRNA; see TSBTx760 of Table 1) targeting the base editor to modify a nucleobase of the B2M gene, where the modification knocked out expression of a functional B2M polypeptide encoded by the unedited B2M gene and yielded B2M knock-out cells (B2M KO). Next, B2M expression was reconstituted in the B2M KO cells by 1) expressing B2M in the cells under the control of a promoter that directed expression of reduced levels of B2M in the cells relative to levels in a wild-type cell; and 2) expressing in the cells a B2M polypeptide containing an amino acid alteration(s) (e.g., W60G, W95G) that reduces the affinity of B2M for HLA class-I heavy chain polypeptides, HLA-A, HLA-B, and HLA-C.

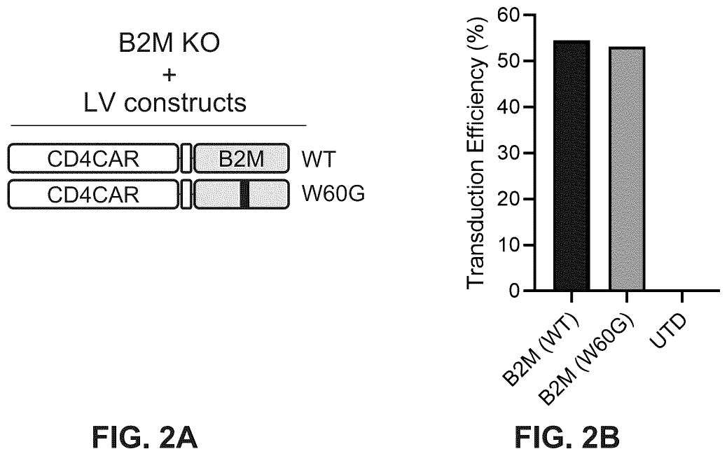

Lentiviral vectors were prepared containing polynucleotides encoding a chimeric antigen receptor (CAR) containing a CD4 extracellular domain and either a wild type B2M (B2M (WT)) polypeptide or a B2M polypeptide containing a W60G modification (B2M (W60G)) expressed under the control of the human EF1-alpha promoter (FIG. 2A; GCTCCGGTGCCCGTCAGTGGGCAGAGCGCACATCGCCCACAGTCCCCGAGAAGTTGGGGGGAGG GGTCGGCAATTGAACCGGTGCCTAGAGAAGGTGGCGCGGGGTAAACTGGGAAAGTGATGTCGTG TACTGGCTCCGCCTTTTTCCCGAGGGTGGGGGAGAACCGTATATAAGTGCAGTAGTCGCCGTGA ACGTTCTTTTTCGCAACGGGTTTGCCGCCAGAACACAGGTAAGTGCCGTGTGTGGTTCCCGCGG GCCTGGCCTCTTTACGGGTTATGGCCCTTGCGTGCCTTGAATTACTTCCACGCCCCTGGCTGCA GTACGTGATTCTTGATCCCGAGCTTCGGGTTGGAAGTGGGTGGGAGAGTTCGAGGCCTTGCGCT TAAGGAGCCCCTTCGCCTCGTGCTTGAGTTGAGGCCTGGCCTGGGCGCTGGGGCCGCCGCGTGC GAATCTGGTGGCACCTTCGCGCCTGTCTCGCTGCTTTCGATAAGTCTCTAGCCATTTAAAATTT TTGATGACCTGCTGCGACGCTTTTTTTCTGGCAAGATAGTCTTGTAAATGCGGGCCAAGATCTG CACACTGGTATTTCGGTTTTTGGGGCCGCGGGCGGCGACGGGGCCCGTGCGTCCCAGCGCACAT GTTCGGCGAGGCGGGGCCTGCGAGCGCGGCCACCGAGAATCGGACGGGGGTAGTCTCAAGCTGG CCGGCCTGCTCTGGTGCCTGGCCTCGCGCCGCCGTGTATCGCCCCGCCCTGGGCGGCAAGGCTG GCCCGGTCGGCACCAGTTGCGTGAGCGGAAAGATGGCCGCTTCCCGGCCCTGCTGCAGGGAGCT CAAAATGGAGGACGCGGCGCTCGGGAGAGCGGGGGGGTGAGTCACCCACACAAAGGAAAAGGGC CTTTCCGTCCTCAGCCGTCGCTTCATGTGACTCCACGGAGTACCGGGCGCCGTCCAGGCACCTC GATTAGTTCTCGAGCTTTTGGAGTACGTCGTCTTTAGGTTGGGGGGAGGGGTTTTATGCGATGG AGTTTCCCCACACTGAGTGGGTGGAGACTGAAGTTAGGCCAGCTTGGCACTTGATGTAATTCTC CTTGGAATTTGCCCTTTTTGAGTTTGGATCTTGGTTCATTCTCAAGCCTCAGACAGTGGTTCAA AGTTTTTTTCTTCCATTTCAGGTGTCGTGA (SEQ ID NO: 627)). The lentiviral vectors had a transduction efficiency of at least 50% (FIG. 2B). The lentiviral vectors were used to transfect β2M KO cells and reconstitute β2M expression in the cells.

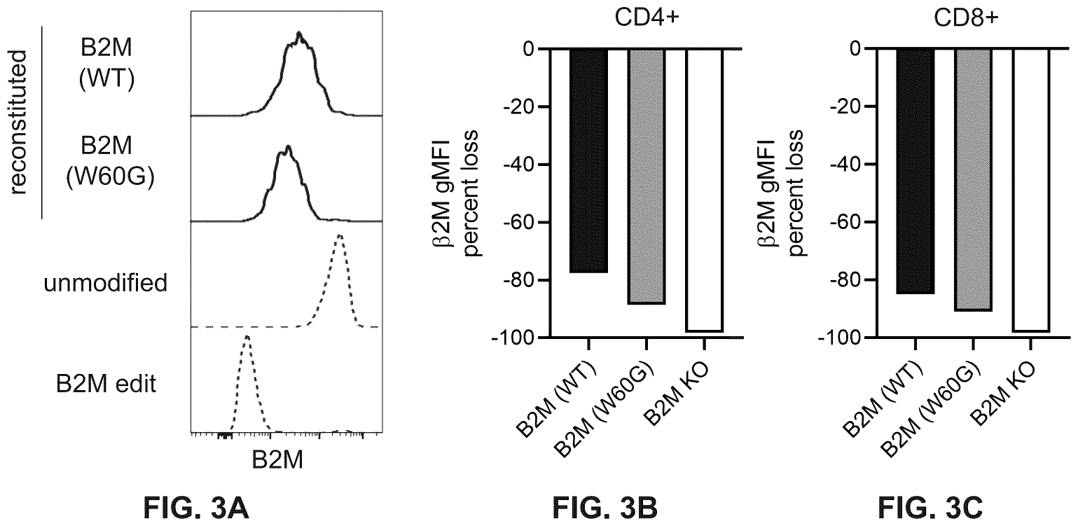

Surface-expression of endogenous and transfected β2M was measured using FACS analysis. The β2M KO cells transfected using the lentiviral vectors showed reduced levels of β2M surface-expression relative to wild-type T cells (FIGS. 3A-3C). In particular, surface-expression of β2M in β2M KO CD4+ T cells or CD8+ T cells expressing β2M WT under the control of the EF1a promoter was lower than that of wild-type T cells (FIGS. 3B and 3C), and surface expression of β2M in β2M KO CD4+ T cells or CD8+ T cells expressing β2M (W60G) under the control of the EF1a promoter was even lower (FIGS. 3B and 3C).

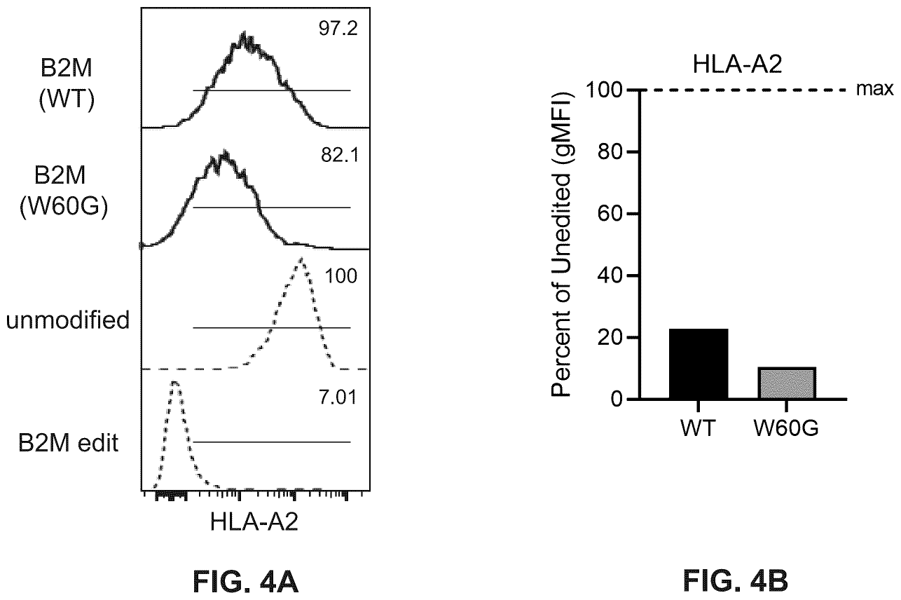

Reconstitution of β2M expression in the β2M KO cells restored surface-expression of HLA-A2 in the cells (FIGS. 4A and 4B), but at reduced levels relative to wild type cells (between about 10% and 25% of levels in the wild type cells).

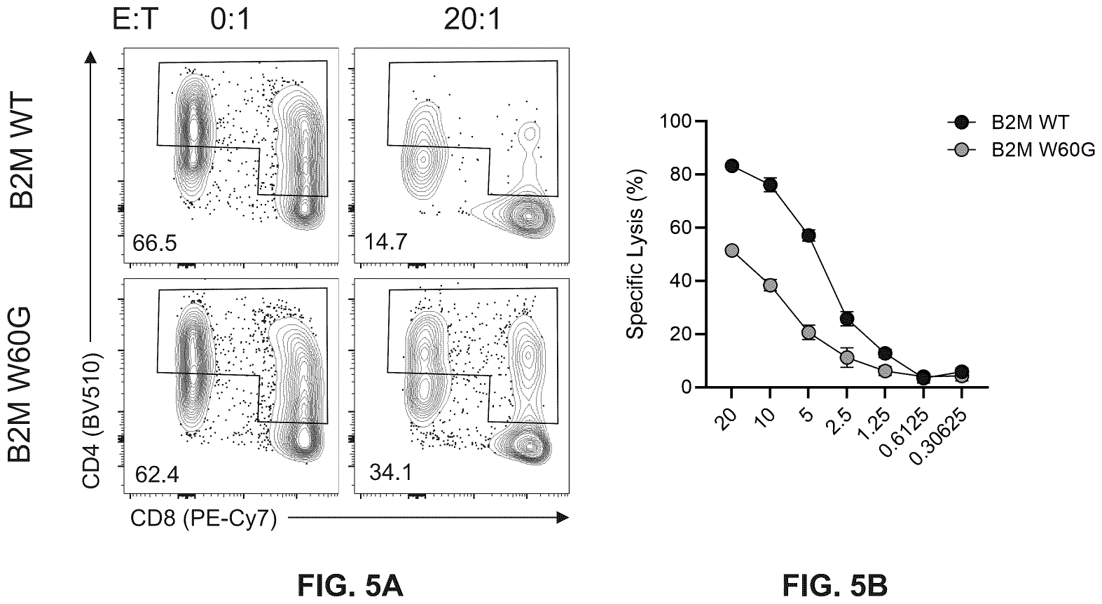

Experiments were next undertaken to determine if the B2M KO cells expressing B2M (W60G) under the control of the EF1a promoter showed improved resistance to lysis by alloreactive T cells relative to B2M KO cells expressing B2M (WT) under the control of the EF1a promoter (FIGS. 5A and 5B). Cell lysis was evaluated using a T cell mixed leukocyte reaction, where the effector cells (E) were mismatched (i.e., alloreactive) T cells and the target cells (T) were the B2M KO cells expressing B2M (W60G) or B2M (WT) to reconstitute B2M expression. Cell lysis was measured after 48 hours of co-culture. Cells were co-cultured at effector cell (E) to target cell (T) ratios of 0, 0.30625, 0.6125, 1.25, 2.5, 5, 10, and 20. It was determined that B2M KO T cells expressing B2M (W60G) were less susceptible to lysis by alloreactive T cells than B2M KO T cells expressing B2M (WT). Therefore, the B2M (W60G) polypeptides protected the B2M KO T cells from alloreactive T cell lysis.

### Example 2: Expression of HLA Class-I Single Chain Dimer Variants Protected Allogeneic Immune Cells from Lysis by Alloreactive T Cells and Natural Killer (NK) Cells

Another approach to avoid activation of a subject's alloreactive T cells by allogeneic immune cells administered to the subject is through the interruption of interactions between mismatched HLA class-I polypeptides expressed on the surface of the allogeneic immune cells and T cell receptor complexes expressed on the surface of the alloreactive T cells (FIGS. 6 and 7C). In particular, activation of the alloreactive T cells can be reduced by modifying the allogeneic immune cells to express HLA class-I polypeptides with reduced affinity for CD8 polypeptides expressed on the surface of the allogeneic immune cells. For example, affinity of a beta-2-microglobulin (B2M) polypeptide for interaction with CD8 can be reduced by introducing a K58E alteration to the B2M polypeptide (FIG. 6), and affinity of an HLA-A, -B, or -C polypeptide for interactions with CD8 can be reduced by introducing a D227K, T228A, and/or A245V alteration to the HLA-A, -B, or -C polypeptide, respectively (FIG. 7C). Therefore, experiments were undertaken to improve resistance of allogeneic immune cells to lysis by alloreactive T cells by modifying the allogeneic immune cells to express HLA class-I polypeptides containing one or more of these alterations. Throughout the present Example, the allogeneic T cells were isolated from a leukopak blood collection from a de-identified healthy human donor.

As a first step in preparing the modified allogeneic immune cells with improved resistance to lysis by alloreactive T cells, HLA class-I single-chain dimers (FIG. 7A) and HLA class-I single-chain trimers (FIG. 7B) were designed. Amino acid and nucleotide sequences for the HLA class-I single chain dimers and trimers are provided in Tables 8A and 8B below, respectively. The HLA class-I single chain dimers contained, from N-terminus to C-terminus, a signal peptide, a B2M domain, a linker, and an HLA class-I heavy chain domain (HLA-X, where HLA-X represents a polypeptide derived from HLA-A, -B, or -C). The HLA class-I single chain trimers contained, from N-terminus to C-terminus, a signal peptide, a cognate peptide, a linker, a B2M domain, a linker, an HLA class-I heavy chain domain (HLA-X, where HLA-X represents a polypeptide derived from HLA-A, -B, or -C). Some of the HLA class-I heavy chain domains contained alterations (e.g., D227K, T228A, and/or A245V) that reduced or abrogated CD8 binding.

TABLE 8A
Amino acid sequences for HLA class-I single-chain dimers and trimers.1

| Description | | | | |
|---|---|---|---|---|
| of | | | SEQ | |
| polypeptide | ID# | Amino Acid Seq | ID NO | Format |
| B2M/HLA- | 230 | MSRSVALAVLALLSLSGLEAIQRTPKIQVYSRHPAENGK | 454 | single |
| A*02 | | SNFLNCYVSGFHPSDIEVDLLKNGERIEKVEHSDLSFSK | | chain |
| | | DWSFYLLYYTEFTPTEKDEYACRVNHVTLSQPKIVKWDR | | dimer |
| | | DMGGGGSGGGGSGGGGSGGGGSGSHSMRYFFTSVSRPGR | | |
| | | GEPRFIAVGYVDDTQFVRFDSDAASQRMEPRAPWIEQEG | | |
| | | PEYWDGETRKVKAHSQTHRVDLGTLRGYYNQSEAGSHTV | | |
| | | QRMYGCDVGSDWRFLRGYHQYAYDGKDYIALKEDLRSWT | | |
| | | AADMAAQTTKHKWEAAHVAEQLRAYLEGTCVEWLRRYLE | | |
| | | NGKETLQRTDAPKTHMTHHAVSDHEATLRCWALSFYPAE | | |
| | | ITLTWQRDGEDQTQDTELVETRPAGDGTFQKWAAVVVPS | | |
| | | GQEQRYTCHVQHEGLPKPLTLRWEPSSQPTIPIVGIIAG | | |
| | | LVLFGAVITGAVVAAVMWRRKSSDRKGGSYSQAASSDSA | | |
| | | QGSDVSLTACKV | | |
| B2M/HLA- | 231 | MSRSVALAVLALLSLSGLEAIQRTPKIQVYSRHPAENGK | 455 | single |
| A*02 | | SNFLNCYVSGFHPSDIEVDLLKNGERIEKVEHSDLSFSK | | chain |
| (D227K/T2 | | DWSFYLLYYTEFTPTEKDEYACRVNHVTLSQPKIVKWDR | | dimer |
| 28A) | | DMGGGGSGGGGSGGGGSGGGGSGSHSMRYFFTSVSRPGR | | |
| | | GEPRFIAVGYVDDTQFVRFDSDAASQRMEPRAPWIEQEG | | |
| | | PEYWDGETRKVKAHSQTHRVDLGTLRGYYNQSEAGSHTV | | |
| | | QRMYGCDVGSDWRFLRGYHQYAYDGKDYIALKEDLRSWT | | |
| | | AADMAAQTTKHKWEAAHVAEQLRAYLEGTCVEWLRRYLE | | |
| | | NGKETLQRTDAPKTHMTHHAVSDHEATLRCWALSFYPAE | | |
| | | ITLTWQRDGEDQTQKAELVETRPAGDGTFQKWAAVVVPS | | |
| | | GQEQRYTCHVQHEGLPKPLTLRWEPSSQPTIPIVGIIAG | | |
| | | LVLFGAVITGAVVAAVMWRRKSSDRKGGSYSQAASSDSA | | |
| | | QGSDVSLTACKV | | |
| B2M/HLA- | 232 | MSRSVALAVLALLSLSGLEAIQRTPKIQVYSRHPAENGK | 456 | single |
| A*02 | | SNFLNCYVSGFHPSDIEVDLLKNGERIEKVEHSDLSFSK | | chain |
| (A245V) | | DWSFYLLYYTEFTPTEKDEYACRVNHVTLSQPKIVKWDR | | dimer |
| | | DMGGGGSGGGGSGGGGSGGGGSGSHSMRYFFTSVSRPGR | | |
| | | GEPRFIAVGYVDDTQFVRFDSDAASQRMEPRAPWIEQEG | | |
| | | PEYWDGETRKVKAHSQTHRVDLGTLRGYYNQSEAGSHTV | | |
| | | QRMYGCDVGSDWRFLRGYHQYAYDGKDYIALKEDLRSWT | | |
| | | AADMAAQTTKHKWEAAHVAEQLRAYLEGTCVEWLRRYLE | | |
| | | NGKETLQRTDAPKTHMTHHAVSDHEATLRCWALSFYPAE | | |
| | | ITLTWQRDGEDQTQDTELVETRPAGDGTFQKWVAVVVPS | | |
| | | GQEQRYTCHVQHEGLPKPLTLRWEPSSQPTIPIVGIIAG | | |
| | | LVLFGAVITGAVVAAVMWRRKSSDRKGGSYSQAASSDSA | | |
| | | QGSDVSLTACKV | | |
| B2M/HLA- | 233 | MSRSVALAVLALLSLSGLEAIQRTPKIQVYSRHPAENGK | 457 | single |
| A*02 | | SNFLNCYVSGFHPSDIEVDLLKNGERIEKVEHSDLSFSK | | chain |
| (D227K/ | | DWSFYLLYYTEFTPTEKDEYACRVNHVTLSQPKIVKWDR | | dimer |
| T228A/A245V) | | DMGGGGSGGGGSGGGGSGGGGSGSHSMRYFFTSVSRPGR | | |
| | | GEPRFIAVGYVDDTQFVRFDSDAASQRMEPRAPWIEQEG | | |
| | | PEYWDGETRKVKAHSQTHRVDLGTLRGYYNQSEAGSHTV | | |
| | | QRMYGCDVGSDWRFLRGYHQYAYDGKDYIALKEDLRSWT | | |
| | | AADMAAQTTKHKWEAAHVAEQLRAYLEGTCVEWLRRYLE | | |
| | | NGKETLQRTDAPKTHMTHHAVSDHEATLRCWALSFYPAE | | |
| | | ITLTWQRDGEDQTQKAELVETRPAGDGTFQKWVAVVVPS | | |
| | | GQEQRYTCHVQHEGLPKPLTLRWEPSSQPTIPIVGIIAG | | |
| | | LVLFGAVITGAVVAAVMWRRKSSDRKGGSYSQAASSDSA | | |
| | | QGSDVSLTACKV | | |
| B2M(K58E)/ | 234 | MSRSVALAVLALLSLSGLEAIQRTPKIQVYSRHPAENGK | 458 | single |
| HLA-A*02 | | SNFLNCYVSGFHPSDIEVDLLKNGERIEKVEHSDLSFSE | | chain |
| | | DWSFYLLYYTEFTPTEKDEYACRVNHVTLSQPKIVKWDR | | dimer |
| | | DMGGGGSGGGGSGGGGSGGGGSGSHSMRYFFTSVSRPGR | | |
| | | GEPRFIAVGYVDDTQFVRFDSDAASQRMEPRAPWIEQEG | | |
| | | PEYWDGETRKVKAHSQTHRVDLGTLRGYYNQSEAGSHTV | | |
| | | QRMYGCDVGSDWRFLRGYHQYAYDGKDYIALKEDLRSWT | | |
| | | AADMAAQTTKHKWEAAHVAEQLRAYLEGTCVEWLRRYLE | | |
| | | NGKETLQRTDAPKTHMTHHAVSDHEATLRCWALSFYPAE | | |
| | | ITLTWQRDGEDQTQDTELVETRPAGDGTFQKWAAVVVPS | | |
| | | GQEQRYTCHVQHEGLPKPLTLRWEPSSQPTIPIVGIIAG | | |
| | | LVLFGAVITGAVVAAVMWRRKSSDRKGGSYSQAASSDSA | | |
| | | QGSDVSLTACKV | | |
| B2M(K58E)/ | 235 | MSRSVALAVLALLSLSGLEAIQRTPKIQVYSRHPAENGK | 459 | single |
| HLA- | | SNFLNCYVSGFHPSDIEVDLLKNGERIEKVEHSDLSFSE | | chain |
| A*02 | | DWSFYLLYYTEFTPTEKDEYACRVNHVTLSQPKIVKWDR | | dimer |
| (D227K/T228A) | | DMGGGGSGGGGSGGGGSGGGGSGSHSMRYFFTSVSRPGR | | |
| | | GEPRFIAVGYVDDTQFVRFDSDAASQRMEPRAPWIEQEG | | |
| | | PEYWDGETRKVKAHSQTHRVDLGTLRGYYNQSEAGSHTV | | |
| | | QRMYGCDVGSDWRFLRGYHQYAYDGKDYIALKEDLRSWT | | |
| | | AADMAAQTTKHKWEAAHVAEQLRAYLEGTCVEWLRRYLE | | |
| | | NGKETLQRTDAPKTHMTHHAVSDHEATLRCWALSFYPAE | | |
| | | ITLTWQRDGEDQTQKAELVETRPAGDGTFQKWAAVVVPS | | |
| | | GQEQRYTCHVQHEGLPKPLTLRWEPSSQPTIPIVGIIAG | | |
| | | LVLFGAVITGAVVAAVMWRRKSSDRKGGSYSQAASSDSA | | |
| | | QGSDVSLTACKV | | |
| B2M(K58E)/ | 236 | MSRSVALAVLALLSLSGLEAIQRTPKIQVYSRHPAENGK | 460 | single |
| HLA- | | SNFLNCYVSGFHPSDIEVDLLKNGERIEKVEHSDLSFSE | | chain |
| A*02(A245V) | | DWSFYLLYYTEFTPTEKDEYACRVNHVTLSQPKIVKWDR | | dimer |
| | | DMGGGGSGGGGSGGGGSGGGGSGSHSMRYFFTSVSRPGR | | |
| | | GEPRFIAVGYVDDTQFVRFDSDAASQRMEPRAPWIEQEG | | |
| | | PEYWDGETRKVKAHSQTHRVDLGTLRGYYNQSEAGSHTV | | |
| | | QRMYGCDVGSDWRFLRGYHQYAYDGKDYIALKEDLRSWT | | |
| | | AADMAAQTTKHKWEAAHVAEQLRAYLEGTCVEWLRRYLE | | |
| | | NGKETLQRTDAPKTHMTHHAVSDHEATLRCWALSFYPAE | | |
| | | ITLTWQRDGEDQTQDTELVETRPAGDGTFQKWVAVVVPS | | |
| | | GQEQRYTCHVQHEGLPKPLTLRWEPSSQPTIPIVGIIAG | | |
| | | LVLFGAVITGAVVAAVMWRRKSSDRKGGSYSQAASSDSA | | |
| | | QGSDVSLTACKV | | |
| B2M(K58E)/ | 237 | MSRSVALAVLALLSLSGLEAIQRTPKIQVYSRHPAENGK | 461 | single |
| HLA- | | SNFLNCYVSGFHPSDIEVDLLKNGERIEKVEHSDLSFSE | | chain |
| A*02(D227K/ | | DWSFYLLYYTEFTPTEKDEYACRVNHVTLSQPKIVKWDR | | dimer |
| T228A/A245V) | | DMGGGGSGGGGSGGGGSGGGGSGSHSMRYFFTSVSRPGR | | |
| | | GEPRFIAVGYVDDTQFVRFDSDAASQRMEPRAPWIEQEG | | |
| | | PEYWDGETRKVKAHSQTHRVDLGTLRGYYNQSEAGSHTV | | |
| | | QRMYGCDVGSDWRFLRGYHQYAYDGKDYIALKEDLRSWT | | |
| | | AADMAAQTTKHKWEAAHVAEQLRAYLEGTCVEWLRRYLE | | |
| | | NGKETLQRTDAPKTHMTHHAVSDHEATLRCWALSFYPAE | | |
| | | ITLTWQRDGEDQTQKAELVETRPAGDGTFQKWVAVVVPS | | |
| | | GQEQRYTCHVQHEGLPKPLTLRWEPSSQPTIPIVGIIAG | | |
| | | LVLFGAVITGAVVAAVMWRRKSSDRKGGSYSQAASSDSA | | |
| | | QGSDVSLTACKV | | |
| B2M/HLA- | 238 | MSRSVALAVLALLSLSGLEAIQRTPKIQVYSRHPAENGK | 462 | single |
| A*02- | | SNFLNCYVSGFHPSDIEVDLLKNGERIEKVEHSDLSFSK | | chain |
| CD4TM | | DWSFYLLYYTEFTPTEKDEYACRVNHVTLSQPKIVKWDR | | dimer |
| | | DMGGGGSGGGGSGGGGSGGGGSGSHSMRYFFTSVSRPGR | | |
| | | GEPRFIAVGYVDDTQFVRFDSDAASQRMEPRAPWIEQEG | | |
| | | PEYWDGETRKVKAHSQTHRVDLGTLRGYYNQSEAGSHTV | | |
| | | QRMYGCDVGSDWRFLRGYHQYAYDGKDYIALKEDLRSWT | | |
| | | AADMAAQTTKHKWEAAHVAEQLRAYLEGTCVEWLRRYLE | | |
| | | NGKETLQRTDAPKTHMTHHAVSDHEATLRCWALSFYPAE | | |
| | | ITLTWQRDGEDQTQDTELVETRPAGDGTFQKWAAVVVPS | | |
| | | GQEQRYTCHVQHEGLPKPLTLRWEPSSQPTIPIMALIVL | | |
| | | GGVAGLLLFIGLGIFFCVRC | | |
| B2M/HLA- | 239 | MSRSVALAVLALLSLSGLEAIQRTPKIQVYSRHPAENGK | 463 | single |
| A*02(D227K/ | | SNFLNCYVSGFHPSDIEVDLLKNGERIEKVEHSDLSFSK | | chain |
| T228A)- | | DWSFYLLYYTEFTPTEKDEYACRVNHVTLSQPKIVKWDR | | dimer |
| CD4TM | | DMGGGGSGGGGSGGGGSGGGGSGSHSMRYFFTSVSRPGR | | |
| | | GEPRFIAVGYVDDTQFVRFDSDAASQRMEPRAPWIEQEG | | |
| | | PEYWDGETRKVKAHSQTHRVDLGTLRGYYNQSEAGSHTV | | |
| | | QRMYGCDVGSDWRFLRGYHQYAYDGKDYIALKEDLRSWT | | |
| | | AADMAAQTTKHKWEAAHVAEQLRAYLEGTCVEWLRRYLE | | |
| | | NGKETLQRTDAPKTHMTHHAVSDHEATLRCWALSFYPAE | | |
| | | ITLTWQRDGEDQTQKAELVETRPAGDGTFQKWAAVVVPS | | |
| | | GQEQRYTCHVQHEGLPKPLTLRWEPSSQPTIPIMALIVL | | |
| | | GGVAGLLLFIGLGIFFCVRC | | |
| B2M/HLA- | 240 | MSRSVALAVLALLSLSGLEAIQRTPKIQVYSRHPAENGK | 464 | single |
| A*02(A245V)- | | SNFLNCYVSGFHPSDIEVDLLKNGERIEKVEHSDLSFSK | | chain |
| CD4TM | | DWSFYLLYYTEFTPTEKDEYACRVNHVTLSQPKIVKWDR | | dimer |
| | | DMGGGGSGGGGSGGGGSGGGGSGSHSMRYFFTSVSRPGR | | |
| | | GEPRFIAVGYVDDTQFVRFDSDAASQRMEPRAPWIEQEG | | |
| | | PEYWDGETRKVKAHSQTHRVDLGTLRGYYNQSEAGSHTV | | |
| | | QRMYGCDVGSDWRFLRGYHQYAYDGKDYIALKEDLRSWT | | |
| | | AADMAAQTTKHKWEAAHVAEQLRAYLEGTCVEWLRRYLE | | |
| | | NGKETLQRTDAPKTHMTHHAVSDHEATLRCWALSFYPAE | | |
| | | ITLTWQRDGEDQTQDTELVETRPAGDGTFQKWVAVVVPS | | |
| | | GQEQRYTCHVQHEGLPKPLTLRWEPSSQPTIPIMALIVL | | |
| | | GGVAGLLLFIGLGIFFCVRC | | |
| B2M/HLA- | 241 | MSRSVALAVLALLSLSGLEAIQRTPKIQVYSRHPAENGK | 465 | single |
| A*02(D227K/ | | SNFLNCYVSGFHPSDIEVDLLKNGERIEKVEHSDLSFSK | | chain |
| T228A/A245V)- | | DWSFYLLYYTEFTPTEKDEYACRVNHVTLSQPKIVKWDR | | dimer |
| CD4TM | | DMGGGGSGGGGSGGGGSGGGGSGSHSMRYFFTSVSRPGR | | |
| | | GEPRFIAVGYVDDTQFVRFDSDAASQRMEPRAPWIEQEG | | |
| | | PEYWDGETRKVKAHSQTHRVDLGTLRGYYNQSEAGSHTV | | |
| | | QRMYGCDVGSDWRFLRGYHQYAYDGKDYIALKEDLRSWT | | |
| | | AADMAAQTTKHKWEAAHVAEQLRAYLEGTCVEWLRRYLE | | |
| | | NGKETLQRTDAPKTHMTHHAVSDHEATLRCWALSFYPAE | | |
| | | ITLTWQRDGEDQTQKAELVETRPAGDGTFQKWVAVVVPS | | |
| | | GQEQRYTCHVQHEGLPKPLTLRWEPSSQPTIPIMALIVL | | |
| | | GGVAGLLLFIGLGIFFCVRC | | |
| B2M(K58E)/ | 242 | MSRSVALAVLALLSLSGLEAIQRTPKIQVYSRHPAENGK | 466 | single |
| HLA-A*02- | | SNFLNCYVSGFHPSDIEVDLLKNGERIEKVEHSDLSFSE | | chain |
| CD4TM | | DWSFYLLYYTEFTPTEKDEYACRVNHVTLSQPKIVKWDR | | dimer |
| | | DMGGGGSGGGGSGGGGSGGGGSGSHSMRYFFTSVSRPGR | | |
| | | GEPRFIAVGYVDDTQFVRFDSDAASQRMEPRAPWIEQEG | | |
| | | PEYWDGETRKVKAHSQTHRVDLGTLRGYYNQSEAGSHTV | | |
| | | QRMYGCDVGSDWRFLRGYHQYAYDGKDYIALKEDLRSWT | | |
| | | AADMAAQTTKHKWEAAHVAEQLRAYLEGTCVEWLRRYLE | | |
| | | NGKETLQRTDAPKTHMTHHAVSDHEATLRCWALSFYPAE | | |
| | | ITLTWQRDGEDQTQDTELVETRPAGDGTFQKWAAVVVPS | | |
| | | GQEQRYTCHVQHEGLPKPLTLRWEPSSQPTIPIMALIVL | | |
| | | GGVAGLLLFIGLGIFFCVRC | | |
| B2M(K58E)/ | 243 | MSRSVALAVLALLSLSGLEAIQRTPKIQVYSRHPAENGK | 467 | single |
| HLA- | | SNFLNCYVSGFHPSDIEVDLLKNGERIEKVEHSDLSFSE | | chain |
| A*02(D227K/ | | DWSFYLLYYTEFTPTEKDEYACRVNHVTLSQPKIVKWDR | | dimer |
| T228A)- | | DMGGGGSGGGGSGGGGSGGGGSGSHSMRYFFTSVSRPGR | | |
| CD4TM | | GEPRFIAVGYVDDTQFVRFDSDAASQRMEPRAPWIEQEG | | |
| | | PEYWDGETRKVKAHSQTHRVDLGTLRGYYNQSEAGSHTV | | |
| | | QRMYGCDVGSDWRFLRGYHQYAYDGKDYIALKEDLRSWT | | |
| | | AADMAAQTTKHKWEAAHVAEQLRAYLEGTCVEWLRRYLE | | |
| | | NGKETLQRTDAPKTHMTHHAVSDHEATLRCWALSFYPAE | | |
| | | ITLTWQRDGEDQTQKAELVETRPAGDGTFQKWAAVVVPS | | |
| | | GQEQRYTCHVQHEGLPKPLTLRWEPSSQPTIPIMALIVL | | |
| | | GGVAGLLLFIGLGIFFCVRC | | |
| B2M(K58E)/ | 244 | MSRSVALAVLALLSLSGLEAIQRTPKIQVYSRHPAENGK | 468 | single |
| HLA- | | SNFLNCYVSGFHPSDIEVDLLKNGERIEKVEHSDLSFSE | | chain |
| A*02(A245V)- | | DWSFYLLYYTEFTPTEKDEYACRVNHVTLSQPKIVKWDR | | dimer |
| CD4TM | | DMGGGGSGGGGSGGGGSGGGGSGSHSMRYFFTSVSRPGR | | |
| | | GEPRFIAVGYVDDTQFVRFDSDAASQRMEPRAPWIEQEG | | |
| | | PEYWDGETRKVKAHSQTHRVDLGTLRGYYNQSEAGSHTV | | |
| | | QRMYGCDVGSDWRFLRGYHQYAYDGKDYIALKEDLRSWT | | |
| | | AADMAAQTTKHKWEAAHVAEQLRAYLEGTCVEWLRRYLE | | |
| | | NGKETLQRTDAPKTHMTHHAVSDHEATLRCWALSFYPAE | | |
| | | ITLTWQRDGEDQTQDTELVETRPAGDGTFQKWVAVVVPS | | |
| | | GQEQRYTCHVQHEGLPKPLTLRWEPSSQPTIPIMALIVL | | |
| | | GGVAGLLLFIGLGIFFCVRC | | |
| B2M(K58E)/ | 245 | MSRSVALAVLALLSLSGLEAIQRTPKIQVYSRHPAENGK | 469 | single |
| HLA- | | SNFLNCYVSGFHPSDIEVDLLKNGERIEKVEHSDLSFSE | | chain |
| A*02(D227K/ | | DWSFYLLYYTEFTPTEKDEYACRVNHVTLSQPKIVKWDR | | dimer |
| T228A/A245V)- | | DMGGGGSGGGGSGGGGSGGGGSGSHSMRYFFTSVSRPGR | | |
| CD4TM | | GEPRFIAVGYVDDTQFVRFDSDAASQRMEPRAPWIEQEG | | |
| | | PEYWDGETRKVKAHSQTHRVDLGTLRGYYNQSEAGSHTV | | |
| | | QRMYGCDVGSDWRFLRGYHQYAYDGKDYIALKEDLRSWT | | |
| | | AADMAAQTTKHKWEAAHVAEQLRAYLEGTCVEWLRRYLE | | |
| | | NGKETLQRTDAPKTHMTHHAVSDHEATLRCWALSFYPAE | | |
| | | ITLTWQRDGEDQTQKAELVETRPAGDGTFQKWVAVVVPS | | |
| | | GQEQRYTCHVQHEGLPKPLTLRWEPSSQPTIPIMALIVL | | |
| | | GGVAGLLLFIGLGIFFCVRC | | |
| B2M/HLA- | 246 | MSRSVALAVLALLSLSGLEAIQRTPKIQVYSRHPAENGK | 470 | single |
| C*05(C1G) | | SNFLNCYVSGFHPSDIEVDLLKNGERIEKVEHSDLSFSK | | chain |
| | | DWSFYLLYYTEFTPTEKDEYACRVNHVTLSQPKIVKWDR | | dimer |
| | | DMGGGGSGGGGSGGGGSGGGGSGSHSMRYFYTAVSRPGR | | |
| | | GEPRFIAVGYVDDTQFVQFDSDAASPRGEPRAPWVEQEG | | |
| | | PEYWDRETQKYKRQAQTDRVNLRKLRGYYNQSEAGSHTL | | |
| | | QRMYGCDLGPDGRLLRGYNQFAYDGKDYIALNEDLRSWT | | |
| | | AADKAAQITQRKWEAAREAEQRRAYLEGTCVEWLRRYLE | | |
| | | NGKKTLQRAEHPKTHVTHHPVSDHEATLRCWALGFYPAE | | |
| | | ITLTWQRDGEDQTQDTELVETRPAGDGTFQKWAAVVVPS | | |
| | | GEEQRYTCHVQHEGLPEPLTLRWGPSSQPTIPIVGIVAG | | |
| | | LAVLAVLAVLGAVMAVVMCRRKSSGGKGGSCSQAASSNS | | |
| | | AQGSDESLIACKA | | |
| B2M/HLA- | 247 | MSRSVALAVLALLSLSGLEAIQRTPKIQVYSRHPAENGK | 471 | single |
| C*05(C1G)- | | SNFLNCYVSGFHPSDIEVDLLKNGERIEKVEHSDLSFSK | | chain |
| CD4TM | | DWSFYLLYYTEFTPTEKDEYACRVNHVTLSQPKIVKWDR | | dimer |
| | | DMGGGGSGGGGSGGGGSGGGGSGSHSMRYFYTAVSRPGR | | |
| | | GEPRFIAVGYVDDTQFVQFDSDAASPRGEPRAPWVEQEG | | |
| | | PEYWDRETQKYKRQAQTDRVNLRKLRGYYNQSEAGSHTL | | |
| | | QRMYGCDLGPDGRLLRGYNQFAYDGKDYIALNEDLRSWT | | |
| | | AADKAAQITQRKWEAAREAEQRRAYLEGTCVEWLRRYLE | | |
| | | NGKKTLQRAEHPKTHVTHHPVSDHEATLRCWALGFYPAE | | |
| | | ITLTWQRDGEDQTQDTELVETRPAGDGTFQKWAAVVVPS | | |
| | | GEEQRYTCHVQHEGLPEPLTLRWGPSSQPTIPIMALIVL | | |
| | | GGVAGLLLFIGLGIFFCVRC | | |
| B2M/HLA- | 248 | MSRSVALAVLALLSLSGLEAIQRTPKIQVYSRHPAENGK | 472 | single |
| C*04 | | SNFLNCYVSGFHPSDIEVDLLKNGERIEKVEHSDLSFSK | | chain |
| | | DWSFYLLYYTEFTPTEKDEYACRVNHVTLSQPKIVKWDR | | dimer |
| | | DMGGGGSGGGGSGGGGSGGGGSGSHSMRYFSTSVSWPGR | | |
| | | GEPRFIAVGYVDDTQFVRFDSDAASPRGEPREPWVEQEG | | |
| | | PEYWDRETQKYKRQAQADRVNLRKLRGYYNQSEDGSHTL | | |
| | | QRMFGCDLGPDGRLLRGYNQFAYDGKDYIALNEDLRSWT | | |
| | | AADTAAQITQRKWEAAREAEQRRAYLEGTCVEWLRRYLE | | |
| | | NGKETLQRAEHPKTHVTHHPVSDHEATLRCWALGFYPAE | | |
| | | ITLTWQWDGEDQTQDTELVETRPAGDGTFQKWAAVVVPS | | |
| | | GEEQRYTCHVQHEGLPEPLTLRWKPSSQPTIPIVGIVAG | | |
| | | LAVLAVLAVLGAMVAVVMCRRKSSGGKGGSCSQAASSNS | | |
| | | AQGSDESLIACKA | | |
| B2M/HLA- | 249 | MSRSVALAVLALLSLSGLEAIQRTPKIQVYSRHPAENGK | 473 | single |
| C*04- | | SNFLNCYVSGFHPSDIEVDLLKNGERIEKVEHSDLSFSK | | chain |
| CD4TM | | DWSFYLLYYTEFTPTEKDEYACRVNHVTLSQPKIVKWDR | | dimer |
| | | DMGGGGSGGGGSGGGGSGGGGSGSHSMRYFSTSVSWPGR | | |
| | | GEPRFIAVGYVDDTQFVRFDSDAASPRGEPREPWVEQEG | | |
| | | PEYWDRETQKYKRQAQADRVNLRKLRGYYNQSEDGSHTL | | |
| | | QRMFGCDLGPDGRLLRGYNQFAYDGKDYIALNEDLRSWT | | |
| | | AADTAAQITQRKWEAAREAEQRRAYLEGTCVEWLRRYLE | | |
| | | NGKETLQRAEHPKTHVTHHPVSDHEATLRCWALGFYPAE | | |
| | | ITLTWQWDGEDQTQDTELVETRPAGDGTFQKWAAVVVPS | | |
| | | GEEQRYTCHVQHEGLPEPLTLRWKPSSQPTIPIMALIVL | | |
| | | GGVAGLLLFIGLGIFF | | |
| cPep/B2M/ | 250 | MSRSVALAVLALLSLSGLEAQYDDAVYKLGGGGSGGGGS | 474 | single |
| HLA-C*04 | | GGGGSIQRTPKIQVYSRHPAENGKSNFLNCYVSGFHPSD | | chain |
| | | IEVDLLKNGERIEKVEHSDLSFSKDWSFYLLYYTEFTPT | | trimer |
| | | EKDEYACRVNHVTLSQPKIVKWDRDMGGGGSGGGGSGGG | | |
| | | GSGGGGSGSHSMRYFSTSVSWPGRGEPRFIAVGYVDDTQ | | |
| | | FVRFDSDAASPRGEPREPWVEQEGPEYWDRETQKYKRQA | | |
| | | QADRVNLRKLRGYYNQSEDGSHTLQRMFGCDLGPDGRLL | | |
| | | RGYNQFAYDGKDYIALNEDLRSWTAADTAAQITQRKWEA | | |
| | | AREAEQRRAYLEGTCVEWLRRYLENGKETLQRAEHPKTH | | |
| | | VTHHPVSDHEATLRCWALGFYPAEITLTWQWDGEDQTQD | | |
| | | TELVETRPAGDGTFQKWAAVVVPSGEEQRYTCHVQHEGL | | |
| | | PEPLTLRWKPSSQPTIPIVGIVAGLAVLAVLAVLGAMVA | | |
| | | VVMCRRKSSGGKGGSCSQAASSNSAQGSDESLIACKA | | |
| cPep/B2M/ | 251 | MSRSVALAVLALLSLSGLEAQYDDAVYKLGGGGSGGGGS | 475 | single |
| HLA-C*04- | | GGGGSIQRTPKIQVYSRHPAENGKSNFLNCYVSGFHPSD | | chain |
| CD4TM | | IEVDLLKNGERIEKVEHSDLSFSKDWSFYLLYYTEFTPT | | trimer |
| | | EKDEYACRVNHVTLSQPKIVKWDRDMGGGGSGGGGSGGG | | |
| | | GSGGGGSGSHSMRYFSTSVSWPGRGEPRFIAVGYVDDTQ | | |
| | | FVRFDSDAASPRGEPREPWVEQEGPEYWDRETQKYKRQA | | |
| | | QADRVNLRKLRGYYNQSEDGSHTLQRMFGCDLGPDGRLL | | |
| | | RGYNQFAYDGKDYIALNEDLRSWTAADTAAQITQRKWEA | | |
| | | AREAEQRRAYLEGTCVEWLRRYLENGKETLQRAEHPKTH | | |
| | | VTHHPVSDHEATLRCWALGFYPAEITLTWQWDGEDQTQD | | |
| | | TELVETRPAGDGTFQKWAAVVVPSGEEQRYTCHVQHEGL | | |
| | | PEPLTLRWKPSSQPTIPIMALIVLGGVAGLLLFIGLGIF | | |
| | | FCVRC | | |
| B2M/HLA- | 252 | MSRSVALAVLALLSLSGLEAIQRTPKIQVYSRHPAENGK | 476 | single |
| C*07(C1G) | | SNFLNCYVSGFHPSDIEVDLLKNGERIEKVEHSDLSFSK | | chain |
| | | DWSFYLLYYTEFTPTEKDEYACRVNHVTLSQPKIVKWDR | | dimer |
| | | DMGGGGSGGGGSGGGGSGGGGSGSHSMRYFDTAVSRPGR | | |
| | | GEPRFISVGYVDDTQFVRFDSDAASPRGEPRAPWVEQEG | | |
| | | PEYWDRETQKYKRQAQADRVSLRNLRGYYNQSEDGSHTL | | |
| | | QRMSGCDLGPDGRLLRGYDQSAYDGKDYIALNEDLRSWT | | |
| | | AADTAAQITQRKLEAARAAEQLRAYLEGTCVEWLRRYLE | | |
| | | NGKETLQRAEPPKTHVTHHPLSDHEATLRCWALGFYPAE | | |
| | | ITLTWQRDGEDQTQDTELVETRPAGDGTFQKWAAVVVPS | | |
| | | GQEQRYTCHMQHEGLQEPLTLSWEPSSQPTIPIMGIVAG | | |
| | | LAVLVVLAVLGAVVTAMMCRRKSSGGKGGSCSQAACSNS | | |
| | | AQGSDESLITCKA | | |
| B2M/HLA- | 253 | MSRSVALAVLALLSLSGLEAIQRTPKIQVYSRHPAENGK | 477 | single |
| C*07(C1G)- | | SNFLNCYVSGFHPSDIEVDLLKNGERIEKVEHSDLSFSK | | chain |
| CD4TM | | DWSFYLLYYTEFTPTEKDEYACRVNHVTLSQPKIVKWDR | | dimer |
| | | DMGGGGSGGGGSGGGGSGGGGSGSHSMRYFDTAVSRPGR | | |
| | | GEPRFISVGYVDDTQFVRFDSDAASPRGEPRAPWVEQEG | | |
| | | PEYWDRETQKYKRQAQADRVSLRNLRGYYNQSEDGSHTL | | |
| | | QRMSGCDLGPDGRLLRGYDQSAYDGKDYIALNEDLRSWT | | |
| | | AADTAAQITQRKLEAARAAEQLRAYLEGTCVEWLRRYLE | | |
| | | NGKETLQRAEPPKTHVTHHPLSDHEATLRCWALGFYPAE | | |
| | | ITLTWQRDGEDQTQDTELVETRPAGDGTFQKWAAVVVPS | | |
| | | GQEQRYTCHMQHEGLQEPLTLSWEPSSQPTIPIMALIVL | | |
| | | GGVAGLLLFIGLGIFFCVRC | | |
| cPep/B2M/ | 254 | MSRSVALAVLALLSLSGLEARYRPGTVALGGGGSGGGGS | 478 | single |
| HLA- | | GGGGSIQRTPKIQVYSRHPAENGKSNFLNCYVSGFHPSD | | chain |
| C*07(C1G) | | IEVDLLKNGERIEKVEHSDLSFSKDWSFYLLYYTEFTPT | | trimer |
| | | EKDEYACRVNHVTLSQPKIVKWDRDMGGGGSGGGGSGGG | | |
| | | GSGGGGSGSHSMRYFDTAVSRPGRGEPRFISVGYVDDTQ | | |
| | | FVRFDSDAASPRGEPRAPWVEQEGPEYWDRETQKYKRQA | | |
| | | QADRVSLRNLRGYYNQSEDGSHTLQRMSGCDLGPDGRLL | | |
| | | RGYDQSAYDGKDYIALNEDLRSWTAADTAAQITQRKLEA | | |
| | | ARAAEQLRAYLEGTCVEWLRRYLENGKETLQRAEPPKTH | | |
| | | VTHHPLSDHEATLRCWALGFYPAEITLTWQRDGEDQTQD | | |
| | | TELVETRPAGDGTFQKWAAVVVPSGQEQRYTCHMQHEGL | | |
| | | QEPLTLSWEPSSQPTIPIMGIVAGLAVLVVLAVLGAVVT | | |
| | | AMMCRRKSSGGKGGSCSQAACSNSAQGSDESLITCKA | | |
| cPep/B2M/ | 255 | MSRSVALAVLALLSLSGLEARYRPGTVALGGGGSGGGGS | 479 | single |
| HLA- | | GGGGSIQRTPKIQVYSRHPAENGKSNFLNCYVSGFHPSD | | chain |
| C*07(C1G)- | | IEVDLLKNGERIEKVEHSDLSFSKDWSFYLLYYTEFTPT | | trimer |
| CD4TM | | EKDEYACRVNHVTLSQPKIVKWDRDMGGGGSGGGGSGGG | | |
| | | GSGGGGSGSHSMRYFDTAVSRPGRGEPRFISVGYVDDTQ | | |
| | | FVRFDSDAASPRGEPRAPWVEQEGPEYWDRETQKYKRQA | | |
| | | QADRVSLRNLRGYYNQSEDGSHTLQRMSGCDLGPDGRLL | | |
| | | RGYDQSAYDGKDYIALNEDLRSWTAADTAAQITQRKLEA | | |
| | | ARAAEQLRAYLEGTCVEWLRRYLENGKETLQRAEPPKTH | | |
| | | VTHHPLSDHEATLRCWALGFYPAEITLTWQRDGEDQTQD | | |
| | | TELVETRPAGDGTFQKWAAVVVPSGQEQRYTCHMQHEGL | | |
| | | QEPLTLSWEPSSQPTIPIMALIVLGGVAGLLLFIGLGIF | | |
| | | FCVRC | | |
| B2M/HLA- | 256 | MSRSVALAVLALLSLSGLEAIQRTPKIQVYSRHPAENGK | 480 | single |
| B*57 | | SNFLNCYVSGFHPSDIEVDLLKNGERIEKVEHSDLSFSK | | chain |
| | | DWSFYLLYYTEFTPTEKDEYACRVNHVTLSQPKIVKWDR | | dimer |
| | | DMGGGGSGGGGSGGGGSGGGGSGSHSMRYFYTAMSRPGR | | |
| | | GEPRFIAVGYVDDTQFVRFDSDAASPRMAPRAPWIEQEG | | |
| | | PEYWDGETRNMKASAQTYRENLRIALRYYNQSEAGSHII | | |
| | | QVMYGCDVGPDGRLLRGHDQSAYDGKDYIALNEDLSSWT | | |
| | | AADTAAQITQRKWEAARVAEQLRAYLEGLCVEWLRRYLE | | |
| | | NGKETLQRADPPKTHVTHHPISDHEATLRCWALGFYPAE | | |
| | | ITLTWQRDGEDQTQDTELVETRPAGDRTFQKWAAVVVPS | | |
| | | GEEQRYTCHVQHEGLPKPLTLRWEPSSQSTVPIVGIVAG | | |
| | | LAVLAVVVIGAVVAAVMCRRKSSGGKGGSYSQAACSDSA | | |
| | | QGSDVSLTA | | |
| B2M/HLA- | 257 | MSRSVALAVLALLSLSGLEAIQRTPKIQVYSRHPAENGK | 481 | single |
| B*57- | | SNFLNCYVSGFHPSDIEVDLLKNGERIEKVEHSDLSFSK | | chain |
| CD4TM | | DWSFYLLYYTEFTPTEKDEYACRVNHVTLSQPKIVKWDR | | dimer |
| | | DMGGGGSGGGGSGGGGSGGGGSGSHSMRYFYTAMSRPGR | | |
| | | GEPRFIAVGYVDDTQFVRFDSDAASPRMAPRAPWIEQEG | | |
| | | PEYWDGETRNMKASAQTYRENLRIALRYYNQSEAGSHII | | |
| | | QVMYGCDVGPDGRLLRGHDQSAYDGKDYIALNEDLSSWT | | |
| | | AADTAAQITQRKWEAARVAEQLRAYLEGLCVEWLRRYLE | | |
| | | NGKETLQRADPPKTHVTHHPISDHEATLRCWALGFYPAE | | |
| | | ITLTWQRDGEDQTQDTELVETRPAGDRTFQKWAAVVVPS | | |
| | | GEEQRYTCHVQHEGLPKPLTLRWEPSSQSTVPIMALIVL | | |
| | | GGVAGLLLFIGLGIFFCVRC | | |
| cPep/B2M/ | 258 | MSRSVALAVLALLSLSGLEALSSPVTKSFGGGGSGGGGS | 482 | single |
| HLA-B*57 | | GGGGSIQRTPKIQVYSRHPAENGKSNFLNCYVSGFHPSD | | chain |
| | | IEVDLLKNGERIEKVEHSDLSFSKDWSFYLLYYTEFTPT | | trimer |
| | | EKDEYACRVNHVTLSQPKIVKWDRDMGGGGSGGGGSGGG | | |
| | | GSGGGGSGSHSMRYFYTAMSRPGRGEPRFIAVGYVDDTQ | | |
| | | FVRFDSDAASPRMAPRAPWIEQEGPEYWDGETRNMKASA | | |
| | | QTYRENLRIALRYYNQSEAGSHIIQVMYGCDVGPDGRLL | | |
| | | RGHDQSAYDGKDYIALNEDLSSWTAADTAAQITQRKWEA | | |
| | | ARVAEQLRAYLEGLCVEWLRRYLENGKETLQRADPPKTH | | |
| | | VTHHPISDHEATLRCWALGFYPAEITLTWQRDGEDQTQD | | |
| | | TELVETRPAGDRTFQKWAAVVVPSGEEQRYTCHVQHEGL | | |
| | | PKPLTLRWEPSSQSTVPIVGIVAGLAVLAVVVIGAVVAA | | |
| | | VMCRRKSSGGKGGSYSQAACSDSAQGSDVSLTA | | |
| cPep/B2M/ | 259 | MSRSVALAVLALLSLSGLEALSSPVTKSFGGGGSGGGGS | 483 | single |
| HLA-B*57- | | GGGGSIQRTPKIQVYSRHPAENGKSNFLNCYVSGFHPSD | | chain |
| CD4TM | | IEVDLLKNGERIEKVEHSDLSFSKDWSFYLLYYTEFTPT | | trimer |
| | | EKDEYACRVNHVTLSQPKIVKWDRDMGGGGSGGGGSGGG | | |
| | | GSGGGGSGSHSMRYFYTAMSRPGRGEPRFIAVGYVDDTQ | | |
| | | FVRFDSDAASPRMAPRAPWIEQEGPEYWDGETRNMKASA | | |
| | | QTYRENLRIALRYYNQSEAGSHIIQVMYGCDVGPDGRLL | | |
| | | RGHDQSAYDGKDYIALNEDLSSWTAADTAAQITQRKWEA | | |
| | | ARVAEQLRAYLEGLCVEWLRRYLENGKETLQRADPPKTH | | |
| | | VTHHPISDHEATLRCWALGFYPAEITLTWQRDGEDQTQD | | |
| | | TELVETRPAGDRTFQKWAAVVVPSGEEQRYTCHVQHEGL | | |
| | | PKPLTLRWEPSSQSTVPIMALIVLGGVAGLLLFIGLGIF | | |
| | | FCVRC | | |
| B2M/HLA- | 260 | MSRSVALAVLALLSLSGLEAIQRTPKIQVYSRHPAENGK | 484 | single |
| B*44 | | SNFLNCYVSGFHPSDIEVDLLKNGERIEKVEHSDLSFSK | | chain |
| | | DWSFYLLYYTEFTPTEKDEYACRVNHVTLSQPKIVKWDR | | dimer |
| | | DMGGGGSGGGGSGGGGSGGGGSGSHSMRYFYTAMSRPGR | | |
| | | GEPRFITVGYVDDTLFVRFDSDATSPRKEPRAPWIEQEG | | |
| | | PEYWDRETQISKTNTQTYRENLRTALRYYNQSEAGSHII | | |
| | | QRMYGCDVGPDGRLLRGYDQDAYDGKDYIALNEDLSSWT | | |
| | | AADTAAQITQRKWEAARVAEQDRAYLEGLCVESLRRYLE | | |
| | | NGKETLQRADPPKTHVTHHPISDHEVTLRCWALGFYPAE | | |
| | | ITLTWQRDGEDQTQDTELVETRPAGDRTFQKWAAVVVPS | | |
| | | GEEQRYTCHVQHEGLPKPLTLRWEPSSQSTVPIVGIVAG | | |
| | | LAVLAVVVIGAVVAAVMCRRKSSGGKGGSYSQAACSDSA | | |
| | | QGSDVSLTA | | |
| B2M/HLA- | 261 | MSRSVALAVLALLSLSGLEAIQRTPKIQVYSRHPAENGK | 485 | single |
| B*44- | | SNFLNCYVSGFHPSDIEVDLLKNGERIEKVEHSDLSFSK | | chain |
| CD4TM | | DWSFYLLYYTEFTPTEKDEYACRVNHVTLSQPKIVKWDR | | dimer |
| | | DMGGGGSGGGGSGGGGSGGGGSGSHSMRYFYTAMSRPGR | | |
| | | GEPRFITVGYVDDTLFVRFDSDATSPRKEPRAPWIEQEG | | |
| | | PEYWDRETQISKTNTQTYRENLRTALRYYNQSEAGSHII | | |
| | | QRMYGCDVGPDGRLLRGYDQDAYDGKDYIALNEDLSSWT | | |
| | | AADTAAQITQRKWEAARVAEQDRAYLEGLCVESLRRYLE | | |
| | | NGKETLQRADPPKTHVTHHPISDHEVTLRCWALGFYPAE | | |
| | | ITLTWQRDGEDQTQDTELVETRPAGDRTFQKWAAVVVPS | | |
| | | GEEQRYTCHVQHEGLPKPLTLRWEPSSQSTVPIMALIVL | | |
| | | GGVAGLLLFIGLGIFFCVRC | | |
| cPep/B2M/ | 262 | MSRSVALAVLALLSLSGLEAEEVHDLERKYGGGGSGGGG | 486 | single |
| HLA-B*44 | | SGGGGSIQRTPKIQVYSRHPAENGKSNFLNCYVSGFHPS | | chain |
| | | DIEVDLLKNGERIEKVEHSDLSFSKDWSFYLLYYTEFTP | | trimer |
| | | TEKDEYACRVNHVTLSQPKIVKWDRDMGGGGSGGGGSGG | | |
| | | GGSGGGGSGSHSMRYFYTAMSRPGRGEPRFITVGYVDDT | | |
| | | LFVRFDSDATSPRKEPRAPWIEQEGPEYWDRETQISKTN | | |
| | | TQTYRENLRTALRYYNQSEAGSHIIQRMYGCDVGPDGRL | | |
| | | LRGYDQDAYDGKDYIALNEDLSSWTAADTAAQITQRKWE | | |
| | | AARVAEQDRAYLEGLCVESLRRYLENGKETLQRADPPKT | | |
| | | HVTHHPISDHEVTLRCWALGFYPAEITLTWQRDGEDQTQ | | |
| | | DTELVETRPAGDRTFQKWAAVVVPSGEEQRYTCHVQHEG | | |
| | | LPKPLTLRWEPSSQSTVPIVGIVAGLAVLAVVVIGAVVA | | |
| | | AVMCRRKSSGGKGGSYSQAACSDSAQGSDVSLTA | | |
| cPep/B2M/ | 263 | MSRSVALAVLALLSLSGLEAEEVHDLERKYGGGGSGGGG | 487 | single |
| HLA-B*44- | | SGGGGSIQRTPKIQVYSRHPAENGKSNFLNCYVSGFHPS | | chain |
| CD4TM | | DIEVDLLKNGERIEKVEHSDLSFSKDWSFYLLYYTEFTP | | trimer |
| | | TEKDEYACRVNHVTLSQPKIVKWDRDMGGGGSGGGGSGG | | |
| | | GGSGGGGSGSHSMRYFYTAMSRPGRGEPRFITVGYVDDT | | |
| | | LFVRFDSDATSPRKEPRAPWIEQEGPEYWDRETQISKTN | | |
| | | TQTYRENLRTALRYYNQSEAGSHIIQRMYGCDVGPDGRL | | |
| | | LRGYDQDAYDGKDYIALNEDLSSWTAADTAAQITQRKWE | | |
| | | AARVAEQDRAYLEGLCVESLRRYLENGKETLQRADPPKT | | |
| | | HVTHHPISDHEVTLRCWALGFYPAEITLTWQRDGEDQTQ | | |
| | | DTELVETRPAGDRTFQKWAAVVVPSGEEQRYTCHVQHEG | | |
| | | LPKPLTLRWEPSSQSTVPIMALIVLGGVAGLLLFIGLGI | | |
| | | FFCVRC | | |
| B2M/HLA- | 264 | MSRSVALAVLALLSLSGLEAIQRTPKIQVYSRHPAENGK | 488 | single |
| A*03 | | SNFLNCYVSGFHPSDIEVDLLKNGERIEKVEHSDLSFSK | | chain |
| | | DWSFYLLYYTEFTPTEKDEYACRVNHVTLSQPKIVKWDR | | dimer |
| | | DMGGGGSGGGGSGGGGSGGGGSGSHSMRYFFTSVSRPGR | | |
| | | GEPRFIAVGYVDDTQFVRFDSDAASQRMEPRAPWIEQEG | | |
| | | PEYWDQETRNVKAQSQTDRVDLGTLRGYYNQSEAGSHTI | | |
| | | QIMYGCDVGSDGRFLRGYRQDAYDGKDYIALNEDLRSWT | | |
| | | AADMAAQITKRKWEAAHEAEQLRAYLDGTCVEWLRRYLE | | |
| | | NGKETLQRTDPPKTHMTHHPISDHEATLRCWALGFYPAE | | |
| | | ITLTWQRDGEDQTQDTELVETRPAGDGTFQKWAAVVVPS | | |
| | | GEEQRYTCHVQHEGLPKPLTLRWELSSQPTIPIVGIIAG | | |
| | | LVLLGAVITGAVVAAVMWRRKSSDRKGGSYTQAASSDSA | | |
| | | QGSDVSLTACKV | | |
| B2M/HLA- | 265 | MSRSVALAVLALLSLSGLEAIQRTPKIQVYSRHPAENGK | 489 | single |
| A*03- | | SNFLNCYVSGFHPSDIEVDLLKNGERIEKVEHSDLSFSK | | chain |
| CD4TM | | DWSFYLLYYTEFTPTEKDEYACRVNHVTLSQPKIVKWDR | | dimer |
| | | DMGGGGSGGGGSGGGGSGGGGSGSHSMRYFFTSVSRPGR | | |
| | | GEPRFIAVGYVDDTQFVRFDSDAASQRMEPRAPWIEQEG | | |
| | | PEYWDQETRNVKAQSQTDRVDLGTLRGYYNQSEAGSHTI | | |
| | | QIMYGCDVGSDGRFLRGYRQDAYDGKDYIALNEDLRSWT | | |
| | | AADMAAQITKRKWEAAHEAEQLRAYLDGTCVEWLRRYLE | | |
| | | NGKETLQRTDPPKTHMTHHPISDHEATLRCWALGFYPAE | | |
| | | ITLTWQRDGEDQTQDTELVETRPAGDGTFQKWAAVVVPS | | |
| | | GEEQRYTCHVQHEGLPKPLTLRWELSSQPTIPIMALIVL | | |
| | | GGVAGLLLFIGLGIFFCVRC | | |
| cPep/B2M/ | 266 | MSRSVALAVLALLSLSGLEARLRAEAQVKGGGGSGGGGS | 490 | single |
| HLA-A*03 | | GGGGSIQRTPKIQVYSRHPAENGKSNFLNCYVSGFHPSD | | chain |
| | | IEVDLLKNGERIEKVEHSDLSFSKDWSFYLLYYTEFTPT | | trimer |
| | | EKDEYACRVNHVTLSQPKIVKWDRDMGGGGSGGGGSGGG | | |
| | | GSGGGGSGSHSMRYFFTSVSRPGRGEPRFIAVGYVDDTQ | | |
| | | FVRFDSDAASQRMEPRAPWIEQEGPEYWDQETRNVKAQS | | |
| | | QTDRVDLGTLRGYYNQSEAGSHTIQIMYGCDVGSDGRFL | | |
| | | RGYRQDAYDGKDYIALNEDLRSWTAADMAAQITKRKWEA | | |
| | | AHEAEQLRAYLDGTCVEWLRRYLENGKETLQRTDPPKTH | | |
| | | MTHHPISDHEATLRCWALGFYPAEITLTWQRDGEDQTQD | | |
| | | TELVETRPAGDGTFQKWAAVVVPSGEEQRYTCHVQHEGL | | |
| | | PKPLTLRWELSSQPTIPIVGIIAGLVLLGAVITGAVVAA | | |
| | | VMWRRKSSDRKGGSYTQAASSDSAQGSDVSLTACKV | | |
| cPep/B2M/ | 267 | MSRSVALAVLALLSLSGLEARLRAEAQVKGGGGSGGGGS | 491 | single |
| HLA-A*03- | | GGGGSIQRTPKIQVYSRHPAENGKSNFLNCYVSGFHPSD | | chain |
| CD4TM | | IEVDLLKNGERIEKVEHSDLSFSKDWSFYLLYYTEFTPT | | trimer |
| | | EKDEYACRVNHVTLSQPKIVKWDRDMGGGGSGGGGSGGG | | |
| | | GSGGGGSGSHSMRYFFTSVSRPGRGEPRFIAVGYVDDTQ | | |
| | | FVRFDSDAASQRMEPRAPWIEQEGPEYWDQETRNVKAQS | | |
| | | QTDRVDLGTLRGYYNQSEAGSHTIQIMYGCDVGSDGRFL | | |
| | | RGYRQDAYDGKDYIALNEDLRSWTAADMAAQITKRKWEA | | |
| | | AHEAEQLRAYLDGTCVEWLRRYLENGKETLQRTDPPKTH | | |
| | | MTHHPISDHEATLRCWALGFYPAEITLTWQRDGEDQTQD | | |
| | | TELVETRPAGDGTFQKWAAVVVPSGEEQRYTCHVQHEGL | | |
| | | PKPLTLRWELSSQPTIPIMALIVLGGVAGLLLFIGLGIF | | |
| | | FCVRC | | |
| B2M/HLA- | 287 | MSRSVALAVLALLSLSGLEAIQRTPKIQVYSRHPAENGK | 492 | single |
| C*04(D227K/ | | SNFLNCYVSGFHPSDIEVDLLKNGERIEKVEHSDLSFSK | | chain |
| T228A) | | DWSFYLLYYTEFTPTEKDEYACRVNHVTLSQPKIVKWDR | | dimer |
| | | DMGGGGSGGGGSGGGGSGGGGSGSHSMRYFSTSVSWPGR | | |
| | | GEPRFIAVGYVDDTQFVRFDSDAASPRGEPREPWVEQEG | | |
| | | PEYWDRETQKYKRQAQADRVNLRKLRGYYNQSEDGSHTL | | |
| | | QRMFGCDLGPDGRLLRGYNQFAYDGKDYIALNEDLRSWT | | |
| | | AADTAAQITQRKWEAAREAEQRRAYLEGTCVEWLRRYLE | | |
| | | NGKETLQRAEHPKTHVTHHPVSDHEATLRCWALGFYPAE | | |
| | | ITLTWQWDGEDQTQKAELVETRPAGDGTFQKWAAVVVPS | | |
| | | GEEQRYTCHVQHEGLPEPLTLRWKPSSQPTIPIVGIVAG | | |
| | | LAVLAVLAVLGAMVAVVMCRRKSSGGKGGSCSQAASSNS | | |
| | | AQGSDESLIACKA | | |
| B2M/HLA- | 288 | MSRSVALAVLALLSLSGLEAIQRTPKIQVYSRHPAENGK | 493 | single |
| C*04(A245V) | | SNFLNCYVSGFHPSDIEVDLLKNGERIEKVEHSDLSFSK | | chain |
| | | DWSFYLLYYTEFTPTEKDEYACRVNHVTLSQPKIVKWDR | | dimer |
| | | DMGGGGSGGGGSGGGGSGGGGSGSHSMRYFSTSVSWPGR | | |
| | | GEPRFIAVGYVDDTQFVRFDSDAASPRGEPREPWVEQEG | | |
| | | PEYWDRETQKYKRQAQADRVNLRKLRGYYNQSEDGSHTL | | |
| | | QRMFGCDLGPDGRLLRGYNQFAYDGKDYIALNEDLRSWT | | |
| | | AADTAAQITQRKWEAAREAEQRRAYLEGTCVEWLRRYLE | | |
| | | NGKETLQRAEHPKTHVTHHPVSDHEATLRCWALGFYPAE | | |
| | | ITLTWQWDGEDQTQDTELVETRPAGDGTFQKWVAVVVPS | | |
| | | GEEQRYTCHVQHEGLPEPLTLRWKPSSQPTIPIVGIVAG | | |
| | | LAVLAVLAVLGAMVAVVMCRRKSSGGKGGSCSQAASSNS | | |
| | | AQGSDESLIACKA | | |
| B2M/HLA- | 289 | MSRSVALAVLALLSLSGLEAIQRTPKIQVYSRHPAENGK | 494 | single |
| C*04(D227K/ | | SNFLNCYVSGFHPSDIEVDLLKNGERIEKVEHSDLSFSK | | chain |
| T228A/A245V) | | DWSFYLLYYTEFTPTEKDEYACRVNHVTLSQPKIVKWDR | | dimer |
| | | DMGGGGSGGGGSGGGGSGGGGSGSHSMRYFSTSVSWPGR | | |
| | | GEPRFIAVGYVDDTQFVRFDSDAASPRGEPREPWVEQEG | | |
| | | PEYWDRETQKYKRQAQADRVNLRKLRGYYNQSEDGSHTL | | |
| | | QRMFGCDLGPDGRLLRGYNQFAYDGKDYIALNEDLRSWT | | |
| | | AADTAAQITQRKWEAAREAEQRRAYLEGTCVEWLRRYLE | | |
| | | NGKETLQRAEHPKTHVTHHPVSDHEATLRCWALGFYPAE | | |
| | | ITLTWQWDGEDQTQKAELVETRPAGDGTFQKWVAVVVPS | | |
| | | GEEQRYTCHVQHEGLPEPLTLRWKPSSQPTIPIVGIVAG | | |
| | | LAVLAVLAVLGAMVAVVMCRRKSSGGKGGSCSQAASSNS | | |
| | | AQGSDESLIACKA | | |
| B2M(K58E)/ | 290 | MSRSVALAVLALLSLSGLEAIQRTPKIQVYSRHPAENGK | 495 | single |
| HLA-C*04 | | SNFLNCYVSGFHPSDIEVDLLKNGERIEKVEHSDLSFSE | | chain |
| | | DWSFYLLYYTEFTPTEKDEYACRVNHVTLSQPKIVKWDR | | dimer |
| | | DMGGGGSGGGGSGGGGSGGGGSGSHSMRYFSTSVSWPGR | | |
| | | GEPRFIAVGYVDDTQFVRFDSDAASPRGEPREPWVEQEG | | |
| | | PEYWDRETQKYKRQAQADRVNLRKLRGYYNQSEDGSHTL | | |
| | | QRMFGCDLGPDGRLLRGYNQFAYDGKDYIALNEDLRSWT | | |
| | | AADTAAQITQRKWEAAREAEQRRAYLEGTCVEWLRRYLE | | |
| | | NGKETLQRAEHPKTHVTHHPVSDHEATLRCWALGFYPAE | | |
| | | ITLTWQWDGEDQTQDTELVETRPAGDGTFQKWAAVVVPS | | |
| | | GEEQRYTCHVQHEGLPEPLTLRWKPSSQPTIPIVGIVAG | | |
| | | LAVLAVLAVLGAMVAVVMCRRKSSGGKGGSCSQAASSNS | | |
| | | AQGSDESLIACKA | | |
| B2M(K58E)/ | 291 | MSRSVALAVLALLSLSGLEAIQRTPKIQVYSRHPAENGK | 496 | single |
| HLA- | | SNFLNCYVSGFHPSDIEVDLLKNGERIEKVEHSDLSFSE | | chain |
| C*04(D227K/ | | DWSFYLLYYTEFTPTEKDEYACRVNHVTLSQPKIVKWDR | | dimer |
| T228A) | | DMGGGGSGGGGSGGGGSGGGGSGSHSMRYFSTSVSWPGR | | |
| | | GEPRFIAVGYVDDTQFVRFDSDAASPRGEPREPWVEQEG | | |
| | | PEYWDRETQKYKRQAQADRVNLRKLRGYYNQSEDGSHTL | | |
| | | QRMFGCDLGPDGRLLRGYNQFAYDGKDYIALNEDLRSWT | | |
| | | AADTAAQITQRKWEAAREAEQRRAYLEGTCVEWLRRYLE | | |
| | | NGKETLQRAEHPKTHVTHHPVSDHEATLRCWALGFYPAE | | |
| | | ITLTWQWDGEDQTQKAELVETRPAGDGTFQKWAAVVVPS | | |
| | | GEEQRYTCHVQHEGLPEPLTLRWKPSSQPTIPIVGIVAG | | |
| | | LAVLAVLAVLGAMVAVVMCRRKSSGGKGGSCSQAASSNS | | |
| | | AQGSDESLIACKA | | |
| B2M(K58E)/ | 292 | MSRSVALAVLALLSLSGLEAIQRTPKIQVYSRHPAENGK | 497 | single |
| HLA- | | SNFLNCYVSGFHPSDIEVDLLKNGERIEKVEHSDLSFSE | | chain |
| C*04(A245V) | | DWSFYLLYYTEFTPTEKDEYACRVNHVTLSQPKIVKWDR | | dimer |
| | | DMGGGGSGGGGSGGGGSGGGGSGSHSMRYFSTSVSWPGR | | |
| | | GEPRFIAVGYVDDTQFVRFDSDAASPRGEPREPWVEQEG | | |
| | | PEYWDRETQKYKRQAQADRVNLRKLRGYYNQSEDGSHTL | | |
| | | QRMFGCDLGPDGRLLRGYNQFAYDGKDYIALNEDLRSWT | | |
| | | AADTAAQITQRKWEAAREAEQRRAYLEGTCVEWLRRYLE | | |
| | | NGKETLQRAEHPKTHVTHHPVSDHEATLRCWALGFYPAE | | |
| | | ITLTWQWDGEDQTQDTELVETRPAGDGTFQKWVAVVVPS | | |
| | | GEEQRYTCHVQHEGLPEPLTLRWKPSSQPTIPIVGIVAG | | |
| | | LAVLAVLAVLGAMVAVVMCRRKSSGGKGGSCSQAASSNS | | |
| | | AQGSDESLIACKA | | |
| B2M(K58E)/ | 293 | MSRSVALAVLALLSLSGLEAIQRTPKIQVYSRHPAENGK | 498 | single |
| HLA- | | SNFLNCYVSGFHPSDIEVDLLKNGERIEKVEHSDLSFSE | | chain |
| C*04(D227K/ | | DWSFYLLYYTEFTPTEKDEYACRVNHVTLSQPKIVKWDR | | dimer |
| T228A/A245V) | | DMGGGGSGGGGSGGGGSGGGGSGSHSMRYFSTSVSWPGR | | |
| | | GEPRFIAVGYVDDTQFVRFDSDAASPRGEPREPWVEQEG | | |
| | | PEYWDRETQKYKRQAQADRVNLRKLRGYYNQSEDGSHTL | | |
| | | QRMFGCDLGPDGRLLRGYNQFAYDGKDYIALNEDLRSWT | | |
| | | AADTAAQITQRKWEAAREAEQRRAYLEGTCVEWLRRYLE | | |
| | | NGKETLQRAEHPKTHVTHHPVSDHEATLRCWALGFYPAE | | |
| | | ITLTWQWDGEDQTQKAELVETRPAGDGTFQKWVAVVVPS | | |
| | | GEEQRYTCHVQHEGLPEPLTLRWKPSSQPTIPIVGIVAG | | |
| | | LAVLAVLAVLGAMVAVVMCRRKSSGGKGGSCSQAASSNS | | |
| | | AQGSDESLIACKA | | |
| B2M/HLA- | 294 | MSRSVALAVLALLSLSGLEAIQRTPKIQVYSRHPAENGK | 499 | single |
| C*04(D227K/ | | SNFLNCYVSGFHPSDIEVDLLKNGERIEKVEHSDLSFSK | | chain |
| T228A)- | | DWSFYLLYYTEFTPTEKDEYACRVNHVTLSQPKIVKWDR | | dimer |
| CD4TM | | DMGGGGSGGGGSGGGGSGGGGSGSHSMRYFSTSVSWPGR | | |
| | | GEPRFIAVGYVDDTQFVRFDSDAASPRGEPREPWVEQEG | | |
| | | PEYWDRETQKYKRQAQADRVNLRKLRGYYNQSEDGSHTL | | |
| | | QRMFGCDLGPDGRLLRGYNQFAYDGKDYIALNEDLRSWT | | |
| | | AADTAAQITQRKWEAAREAEQRRAYLEGTCVEWLRRYLE | | |
| | | NGKETLQRAEHPKTHVTHHPVSDHEATLRCWALGFYPAE | | |
| | | ITLTWQWDGEDQTQKAELVETRPAGDGTFQKWAAVVVPS | | |
| | | GEEQRYTCHVQHEGLPEPLTLRWKPSSQPTIPIMALIVL | | |
| | | GGVAGLLLFIGLGIFFCVRC | | |
| B2M/HLA- | 295 | MSRSVALAVLALLSLSGLEAIQRTPKIQVYSRHPAENGK | 500 | single |
| C*04(A245V)- | | SNFLNCYVSGFHPSDIEVDLLKNGERIEKVEHSDLSFSK | | chain |
| CD4TM | | DWSFYLLYYTEFTPTEKDEYACRVNHVTLSQPKIVKWDR | | dimer |
| | | DMGGGGSGGGGSGGGGSGGGGSGSHSMRYFSTSVSWPGR | | |
| | | GEPRFIAVGYVDDTQFVRFDSDAASPRGEPREPWVEQEG | | |
| | | PEYWDRETQKYKRQAQADRVNLRKLRGYYNQSEDGSHTL | | |
| | | QRMFGCDLGPDGRLLRGYNQFAYDGKDYIALNEDLRSWT | | |
| | | AADTAAQITQRKWEAAREAEQRRAYLEGTCVEWLRRYLE | | |
| | | NGKETLQRAEHPKTHVTHHPVSDHEATLRCWALGFYPAE | | |
| | | ITLTWQWDGEDQTQDTELVETRPAGDGTFQKWVAVVVPS | | |
| | | GEEQRYTCHVQHEGLPEPLTLRWKPSSQPTIPIMALIVL | | |
| | | GGVAGLLLFIGLGIFFCVRC | | |
| B2M/HLA- | 296 | MSRSVALAVLALLSLSGLEAIQRTPKIQVYSRHPAENGK | 501 | single |
| C*04(D227K/ | | SNFLNCYVSGFHPSDIEVDLLKNGERIEKVEHSDLSFSK | | chain |
| T228A/A245V)- | | DWSFYLLYYTEFTPTEKDEYACRVNHVTLSQPKIVKWDR | | dimer |
| CD4TM | | DMGGGGSGGGGSGGGGSGGGGSGSHSMRYFSTSVSWPGR | | |
| | | GEPRFIAVGYVDDTQFVRFDSDAASPRGEPREPWVEQEG | | |
| | | PEYWDRETQKYKRQAQADRVNLRKLRGYYNQSEDGSHTL | | |
| | | QRMFGCDLGPDGRLLRGYNQFAYDGKDYIALNEDLRSWT | | |
| | | AADTAAQITQRKWEAAREAEQRRAYLEGTCVEWLRRYLE | | |
| | | NGKETLQRAEHPKTHVTHHPVSDHEATLRCWALGFYPAE | | |
| | | ITLTWQWDGEDQTQKAELVETRPAGDGTFQKWVAVVVPS | | |
| | | GEEQRYTCHVQHEGLPEPLTLRWKPSSQPTIPIMALIVL | | |
| | | GGVAGLLLFIGLGIFFCVRC | | |
| B2M(K58E)/ | 297 | MSRSVALAVLALLSLSGLEAIQRTPKIQVYSRHPAENGK | 502 | single |
| HLA- | | SNFLNCYVSGFHPSDIEVDLLKNGERIEKVEHSDLSFSE | | chain |
| C*04- | | DWSFYLLYYTEFTPTEKDEYACRVNHVTLSQPKIVKWDR | | dimer |
| CD4TM | | DMGGGGSGGGGSGGGGSGGGGSGSHSMRYFSTSVSWPGR | | |
| | | GEPRFIAVGYVDDTQFVRFDSDAASPRGEPREPWVEQEG | | |
| | | PEYWDRETQKYKRQAQADRVNLRKLRGYYNQSEDGSHTL | | |
| | | QRMFGCDLGPDGRLLRGYNQFAYDGKDYIALNEDLRSWT | | |
| | | AADTAAQITQRKWEAAREAEQRRAYLEGTCVEWLRRYLE | | |
| | | NGKETLQRAEHPKTHVTHHPVSDHEATLRCWALGFYPAE | | |
| | | ITLTWQWDGEDQTQDTELVETRPAGDGTFQKWAAVVVPS | | |
| | | GEEQRYTCHVQHEGLPEPLTLRWKPSSQPTIPIMALIVL | | |
| | | GGVAGLLLFIGLGIFFCVRC | | |
| B2M(K58E)/ | 298 | MSRSVALAVLALLSLSGLEAIQRTPKIQVYSRHPAENGK | 503 | single |
| HLA- | | SNFLNCYVSGFHPSDIEVDLLKNGERIEKVEHSDLSFSE | | chain |
| C*04(D227K/ | | DWSFYLLYYTEFTPTEKDEYACRVNHVTLSQPKIVKWDR | | dimer |
| T228A)- | | DMGGGGSGGGGSGGGGSGGGGSGSHSMRYFSTSVSWPGR | | |
| CD4TM | | GEPRFIAVGYVDDTQFVRFDSDAASPRGEPREPWVEQEG | | |
| | | PEYWDRETQKYKRQAQADRVNLRKLRGYYNQSEDGSHTL | | |
| | | QRMFGCDLGPDGRLLRGYNQFAYDGKDYIALNEDLRSWT | | |
| | | AADTAAQITQRKWEAAREAEQRRAYLEGTCVEWLRRYLE | | |
| | | NGKETLQRAEHPKTHVTHHPVSDHEATLRCWALGFYPAE | | |
| | | ITLTWQWDGEDQTQKAELVETRPAGDGTFQKWAAVVVPS | | |
| | | GEEQRYTCHVQHEGLPEPLTLRWKPSSQPTIPIMALIVL | | |
| | | GGVAGLLLFIGLGIFFCVRC | | |
| B2M(K58E)/ | 299 | MSRSVALAVLALLSLSGLEAIQRTPKIQVYSRHPAENGK | 504 | single |
| HLA- | | SNFLNCYVSGFHPSDIEVDLLKNGERIEKVEHSDLSFSE | | chain |
| C*04(A245V)- | | DWSFYLLYYTEFTPTEKDEYACRVNHVTLSQPKIVKWDR | | dimer |
| CD4TM | | DMGGGGSGGGGSGGGGSGGGGSGSHSMRYFSTSVSWPGR | | |
| | | GEPRFIAVGYVDDTQFVRFDSDAASPRGEPREPWVEQEG | | |
| | | PEYWDRETQKYKRQAQADRVNLRKLRGYYNQSEDGSHTL | | |
| | | QRMFGCDLGPDGRLLRGYNQFAYDGKDYIALNEDLRSWT | | |
| | | AADTAAQITQRKWEAAREAEQRRAYLEGTCVEWLRRYLE | | |
| | | NGKETLQRAEHPKTHVTHHPVSDHEATLRCWALGFYPAE | | |
| | | ITLTWQWDGEDQTQDTELVETRPAGDGTFQKWVAVVVPS | | |
| | | GEEQRYTCHVQHEGLPEPLTLRWKPSSQPTIPIMALIVL | | |
| | | GGVAGLLLFIGLGIFFCVRC | | |
| B2M(K58E)/ | 300 | MSRSVALAVLALLSLSGLEAIQRTPKIQVYSRHPAENGK | 505 | single |
| HLA- | | SNFLNCYVSGFHPSDIEVDLLKNGERIEKVEHSDLSFSE | | chain |
| C*04(D227K/ | | DWSFYLLYYTEFTPTEKDEYACRVNHVTLSQPKIVKWDR | | dimer |
| T228A/A245V)- | | DMGGGGSGGGGSGGGGSGGGGSGSHSMRYFSTSVSWPGR | | |
| CD4TM | | GEPRFIAVGYVDDTQFVRFDSDAASPRGEPREPWVEQEG | | |
| | | PEYWDRETQKYKRQAQADRVNLRKLRGYYNQSEDGSHTL | | |
| | | QRMFGCDLGPDGRLLRGYNQFAYDGKDYIALNEDLRSWT | | |
| | | AADTAAQITQRKWEAAREAEQRRAYLEGTCVEWLRRYLE | | |
| | | NGKETLQRAEHPKTHVTHHPVSDHEATLRCWALGFYPAE | | |
| | | ITLTWQWDGEDQTQKAELVETRPAGDGTFQKWVAVVVPS | | |
| | | GEEQRYTCHVQHEGLPEPLTLRWKPSSQPTIPIMALIVL | | |
| | | GGVAGLLLFIGLGIFFCVRC | | |
| B2M(W60G)/ | 301 | MSRSVALAVLALLSLSGLEAIQRTPKIQVYSRHPAENGK | 506 | single |
| HLA-A*02 | | SNFLNCYVSGFHPSDIEVDLLKNGERIEKVEHSDLSFSK | | chain |
| | | DGSFYLLYYTEFTPTEKDEYACRVNHVTLSQPKIVKWDR | | dimer |
| | | DMGGGGSGGGGSGGGGSGGGGSGSHSMRYFFTSVSRPGR | | |
| | | GEPRFIAVGYVDDTQFVRFDSDAASQRMEPRAPWIEQEG | | |
| | | PEYWDGETRKVKAHSQTHRVDLGTLRGYYNQSEAGSHTV | | |
| | | QRMYGCDVGSDWRFLRGYHQYAYDGKDYIALKEDLRSWT | | |
| | | AADMAAQTTKHKWEAAHVAEQLRAYLEGTCVEWLRRYLE | | |
| | | NGKETLQRTDAPKTHMTHHAVSDHEATLRCWALSFYPAE | | |
| | | ITLTWQRDGEDQTQDTELVETRPAGDGTFQKWAAVVVPS | | |
| | | GQEQRYTCHVQHEGLPKPLTLRWEPSSQPTIPIVGIIAG | | |
| | | LVLFGAVITGAVVAAVMWRRKSSDRKGGSYSQAASSDSA | | |
| | | QGSDVSLTACKV | | |
| B2M(W60G)/ | 302 | MSRSVALAVLALLSLSGLEAIQRTPKIQVYSRHPAENGK | 507 | single |
| HLA- | | SNFLNCYVSGFHPSDIEVDLLKNGERIEKVEHSDLSFSK | | chain |
| A*02(D227K/ | | DGSFYLLYYTEFTPTEKDEYACRVNHVTLSQPKIVKWDR | | dimer |
| T228A) | | DMGGGGSGGGGSGGGGSGGGGSGSHSMRYFFTSVSRPGR | | |
| | | GEPRFIAVGYVDDTQFVRFDSDAASQRMEPRAPWIEQEG | | |
| | | PEYWDGETRKVKAHSQTHRVDLGTLRGYYNQSEAGSHTV | | |
| | | QRMYGCDVGSDWRFLRGYHQYAYDGKDYIALKEDLRSWT | | |
| | | AADMAAQTTKHKWEAAHVAEQLRAYLEGTCVEWLRRYLE | | |
| | | NGKETLQRTDAPKTHMTHHAVSDHEATLRCWALSFYPAE | | |
| | | ITLTWQRDGEDQTQKAELVETRPAGDGTFQKWAAVVVPS | | |
| | | GQEQRYTCHVQHEGLPKPLTLRWEPSSQPTIPIVGIIAG | | |
| | | LVLFGAVITGAVVAAVMWRRKSSDRKGGSYSQAASSDSA | | |
| | | QGSDVSLTACKV | | |
| B2M(W60G)/ | 303 | MSRSVALAVLALLSLSGLEAIQRTPKIQVYSRHPAENGK | 508 | single |
| HLA-C*04 | | SNFLNCYVSGFHPSDIEVDLLKNGERIEKVEHSDLSFSK | | chain |
| | | DGSFYLLYYTEFTPTEKDEYACRVNHVTLSQPKIVKWDR | | dimer |
| | | DMGGGGSGGGGSGGGGSGGGGSGSHSMRYFSTSVSWPGR | | |
| | | GEPRFIAVGYVDDTQFVRFDSDAASPRGEPREPWVEQEG | | |
| | | PEYWDRETQKYKRQAQADRVNLRKLRGYYNQSEDGSHTL | | |
| | | QRMFGCDLGPDGRLLRGYNQFAYDGKDYIALNEDLRSWT | | |
| | | AADTAAQITQRKWEAAREAEQRRAYLEGTCVEWLRRYLE | | |
| | | NGKETLQRAEHPKTHVTHHPVSDHEATLRCWALGFYPAE | | |
| | | ITLTWQWDGEDQTQDTELVETRPAGDGTFQKWAAVVVPS | | |
| | | GEEQRYTCHVQHEGLPEPLTLRWKPSSQPTIPIVGIVAG | | |
| | | LAVLAVLAVLGAMVAVVMCRRKSSGGKGGSCSQAASSNS | | |
| | | AQGSDESLIACKA | | |
| B2M(W60G)/ | 304 | MSRSVALAVLALLSLSGLEAIQRTPKIQVYSRHPAENGK | 509 | single |
| HLA- | | SNFLNCYVSGFHPSDIEVDLLKNGERIEKVEHSDLSFSK | | chain |
| C*04(D227K/ | | DGSFYLLYYTEFTPTEKDEYACRVNHVTLSQPKIVKWDR | | dimer |
| T228A) | | DMGGGGSGGGGSGGGGSGGGGSGSHSMRYFSTSVSWPGR | | |
| | | GEPRFIAVGYVDDTQFVRFDSDAASPRGEPREPWVEQEG | | |
| | | PEYWDRETQKYKRQAQADRVNLRKLRGYYNQSEDGSHTL | | |
| | | QRMFGCDLGPDGRLLRGYNQFAYDGKDYIALNEDLRSWT | | |
| | | AADTAAQITQRKWEAAREAEQRRAYLEGTCVEWLRRYLE | | |
| | | NGKETLQRAEHPKTHVTHHPVSDHEATLRCWALGFYPAE | | |
| | | ITLTWQWDGEDQTQKAELVETRPAGDGTFQKWAAVVVPS | | |
| | | GEEQRYTCHVQHEGLPEPLTLRWKPSSQPTIPIVGIVAG | | |
| | | LAVLAVLAVLGAMVAVVMCRRKSSGGKGGSCSQAASSNS | | |
| | | AQGSDESLIACKA | | |
| B2M/HLA- | 306 | MSRSVALAVLALLSLSGLEAIQRTPKIQVYSRHPAENGK | 526 | single |
| C*04(A73T) | | SNFLNCYVSGFHPSDIEVDLLKNGERIEKVEHSDLSFSK | | chain |
| | | DWSFYLLYYTEFTPTEKDEYACRVNHVTLSQPKIVKWDR | | |
| | | DMGGGGSGGGGSGGGGSGGGGSGSHSMRYFSTSVSWPGR | | |
| | | GEPRFIAVGYVDDTQFVRFDSDAASPRGEPREPWVEQEG | | |
| | | PEYWDRETQKYKRQAQTDRVNLRKLRGYYNQSEDGSHTL | | dimer |
| | | QRMFGCDLGPDGRLLRGYNQFAYDGKDYIALNEDLRSWT | | |
| | | AADTAAQITQRKWEAAREAEQRRAYLEGTCVEWLRRYLE | | |
| | | NGKETLQRAEHPKTHVTHHPVSDHEATLRCWALGFYPAE | | |
| | | ITLTWQWDGEDQTQDTELVETRPAGDGTFQKWAAVVVPS | | |
| | | GEEQRYTCHVQHEGLPEPLTLRWKPSSQPTIPIVGIVAG | | |
| | | LAVLAVLAVLGAMVAVVMCRRKSSGGKGGSCSQAASSNS | | |
| | | AQGSDESLIACKA | | |
| B2M/HLA- | 307 | MSRSVALAVLALLSLSGLEAIQRTPKIQVYSRHPAENGK | 527 | single |
| C*04(A73T/ | | SNFLNCYVSGFHPSDIEVDLLKNGERIEKVEHSDLSFSK | | chain |
| D227K/T228A) | | DWSFYLLYYTEFTPTEKDEYACRVNHVTLSQPKIVKWDR | | dimer |
| | | DMGGGGSGGGGSGGGGSGGGGSGSHSMRYFSTSVSWPGR | | |
| | | GEPRFIAVGYVDDTQFVRFDSDAASPRGEPREPWVEQEG | | |
| | | PEYWDRETQKYKRQAQTDRVNLRKLRGYYNQSEDGSHTL | | |
| | | QRMFGCDLGPDGRLLRGYNQFAYDGKDYIALNEDLRSWT | | |
| | | AADTAAQITQRKWEAAREAEQRRAYLEGTCVEWLRRYLE | | |
| | | NGKETLQRAEHPKTHVTHHPVSDHEATLRCWALGFYPAE | | |
| | | ITLTWQWDGEDQTQKAELVETRPAGDGTFQKWAAVVVPS | | |
| | | GEEQRYTCHVQHEGLPEPLTLRWKPSSQPTIPIVGIVAG | | |
| | | LAVLAVLAVLGAMVAVVMCRRKSSGGKGGSCSQAASSNS | | |
| | | AQGSDESLIACKA | | |
| cPep/B2M/ | 322 | MSRSVALAVLALLSLSGLEARYRPGTVALGGGGSGGGGS | 528 | single |
| HLA- | | GGGGSIQRTPKIQVYSRHPAENGKSNFLNCYVSGFHPSD | | chain |
| C*07(C1G/ | | IEVDLLKNGERIEKVEHSDLSFSKDWSFYLLYYTEFTPT | | trimer |
| A73T) | | EKDEYACRVNHVTLSQPKIVKWDRDMGGGGSGGGGSGGG | | |
| | | GSGGGGSGSHSMRYFDTAVSRPGRGEPRFISVGYVDDTQ | | |
| | | FVRFDSDAASPRGEPRAPWVEQEGPEYWDRETQKYKRQA | | |
| | | QTDRVSLRNLRGYYNQSEDGSHTLQRMSGCDLGPDGRLL | | |
| | | RGYDQSAYDGKDYIALNEDLRSWTAADTAAQITQRKLEA | | |
| | | ARAAEQLRAYLEGTCVEWLRRYLENGKETLQRAEPPKTH | | |
| | | VTHHPLSDHEATLRCWALGFYPAEITLTWQRDGEDQTQD | | |
| | | TELVETRPAGDGTFQKWAAVVVPSGQEQRYTCHMQHEGL | | |
| | | QEPLTLSWEPSSQPTIPIMGIVAGLAVLVVLAVLGAVVT | | |
| | | AMMCRRKSSGGKGGSCSQAACSNSAQGSDESLITCKA | | |
| IV9(AA)/B2M/ | 337 | MSRSVALAVLALLSLSGLEAIIDKSGAAVGGGGSG | 533 | single |
| HLA- | | GGGSGGGGSIQRTPKIQVYSRHPAENGKSNFLNCY | | chain |
| C*05(C1G) | | VSGFHPSDIEVDLLKNGERIEKVEHSDLSFSKDWS | | trimer |
| | | FYLLYYTEFTPTEKDEYACRVNHVTLSQPKIVKWD | | |
| | | RDMGGGGSGGGGSGGGGSGGGGSGSHSMRYFYTAV | | |
| | | SRPGRGEPRFIAVGYVDDTQFVQFDSDAASPRGEP | | |
| | | RAPWVEQEGPEYWDRETQKYKRQAQTDRVNLRKLR | | |
| | | GYYNQSEAGSHTLQRMYGCDLGPDGRLLRGYNQFA | | |
| | | YDGKDYIALNEDLRSWTAADKAAQITQRKWEAARE | | |
| | | AEQRRAYLEGTCVEWLRRYLENGKKTLQRAEHPKT | | |
| | | HVTHHPVSDHEATLRCWALGFYPAEITLTWQRDGE | | |
| | | DQTQDTELVETRPAGDGTFQKWAAVVVPSGEEQRY | | |
| | | TCHVQHEGLPEPLTLRWGPSSQPTIPIVGIVAGLA | | |
| | | VLAVLAVLGAVMAVVMCRRKSSGGKGGSCSQAASS | | |
| | | NSAQGSDESLIACKA | | |
| IV9(EE)/B2M/ | 339 | MSRSVALAVLALLSLSGLEAIIDKSGEEVGGGGSG | 534 | single |
| HLA- | | GGGSGGGGSIQRTPKIQVYSRHPAENGKSNFLNCY | | chain |
| C*05(C1G) | | VSGFHPSDIEVDLLKNGERIEKVEHSDLSFSKDWS | | trimer |
| | | FYLLYYTEFTPTEKDEYACRVNHVTLSQPKIVKWD | | |
| | | RDMGGGGSGGGGSGGGGSGGGGSGSHSMRYFYTAV | | |
| | | SRPGRGEPRFIAVGYVDDTQFVQFDSDAASPRGEP | | |
| | | RAPWVEQEGPEYWDRETQKYKRQAQTDRVNLRKLR | | |
| | | GYYNQSEAGSHTLQRMYGCDLGPDGRLLRGYNQFA | | |
| | | YDGKDYIALNEDLRSWTAADKAAQITQRKWEAARE | | |
| | | AEQRRAYLEGTCVEWLRRYLENGKKTLQRAEHPKT | | |
| | | HVTHHPVSDHEATLRCWALGFYPAEITLTWQRDGE | | |
| | | DQTQDTELVETRPAGDGTFQKWAAVVVPSGEEQRY | | |
| | | TCHVQHEGLPEPLTLRWGPSSQPTIPIVGIVAGLA | | |
| | | VLAVLAVLGAVMAVVMCRRKSSGGKGGSCSQAASS | | |
| | | NSAQGSDESLIACKA | | |
| IV9(LA)/B2M/ | 338 | MSRSVALAVLALLSLSGLEAIIDKSGLAVGGGGSG | 535 | single |
| HLA- | | GGGSGGGGSIQRTPKIQVYSRHPAENGKSNFLNCY | | chain |
| C*05(C1G) | | VSGFHPSDIEVDLLKNGERIEKVEHSDLSFSKDWS | | trimer |
| | | FYLLYYTEFTPTEKDEYACRVNHVTLSQPKIVKWD | | |
| | | RDMGGGGSGGGGSGGGGSGGGGSGSHSMRYFYTAV | | |
| | | SRPGRGEPRFIAVGYVDDTQFVQFDSDAASPRGEP | | |
| | | RAPWVEQEGPEYWDRETQKYKRQAQTDRVNLRKLR | | |
| | | GYYNQSEAGSHTLQRMYGCDLGPDGRLLRGYNQFA | | |
| | | YDGKDYIALNEDLRSWTAADKAAQITQRKWEAARE | | |
| | | AEQRRAYLEGTCVEWLRRYLENGKKTLQRAEHPKT | | |
| | | HVTHHPVSDHEATLRCWALGFYPAEITLTWQRDGE | | |
| | | DQTQDTELVETRPAGDGTFQKWAAVVVPSGEEQRY | | |
| | | TCHVQHEGLPEPLTLRWGPSSQPTIPIVGIVAGLA | | |
| | | VLAVLAVLGAVMAVVMCRRKSSGGKGGSCSQAASS | | |
| | | NSAQGSDESLIACKA | | |
| IV9(WT)/ | 336 | MSRSVALAVLALLSLSGLEAIIDKSGSTVGGGGSG | 536 | single |
| B2M/HLA- | | GGGSGGGGSIQRTPKIQVYSRHPAENGKSNFLNCY | | chain |
| C*05(C1G) | | VSGFHPSDIEVDLLKNGERIEKVEHSDLSFSKDWS | | trimer |
| | | FYLLYYTEFTPTEKDEYACRVNHVTLSQPKIVKWD | | |
| | | RDMGGGGSGGGGSGGGGSGGGGSGSHSMRYFYTAV | | |
| | | SRPGRGEPRFIAVGYVDDTQFVQFDSDAASPRGEP | | |
| | | RAPWVEQEGPEYWDRETQKYKRQAQTDRVNLRKLR | | |
| | | GYYNQSEAGSHTLQRMYGCDLGPDGRLLRGYNQFA | | |
| | | YDGKDYIALNEDLRSWTAADKAAQITQRKWEAARE | | |
| | | AEQRRAYLEGTCVEWLRRYLENGKKTLQRAEHPKT | | |
| | | HVTHHPVSDHEATLRCWALGFYPAEITLTWQRDGE | | |
| | | DQTQDTELVETRPAGDGTFQKWAAVVVPSGEEQRY | | |
| | | TCHVQHEGLPEPLTLRWGPSSQPTIPIVGIVAGLA | | |
| | | VLAVLAVLGAVMAVVMCRRKSSGGKGGSCSQAASS | | |
| | | NSAQGSDESLIACKA | | |
| B2M/HLA- | 268 | MSRSVALAVLALLSLSGLEAIQRTPKIQVYSRHPA | 659 | single |
| E-CD4TM | | ENGKSNFLNCYVSGFHPSDIEVDLLKNGERIEKVE | | chain |
| | | HSDLSFSKDWSFYLLYYTEFTPTEKDEYACRVNHV | | dimer |
| | | TLSQPKIVKWDRDMGGGGSGGGGSGGGGSGGGGSG | | |
| | | SHSLKYFHTSVSRPGRGEPRFISVGYVDDTQFVRF | | |
| | | DNDAASPRMVPRAPWMEQEGSEYWDRETRSARDTA | | |
| | | QIFRVNLRTLRGYYNQSEAGSHTLQWMHGCELGPD | | |
| | | GRFLRGYEQFAYDGKDYLTLNEDLRSWTAVDTAAQ | | |
| | | ISEQKSNDASEAEHQRAYLEDTCVEWLHKYLEKGK | | |
| | | ETLLHLEPPKTHVTHHPISDHEATLRCWALGFYPA | | |
| | | EITLTWQQDGEGHTQDTELVETRPAGDGTFQKWAA | | |
| | | VVVPSGEEQRYTCHVQHEGLPEPVTLRWKPASQPT | | |
| | | IPIMALIVLGGVAGLLLFIGLGIFFCVRC | | |
| cPep/B2M/ | 269 | MSRSVALAVLALLSLSGLEAVMAPRTLFLGGGGSG | 660 | single |
| HLA-E- | | GGGSGGGGSIQRTPKIQVYSRHPAENGKSNFLNCY | | chain |
| CD4TM | | VSGFHPSDIEVDLLKNGERIEKVEHSDLSFSKDWS | | trimer |
| | | FYLLYYTEFTPTEKDEYACRVNHVTLSQPKIVKWD | | |
| | | RDMGGGGSGGGGSGGGGSGGGGSGSHSLKYFHTSV | | |
| | | SRPGRGEPRFISVGYVDDTQFVRFDNDAASPRMVP | | |
| | | RAPWMEQEGSEYWDRETRSARDTAQIFRVNLRTLR | | |
| | | GYYNQSEAGSHTLQWMHGCELGPDGRFLRGYEQFA | | |
| | | YDGKDYLTLNEDLRSWTAVDTAAQISEQKSNDASE | | |
| | | AEHQRAYLEDTCVEWLHKYLEKGKETLLHLEPPKT | | |
| | | HVTHHPISDHEATLRCWALGFYPAEITLTWQQDGE | | |
| | | GHTQDTELVETRPAGDGTFQKWAAVVVPSGEEQRY | | |
| | | TCHVQHEGLPEPVTLRWKPASQPTIPIMALIVLGG | | |
| | | VAGLLLFIGLGIFFCVRC | | |
| CTLA4(ECD)/ | 325 | MSRSVALAVLALLSLSGLEAKAMHVAQPAVVLASS | 661 | functio |
| B2M/ | | RGIASFVCEYASPGKATEVRVTVLRQADSQVTEVC | | nalized |
| HLA-B*57 | | AATYMMGNELTFLDDSICTGTSSGNQVNLTIQGLR | | single |
| | | AMDTGLYICKVELMYPPPYYLGIGNGTQIYVIDPE | | chain |
| | | PCPDSDGGGGSGGGGSGGGGSGGGGSIQRTPKIQV | | dimer |
| | | YSRHPAENGKSNFLNCYVSGFHPSDIEVDLLKNGE | | |
| | | RIEKVEHSDLSFSKDWSFYLLYYTEFTPTEKDEYA | | |
| | | CRVNHVTLSQPKIVKWDRDMGGGGSGGGGSGGGGS | | |
| | | GGGGSGSHSMRYFYTAMSRPGRGEPRFIAVGYVDD | | |
| | | TQFVRFDSDAASPRMAPRAPWIEQEGPEYWDGETR | | |
| | | NMKASAQTYRENLRIALRYYNQSEAGSHIIQVMYG | | |
| | | CDVGPDGRLLRGHDQSAYDGKDYIALNEDLSSWTA | | |
| | | ADTAAQITQRKWEAARVAEQLRAYLEGLCVEWLRR | | |
| | | YLENGKETLQRADPPKTHVTHHPISDHEATLRCWA | | |
| | | LGFYPAEITLTWQRDGEDQTQDTELVETRPAGDRT | | |
| | | FQKWAAVVVPSGEEQRYTCHVQHEGLPKPLTLRWE | | |
| | | PSSQSTVPIVGIVAGLAVLAVVVIGAVVAAVMCRR | | |
| | | KSSGGKGGSYSQAACSDSAQGSDVSLTA | | |
| B2M/HLA- | 327 | MSRSVALAVLALLSLSGLEAIQRTPKIQVYSRHPA | 662 | functio |
| B*57(ΔTM)- | | ENGKSNFLNCYVSGFHPSDIEVDLLINGERIEKVE | | nalized |
| CD47 | | HSDLSFSKDWSFYLLYYTEFTPTEKDEYACRVNHV | | single |
| | | TLSQPKIVKWDRDMGGGGSGGGGSGGGGSGGGGSG | | chain |
| | | SHSMRYFYTAMSRPGRGEPRFIAVGYVDDTQFVRF | | dimer |
| | | DSDAASPRMAPRAPWIEQEGPEYWDGETRNMKASA | | |
| | | QTYRENLRIALRYYNQSEAGSHIIQVMYGCDVGPD | | |
| | | GRLLRGHDQSAYDGKDYIALNEDLSSWTAADTAAQ | | |
| | | ITQRKWEAARVAEQLRAYLEGLCVEWLRRYLENGK | | |
| | | ETLQRADPPKTHVTHHPISDHEATLRCWALGFYPA | | |
| | | EITLTWQRDGEDQTQDTELVETRPAGDRTFQKWAA | | |
| | | VVVPSGEEQRYTCHVQHEGLPKPLTLRWEAAAGGQ | | |
| | | LLFNKTKSVEFTFCNDTVVIPCFVTNMEAQNTTEV | | |
| | | YVKWKFKGRDIYTFDGALNKSTVPTDFSSAKIEVS | | |
| | | QLLKGDASLKMDKSDAVSHTGNYTCEVTELTREGE | | |
| | | TIIELKYRVVSWFSPNENILIVIFPIFAILLFWGQ | | |
| | | FGIKTLKYRSGGMDEKTIALLVAGLVITVIVIVGA | | |
| | | ILFVPGEYSLKNATGLGLIVTSTGILILLHYYVFS | | |
| | | TAIGLTSFVIAILVIQVIAYILAVVGLSLCIAACI | | |
| | | PMHGPLLISGLSILALAQLLGLVYMKFVASNQKTI | | |
| | | QPPRKAVEEPLNAFKESKGMMNDE | | |
| B2M/HLA | 328 | MSRSVALAVLALLSLSGLEAIQRTPKIQVYSRHPA | 663 | functio |
| B*57(ΔTM)/ | | ENGKSNFLNCYVSGFHPSDIEVDLLKNGERIEKVE | | nalized |
| CD47 | | HSDLSFSKDWSFYLLYYTEFTPTEKDEYACRVNHV | | single |
| | | TLSQPKIVKWDRDMGGGGSGGGGSGGGGSGGGGSG | | chain |
| | | SHSMRYFYTAMSRPGRGEPRFIAVGYVDDTQFVRF | | dimer |
| | | DSDAASPRMAPRAPWIEQEGPEYWDGETRNMKASA | | |
| | | QTYRENLRIALRYYNQSEAGSHIIQVMYGCDVGPD | | |
| | | GRLLRGHDQSAYDGKDYIALNEDLSSWTAADTAAQ | | |
| | | ITQRKWEAARVAEQLRAYLEGLCVEWLRRYLENGK | | |
| | | ETLQRADPPKTHVTHHPISDHEATLRCWALGFYPA | | |
| | | EITLTWQRDGEDQTQDTELVETRPAGDRTFQKWAA | | |
| | | VVVPSGEEQRYTCHVQHEGLPKPLTLRWEGGGGSG | | |
| | | GGGSGGGGSGGGGSQLLFNKTKSVEFTFCNDTVVI | | |
| | | PCFVTNMEAQNTTEVYVKWKFKGRDIYTFDGALNK | | |
| | | STVPTDFSSAKIEVSQLLKGDASLKMDKSDAVSHT | | |
| | | GNYTCEVTELTREGETIIELKYRVVSWFSPNENIL | | |
| | | IVIFPIFAILLFWGQFGIKTLKYRSGGMDEKTIAL | | |
| | | LVAGLVITVIVIVGAILFVPGEYSLKNATGLGLIV | | |
| | | TSTGILILLHYYVFSTAIGLTSFVIAILVIQVIAY | | |
| | | ILAVVGLSLCIAACIPMHGPLLISGLSILALAQLL | | |
| | | GLVYMKFVASNQKTIQPPRKAVEEPLNAFKESKGM | | |
| | | MNDE | | |
| B2M/HLA- | 332 | MSRSVALAVLALLSLSGLEAIQRTPKIQVYSRHPA | 664 | functio |
| C*05(ΔTM)/ | | ENGKSNFLNCYVSGFHPSDIEVDLLKNGERIEKVE | | nalized |
| B2M/HLA- | | HSDLSFSKDWSFYLLYYTEFTPTEKDEYACRVNHV | | single |
| B*58 | | TLSQPKIVKWDRDMGGGGSGGGGSGGGGSGGGGSG | | chain |
| | | SHSMRYFYTAVSRPGRGEPRFIAVGYVDDTQFVQF | | dimer |
| | | DSDAASPRGEPRAPWVEQEGPEYWDRETQKYKRQA | | |
| | | QTDRVNLRKLRGYYNQSEAGSHTLQRMYGCDLGPD | | |
| | | GRLLRGYNQFAYDGKDYIALNEDLRSWTAADKAAQ | | |
| | | ITQRKWEAAREAEQRRAYLEGTCVEWLRRYLENGK | | |
| | | KTLQRAEHPKTHVTHHPVSDHEATLRCWALGFYPA | | |
| | | EITLTWQRDGEDQTQDTELVETRPAGDGTFQKWAA | | |
| | | VVVPSGEEQRYTCHVQHEGLPEPLTLRWGGGGGSG | | |
| | | GGGSGGGGSGGGGSIQRTPKIQVYSRHPAENGKSN | | |
| | | FLNCYVSGFHPSDIEVDLLKNGERIEKVEHSDLSF | | |
| | | SKDWSFYLLYYTEFTPTEKDEYACRVNHVTLSQPK | | |
| | | IVKWDRDMGGGGSGGGGSGGGGSGGGGSGSHSMRY | | |
| | | FYTAMSRPGRGEPRFIAVGYVDDTQFVRFDSDAAS | | |
| | | PRMAPRAPWIEQEGPEYWDGETRNMKASAQTYREN | | |
| | | LRIALRYYNQSEAGSHIIQVMYGCDVGPDGRLLRG | | |
| | | HDQSAYDGKDYIALNEDLSSWTAADTAAQITQRKW | | |
| | | EAARVAEQLRAYLEGLCVEWLRRYLENGKETLQRA | | |
| | | DPPKTHVTHHPISDHEATLRCWALGFYPAEITLTW | | |
| | | QRDGEDQTQDTELVETRPAGDRTFQKWAAVVVPSG | | |
| | | EEQRYTCHVQHEGLPKPLTLRWEPSSQSTVPIVGI | | |
| | | VAGLAVLAVVVIGAVVAAVMCRRKSSGGKGGSYSQ | | |
| | | AACSDSAQGSDVSLTA | | |
| cPep/B2M/ | 330 | MSRSVALAVLALLSLSGLEAVMAPRTLFLGGGGSG | 665 | function- |
| HLA-E | | GGGSGGGGSIQRTPKIQVYSRHPAENGKSNFLNCY | | alized |
| (ΔTM)/B2M/ | | VSGFHPSDIEVDLLKNGERIEKVEHSDLSFSKDWS | | single |
| HLA-B*57 | | FYLLYYTEFTPTEKDEYACRVNHVTLSQPKIVKWD | | chain |
| | | RDMGGGGSGGGGSGGGGSGGGGSGSHSLKYFHTSV | | dimer |
| | | SRPGRGEPRFISVGYVDDTQFVRFDNDAASPRMVP | | |
| | | RAPWMEQEGSEYWDRETRSARDTAQIFRVNLRTLR | | |
| | | GYYNQSEAGSHTLQWMHGCELGPDGRFLRGYEQFA | | |
| | | YDGKDYLTLNEDLRSWTAVDTAAQISEQKSNDASE | | |
| | | AEHQRAYLEDTCVEWLHKYLEKGKETLLHLEPPKT | | |
| | | HVTHHPISDHEATLRCWALGFYPAEITLTWQQDGE | | |
| | | GHTQDTELVETRPAGDGTFQKWAAVVVPSGEEQRY | | |
| | | TCHVQHEGLPEPVTLRWKGGGGSGGGGSGGGGSGG | | |
| | | GGSIQRTPKIQVYSRHPAENGKSNFLNCYVSGFHP | | |
| | | SDIEVDLLKNGERIEKVEHSDLSFSKDWSFYLLYY | | |
| | | TEFTPTEKDEYACRVNHVTLSQPKIVKWDRDMGGG | | |
| | | GSGGGGSGGGGSGGGGSGSHSMRYFYTAMSRPGRG | | |
| | | EPRFIAVGYVDDTQFVRFDSDAASPRMAPRAPWIE | | |
| | | QEGPEYWDGETRNMKASAQTYRENLRIALRYYNQS | | |
| | | EAGSHIIQVMYGCDVGPDGRLLRGHDQSAYDGKDY | | |
| | | IALNEDLSSWTAADTAAQITQRKWEAARVAEQLRA | | |
| | | YLEGLCVEWLRRYLENGKETLQRADPPKTHVTHHP | | |
| | | ISDHEATLRCWALGFYPAEITLTWQRDGEDQTQDT | | |
| | | ELVETRPAGDRTFQKWAAVVVPSGEEQRYTCHVQH | | |
| | | EGLPKPLTLRWEPSSQSTVPIVGIVAGLAVLAVVV | | |
| | | IGAVVAAVMCRRKSSGGKGGSYSQAACSDSAQGSD | | |
| | | VSLTA | | |

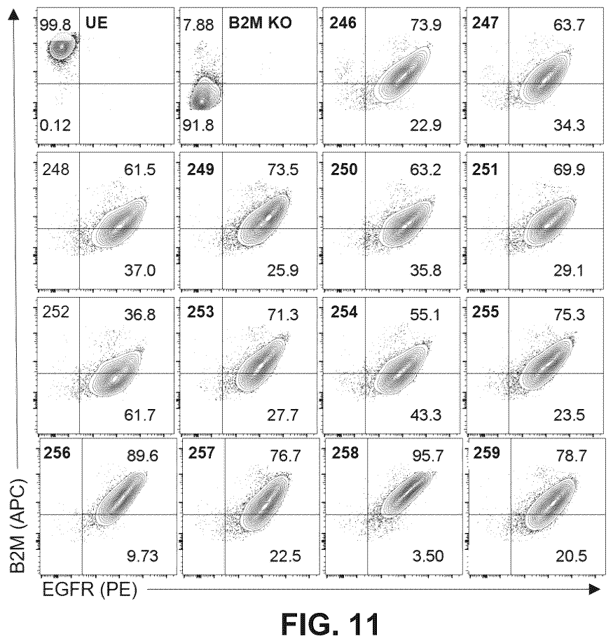

¹In the descriptions of each polypeptide, the domains contained within the polypeptide are listed in order from N-terminus to C-terminus and backslashes (“/”) that are not contained within parenthesis represent linkers). Signal peptides are indicated by bold italic text, linker amino acid sequences are indicated by bold text, and transmembrane domains (e.g., a WT transmembrane domain, such as in B2M/HLA-A*02, or a CD4 transmembrane domain, such as in B2M/HLA- A*02-CD4TM) are indicated by underlined text. In Table 8A “cPep” indicates a cognate peptide. In Table 8A, IV9 (AA), IV9 (EE), IV9 (LA), IV9 (WT) represent alternative cPep sequences (see. e.g., Sim, et al. “Canonical and Cross-reactive Binding of NK Cell Inhibitory Receptors to Hla-C Allotypes is Dictated by Peptides Bound to HLA-C, Frontiers in Immunology, vol. 8, art. 193 (2017), the disclosure of which is incorporated herein by reference in its entirety for all purposes). “Functionalized” single chain dimers and trimers are single-chain dimers and trimers that have been fused to an effector domain that inhibits activation of alloreactive T cells or natural killer cells (e.g., a CTLA4 extracellular domain (ECD), or an additional single-chain trimer or dimer, a CD47 domain). ΔTM indicates that the transmembrane (TM) domain of the indicated HLA class-I heavy chain domain has been deleted.
The identification numbers (ID#′s) correspond to those used in FIGs. 11-13.

TABLE 8B
Amino acid sequences for HLA class-I single-chain dimers.

| | | SEQ | |
|---|---|---|---|
| Description | Codon-optimized nucleotide sequence | ID NO | Format |
| B2M/HLA- | ATGAGCCGGAGCGTGGCTCTGGCCGTGCTGGCCCTATTGTCTCT | 510 | single |
| A*02 | GAGCGGACTGGAAGCCATCCAGAGAACCCCAAAGATCCAAGTGT | | chain |
| | ACAGCAGACACCCTGCCGAGAACGGCAAGAGCAATTTCCTGAAC | | dimer |
| | TGCTACGTGTCCGGCTTTCACCCCAGCGACATCGAGGTGGATCT | | |
| | GCTGAAGAACGGCGAGAGAATCGAGAAGGTCGAACACAGCGATC | | |
| | TGAGCTTCAGCAAGGATTGGTCCTTCTACCTGCTGTACTACACC | | |
| | GAGTTCACCCCTACAGAGAAGGACGAGTACGCCTGCAGAGTGAA | | |
| | CCACGTGACCCTGAGCCAGCCTAAGATCGTGAAGTGGGATAGAG | | |
| | ATATGGGCGGCGGAGGCTCTGGAGGCGGAGGCTCTGGAGGAGGC | | |
| | GGCTCCGGCGGAGGCGGCTCCGGCAGCCACTCCATGCGATATTT | | |
| | TTTTACCAGCGTATCTCGACCAGGCAGAGGCGAGCCCCGCTTCA | | |
| | TAGCGGTTGGCTACGTTGATGACACCCAATTTGTAAGATTCGAC | | |
| | TCAGATGCTGCGTCACAGAGAATGGAGCCAAGAGCCCCCTGGAT | | |
| | AGAACAGGAGGGTCCTGAATATTGGGATGGTGAGACAAGAAAGG | | |
| | TGAAAGCACATTCTCAGACTCATAGGGTAGATTTGGGTACTTTG | | |
| | AGAGGGTATTACAACCAGTCTGAAGCCGGTAGTCACACTGTTCA | | |
| | ACGCATGTACGGTTGTGATGTCGGATCAGATTGGCGCTTTCTCA | | |
| | GAGGGTATCACCAATATGCTTACGACGGGAAAGATTATATTGCG | | |
| | TTGAAGGAGGACCTCAGGAGTTGGACCGCAGCAGATATGGCCGC | | |
| | ACAGACTACGAAGCACAAGTGGGAGGCGGCCCATGTGGCGGAGC | | |
| | AACTGCGAGCGTATCTTGAGGGCACATGCGTAGAGTGGCTTAGG | | |
| | AGATATCTGGAGAATGGGAAGGAAACTTTGCAACGCACAGACGC | | |
| | TCCAAAGACGCACATGACTCATCATGCAGTGTCCGATCACGAAG | | |
| | CTACTCTCAGGTGTTGGGCTTTGTCATTCTATCCGGCTGAGATC | | |
| | ACCCTCACGTGGCAGCGGGATGGAGAAGATCAAACACAAGACAC | | |
| | AGAGCTTGTAGAGACACGCCCCGCTGGGGACGGCACCTTCCAAA | | |
| | AGTGGGCCGCTGTTGTTGTACCCTCAGGACAGGAACAGCGCTAT | | |
| | ACGTGCCACGTGCAGCATGAGGGCCTGCCGAAACCGCTGACACT | | |
| | TCGATGGGAGCCATCTTCTCAACCCACCATACCAATCGTGGGAA | | |
| | TCATAGCTGGATTGGTTCTTTTTGGTGCCGTAATCACCGGTGCT | | |
| | GTGGTTGCGGCGGTAATGTGGCGGCGCAAGAGCTCCGACAGAAA | | |
| | GGGTGGTAGCTATAGTCAGGCCGCTTCCAGTGACTCCGCACAGG | | |
| | GTAGCGACGTATCACTGACAGCGTGCAAGGTA | | |
| B2M/HLA- | ATGAGCCGGAGCGTGGCTCTGGCCGTGCTGGCCCTATTGTCTCT | 511 | single |
| A*02 | GAGCGGACTGGAAGCCATCCAGAGAACCCCAAAGATCCAAGTGT | | chain |
| (D227K/ | ACAGCAGACACCCTGCCGAGAACGGCAAGAGCAATTTCCTGAAC | | dimer |
| T228A) | TGCTACGTGTCCGGCTTTCACCCCAGCGACATCGAGGTGGATCT | | |
| | GCTGAAGAACGGCGAGAGAATCGAGAAGGTCGAACACAGCGATC | | |
| | TGAGCTTCAGCAAGGATTGGTCCTTCTACCTGCTGTACTACACC | | |
| | GAGTTCACCCCTACAGAGAAGGACGAGTACGCCTGCAGAGTGAA | | |
| | CCACGTGACCCTGAGCCAGCCTAAGATCGTGAAGTGGGATAGAG | | |
| | ATATGGGCGGCGGAGGCTCTGGAGGCGGAGGCTCTGGAGGAGGC | | |
| | GGCTCCGGCGGAGGCGGCTCCGGCAGCCACTCCATGCGATATTT | | |
| | TTTTACCAGCGTATCTCGACCAGGCAGAGGCGAGCCCCGCTTCA | | |
| | TAGCGGTTGGCTACGTTGATGACACCCAATTTGTAAGATTCGAC | | |
| | TCAGATGCTGCGTCACAGAGAATGGAGCCAAGAGCCCCCTGGAT | | |
| | AGAACAGGAGGGTCCTGAATATTGGGATGGTGAGACAAGAAAGG | | |
| | TGAAAGCACATTCTCAGACTCATAGGGTAGATTTGGGTACTTTG | | |
| | AGAGGGTATTACAACCAGTCTGAAGCCGGTAGTCACACTGTTCA | | |
| | ACGCATGTACGGTTGTGATGTCGGATCAGATTGGCGCTTTCTCA | | |
| | GAGGGTATCACCAATATGCTTACGACGGGAAAGATTATATTGCG | | |
| | TTGAAGGAGGACCTCAGGAGTTGGACCGCAGCAGATATGGCCGC | | |
| | ACAGACTACGAAGCACAAGTGGGAGGCGGCCCATGTGGCGGAGC | | |
| | AACTGCGAGCGTATCTTGAGGGCACATGCGTAGAGTGGCTTAGG | | |
| | AGATATCTGGAGAATGGGAAGGAAACTTTGCAACGCACAGACGC | | |
| | TCCAAAGACGCACATGACTCATCATGCAGTGTCCGATCACGAAG | | |
| | CTACTCTCAGGTGTTGGGCTTTGTCATTCTATCCGGCTGAGATC | | |
| | ACCCTCACGTGGCAGCGGGATGGAGAAGATCAAACACAAAAAGC | | |
| | GGAGCTTGTAGAGACACGCCCCGCTGGGGACGGCACCTTCCAAA | | |
| | AGTGGGCCGCTGTTGTTGTACCCTCAGGACAGGAACAGCGCTAT | | |
| | ACGTGCCACGTGCAGCATGAGGGCCTGCCGAAACCGCTGACACT | | |
| | TCGATGGGAGCCATCTTCTCAACCCACCATACCAATCGTGGGAA | | |
| | TCATAGCTGGATTGGTTCTTTTTGGTGCCGTAATCACCGGTGCT | | |
| | GTGGTTGCGGCGGTAATGTGGCGGCGCAAGAGCTCCGACAGAAA | | |
| | GGGTGGTAGCTATAGTCAGGCCGCTTCCAGTGACTCCGCACAGG | | |
| | GTAGCGACGTATCACTGACAGCGTGCAAGGTA | | |
| B2M/HLA- | ATGAGCCGGAGCGTGGCTCTGGCCGTGCTGGCCCTATTGTCTCT | 512 | single |
| A*02 | GAGCGGACTGGAAGCCATCCAGAGAACCCCAAAGATCCAAGTGT | | chain |
| (A245V) | ACAGCAGACACCCTGCCGAGAACGGCAAGAGCAATTTCCTGAAC | | dimer |
| | TGCTACGTGTCCGGCTTTCACCCCAGCGACATCGAGGTGGATCT | | |
| | GCTGAAGAACGGCGAGAGAATCGAGAAGGTCGAACACAGCGATC | | |
| | TGAGCTTCAGCAAGGATTGGTCCTTCTACCTGCTGTACTACACC | | |
| | GAGTTCACCCCTACAGAGAAGGACGAGTACGCCTGCAGAGTGAA | | |
| | CCACGTGACCCTGAGCCAGCCTAAGATCGTGAAGTGGGATAGAG | | |
| | ATATGGGCGGCGGAGGCTCTGGAGGCGGAGGCTCTGGAGGAGGC | | |
| | GGCTCCGGCGGAGGCGGCTCCGGCAGCCACTCCATGCGATATTT | | |
| | TTTTACCAGCGTATCTCGACCAGGCAGAGGCGAGCCCCGCTTCA | | |
| | TAGCGGTTGGCTACGTTGATGACACCCAATTTGTAAGATTCGAC | | |
| | TCAGATGCTGCGTCACAGAGAATGGAGCCAAGAGCCCCCTGGAT | | |
| | AGAACAGGAGGGTCCTGAATATTGGGATGGTGAGACAAGAAAGG | | |
| | TGAAAGCACATTCTCAGACTCATAGGGTAGATTTGGGTACTTTG | | |
| | AGAGGGTATTACAACCAGTCTGAAGCCGGTAGTCACACTGTTCA | | |
| | ACGCATGTACGGTTGTGATGTCGGATCAGATTGGCGCTTTCTCA | | |
| | GAGGGTATCACCAATATGCTTACGACGGGAAAGATTATATTGCG | | |
| | TTGAAGGAGGACCTCAGGAGTTGGACCGCAGCAGATATGGCCGC | | |
| | ACAGACTACGAAGCACAAGTGGGAGGCGGCCCATGTGGCGGAGC | | |
| | AACTGCGAGCGTATCTTGAGGGCACATGCGTAGAGTGGCTTAGG | | |
| | AGATATCTGGAGAATGGGAAGGAAACTTTGCAACGCACAGACGC | | |
| | TCCAAAGACGCACATGACTCATCATGCAGTGTCCGATCACGAAG | | |
| | CTACTCTCAGGTGTTGGGCTTTGTCATTCTATCCGGCTGAGATC | | |
| | ACCCTCACGTGGCAGCGGGATGGAGAAGATCAAACACAAGACAC | | |
| | AGAGCTTGTAGAGACACGCCCCGCTGGGGACGGCACCTTCCAAA | | |
| | AGTGGGTGGCTGTTGTTGTACCCTCAGGACAGGAACAGCGCTAT | | |
| | ACGTGCCACGTGCAGCATGAGGGCCTGCCGAAACCGCTGACACT | | |
| | TCGATGGGAGCCATCTTCTCAACCCACCATACCAATCGTGGGAA | | |
| | TCATAGCTGGATTGGTTCTTTTTGGTGCCGTAATCACCGGTGCT | | |
| | GTGGTTGCGGCGGTAATGTGGCGGCGCAAGAGCTCCGACAGAAA | | |
| | GGGTGGTAGCTATAGTCAGGCCGCTTCCAGTGACTCCGCACAGG | | |
| | GTAGCGACGTATCACTGACAGCGTGCAAGGTA | | |
| B2M/HLA- | ATGAGCCGGAGCGTGGCTCTGGCCGTGCTGGCCCTATTGTCTCT | 513 | single |
| A*02 | GAGCGGACTGGAAGCCATCCAGAGAACCCCAAAGATCCAAGTGT | | chain |
| (D227K/ | ACAGCAGACACCCTGCCGAGAACGGCAAGAGCAATTTCCTGAAC | | dimer |
| T228A/A245V) | TGCTACGTGTCCGGCTTTCACCCCAGCGACATCGAGGTGGATCT | | |
| | GCTGAAGAACGGCGAGAGAATCGAGAAGGTCGAACACAGCGATC | | |
| | TGAGCTTCAGCAAGGATTGGTCCTTCTACCTGCTGTACTACACC | | |
| | GAGTTCACCCCTACAGAGAAGGACGAGTACGCCTGCAGAGTGAA | | |
| | CCACGTGACCCTGAGCCAGCCTAAGATCGTGAAGTGGGATAGAG | | |
| | ATATGGGCGGCGGAGGCTCTGGAGGCGGAGGCTCTGGAGGAGGC | | |
| | GGCTCCGGCGGAGGCGGCTCCGGCAGCCACTCCATGCGATATTT | | |
| | TTTTACCAGCGTATCTCGACCAGGCAGAGGCGAGCCCCGCTTCA | | |
| | TAGCGGTTGGCTACGTTGATGACACCCAATTTGTAAGATTCGAC | | |
| | TCAGATGCTGCGTCACAGAGAATGGAGCCAAGAGCCCCCTGGAT | | |
| | AGAACAGGAGGGTCCTGAATATTGGGATGGTGAGACAAGAAAGG | | |
| | TGAAAGCACATTCTCAGACTCATAGGGTAGATTTGGGTACTTTG | | |
| | AGAGGGTATTACAACCAGTCTGAAGCCGGTAGTCACACTGTTCA | | |
| | ACGCATGTACGGTTGTGATGTCGGATCAGATTGGCGCTTTCTCA | | |
| | GAGGGTATCACCAATATGCTTACGACGGGAAAGATTATATTGCG | | |
| | TTGAAGGAGGACCTCAGGAGTTGGACCGCAGCAGATATGGCCGC | | |
| | ACAGACTACGAAGCACAAGTGGGAGGCGGCCCATGTGGCGGAGC | | |
| | AACTGCGAGCGTATCTTGAGGGCACATGCGTAGAGTGGCTTAGG | | |
| | AGATATCTGGAGAATGGGAAGGAAACTTTGCAACGCACAGACGC | | |
| | TCCAAAGACGCACATGACTCATCATGCAGTGTCCGATCACGAAG | | |
| | CTACTCTCAGGTGTTGGGCTTTGTCATTCTATCCGGCTGAGATC | | |
| | ACCCTCACGTGGCAGCGGGATGGAGAAGATCAAACACAAAAAGC | | |
| | GGAGCTTGTAGAGACACGCCCCGCTGGGGACGGCACCTTCCAAA | | |
| | AGTGGGTGGCTGTTGTTGTACCCTCAGGACAGGAACAGCGCTAT | | |
| | ACGTGCCACGTGCAGCATGAGGGCCTGCCGAAACCGCTGACACT | | |
| | TCGATGGGAGCCATCTTCTCAACCCACCATACCAATCGTGGGAA | | |
| | TCATAGCTGGATTGGTTCTTTTTGGTGCCGTAATCACCGGTGCT | | |
| | GTGGTTGCGGCGGTAATGTGGCGGCGCAAGAGCTCCGACAGAAA | | |
| | GGGTGGTAGCTATAGTCAGGCCGCTTCCAGTGACTCCGCACAGG | | |
| | GTAGCGACGTATCACTGACAGCGTGCAAGGTA | | |
| B2M(K58E)/ | ATGAGCCGGAGCGTGGCTCTGGCCGTGCTGGCCCTATTGTCTCT | 514 | single |
| HLA-A*02 | GAGCGGACTGGAAGCCATCCAGAGAACCCCAAAGATCCAAGTGT | | chain |
| | ACAGCAGACACCCTGCCGAGAACGGCAAGAGCAATTTCCTGAAC | | dimer |
| | TGCTACGTGTCCGGCTTTCACCCCAGCGACATCGAGGTGGATCT | | |
| | GCTGAAGAACGGCGAGAGAATCGAGAAGGTCGAACACAGCGATC | | |
| | TGAGCTTCAGCGAAGATTGGTCCTTCTACCTGCTGTACTACACC | | |
| | GAGTTCACCCCTACAGAGAAGGACGAGTACGCCTGCAGAGTGAA | | |
| | CCACGTGACCCTGAGCCAGCCTAAGATCGTGAAGTGGGATAGAG | | |
| | ATATGGGCGGCGGAGGCTCTGGAGGCGGAGGCTCTGGAGGAGGC | | |
| | GGCTCCGGCGGAGGCGGCTCCGGCAGCCACTCCATGCGATATTT | | |
| | TTTTACCAGCGTATCTCGACCAGGCAGAGGCGAGCCCCGCTTCA | | |
| | TAGCGGTTGGCTACGTTGATGACACCCAATTTGTAAGATTCGAC | | |
| | TCAGATGCTGCGTCACAGAGAATGGAGCCAAGAGCCCCCTGGAT | | |
| | AGAACAGGAGGGTCCTGAATATTGGGATGGTGAGACAAGAAAGG | | |
| | TGAAAGCACATTCTCAGACTCATAGGGTAGATTTGGGTACTTTG | | |
| | AGAGGGTATTACAACCAGTCTGAAGCCGGTAGTCACACTGTTCA | | |
| | ACGCATGTACGGTTGTGATGTCGGATCAGATTGGCGCTTTCTCA | | |
| | GAGGGTATCACCAATATGCTTACGACGGGAAAGATTATATTGCG | | |
| | TTGAAGGAGGACCTCAGGAGTTGGACCGCAGCAGATATGGCCGC | | |
| | ACAGACTACGAAGCACAAGTGGGAGGCGGCCCATGTGGCGGAGC | | |
| | AACTGCGAGCGTATCTTGAGGGCACATGCGTAGAGTGGCTTAGG | | |
| | AGATATCTGGAGAATGGGAAGGAAACTTTGCAACGCACAGACGC | | |
| | TCCAAAGACGCACATGACTCATCATGCAGTGTCCGATCACGAAG | | |
| | CTACTCTCAGGTGTTGGGCTTTGTCATTCTATCCGGCTGAGATC | | |
| | ACCCTCACGTGGCAGCGGGATGGAGAAGATCAAACACAAGACAC | | |
| | AGAGCTTGTAGAGACACGCCCCGCTGGGGACGGCACCTTCCAAA | | |
| | AGTGGGCCGCTGTTGTTGTACCCTCAGGACAGGAACAGCGCTAT | | |
| | ACGTGCCACGTGCAGCATGAGGGCCTGCCGAAACCGCTGACACT | | |
| | TCGATGGGAGCCATCTTCTCAACCCACCATACCAATCGTGGGAA | | |
| | TCATAGCTGGATTGGTTCTTTTTGGTGCCGTAATCACCGGTGCT | | |
| | GTGGTTGCGGCGGTAATGTGGCGGCGCAAGAGCTCCGACAGAAA | | |
| | GGGTGGTAGCTATAGTCAGGCCGCTTCCAGTGACTCCGCACAGG | | |
| | GTAGCGACGTATCACTGACAGCGTGCAAGGTA | | |
| B2M(K58E)/ | ATGAGCCGGAGCGTGGCTCTGGCCGTGCTGGCCCTATTGTCTCT | 515 | single |
| HLA- | GAGCGGACTGGAAGCCATCCAGAGAACCCCAAAGATCCAAGTGT | | chain |
| A*02 | ACAGCAGACACCCTGCCGAGAACGGCAAGAGCAATTTCCTGAAC | | dimer |
| (D227K/T228A) | TGCTACGTGTCCGGCTTTCACCCCAGCGACATCGAGGTGGATCT | | |
| | GCTGAAGAACGGCGAGAGAATCGAGAAGGTCGAACACAGCGATC | | |
| | TGAGCTTCAGCGAAGATTGGTCCTTCTACCTGCTGTACTACACC | | |
| | GAGTTCACCCCTACAGAGAAGGACGAGTACGCCTGCAGAGTGAA | | |
| | CCACGTGACCCTGAGCCAGCCTAAGATCGTGAAGTGGGATAGAG | | |
| | ATATGGGCGGCGGAGGCTCTGGAGGCGGAGGCTCTGGAGGAGGC | | |
| | GGCTCCGGCGGAGGCGGCTCCGGCAGCCACTCCATGCGATATTT | | |
| | TTTTACCAGCGTATCTCGACCAGGCAGAGGCGAGCCCCGCTTCA | | |
| | TAGCGGTTGGCTACGTTGATGACACCCAATTTGTAAGATTCGAC | | |
| | TCAGATGCTGCGTCACAGAGAATGGAGCCAAGAGCCCCCTGGAT | | |
| | AGAACAGGAGGGTCCTGAATATTGGGATGGTGAGACAAGAAAGG | | |
| | TGAAAGCACATTCTCAGACTCATAGGGTAGATTTGGGTACTTTG | | |
| | AGAGGGTATTACAACCAGTCTGAAGCCGGTAGTCACACTGTTCA | | |
| | ACGCATGTACGGTTGTGATGTCGGATCAGATTGGCGCTTTCTCA | | |
| | GAGGGTATCACCAATATGCTTACGACGGGAAAGATTATATTGCG | | |
| | TTGAAGGAGGACCTCAGGAGTTGGACCGCAGCAGATATGGCCGC | | |
| | ACAGACTACGAAGCACAAGTGGGAGGCGGCCCATGTGGCGGAGC | | |
| | AACTGCGAGCGTATCTTGAGGGCACATGCGTAGAGTGGCTTAGG | | |
| | AGATATCTGGAGAATGGGAAGGAAACTTTGCAACGCACAGACGC | | |
| | TCCAAAGACGCACATGACTCATCATGCAGTGTCCGATCACGAAG | | |
| | CTACTCTCAGGTGTTGGGCTTTGTCATTCTATCCGGCTGAGATC | | |
| | ACCCTCACGTGGCAGCGGGATGGAGAAGATCAAACACAAAAAGC | | |
| | GGAGCTTGTAGAGACACGCCCCGCTGGGGACGGCACCTTCCAAA | | |
| | AGTGGGCCGCTGTTGTTGTACCCTCAGGACAGGAACAGCGCTAT | | |
| | ACGTGCCACGTGCAGCATGAGGGCCTGCCGAAACCGCTGACACT | | |
| | TCGATGGGAGCCATCTTCTCAACCCACCATACCAATCGTGGGAA | | |
| | TCATAGCTGGATTGGTTCTTTTTGGTGCCGTAATCACCGGTGCT | | |
| | GTGGTTGCGGCGGTAATGTGGCGGCGCAAGAGCTCCGACAGAAA | | |
| | GGGTGGTAGCTATAGTCAGGCCGCTTCCAGTGACTCCGCACAGG | | |
| | GTAGCGACGTATCACTGACAGCGTGCAAGGTA | | |
| B2M(K58E)/ | ATGAGCCGGAGCGTGGCTCTGGCCGTGCTGGCCCTATTGTCTCT | 516 | single |
| HLA- | GAGCGGACTGGAAGCCATCCAGAGAACCCCAAAGATCCAAGTGT | | chain |
| A*02(A245V) | ACAGCAGACACCCTGCCGAGAACGGCAAGAGCAATTTCCTGAAC | | dimer |
| | TGCTACGTGTCCGGCTTTCACCCCAGCGACATCGAGGTGGATCT | | |
| | GCTGAAGAACGGCGAGAGAATCGAGAAGGTCGAACACAGCGATC | | |
| | TGAGCTTCAGCGAAGATTGGTCCTTCTACCTGCTGTACTACACC | | |
| | GAGTTCACCCCTACAGAGAAGGACGAGTACGCCTGCAGAGTGAA | | |
| | CCACGTGACCCTGAGCCAGCCTAAGATCGTGAAGTGGGATAGAG | | |
| | ATATGGGCGGCGGAGGCTCTGGAGGCGGAGGCTCTGGAGGAGGC | | |
| | GGCTCCGGCGGAGGCGGCTCCGGCAGCCACTCCATGCGATATTT | | |
| | TTTTACCAGCGTATCTCGACCAGGCAGAGGCGAGCCCCGCTTCA | | |
| | TAGCGGTTGGCTACGTTGATGACACCCAATTTGTAAGATTCGAC | | |
| | TCAGATGCTGCGTCACAGAGAATGGAGCCAAGAGCCCCCTGGAT | | |
| | AGAACAGGAGGGTCCTGAATATTGGGATGGTGAGACAAGAAAGG | | |
| | TGAAAGCACATTCTCAGACTCATAGGGTAGATTTGGGTACTTTG | | |
| | AGAGGGTATTACAACCAGTCTGAAGCCGGTAGTCACACTGTTCA | | |
| | ACGCATGTACGGTTGTGATGTCGGATCAGATTGGCGCTTTCTCA | | |
| | GAGGGTATCACCAATATGCTTACGACGGGAAAGATTATATTGCG | | |
| | TTGAAGGAGGACCTCAGGAGTTGGACCGCAGCAGATATGGCCGC | | |
| | ACAGACTACGAAGCACAAGTGGGAGGCGGCCCATGTGGCGGAGC | | |
| | AACTGCGAGCGTATCTTGAGGGCACATGCGTAGAGTGGCTTAGG | | |
| | AGATATCTGGAGAATGGGAAGGAAACTTTGCAACGCACAGACGC | | |
| | TCCAAAGACGCACATGACTCATCATGCAGTGTCCGATCACGAAG | | |
| | CTACTCTCAGGTGTTGGGCTTTGTCATTCTATCCGGCTGAGATC | | |
| | ACCCTCACGTGGCAGCGGGATGGAGAAGATCAAACACAAGACAC | | |
| | AGAGCTTGTAGAGACACGCCCCGCTGGGGACGGCACCTTCCAAA | | |
| | AGTGGGTGGCTGTTGTTGTACCCTCAGGACAGGAACAGCGCTAT | | |
| | ACGTGCCACGTGCAGCATGAGGGCCTGCCGAAACCGCTGACACT | | |
| | TCGATGGGAGCCATCTTCTCAACCCACCATACCAATCGTGGGAA | | |
| | TCATAGCTGGATTGGTTCTTTTTGGTGCCGTAATCACCGGTGCT | | |
| | GTGGTTGCGGCGGTAATGTGGCGGCGCAAGAGCTCCGACAGAAA | | |
| | GGGTGGTAGCTATAGTCAGGCCGCTTCCAGTGACTCCGCACAGG | | |
| | GTAGCGACGTATCACTGACAGCGTGCAAGGTA | | |
| B2M(K58E)/ | ATGAGCCGGAGCGTGGCTCTGGCCGTGCTGGCCCTATTGTCTCT | 517 | single |
| HLA- | GAGCGGACTGGAAGCCATCCAGAGAACCCCAAAGATCCAAGTGT | | chain |
| A*02 | ACAGCAGACACCCTGCCGAGAACGGCAAGAGCAATTTCCTGAAC | | dimer |
| (D227K/ | TGCTACGTGTCCGGCTTTCACCCCAGCGACATCGAGGTGGATCT | | |
| T228A/A245V) | GCTGAAGAACGGCGAGAGAATCGAGAAGGTCGAACACAGCGATC | | |
| | TGAGCTTCAGCGAAGATTGGTCCTTCTACCTGCTGTACTACACC | | |
| | GAGTTCACCCCTACAGAGAAGGACGAGTACGCCTGCAGAGTGAA | | |
| | CCACGTGACCCTGAGCCAGCCTAAGATCGTGAAGTGGGATAGAG | | |
| | ATATGGGCGGCGGAGGCTCTGGAGGCGGAGGCTCTGGAGGAGGC | | |
| | GGCTCCGGCGGAGGCGGCTCCGGCAGCCACTCCATGCGATATTT | | |
| | TTTTACCAGCGTATCTCGACCAGGCAGAGGCGAGCCCCGCTTCA | | |
| | TAGCGGTTGGCTACGTTGATGACACCCAATTTGTAAGATTCGAC | | |
| | TCAGATGCTGCGTCACAGAGAATGGAGCCAAGAGCCCCCTGGAT | | |
| | AGAACAGGAGGGTCCTGAATATTGGGATGGTGAGACAAGAAAGG | | |
| | TGAAAGCACATTCTCAGACTCATAGGGTAGATTTGGGTACTTTG | | |
| | AGAGGGTATTACAACCAGTCTGAAGCCGGTAGTCACACTGTTCA | | |
| | ACGCATGTACGGTTGTGATGTCGGATCAGATTGGCGCTTTCTCA | | |
| | GAGGGTATCACCAATATGCTTACGACGGGAAAGATTATATTGCG | | |
| | TTGAAGGAGGACCTCAGGAGTTGGACCGCAGCAGATATGGCCGC | | |
| | ACAGACTACGAAGCACAAGTGGGAGGCGGCCCATGTGGCGGAGC | | |
| | AACTGCGAGCGTATCTTGAGGGCACATGCGTAGAGTGGCTTAGG | | |
| | AGATATCTGGAGAATGGGAAGGAAACTTTGCAACGCACAGACGC | | |
| | TCCAAAGACGCACATGACTCATCATGCAGTGTCCGATCACGAAG | | |
| | CTACTCTCAGGTGTTGGGCTTTGTCATTCTATCCGGCTGAGATC | | |
| | ACCCTCACGTGGCAGCGGGATGGAGAAGATCAAACACAAAAAGC | | |
| | GGAGCTTGTAGAGACACGCCCCGCTGGGGACGGCACCTTCCAAA | | |
| | AGTGGGTGGCTGTTGTTGTACCCTCAGGACAGGAACAGCGCTAT | | |
| | ACGTGCCACGTGCAGCATGAGGGCCTGCCGAAACCGCTGACACT | | |
| | TCGATGGGAGCCATCTTCTCAACCCACCATACCAATCGTGGGAA | | |
| | TCATAGCTGGATTGGTTCTTTTTGGTGCCGTAATCACCGGTGCT | | |
| | GTGGTTGCGGCGGTAATGTGGCGGCGCAAGAGCTCCGACAGAAA | | |
| | GGGTGGTAGCTATAGTCAGGCCGCTTCCAGTGACTCCGCACAGG | | |
| | GTAGCGACGTATCACTGACAGCGTGCAAGGTA | | |
| B2M/HLA- | ATGAGCCGGAGCGTGGCTCTGGCCGTGCTGGCCCTATTGTCTCT | 518 | single |
| A*02- | GAGCGGACTGGAAGCCATCCAGAGAACCCCAAAGATCCAAGTGT | | chain |
| CD4TM | ACAGCAGACACCCTGCCGAGAACGGCAAGAGCAATTTCCTGAAC | | dimer |
| | TGCTACGTGTCCGGCTTTCACCCCAGCGACATCGAGGTGGATCT | | |
| | GCTGAAGAACGGCGAGAGAATCGAGAAGGTCGAACACAGCGATC | | |
| | TGAGCTTCAGCAAGGATTGGTCCTTCTACCTGCTGTACTACACC | | |
| | GAGTTCACCCCTACAGAGAAGGACGAGTACGCCTGCAGAGTGAA | | |
| | CCACGTGACCCTGAGCCAGCCTAAGATCGTGAAGTGGGATAGAG | | |
| | ATATGGGCGGCGGAGGCTCTGGAGGCGGAGGCTCTGGAGGAGGC | | |
| | GGCTCCGGCGGAGGCGGCTCCGGCAGCCACTCCATGCGATATTT | | |
| | TTTTACCAGCGTATCTCGACCAGGCAGAGGCGAGCCCCGCTTCA | | |
| | TAGCGGTTGGCTACGTTGATGACACCCAATTTGTAAGATTCGAC | | |
| | TCAGATGCTGCGTCACAGAGAATGGAGCCAAGAGCCCCCTGGAT | | |
| | AGAACAGGAGGGTCCTGAATATTGGGATGGTGAGACAAGAAAGG | | |
| | TGAAAGCACATTCTCAGACTCATAGGGTAGATTTGGGTACTTTG | | |
| | AGAGGGTATTACAACCAGTCTGAAGCCGGTAGTCACACTGTTCA | | |
| | ACGCATGTACGGTTGTGATGTCGGATCAGATTGGCGCTTTCTCA | | |
| | GAGGGTATCACCAATATGCTTACGACGGGAAAGATTATATTGCG | | |
| | TTGAAGGAGGACCTCAGGAGTTGGACCGCAGCAGATATGGCCGC | | |
| | ACAGACTACGAAGCACAAGTGGGAGGCGGCCCATGTGGCGGAGC | | |
| | AACTGCGAGCGTATCTTGAGGGCACATGCGTAGAGTGGCTTAGG | | |
| | AGATATCTGGAGAATGGGAAGGAAACTTTGCAACGCACAGACGC | | |
| | TCCAAAGACGCACATGACTCATCATGCAGTGTCCGATCACGAAG | | |
| | CTACTCTCAGGTGTTGGGCTTTGTCATTCTATCCGGCTGAGATC | | |
| | ACCCTCACGTGGCAGCGGGATGGAGAAGATCAAACACAAGACAC | | |
| | AGAGCTTGTAGAGACACGCCCCGCTGGGGACGGCACCTTCCAAA | | |
| | AGTGGGCCGCTGTTGTTGTACCCTCAGGACAGGAACAGCGCTAT | | |
| | ACGTGCCACGTGCAGCATGAGGGCCTGCCGAAACCGCTGACACT | | |
| | TCGATGGGAGCCATCTTCTCAACCCACCATACCAATCATGGCCC | | |
| | TGATCGTGCTGGGCGGCGTGGCCGGCCTGCTGCTGTTCATCGGC | | |
| | CTGGGCATTTTTTTCTGCGTGCGGTGC | | |
| B2M/HLA- | ATGAGCCGGAGCGTGGCTCTGGCCGTGCTGGCCCTATTGTCTCT | 519 | single |
| A*02(D227K/ | GAGCGGACTGGAAGCCATCCAGAGAACCCCAAAGATCCAAGTGT | | chain |
| T228A)- | ACAGCAGACACCCTGCCGAGAACGGCAAGAGCAATTTCCTGAAC | | dimer |
| CD4TM | TGCTACGTGTCCGGCTTTCACCCCAGCGACATCGAGGTGGATCT | | |
| | GCTGAAGAACGGCGAGAGAATCGAGAAGGTCGAACACAGCGATC | | |
| | TGAGCTTCAGCAAGGATTGGTCCTTCTACCTGCTGTACTACACC | | |
| | GAGTTCACCCCTACAGAGAAGGACGAGTACGCCTGCAGAGTGAA | | |
| | CCACGTGACCCTGAGCCAGCCTAAGATCGTGAAGTGGGATAGAG | | |
| | ATATGGGCGGCGGAGGCTCTGGAGGCGGAGGCTCTGGAGGAGGC | | |
| | GGCTCCGGCGGAGGCGGCTCCGGCAGCCACTCCATGCGATATTT | | |
| | TTTTACCAGCGTATCTCGACCAGGCAGAGGCGAGCCCCGCTTCA | | |
| | TAGCGGTTGGCTACGTTGATGACACCCAATTTGTAAGATTCGAC | | |
| | TCAGATGCTGCGTCACAGAGAATGGAGCCAAGAGCCCCCTGGAT | | |
| | AGAACAGGAGGGTCCTGAATATTGGGATGGTGAGACAAGAAAGG | | |
| | TGAAAGCACATTCTCAGACTCATAGGGTAGATTTGGGTACTTTG | | |
| | AGAGGGTATTACAACCAGTCTGAAGCCGGTAGTCACACTGTTCA | | |
| | ACGCATGTACGGTTGTGATGTCGGATCAGATTGGCGCTTTCTCA | | |
| | GAGGGTATCACCAATATGCTTACGACGGGAAAGATTATATTGCG | | |
| | TTGAAGGAGGACCTCAGGAGTTGGACCGCAGCAGATATGGCCGC | | |
| | ACAGACTACGAAGCACAAGTGGGAGGCGGCCCATGTGGCGGAGC | | |
| | AACTGCGAGCGTATCTTGAGGGCACATGCGTAGAGTGGCTTAGG | | |
| | AGATATCTGGAGAATGGGAAGGAAACTTTGCAACGCACAGACGC | | |
| | TCCAAAGACGCACATGACTCATCATGCAGTGTCCGATCACGAAG | | |
| | CTACTCTCAGGTGTTGGGCTTTGTCATTCTATCCGGCTGAGATC | | |
| | ACCCTCACGTGGCAGCGGGATGGAGAAGATCAAACACAAAAAGC | | |
| | GGAGCTTGTAGAGACACGCCCCGCTGGGGACGGCACCTTCCAAA | | |
| | AGTGGGCCGCTGTTGTTGTACCCTCAGGACAGGAACAGCGCTAT | | |
| | ACGTGCCACGTGCAGCATGAGGGCCTGCCGAAACCGCTGACACT | | |
| | TCGATGGGAGCCATCTTCTCAACCCACCATACCAATCATGGCCC | | |
| | TGATCGTGCTGGGCGGCGTGGCCGGCCTGCTGCTGTTCATCGGC | | |
| | CTGGGCATTTTTTTCTGCGTGCGGTGC | | |

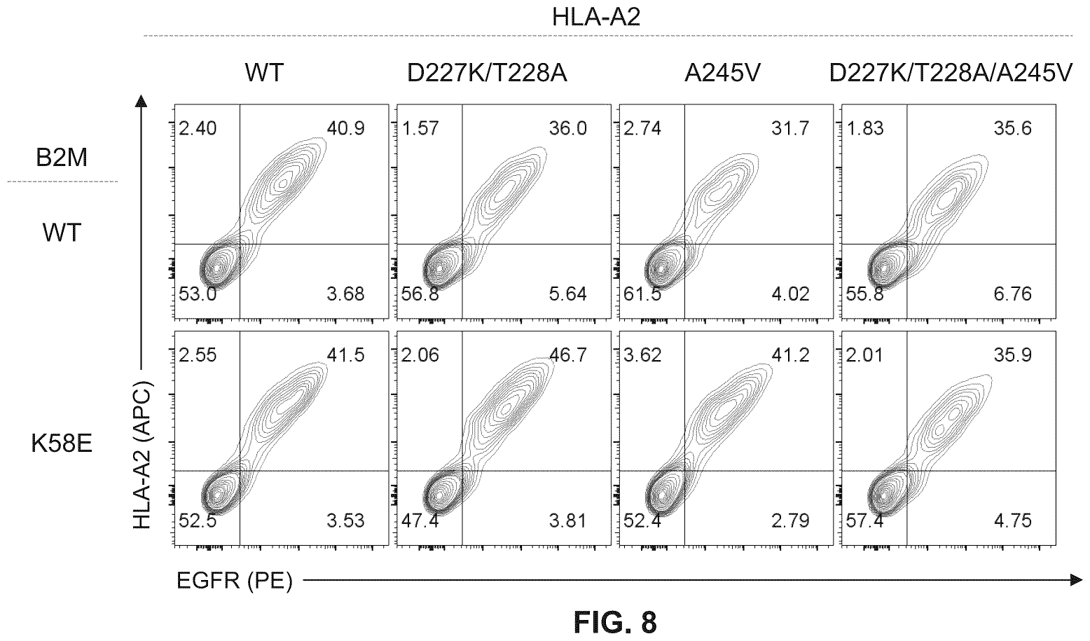

Lentiviral vectors were prepared containing polynucleotides encoding the following HLA-A2 single chain dimers (see Tables 8A and 8B): B2M/HLA-A*02; B2M/HLA-A*02 (D227K/T228A); B2M/HLA-A*02 (A245V); B2M/HLA-A*02 (D227K/T228A/A245V); B2M (K58E)/HLA-A*02; B2M (K58E)/HLA-A*02 (D227K/T228A); B2M (K58E)/HLA-A*02 (A245V); and B2M (K58E)/HLA-A*02 (D227K/T228A/A245V). Expression of the HLA-A2 single-chain dimers was controlled using an MND promoter (AATGAAAGACCCCACCTGTAGGTTTGGCAAGCTAGGATCAAGGTTAGGAACAGAGAGACAGCAG AATATGGGCCAAACAGGATATCTGTGGTAAGCAGTTCCTGCCCCGGCTCAGGGCCAAGAACAGT TGGAACAGCAGAATATGGGCCAAACAGGATATCTGTGGTAAGCAGTTCCTGCCCCGGCTCAGGG CCAAGAACAGATGGTCCCCAGATGCGGTCCCGCCCTCAGCAGTTTCTAGAGAACCATCAGATGT TTCCAGGGTGCCCCAAGGACCTGAAATGACCCTGTGCCTTATTTGAACTAACCAATCAGTTCGC TTCTCGCTTCTGTTCGCGCGCTTCTGCTCCCCGAGCTCAATAAAAGAGCCCACAACCCCTCACT CGGC (SEQ ID NO: 628)). To evaluate the efficiency of the lentiviral vectors, B2M KO T cells prepared as described above using base editing were contacted with the lentiviral vectors (i.e., transduced using the lentiviral vectors) and expression of the HLA-A2 single-chain dimers in the cells was then evaluated using flow cytometry (FIG. 8). All of the lentiviral vectors were effective at introducing the polynucleotides into the B2M KO T cells and the transduced B2M KO T cells expressed and surface-displayed the encoded polypeptides.

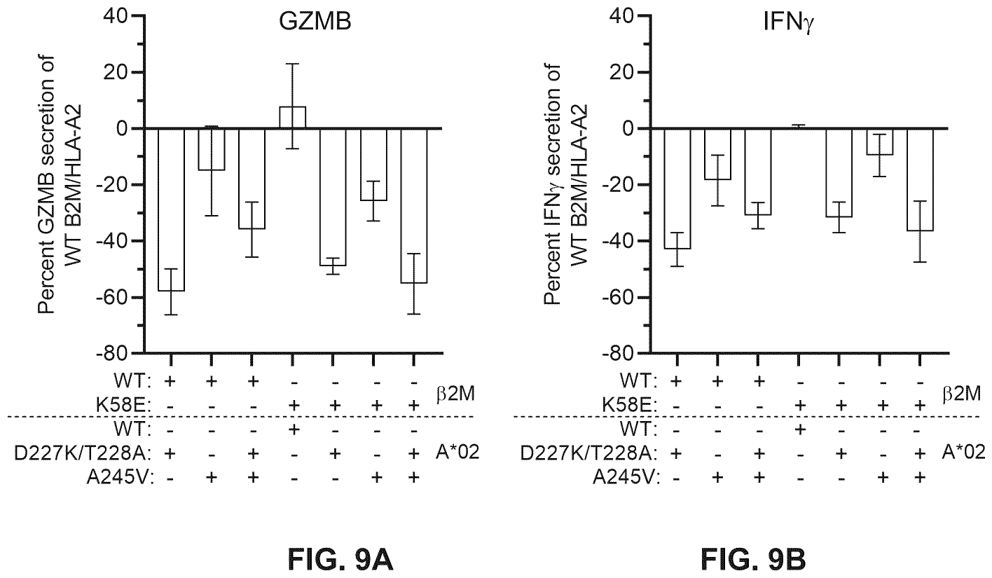

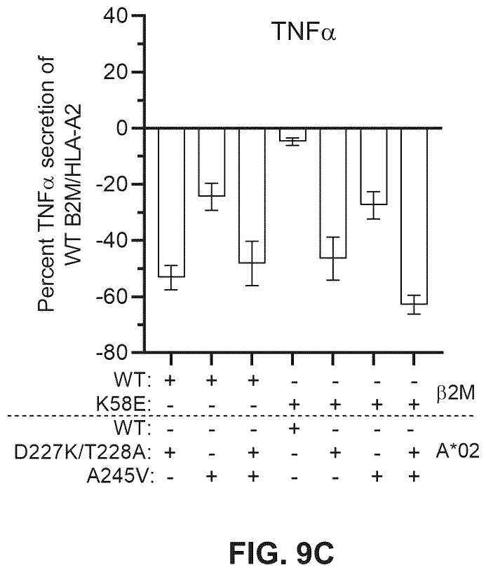

Next, experiments were undertaken to evaluate relative levels of activation of alloreactive T cells by the transduced B2M KO T cells expressing the HLA-A2 single chain dimers (FIGS. 9A-9C). The alloreactive T cells (the effector (E) cells) were co-cultured at an effector-to-target cell ratio of 1:1 with the transduced cells (the target (T) cells) for 24 hours, after which the cell culture medium was centrifuged and the supernatant was evaluated for levels of the following markers of activation of the alloreactive T cells: granzyme B (BZMB) (FIG. 9A), IFN-gamma (FIG. 9B), and TNF-alpha (FIG. 9C). Levels of the markers were measured relative to levels measured for B2M KO T cells transduced with an HLA-A2 single chain dimer containing wild type B2M and HLA-A2 domains. It was found that transduced cells expressing HLA-A2 single chain dimers containing a B2M (K58E) domain and/or an HLA-A2 (D227K/T228A), HLA-A2 (A245V), or HLA-A2 (D227K/T228A/A245V) domain showed reduced levels of alloreactive T cell activation relative to cells expressing HLA-A2 single chain dimers containing B2M (WT) and HLA-A2 (WT) domains, as shown by reduced levels of alloreactive T cell cytokine production in co-culture.

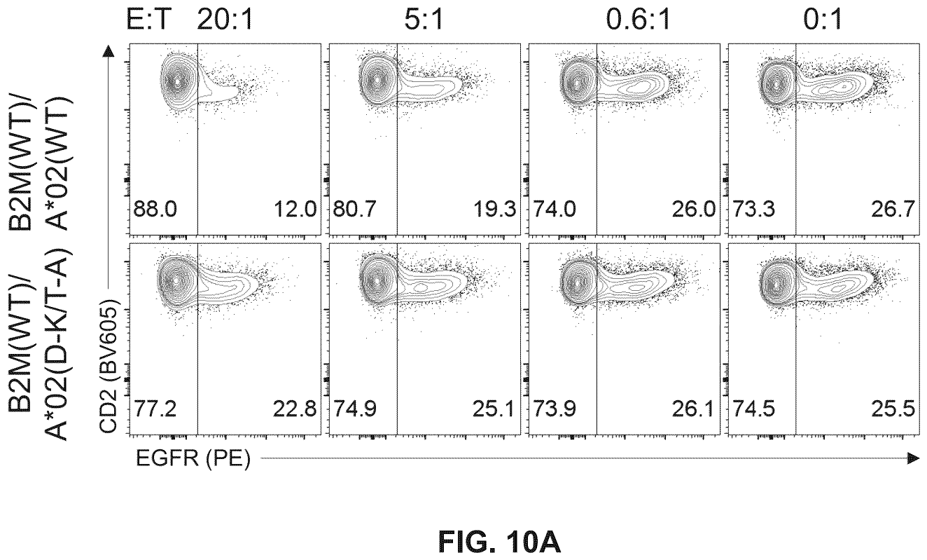

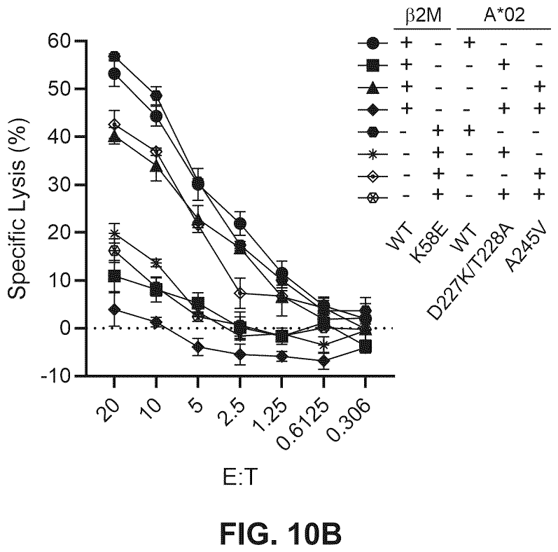

A cytotoxicity assay was undertaken to evaluate whether the transduced B2M KO T cells expressing the HLA-A2 single chain dimers were protected from alloreactive T cell lysis, and to what degree (FIGS. 10A and 10B). The B2M KO T cells expressing the HLA-A2 single chain dimers (i.e., B2M/HLA-A*02; B2M/HLA-A*02 (D227K/T228A); B2M/HLA-A*02 (A245V); B2M/HLA-A*02 (D227K/T228A/A245V); B2M (K58E)/HLA-A*02; B2M (K58E)/HLA-A*02 (D227K/T228A); B2M (K58E)/HLA-A*02 (A245V); and B2M (K58E)/HLA-A*02 (D227K/T228A/A245V)) were co-cultured for 48 hours with HLA-A2 alloreactive T cells and cell counts were then performed using flow cytometry to evaluate levels of cell lysis. B2M KO T cells expressing HLA-A2 single-chain dimers containing an HLA-A2 (D227K/T228A) domain were found to show relatively high levels of protection from alloreactive T cell lysis (FIGS. 10A and 10B).

Surface-expression of the HLA class-I single-chain dimers and trimers listed in Table 9 in B2M knock-out T cells was confirmed using flow cytometry (FIG. 11). All of the HLA class-I single-chain dimers and trimers were surface-expressed within the B2M knock-out T cells.

TABLE 9
Description of exemplary HLA class-I single-chain dimers and trimers.²

| | | | | For- | | NK |
|---|---|---|---|---|---|---|
| Description | ID# | HLA-X | TMD | mat | Motif | Ligand |
| B2M/HLA-C*05(C1G) | 246 | C*05:01 | WT | SCD | | |
| B2M/HLA-C*05(C1G)- | 247 | C*05:01 | CD4 | SCD | | |
| CD4TM | | | | | | |
| B2M/HLA-C*04 | 248 | C*04:01 | WT | SCD | C2 | KIR2DL1 |
| B2M/HLA-C*04- | 249 | C*04:01 | CD4 | SCD | | (CD158a) |
| CD4TM | | | | | | |
| cPep/B2M/HLA-C*04 | 250 | C*04:01 | WT | SCT | | |
| cPep/B2M/HLA- | 251 | C*04:01 | CD4 | SCT | | |
| C*04-CD4TM | | | | | | |
| B2M/HLA-C*07(C1G) | 252 | C*07:01 | WT | SCD | C1 | KIR2DL2 |
| B2M/HLA-C*07(C1G)- | 253 | C*07:01 | CD4 | SCD | | (CD158b1) |
| CD4TM | | | | | | & |
| cPep/B2M/HLA- | 254 | C*07:01 | WT | SCT | | KIR2DL3 |
| C*07(C1G) | | | | | | (CD158b2) |
| cPep/B2M/HLA- | 255 | C*07:01 | CD4 | SCT | | |
| C*07(C1G)-CD4TM | | | | | | |
| B2M/HLA-B*57 | 256 | B*57:01 | WT | SCD | Bw4 | KIR3DL1 |
| B2M/HLA-B*57- | 257 | B*57:01 | CD4 | SCD | | (CD158e1) |
| CD4TM | | | | | | |
| cPep/B2M/HLA-B*57 | 258 | B*57:01 | WT | SCT | | |
| cPep/B2M/HLA- | 259 | B*57:01 | CD4 | SCT | | |
| B*57-CD4TM | | | | | | |

²Table 9 lists the natural killer (NK) cell inhibitor motif (see. e.g., Sim, et al. “Canonical and Cross-reactive Binding of NK Cell Inhibitory Receptors to Hla-C Allotypes is Dictated by Peptides Bound to HLA-C, Frontiers in Immunology, vol. 8, art. 193 (2017), the disclosure of which is incorporated herein by reference in its entirety for all purposes) comprised by each HLA-X domain and the corresponding NK cell ligand. In Table 9, “TMD” indicates a transmembrane domain, which is either a wild-type (WT) transmembrane domain corresponding to the indicated HLA-X domain, or a CD4 domain. The identification numbers (ID#'s) correspond to those used in FIGS. 11-13. In Table 9, “SCD” indicates “single-chain dimer” and “SCT” indicates “single-chain trimer.”

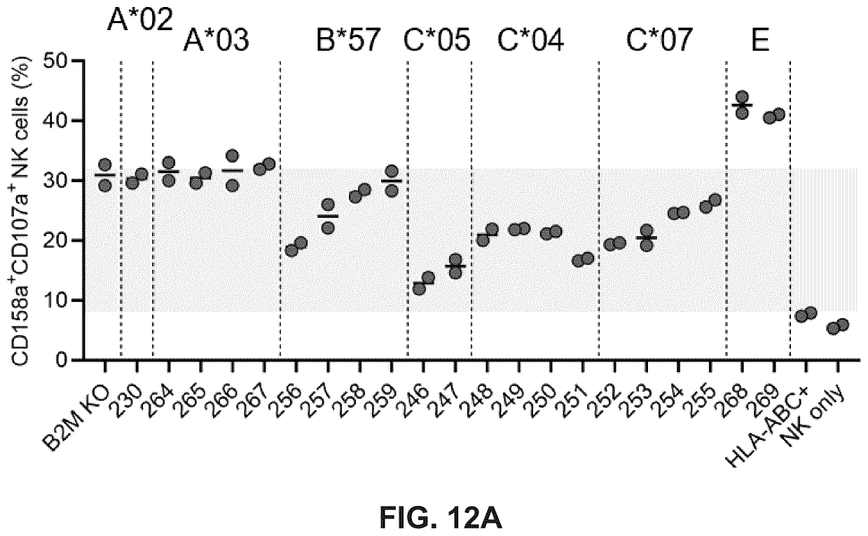

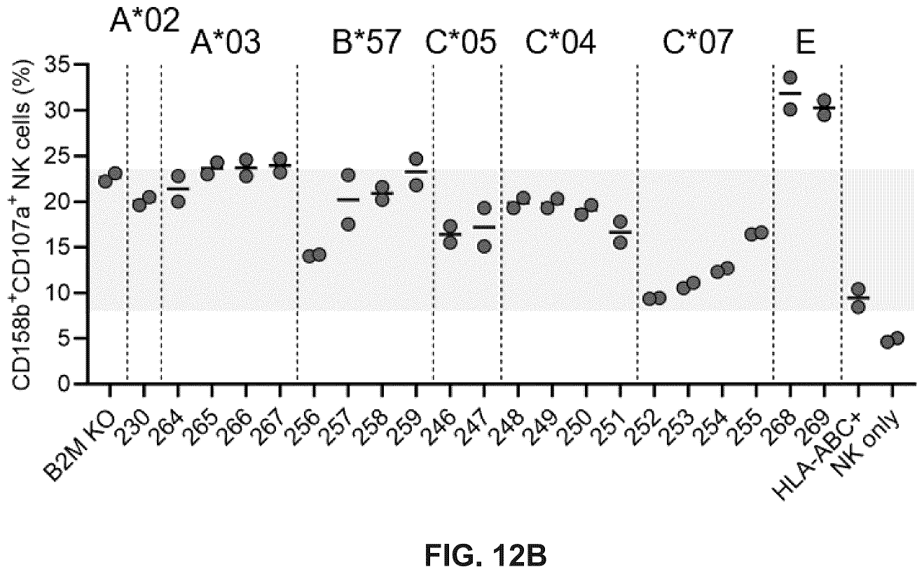

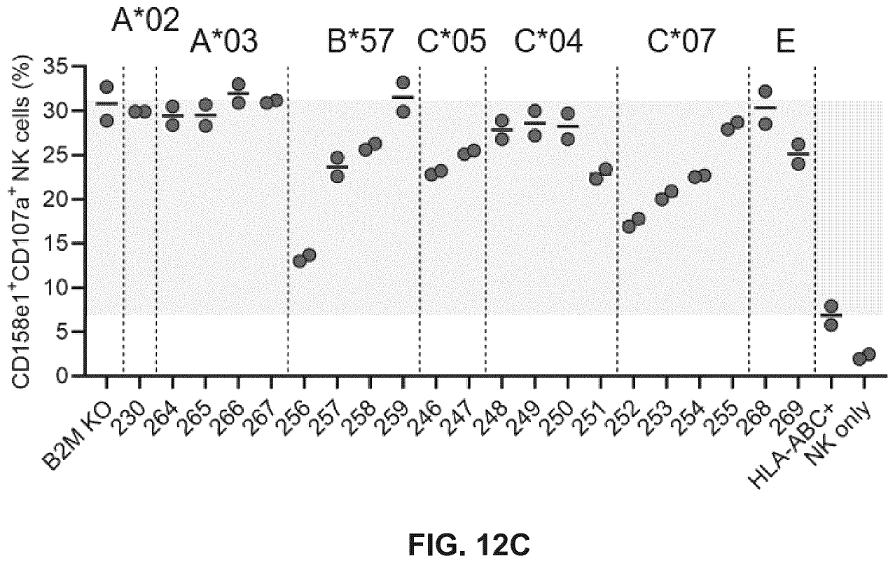

Experiments were next undertaken to determine whether expression of the HLA class-I single-chain dimers and trimers in B2M knock-out T cells was protective against lysis by natural killer cells. B2M knock-out T cells expressing the following HLA class-I single-chain dimers and trimers were co-cultured with primary human NK cells at a 1:1 effector-to-target ratio for 6 hours: B2M/HLA-A*02; B2M/HLA-C*05 (C1G); B2M/HLA-C*05 (C1G)-CD4TM; B2M/HLA-C*04; B2M/HLA-C*04-CD4TM; cPep/B2M/HLA-C*04; cPep/B2M/HLA-C*04-CD4TM; B2M/HLA-C*07 (C1G); B2M/HLA-C*07 (C1G)-CD4TM; cPep/B2M/HLA-C*07 (C1G); cPep/B2M/HLA-C*07 (C1G)-CD4TM; B2M/HLA-B*57; B2M/HLA-B*57-CD4TM; cPep/B2M/HLA-B*57; cPep/B2M/HLA-B*57-CD4TM; B2M/HLA-A*03; B2M/HLA-A*03-CD4TM; cPep/B2M/HLA-A*03; cPep/B2M/HLA-A*03-CD4TM; B2M/HLA-E-CD4TM, and cPep/B2M/HLA-E-CD4TM (see Tables 8A and 9). The frequency of degranulating (CD107a+) NK cells was measured using flow cytometry staining the cells using an anti-CD107a BV650 antibody. Also, the NK cells were stained using antibodies against the inhibitor receptors CD158a (FIG. 12A), CD158b (FIG. 12B), and CD158e1 (FIG. 12C) to measure degranulation in the CD158a+, CD158b+, and CD158e1+ natural killer cell sub-populations (i.e., NK cells of different “KIR types”), respectively. An NK cell surface-expressing a particular KIR ligand (e.g., KIR2DL1 (CD158a), KIR2DL2 (CD158b1), KIR2DL3 (CD158b2), or KIR3DL1 (CD158e1)) can be referred to as having a “KIR-type” corresponding to the ligand(s) that is surface-expressed. As indicated in Table 9 and as shown in FIGS. 12A-12C, particular HLA-X polypeptides (e.g., C*05:01, C*04:01, C*07:01, and B*57:01) inhibited activation of NK cells having particular KIR types to varying degrees depending upon the inhibitory motifs (e.g., C1, C2, or Bw4) contained in the HLA-X polypeptide. It was found that expression of the HLA class-I single-chain dimers and timers containing an HLA-B or HLA-C class-I heavy chain polypeptide domain inhibited NK cell degranulation.

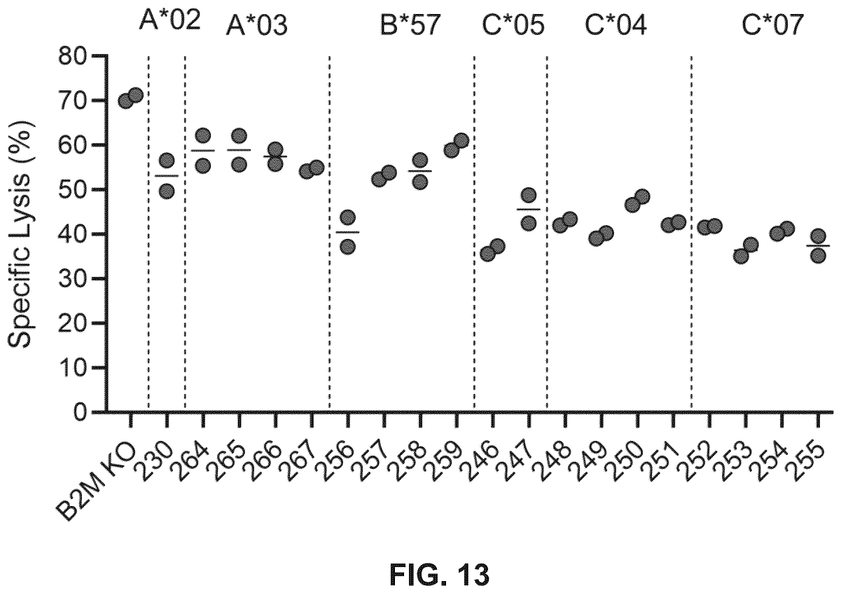

To further evaluate whether expression of the HLA class-I single-chain dimers and trimers in B2M knock-out T cells was protective against lysis by natural killer cells, B2M knock-out T cells expressing the following HLA class-I single-chain dimers (on-target cells) were evenly mixed with unmodified HLA-A+, -B+, C+ cells (off-target cells), and then co-cultured with primary human NK cells at 1:1 and 0:1 (control experiment) effector-to-target ratios for 48 hours: B2M/HLA-A*02; B2M/HLA-C*05 (C1G); B2M/HLA-C*05 (C1G)-CD4TM; B2M/HLA-C*04; B2M/HLA-C*04-CD4TM; cPep/B2M/HLA-C*04; cPep/B2M/HLA-C*04-CD4TM; B2M/HLA-C*07 (C1G); B2M/HLA-C*07 (C1G)-CD4TM; cPep/B2M/HLA-C*07 (C1G); cPep/B2M/HLA-C*07 (C1G)-CD4TM; B2M/HLA-B*57; B2M/HLA-B*57-CD4TM; cPep/B2M/HLA-B*57; cPep/B2M/HLA-B*57-CD4TM; B2M/HLA-A*03; B2M/HLA-A*03-CD4TM; cPep/B2M/HLA-A*03; and cPep/B2M/HLA-A*03-CD4TM (FIG. 13 and Tables 8A and 9). Specific lysis of on-target cells was calculated by measuring the residual frequency of on-target cells in the presence of effector natural killer (NK) cells (1:1 effector-to-target cell ratio) relative to a control containing no effector natural killer cells (i.e., 0:1 effector-to-target cell ratio). FIG. 13 shows the frequency of specific lysis of on-target cells that was mediated by the natural killer cells. Surface-expression in the B2M knock-out T cells of the HLA class-I single-chain dimers conferred protection against NK cell lysis relative to on-target B2M knock-out base edited cells that did not express the HLA class-I single-chain dimers.

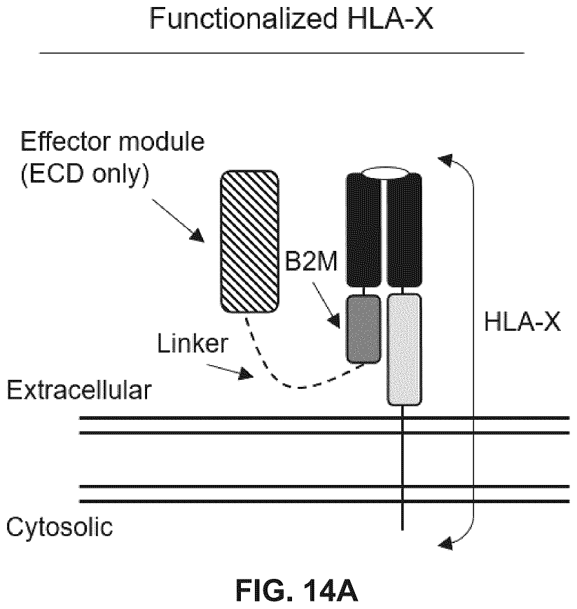

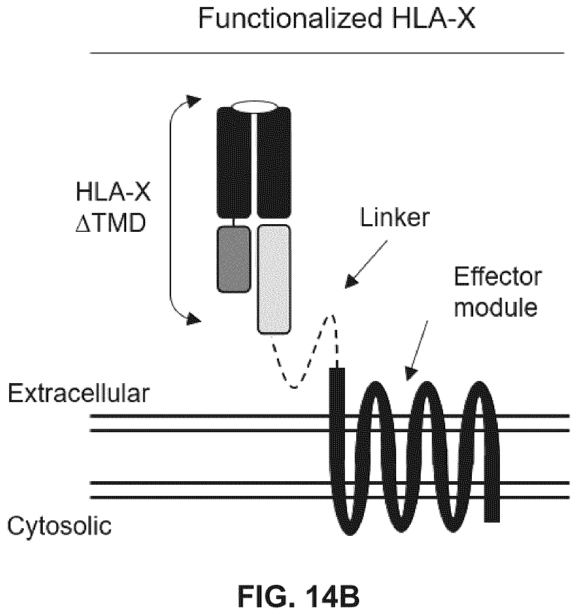

To further improved the ability of the HLA class-I single-chain dimers and trimers to inhibit cell lysis by natural killer or alloreactive T cells, HLA class-I single chain dimers and trimers were prepared that further contained effector domains whose function increased inhibition of alloreactive T and NK cells (FIGS. 14A and 14B). The HLA class-I single-chain dimers and trimers containing the effector domains were referred to as “functionalized” single-chain dimers and trimers. Some functionalized single-chain dimers and trimers were prepared by fusing an HLA class-I single-chain dimer or trimer at the N-terminus to the extracellular domain of a polypeptide that inhibits NK cell and/or T cell activation (e.g., a PD-L1, CTLA4, an additional HLA class-I single chain dimer or trimer, an HLA class-I heavy chain domain, or an HLA-E domain) (see Table 8B). Some functionalized single-chain dimers and trimers were prepared by fusing an HLA class-I single-chain dimer or trimer at the C-terminus to a membrane-bound domain that inhibits NK cell and/or T cell activation (e.g., a CD47 domain) (see Table 8B). In particular, the following functionalized single-chain dimers and trimers were prepared: CTLA4 (ECD)/B2M/HLA-B*57; B2M/HLA-B*57 (ΔTM)-CD47; B2M/HLA-B*57 (ΔTM)/CD47; B2M/HLA-C*05 (ΔTM)/B2M/HLA-B*58; and cPep/B2M/HLA-E (ΔTM)/B2M/HLA-B*57, where ΔTM indicates a deletion of the transmembrane domain of the indicated HLA class-I heavy chain domain.

### Other Embodiments

From the foregoing description, it will be apparent that variations and modifications may be made to the aspects or embodiments described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference. The disclosure may be related to International Patent Applications No. PCT/US22/75021, PCT/US20/13964, PCT/US20/52822, PCT/US20/18178, PCT/US21/52035, PCT/US22/81241, PCT/US23/67780, PCT/US23/68543, the disclosures of which is incorporated herein by reference in their entirety for all purposes.

What is claimed:

1. A fusion polypeptide comprising an HLA heavy chain polypeptide, or a functional fragment thereof, and a beta-2-microglobulin (B2M) polypeptide, or a functional fragment thereof.

2. The fusion polypeptide of claim 1, wherein the HLA heavy chain polypeptide and/or the B2M polypeptide comprise an amino acid alteration that reduces affinity of binding to a CD8 polypeptide relative to a wild-type HLA heavy chain polypeptide and/or B2M polypeptide.

3. The fusion polypeptide of claim 1, wherein the HLA heavy chain polypeptide is an HLA-A, HLA-B, or HLA-C polypeptide.

4. The fusion polypeptide of claim 3, wherein the HLA heavy chain polypeptide comprises one or more amino acid alterations to one or more of amino acid positions 183-274.

5. The fusion polypeptide of claim 4, wherein the HLA heavy chain polypeptide comprises one or more amino acid alterations at an amino acid position selected from the group consisting of A73, D227, T228, and A245.

6. The fusion polypeptide of any one of claims 1-5, wherein the HLA heavy chain polypeptide comprises one or more amino acid alterations selected from the group consisting of A73T, D227K, T228A, and A245V.

7. The fusion polypeptide of claim 6, wherein the HLA heavy chain polypeptide comprises the alterations D227K and T228A, or the alterations D227K, T228A, and A245V.

8. The fusion polypeptide of any one of claims 1-7, wherein the B2M polypeptide comprises an amino acid alteration.

9. The fusion polypeptide of claim 8, wherein the B2M polypeptide comprises an amino acid alteration at position K58.

10. The fusion polypeptide of claim 9, wherein the B2M polypeptide comprises the amino acid alteration K58E.

11. The fusion polypeptide of claim 1 further comprising a cognate peptide (cPep).

12. The fusion polypeptide of claim 11, wherein the cPep comprises from about 8 to about 10 amino acids.

13. The fusion polypeptide of claim 12, wherein the cPep comprises 9 amino acids.

14. The fusion polypeptide of claim 12 or 13, wherein the cPep comprises an amino acid sequence selected from the group consisting of QYDDAVYKL (SEQ ID NO: 520), RYRPGTVAL (SEQ ID NO: 521), LSSPVTKSF (SEQ ID NO: 522), EEVHDLERKY (SEQ ID NO: 523), RLRAEAQVK (SEQ ID NO: 524), IIDKSGAAV (SEQ ID NO: 529; IV9 (AA)), IIDKSGEEV (SEQ ID NO: 530; IV9 (EE)), IIDKSGLAV (SEQ ID NO: 531; IV9 (LA)), IIDKSGSTV (SEQ ID NO: 532; IV9 (WT)), and the cPep sequences listed in Table A.

15. The fusion polypeptide of any one of claims 1-14, wherein the fusion polypeptide comprises one or more linkers.

16. The fusion polypeptide of claim 15, wherein the one or more linkers comprise the amino acid sequence (GGGGS)ₙ (SEQ ID NO: 247).

17. The fusion polypeptide of claim 16, wherein n is 3 or 4.

18. The fusion polypeptide any one of claims 1-17 further comprising a signal peptide.

19. The fusion polypeptide of claim 18, wherein the signal peptide is a B2M signal peptide.

20. The fusion polypeptide of claim 19, wherein the signal peptide comprises the amino acid sequence MSRSVALAVLALLSLSGLEA (SEQ ID NO: 525).

21. The fusion polypeptide of any one of claims 1-20, further comprising a transmembrane domain.

22. The fusion polypeptide of claim 21, wherein the transmembrane domain is a cluster of differentiation 4 (CD4) transmembrane (CD4TM) domain.

23. The fusion polypeptide of claim 22, wherein the CD4TM domain comprises an amino acid sequence with at least about 85% sequence identity to the amino acid sequence MALIVLGGVAGLLLFIGLGIFFCVRC (SEQ ID NO: 437).

24. The fusion polypeptide of any one of claims 1-23, wherein the HLA heavy chain polypeptide comprises a sequence with at least 85% sequence identity to an amino acid sequence selected from the group consisting of:
HLA-A*02 (HLA-A2)
(SEQ ID NO: 441)
GSHSMRYFFTSVSRPGRGEPRFIAVGYVDDTQFVRFDSDAASQRMEPRAPWIEQEGPEYWDGET
RKVKAHSQTHRVDLGTLRGYYNQSEAGSHTVQRMYGCDVGSDWRFLRGYHQYAYDGKDYIALKE
DLRSWTAADMAAQTTKHKWEAAHVAEQLRAYLEGTCVEWLRRYLENGKETLQRTDAPKTHMTHH
AVSDHEATLRCWALSFYPAEITLTWQRDGEDQTQDTELVETRPAGDGTFQKWAAVVVPSGQEQR
YTCHVQHEGLPKPLTLRWEPSSQPTIPIVGIIAGLVLFGAVITGAVVAAVMWRRKSSDRKGGSY
SQAASSDSAQGSDVSLTACKV;
HLA-A*03
(SEQ ID NO: 442)
GSHSMRYFFTSVSRPGRGEPRFIAVGYVDDTQFVRFDSDAASQRMEPRAPWIEQEGPEYWDQET
RNVKAQSQTDRVDLGTLRGYYNQSEAGSHTIQIMYGCDVGSDGRFLRGYRQDAYDGKDYIALNE
DLRSWTAADMAAQITKRKWEAAHEAEQLRAYLDGTCVEWLRRYLENGKETLQRTDPPKTHMTHH
PISDHEATLRCWALGFYPAEITLTWQRDGEDQTQDTELVETRPAGDGTFQKWAAVVVPSGEEQR
YTCHVQHEGLPKPLTLRWELSSQPTIPIVGIIAGLVLLGAVITGAVVAAVMWRRKSSDRKGGSY
TQAASSDSAQGSDVSLTACKV;
HLA-B*57
(SEQ ID NO: 445)
GSHSMRYFYTAMSRPGRGEPRFIAVGYVDDTQFVRFDSDAASPRMAPRAPWIEQEGPEYWDGET
RNMKASAQTYRENLRIALRYYNQSEAGSHIIQVMYGCDVGPDGRLLRGHDQSAYDGKDYIALNE
DLSSWTAADTAAQITQRKWEAARVAEQLRAYLEGLCVEWLRRYLENGKETLQRADPPKTHVTHH
PISDHEATLRCWALGFYPAEITLTWQRDGEDQTQDTELVETRPAGDRTFQKWAAVVVPSGEEQR
YTCHVQHEGLPKPLTLRWEPSSQSTVPIVGIVAGLAVLAVVVIGAVVAAVMCRRKSSGGKGGSY
SQAACSDSAQGSDVSLTA;
HLA-B*44
(SEQ ID NO: 446)
GSHSMRYFYTAMSRPGRGEPRFITVGYVDDTLFVRFDSDATSPRKEPRAPWIEQEGPEYWDRET
QISKTNTQTYRENLRTALRYYNQSEAGSHIIQRMYGCDVGPDGRLLRGYDQDAYDGKDYIALNE
DLSSWTAADTAAQITQRKWEAARVAEQDRAYLEGLCVESLRRYLENGKETLQRADPPKTHVTHH
PISDHEVTLRCWALGFYPAEITLTWQRDGEDQTQDTELVETRPAGDRTFQKWAAVVVPSGEEQR
YTCHVQHEGLPKPLTLRWEPSSQSTVPIVGIVAGLAVLAVVVIGAVVAAVMCRRKSSGGKGGSY
SQAACSDSAQGSDVSLTA;
HLA-C*04
(SEQ ID NO: 449)
GSHSMRYFSTSVSWPGRGEPRFIAVGYVDDTQFVRFDSDAASPRGEPREPWVEQEGPEYWDRET
QKYKRQAQADRVNLRKLRGYYNQSEDGSHTLQRMFGCDLGPDGRLLRGYNQFAYDGKDYIALNE
DLRSWTAADTAAQITQRKWEAAREAEQRRAYLEGTCVEWLRRYLENGKETLQRAEHPKTHVTHH
PVSDHEATLRCWALGFYPAEITLTWQWDGEDQTQDTELVETRPAGDGTFQKWAAVVVPSGEEQR
YTCHVQHEGLPEPLTLRWKPSSQPTIPIVGIVAGLAVLAVLAVLGAMVAVVMCRRKSSGGKGGS
CSQAASSNSAQGSDESLIACKA;
HLA-C*05
(SEQ ID NO: 450)
CSHSMRYFYTAVSRPGRGEPRFIAVGYVDDTQFVQFDSDAASPRGEPRAPWVEQEGPEYWDRET
QKYKRQAQTDRVNLRKLRGYYNQSEAGSHTLQRMYGCDLGPDGRLLRGYNQFAYDGKDYIALNE
DLRSWTAADKAAQITQRKWEAAREAEQRRAYLEGTCVEWLRRYLENGKKTLQRAEHPKTHVTHH
PVSDHEATLRCWALGFYPAEITLTWQRDGEDQTQDTELVETRPAGDGTFQKWAAVVVPSGEEQR
YTCHVQHEGLPEPLTLRWGPSSQPTIPIVGIVAGLAVLAVLAVLGAVMAVVMCRRKSSGGKGGS
CSQAASSNSAQGSDESLIACKA;
and
HLA-C*07
(SEQ ID NO: 451)
CSHSMRYFDTAVSRPGRGEPRFISVGYVDDTQFVRFDSDAASPRGEPRAPWVEQEGPEYWDRET
QKYKRQAQADRVSLRNLRGYYNQSEDGSHTLQRMSGCDLGPDGRLLRGYDQSAYDGKDYIALNE
DLRSWTAADTAAQITQRKLEAARAAEQLRAYLEGTCVEWLRRYLENGKETLQRAEPPKTHVTHH
PLSDHEATLRCWALGFYPAEITLTWQRDGEDQTQDTELVETRPAGDGTFQKWAAVVVPSGQEQR
YTCHMQHEGLQEPLTLSWEPSSQPTIPIMGIVAGLAVLVVLAVLGAVVTAMMCRRKSSGGKGGS
CSQAACSNSAQGSDESLITCKA.

25. The fusion polypeptide of any one of claims 1-24, wherein the B2M polypeptide comprises a sequence with at least 85% sequence identity to the amino acid sequence
(SEQ ID NO: 426)
IQRTPKIQVYSRHPAENGKSNFLNCYVSGFHPSDIEVDLLKNGERIEKV
EHSDLSFSKDWSFYLLYYTEFTPTEKDEYACRVNHVTLSQPKIVKWDRD
M.

26. The fusion polypeptide of claim 1, wherein the fusion polypeptide comprises from N-terminus to C-terminus:
A) the B2M polypeptide and the HLA heavy chain polypeptide;
B) the B2M polypeptide, a linker, and the HLA heavy chain polypeptide;
C) a cognate peptide (cPep), the B2M polypeptide, and the HLA heavy chain polypeptide;
D) a cognate peptide (cPep), the B2M polypeptide, a linker, and the HLA heavy chain polypeptide;
E) a cognate peptide (cPep), a linker, the B2M polypeptide, a linker, and the HLA heavy chain polypeptide;
F) a signal peptide, the B2M polypeptide, a linker, and the HLA heavy chain polypeptide;
G) a signal peptide, the B2M polypeptide and the HLA heavy chain polypeptide;
H) a signal peptide, the B2M polypeptide, a linker, and the HLA heavy chain polypeptide;
I) a signal peptide, a cognate peptide (cPep), the B2M polypeptide, and the HLA heavy chain polypeptide;
J) a signal peptide, a cognate peptide (cPep), the B2M polypeptide, a linker, and the HLA heavy chain polypeptide; or
K) a signal peptide, a cognate peptide (cPep), a linker, the B2M polypeptide, a linker, and the HLA heavy chain polypeptide.

27. The fusion polypeptide of claim 26 further comprising a transmembrane domain C-terminal to the HLA heavy chain polypeptide.

28. A fusion polypeptide comprising an amino acid sequence with at least 85% sequence identity to a polypeptide sequence listed in Table 8A.

29. A polynucleotide encoding the fusion polypeptide of any one of claims 1-26.

30. The polynucleotide of claim 29, wherein the polynucleotide comprises a sequence with at least 85% sequence identity to a polynucleotide sequence listed in Table 8B.

31. A vector comprising a polynucleotide encoding the fusion polypeptide of any one of claims 1-26.

32. The vector of claim 31, wherein the vector is a viral vector or a transposon.

33. The vector of claim 32, wherein the vector is a lentiviral vector.

34. A cell comprising the fusion polypeptide of any one of claims 1-28, the polynucleotide of claim 29 or claim 30, or the vector of any one of claims 31-33.

35. The cell of claim 34, wherein the cell is an immune cell, a hepatocyte, a stem cell, an induced pluripotent stem cell, an islet cell, or a progenitor thereof.

36. A pharmaceutical composition comprising the polynucleotide of claim 29 or claim 30, the vector of any one of claims 31-33, or the cell of claim 34 or claim 35, and a pharmaceutically acceptable excipient.

37. A method for preparing a modified cell, the method comprising:
a) modifying a cell to knock-out expression of an endogenous beta-2-microglobulin (B2M) polypeptide in the cell; and
b) contacting the cell with a polynucleotide encoding a fusion polypeptide comprising an HLA heavy chain polypeptide, or a functional fragment thereof, and a beta-2-microglobulin (B2M) polypeptide, or a functional fragment thereof, and expressing the polypeptide in the cell.

38. The method of claim 37, wherein the HLA heavy chain polypeptide and/or the B2M polypeptide comprise an amino acid alteration that that reduces affinity of binding to a CD8 polypeptide relative to a wild-type HLA heavy chain polypeptide and/or B2M polypeptide.

39. The method of claim 37, wherein the HLA heavy chain polypeptide is an HLA-A, HLA-B, HLA-C, or HLA-E polypeptide.

40. The method of claim 37, wherein the HLA heavy chain polypeptide comprises one or more amino acid alterations to one or more of amino acid positions 183-274.

41. The method of claim 37, wherein the HLA heavy chain polypeptide comprises one or more amino acid alterations at an amino acid position selected from the group consisting of A73, D227, T228, and A245.

42. The method of any one of claims 37-41, wherein the HLA heavy chain polypeptide comprises one or more amino acid alterations selected from the group consisting of A73T, D227K, T228A, and A245V.

43. The method of claim 42, wherein the HLA heavy chain polypeptide comprises the alterations D227K and T228A, or the alterations D227K, T228A, and A245V.

44. The method of any one of claims 37-43, wherein the B2M polypeptide comprises an amino acid alteration.

45. The method of claim 44, wherein the B2M polypeptide comprises an amino acid alteration at position K58.

46. The method of claim 45, wherein the B2M polypeptide comprises the amino acid alteration K58E.

47. The method of claim 37 wherein the fusion polypeptide further comprises a cognate peptide (cPep).

48. The method of claim 47, wherein the cPep comprises from about 8 to about 10 amino acids.

49. The method of claim 48, wherein the cPep comprises 9 amino acids.

50. The method of claim 48 or claim 49, wherein the cPep comprises an amino acid sequence selected from the group consisting of QYDDAVYKL (SEQ ID NO: 520), RYRPGTVAL (SEQ ID NO: 521), LSSPVTKSF (SEQ ID NO: 522), EEVHDLERKY (SEQ ID NO: 523), RLRAEAQVK (SEQ ID NO: 524), IIDKSGAAV (SEQ ID NO: 529; IV9 (AA)), IIDKSGEEV (SEQ ID NO: 530; IV9 (EE)), IIDKSGLAV (SEQ ID NO: 531; IV9 (LA)), IIDKSGSTV (SEQ ID NO: 532; IV9 (WT)), and the cPep sequences listed in Table A.

51. The method of any one of claims 37-50, wherein fusion polypeptide comprises one or more linkers.

52. The method of claim 51, wherein the one or more linkers comprise the amino acid sequence (GGGGS)ₙ (SEQ ID NO: 247).

53. The method of claim 52, wherein n is 3 or 4.

54. The method any one of claims 37-53, wherein the fusion polypeptide further comprises a signal peptide.

55. The method of claim 54, wherein the signal peptide is a B2M signal peptide.

56. The method of claim 55, wherein the signal peptide comprises the amino acid sequence MSRSVALAVLALLSLSGLEA (SEQ ID NO: 525).

57. The method of any one of claims 37-56, wherein the fusion polypeptide further comprises a transmembrane domain.

58. The method of claim 57, wherein the transmembrane domain is a cluster of differentiation 4 (CD4) transmembrane (CD4TM) domain.

59. The method of claim 58, wherein the CD4TM domain comprises an amino acid sequence with at least about 85% sequence identity to the amino acid sequence
(SEQ ID NO: 437)
MALIVLGGVAGLLLFIGLGIFFCVRC.

60. The method of any one of claims 37-59, wherein the HLA heavy chain polypeptide comprises a sequence with at least 85% sequence identity to an amino acid sequence selected from the group consisting of:
HLA-A*02 (HLA-A2)
(SEQ ID NO: 441)
GSHSMRYFFTSVSRPGRGEPRFIAVGYVDDTQFVRFDSDAASQRMEPRAPWIEQEGPEYWDGET
RKVKAHSQTHRVDLGTLRGYYNQSEAGSHTVQRMYGCDVGSDWRFLRGYHQYAYDGKDYIALKE
DLRSWTAADMAAQTTKHKWEAAHVAEQLRAYLEGTCVEWLRRYLENGKETLQRTDAPKTHMTHH
AVSDHEATLRCWALSFYPAEITLTWQRDGEDQTQDTELVETRPAGDGTFQKWAAVVVPSGQEQR
YTCHVQHEGLPKPLTLRWEPSSQPTIPIVGIIAGLVLFGAVITGAVVAAVMWRRKSSDRKGGSY
SQAASSDSAQGSDVSLTACKV;
HLA-A*03
(SEQ ID NO: 442)
GSHSMRYFFTSVSRPGRGEPRFIAVGYVDDTQFVRFDSDAASQRMEPRAPWIEQEGPEYWDQET
RNVKAQSQTDRVDLGTLRGYYNQSEAGSHTIQIMYGCDVGSDGRFLRGYRQDAYDGKDYIALNE
DLRSWTAADMAAQITKRKWEAAHEAEQLRAYLDGTCVEWLRRYLENGKETLQRTDPPKTHMTHH
PISDHEATLRCWALGFYPAEITLTWQRDGEDQTQDTELVETRPAGDGTFQKWAAVVVPSGEEQR
YTCHVQHEGLPKPLTLRWELSSQPTIPIVGIIAGLVLLGAVITGAVVAAVMWRRKSSDRKGGSY
TQAASSDSAQGSDVSLTACKV;
HLA-B*57
(SEQ ID NO: 445)
GSHSMRYFYTAMSRPGRGEPRFIAVGYVDDTQFVRFDSDAASPRMAPRAPWIEQEGPEYWDGET
RNMKASAQTYRENLRIALRYYNQSEAGSHIIQVMYGCDVGPDGRLLRGHDQSAYDGKDYIALNE
DLSSWTAADTAAQITQRKWEAARVAEQLRAYLEGLCVEWLRRYLENGKETLQRADPPKTHVTHH
PISDHEATLRCWALGFYPAEITLTWQRDGEDQTQDTELVETRPAGDRTFQKWAAVVVPSGEEQR
YTCHVQHEGLPKPLTLRWEPSSQSTVPIVGIVAGLAVLAVVVIGAVVAAVMCRRKSSGGKGGSY
SQAACSDSAQGSDVSLTA;
HLA-B*44
(SEQ ID NO: 446)
GSHSMRYFYTAMSRPGRGEPRFITVGYVDDTLFVRFDSDATSPRKEPRAPWIEQEGPEYWDRET
QISKTNTQTYRENLRTALRYYNQSEAGSHIIQRMYGCDVGPDGRLLRGYDQDAYDGKDYIALNE
DLSSWTAADTAAQITQRKWEAARVAEQDRAYLEGLCVESLRRYLENGKETLQRADPPKTHVTHH
PISDHEVTLRCWALGFYPAEITLTWQRDGEDQTQDTELVETRPAGDRTFQKWAAVVVPSGEEQR
YTCHVQHEGLPKPLTLRWEPSSQSTVPIVGIVAGLAVLAVVVIGAVVAAVMCRRKSSGGKGGSY
SQAACSDSAQGSDVSLTA;
HLA-C*04
(SEQ ID NO: 449)
GSHSMRYFSTSVSWPGRGEPRFIAVGYVDDTQFVRFDSDAASPRGEPREPWVEQEGPEYWDRET
QKYKRQAQADRVNLRKLRGYYNQSEDGSHTLQRMFGCDLGPDGRLLRGYNQFAYDGKDYIALNE
DLRSWTAADTAAQITQRKWEAAREAEQRRAYLEGTCVEWLRRYLENGKETLQRAEHPKTHVTHH
PVSDHEATLRCWALGFYPAEITLTWQWDGEDQTQDTELVETRPAGDGTFQKWAAVVVPSGEEQR
YTCHVQHEGLPEPLTLRWKPSSQPTIPIVGIVAGLAVLAVLAVLGAMVAVVMCRRKSSGGKGGS
CSQAASSNSAQGSDESLIACKA;
HLA-C*05
(SEQ ID NO: 450)
CSHSMRYFYTAVSRPGRGEPRFIAVGYVDDTQFVQFDSDAASPRGEPRAPWVEQEGPEYWDRET
QKYKRQAQTDRVNLRKLRGYYNQSEAGSHTLQRMYGCDLGPDGRLLRGYNQFAYDGKDYIALNE
DLRSWTAADKAAQITQRKWEAAREAEQRRAYLEGTCVEWLRRYLENGKKTLQRAEHPKTHVTHH
PVSDHEATLRCWALGFYPAEITLTWQRDGEDQTQDTELVETRPAGDGTFQKWAAVVVPSGEEQR
YTCHVQHEGLPEPLTLRWGPSSQPTIPIVGIVAGLAVLAVLAVLGAVMAVVMCRRKSSGGKGGS
CSQAASSNSAQGSDESLIACKA;
and
HLA-C*07
(SEQ ID NO: 451)
CSHSMRYFDTAVSRPGRGEPRFISVGYVDDTQFVRFDSDAASPRGEPRAPWVEQEGPEYWDRET
QKYKRQAQADRVSLRNLRGYYNQSEDGSHTLQRMSGCDLGPDGRLLRGYDQSAYDGKDYIALNE
DLRSWTAADTAAQITQRKLEAARAAEQLRAYLEGTCVEWLRRYLENGKETLQRAEPPKTHVTHH
PLSDHEATLRCWALGFYPAEITLTWQRDGEDQTQDTELVETRPAGDGTFQKWAAVVVPSGQEQR
YTCHMQHEGLQEPLTLSWEPSSQPTIPIMGIVAGLAVLVVLAVLGAVVTAMMCRRKSSGGKGGS
CSQAACSNSAQGSDESLITCKA.

61. The method of any one of claims 37-60, wherein the B2M polypeptide comprises a sequence with at least 85% sequence identity to the amino acid sequence
(SEQ ID NO: 426)
IQRTPKIQVYSRHPAENGKSNFLNCYVSGFHPSDIEVDLLKNGERIEKV
EHSDLSFSKDWSFYLLYYTEFTPTEKDEYACRVNHVTLSQPKIVKWDRD
M.

62. The method of claim 37, wherein the fusion polypeptide comprises from N-terminus to C-terminus:
A) the B2M polypeptide and the HLA heavy chain polypeptide;
B) the B2M polypeptide, a linker, and the HLA heavy chain polypeptide;
C) a cognate peptide (cPep), the B2M polypeptide, and the HLA heavy chain polypeptide;
D) a cognate peptide (cPep), the B2M polypeptide, a linker, and the HLA heavy chain polypeptide;
E) a cognate peptide (cPep), a linker, the B2M polypeptide, a linker, and the HLA heavy chain polypeptide;
F) a signal peptide, the B2M polypeptide, a linker, and the HLA heavy chain polypeptide;
G) a signal peptide, the B2M polypeptide and the HLA heavy chain polypeptide;
H) a signal peptide, the B2M polypeptide, a linker, and the HLA heavy chain polypeptide;
I) a signal peptide, a cognate peptide (cPep), the B2M polypeptide, and the HLA heavy chain polypeptide;
J) a signal peptide, a cognate peptide (cPep), the B2M polypeptide, a linker, and the HLA heavy chain polypeptide; or
K) a signal peptide, a cognate peptide (cPep), a linker, the B2M polypeptide, a linker, and the HLA heavy chain polypeptide.

63. The method of claim 52 further comprising a transmembrane domain C-terminal to the HLA heavy chain polypeptide.

64. The method of any one of claims 37-63, further comprising contacting the cell with a vector comprising the polynucleotide.

65. The method of claim 64, wherein the vector is a viral vector or a transposon.

66. The method of claim 65, wherein the vector is a lentiviral vector.

67. The method of any one of claims 37-66, wherein modifying the cell to knock-out expression of B2M is carried out using base editing.

68. The method of claim 67, wherein the base editing comprises contacting the cell with a base editor and a guide polynucleotide targeting the base editor to effect an alteration of a nucleobase of an endogenous B2M gene in the cell, thereby knocking out expression of the endogenous B2M polypeptide in the cell.

69. The method of claim 68, wherein the base editor is a cytidine deaminase base editor, an adenosine deaminase base editor, or a cytidine adenosine deaminase base editor.

70. The method of claim 69, wherein the adenosine deaminase is TadA or a TadA variant.

71. The method of claim 70, wherein the TadA variant is a TadA*8 or TadA*9.

72. The method of claim 69, wherein the cytidine deaminase is APOBEC or an APOBEC variant.

73. The method of claim 69, wherein the deaminase is TadA*8.20.

74. The method of any one of claims 68-73, wherein the guide polynucleotide comprises a spacer comprising the nucleotide sequence CUUACCCCACUUAACUAUCU (SEQ ID NO: 537).

75. The method of any one of claims 68-74, wherein the guide polynucleotide comprises a scaffold comprising the nucleotide sequence
(SEQ ID NO: 626)
GUUUUAGAGCUAGAAAUAGCAAGUUAAAAUAAGGCUAGUCCGUUAUCA
ACUUGAAAAAGUGGCACCGAGUCGGUGCUUUU.

76. The method of any one of claims 37-75, further comprising expressing a chimeric antigen receptor in the cell.

77. The method of claim 76, wherein the chimeric antigen receptor targets an antigen expressed on the surface of a neoplastic cell.

78. The method of any one of claims 37-60, wherein a natural killer cell contacted with the modified cell shows reduced levels of granulation relative to levels measured when the natural killer cell is contacted with a reference cell.

79. The method of any one of claims 37-78, wherein the cell is a T cell, a natural killer cell, a hepatocyte, a stem cell, an induced pluripotent stem cell, an islet cell, or a progenitor thereof.

80. The method of claim 79, wherein the cell is a CD4+ or CD8+ cell.

81. The method of any one of claims 37-80, wherein the immune cell is an allogeneic cell.

82. The method of any one of claims 37-81, wherein the modified cell shows increased resistance to lysis by an immune effector cell relative to a reference cell.

83. The method of claim 82, wherein lysis is reduced by at least about 60%.

84. The method of claim 82, wherein lysis is reduced by at least about 90%.

85. The method of any one of claims 37-84, wherein an alloreactive T cell contacted with the modified cell shows reduced levels of cytokine production relative to levels produced when the alloreactive T cell is contacted with a reference cell.

86. The method of claim 85, wherein granzyme B (GZMB) secreted by the alloreactive T cell is reduced by at least about 10%.

87. The method of claim 85 or claim 86, wherein granzyme B (GZMB) secreted by the alloreactive T cell is reduced by at least about 50%.

88. The method of any one of any one of claims 85-87, wherein INF-gamma secreted by the alloreactive T cell is reduced by at least about 10%.

89. The method of any one of claims 85-88, wherein INF-gamma secreted by the alloreactive T cell is reduced by at least about 40%.

90. The method any one of claims 85-88, wherein TNF-alpha secreted by the alloreactive T cell is reduced by at least about 10%.

91. The method any one of claims 85-88, wherein TNF-alpha secreted by the alloreactive T cell is reduced by at least about 40%.

92. The method of any one of claims 37-91, wherein the modified cell further comprises virtually undetectable levels of HLA-A and HLA-B.

93. A method for preparing a modified cell, the method comprising:
a) modifying a cell to knock-out expression of an endogenous beta-2-microglobulin (B2M) polypeptide in the cell; and
b) contacting the cell with a polynucleotide encoding a B2M polypeptide, and expressing the B2M polypeptide in the cell, wherein
i) expression of the B2M polypeptide is reduced relative to an unmodified cell; and/or
ii) the B2M polypeptide comprises an alteration at one or more amino acids positions that effects a reduction in binding of the B2M polypeptide to an HLA heavy chain polypeptide

94. The method of claim 93, wherein the B2M polypeptide is expressed in the cell under the control of a promoter.

95. The method of claim 94, wherein the promoter is an EF1a promoter.

96. The method of any one of claims 93-95, wherein the B2M polypeptide comprises an alteration at amino acid position K58, W60, and/or position W95.

97. The method of claim 96, wherein the B2M polypeptide comprises a W60G and/or a W95G amino acid alteration.

98. The method of any one of claims 93-97, wherein modifying the cell to knock-out expression of B2M is carried out using base editing.

99. The method of claim 98, wherein the base editing comprises contacting the cell with a base editor and a guide polynucleotide targeting the base editor to effect an alteration of a nucleobase of an endogenous B2M gene in the cell, thereby knocking out expression of the endogenous B2M polypeptide in the cell.

100. The method of claim 99, wherein the base editor is a cytidine deaminase base editor, an adenosine deaminase base editor, or a cytidine adenosine deaminase base editor.

101. The method of claim 100, wherein the adenosine deaminase is TadA or a TadA variant.

102. The method of claim 101, wherein the TadA variant is a TadA*8 or TadA*9.

103. The method of claim 100, wherein the cytidine deaminase is APOBEC or an APOBEC variant.

104. The method of claim 100, wherein the deaminase is TadA*8.20.

105. The method of any one of claims 99-104, wherein the guide polynucleotide comprises a spacer comprising the nucleotide sequence CUUACCCCACUUAACUAUCU (SEQ ID NO: 537).

106. The method of any one of claims 99-105, wherein the guide polynucleotide comprises a scaffold comprising the nucleotide sequence
(SEQ ID NO: 626)
GUUUUAGAGCUAGAAAUAGCAAGUUAAAAUAAGGCUAGUCCGUUAUCAA
CUUGAAAAAGUGGCACCGAGUCGGUGCUUUU.

107. The method of any one of claims 93-106, further comprising expressing a chimeric antigen receptor in the cell.

108. The method of claim 107, wherein the chimeric antigen receptor targets an antigen expressed on the surface of a neoplastic cell.

109. The method of any one of claims 93-108, wherein a natural killer cell contacted with the modified cell shows reduced levels of granulation relative to levels measured when the natural killer cell is contacted with a reference cell.

110. The method of any one of claims 93-109, wherein the cell is a T cell, a natural killer cell, a hepatocyte, a stem cell, an induced pluripotent stem cell, an islet cell, or a progenitor thereof.

111. The method of claim 110, wherein the cell is a CD4+ or CD8+ cell.

112. The method of any one of claims 93-111, wherein the cell is an allogeneic cell.

113. The method of any one of claims 93-112, wherein the modified immune cell shows increased resistance to lysis by an immune effector cell relative to a reference cell.

114. The method of claim 113, wherein lysis is reduced by at least about 60%.

115. The method of claim 113 or claim 114, wherein lysis is reduced by at least about 90%.

116. The method of any one of claims 93-115, wherein an alloreactive T cell contacted with the modified cell shows reduced levels of cytokine production relative to levels produced when the alloreactive T cell is contacted with a reference cell.

117. The method of claim 116, wherein granzyme B (GZMB) secreted by the alloreactive T cell is reduced by at least about 10%.

118. The method of claim 116 or claim 117, wherein granzyme B (GZMB) secreted by the alloreactive T cell is reduced by at least about 50%.

119. The method of any one of claims 116-118, wherein INF-gamma secreted by the alloreactive T cell is reduced by at least about 10%.

120. The method of any one of claims 116-119, wherein INF-gamma secreted by the alloreactive T cell is reduced by at least about 40%.

121. The method any one of claims 116-120, wherein TNF-alpha secreted by the alloreactive T cell is reduced by at least about 10%.

122. The method any one of claims 116-121, wherein TNF-alpha secreted by the alloreactive T cell is reduced by at least about 40%.

123. The method of any one of claims 93-122, wherein the method effects a reduction in levels of surface expression of an HLA-A, HLA-B, and/or HLA-C polypeptide on the cell relative to a reference cell.

124. The method of claim 123, wherein surface expression is reduced by at least about 40%.

125. The method of claim 123, wherein surface expression is reduced by at least about 80%.

126. The method of any one of claims 93-125, wherein the modified cell further comprises virtually undetectable levels of HLA-A and HLA-B.

127. The method of any one of claims 93-126, wherein expression of the B2M polypeptide is reduced by at least about 75% relative to an unmodified cell.

128. The method of any one of claims 93-127, wherein expression of the B2M polypeptide is reduced by at least about 90% relative to an unmodified cell.

129. A modified cell prepared according to the method of any one of claims 37-128.

130. The modified cell of claim 129, wherein the cell expresses a chimeric antigen receptor that binds an antigen expressed on the surface of a neoplastic cell.

131. The modified cell of claim 130, wherein a natural killer cell contacted with the modified cell shows reduced levels of granulation relative to levels measured when the natural killer cell is contacted with a reference cell.

132. A pharmaceutical composition comprising the modified cell of any one of claims 129-131 and a pharmaceutically acceptable excipient.

133. A method for killing a neoplastic cell, the method comprising contacting the neoplastic cell with the cell of any one of claim 33-35 or 129-131, or the pharmaceutical composition of claim 36 or claim 132.

134. A method for treating a subject having a neoplasia, the method comprising administering to the subject the cell of any one of claim 33-35 or 129-131, or the pharmaceutical composition of claim 36 or claim 132.

135. A kit for use in the method of any one of claim 37-128, 133, or 134, wherein the kit comprises the fusion polypeptide, polynucleotide, vector, pharmaceutical composition, and/or cell of any of the above claims.
